(19) United States
(12) Patent Application Publication
(10) Pub. No.: US 2025/0236607 A1
(43) Pub. Date: Jul. 24, 2025

(54) CYCLIN-DEPENDENT KINASE 9 (CDK9) DEGRADERS AND METHODS OF USING THEREOF

(71) Applicants: OHIO STATE INNOVATION FOUNDATION, Columbus, OH (US); UNIVERSITY OF CINCINNATI, Cincinnati, OH (US)

(72) Inventors: Oluwatosin R. AYINDE, Columbus, OH (US); James R. FUCHS, Columbus, OH (US); John BYRD, Cincinnati, OH (US); Chia SHARPE, Cincinnati, OH (US)

(21) Appl. No.: 18/852,863

(22) PCT Filed: Mar. 30, 2023
(86) PCT No.: PCT/US2023/016989

§ 371 Date: Sep. 30, 2024

Related U.S. Application Data
(60) Provisional application No. 63/325,424, filed on Mar. 30, 2022.
(60) Provisional application No. 63/397,642, filed on Aug. 12, 2022.
(60) Provisional application No. 63/415,718, filed on Oct. 13, 2022.

(51) Int. Cl. C07D 401/14; A61K 47/54

(52) U.S. Cl. CPC C07D 401/14; A61K 47/545

(57) ABSTRACT

Described herein are CDK9 degraders that include a CDK9 binding moiety, such as AT7519 or VIP152, conjugated to a E3 ubiquitin ligase binding moiety, such as thalidomide, lenalidomide, or pomalidomide. These degraders can induce the ubiquitination of CDK9 and promote its degradation in cells. The linker covalently tethering the CDK9 binding moiety to the E3 ubiquitin ligase binding moiety can be selected to tune the solubility profile and potency of the degrader. Accordingly, the present disclosure provides compounds, compositions, kits, uses, and methods for the treatment of cancer (e.g., blood cancers such as acute myeloid leukemia or acute lymphoblastic leukemia).

### CROSS-REFERENCE TO RELATED APPLICATIONS

This application claims benefit of U.S. Provisional Application No. 63/325,424, filed Mar. 30, 2022, U.S. Provisional Application No. 63/397,642, filed Aug. 12, 2022, and U.S. Provisional Application No. 63/415,718, filed Oct. 13, 2022, each of which is hereby incorporated herein by reference in its entirety.

### BACKGROUND

Recently, a new therapeutic strategy to reduce and/or eliminate proteins associated with certain pathological states, PROTACs (proteolysis targeting chimeras; e.g., see U.S.

Patent Application Publication No. 2016/0058872, published Mar. 3, 2016), were developed. PROTACs are heterobifunctional molecules containing two small molecule binding moieties, joined together by a linker. One of the small molecule ligands is designed to bind with high affinity to a target protein in the cell while the other ligand is able to bind with high affinity to an E3 ligase. In the cell, the PROTAC selectively binds to the target protein of interest.

The PROTAC then recruits a specific E3 ligase to the target protein to form a ternary complex with both the target protein and the E3 ligase held in close proximity. The E3 ligase then recruits an E2 conjugating enzyme to the ternary complex. The E2 is then able to ubiquitinate the target protein, labelling an available lysine residue on the protein, and then the E2 dissociates from the ternary complex. The E3 ligase can then recruit additional E2 molecules resulting in poly-ubiquitination of the target protein, labelling the target protein for degradation by the cell's proteasome machinery. The PROTAC can then dissociate from the target protein and initiate another catalytic cycle. The poly-ubiquitinated target protein is recognized and degraded by the proteasome.

The serine/threonine kinase cyclin-dependent kinase 9 (CDK9) facilitates the phosphorylation of specific protein substrates and thereby modulates their stability and/or activation state. CDK9 and its regulatory cyclin T1 assemble the functional positive transcription elongation factor b (P-TEFb) complex, which phosphorylates the C-terminal domain (CTD) of the largest domain of the multiprotein complex RNA polymerase II (Pol II) RPB1/POLR2A. In turn, Pol II transitions from abortive to productive elongation. Therefore, CDK9 is heavily involved in the regulation of transcription. Other CDK9/cyclin T1 phosphorylation targets include EP300, MYOD1, RPB1/POLR2A, and AR as well as the negative elongation factors DSIF and NELF.

Due to its central role transcriptional regulation, which is frequently dysregulated in cancer, CDK9 has become the target of several drug development efforts. Dysregulation of CDK9 has been observed in a number of solid tumors, including prostate cancer, neuroblastoma, hepatocellular carcinoma, and lymphoma. Moreover, osteosarcoma patients with high CDK9 tumor-expression levels have significantly shorter survival than patients with low CDK9 expression. CDK9 pathway dysregulation has likewise been observed in liquid tumors, such as acute myeloid leukemia (AML)
and acute lymphoblastic leukemia (ALL). Even though several CDK9 inhibitors are available, CDK9 is difficult to therapeutically inhibit with small molecules since the structure of its catalytic ATP-binding cleft is similar to many other kinases. Therefore, selective inhibition of CDK9 is challenging.

### SUMMARY

Because kinases such as CDK9 are difficult to target via traditional small molecule inhibition, compounds that can take advantage of cellular machinery involved in protein homeostasis (e.g., ubiquitination and proteasome degradation via PROTAC) can serve as therapeutic agents in targeting CDK9. Described herein are CDK9 degraders that include a CDK9 binding moiety, such as AT7519 or VIP152, conjugated to a E3 ubiquitin ligase binding moiety, such as thalidomide, lenalidomide, or pomalidomide. These degraders can induce the ubiquitination of CDK9 and promote its degradation in cells. The linker covalently tethering the CDK9 binding moiety to the E3 ubiquitin ligase binding moiety can be selected to tune the solubility profile and potency of the degrader. Accordingly, the present disclosure provides compounds, compositions, kits, uses, and methods for the treatment of cancer (e.g., blood cancers such as acute myeloid leukemia or acute lymphoblastic leukemia).

Provided herein are CDK9 degraders defined by Formula I

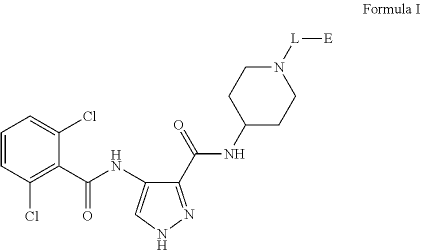

or a pharmaceutically acceptable salt, solvate, hydrate, cocrystal, or prodrug thereof, wherein
L comprises one or more linking groups selected from optionally substituted —C1-10 alkylene-, —O—C1-10 alkylene-, —C1-10 alkenylene-, —O—C1-10 alkenylene-, —C1-10 alkynylene-, —O—C1-10 alkynylene-, -arylene-, -heteroarylene-, -cycloalkylene-, -heterocycloalkylene-, —O—, —S—, —S—S—, —S(O)_{w}—, —C(O)—, —C(O)O—, —OC(O)—, —C(O)S—, —SC(O)—, —OC(O)O—, —N(R^{b})—, —(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—), —O(S)P(OR^{b})O—, and (S)P(O—)₃, wherein w is 1 or 2, and R^{b} is independently hydrogen, optionally substituted alkyl, or optionally substituted aryl; and
E comprises an E3 ubiquitin ligase ligand moiety.

Also provided herein are compounds defined by Formula II below

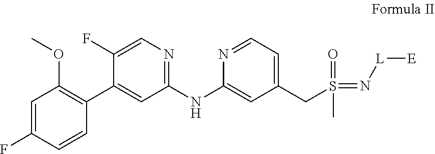

or a pharmaceutically acceptable salt, solvate, hydrate, cocrystal, or prodrug thereof, wherein
L comprises one or more linking groups selected from optionally substituted —C1-10 alkylene-, —O—C1-10 alkylene-, —C1-10 alkenylene-, —O—C1-10 alkenylene-, —C1-10 alkynylene-, —O—C1-10 alkynylene-, -arylene-, -heteroarylene-, -cycloalkylene-, -heterocycloalkylene-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl; and
E comprises an E3 ubiquitin ligase ligand moiety.

In some embodiments of Formula I and Formula II, L comprises one or more linking groups independently selected from optionally substituted —C1-10 alkynylene-, —O—C1-10 alkynylene-, -arylene-, -heteroarylene-, -cycloalkylene-, and -heterocycloalkylene-.

In some embodiments of Formula I and Formula II, L comprises at least two linking groups independently selected from optionally substituted —C1-10 alkynylene-, -heteroarylene-, and -heterocycloalkylene-. In certain embodiments of Formula I and Formula II, L comprises at least two heterocycloalkylene groups.

In some embodiments of Formula I and Formula II, L comprises at least one piperidine moiety.

In some embodiments of Formula I and Formula II, L comprises at least one piperazine moiety.

In some embodiments of Formula I and Formula II, L comprises at least one triazole moiety, such as a 1,2,3-triazole moiety.

In some embodiments of Formula I and Formula II, L comprises at least one alkynyl moiety.

In some embodiments of Formula I and Formula II, L comprises at least one azetidine moiety.

In some embodiments of Formula I and Formula II, L comprises at least two moieties independently selected from a piperidine moiety, a piperazine moiety, a triazole moiety, such as a 1,2,3-triazole moiety, an alkynyl moiety, and an azetidine moiety.

In some embodiments of Formula I and Formula II, L comprises a piperidine moiety, L further comprises at least one additional linking group selected from optionally substituted —C1-10 alkynylene-, —O—C1-10 alkynylene-, -arylene-, -heteroarylene-, -cycloalkylene-, and heterocycloalkylene.

In some embodiments of Formula I and Formula II, L has a length of at least 8 atoms or at least 10 atoms, such as a length of from 8 atoms to 25 atoms, from 10 atoms to atoms, from 8 atoms to 20 atoms, or from 10 atoms to 20 atoms.

In certain embodiments of Formula I and Formula II, L is not defined by the structure below

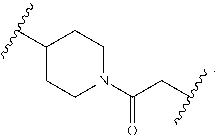 [SMILES: *CC(=O)N1CCC(*)CC1]

Also provided herein are pharmaceutical compositions comprising one or more of CDK9 degraders described herein, or a pharmaceutically acceptable salt thereof, and a physiologically compatible carrier medium.

Also provided are methods of treating or preventing a disease or disorder alleviated by inhibiting and/or indirectly inhibiting CDK9 protein activity in a patient in need of said treatment or prevention, the method comprising administering a therapeutically effective amount of one or more compounds of a CDK9 degrader described herein, or a pharmaceutically acceptable salt thereof.

Also provided are methods of treating cancer in a subject in need thereof, the methods comprising administering a CDK9 degrader described herein, or a pharmaceutically acceptable salt thereof, or a pharmaceutical composition comprising a CDK9 degrader described herein, to the subject. In certain embodiments, the cancer is a solid tumor or a hematological cancer. In certain embodiments, the cancer is a leukemia or a lymphoma. In certain embodiments, the cancer is acute myeloid leukemia or acute lymphoblastic leukemia.

Also provided are methods of promoting the degradation of cyclin-dependent kinase 9 (CDK9) in a subject in need thereof, the methods comprising administering a CDK9 degrader described herein, or a pharmaceutically acceptable salt thereof, or a pharmaceutical composition comprising a CDK9 degrader described herein, to the subject.

Also provided are methods of promoting the ubiquitination of cyclin-dependent kinase 9 (CDK9) by an E3 ubiquitin ligase in a subject in need thereof, the method comprising contacting a CDK9 degrader described herein, or a pharmaceutically acceptable salt thereof, or a pharmaceutical composition comprising a CDK9 degrader described herein, with a tissue. In certain embodiments, the E3 ubiquitin ligase is Cereblon or von Hippel-Findau tumor suppressor (VHF).

Also provided are kits comprising a CDK9 degrader described herein, or a pharmaceutically acceptable salt thereof, or a pharmaceutical composition comprising a CDK9 degrader described herein. In certain embodiments, the kit further comprises instructions for administration (e.g., human administration) and/or use.

### DESCRIPTION OF DRAWINGS

FIG. 1. Western blots showing CDK9 degradation (of both 42 kDa and 55 kDa isoforms) and MCL-1 downstream repression by triazole-containing PROTACs in MV411 cells after 6-hour drugging.

FIGS. 2A-2B. Correlation coefficients (“R²”) and graphical representations of curve fit for triazole-containing PROTACs (except compounds 4.7b and 4.9c) between both Log D and log k′80 (10λ) and − Log(μM IC₅₀) (−1* Log ₁₀ (μM IC₅₀s in MV411 cells (FIG. 2A) and − Log(μM DC₅₀) (−1* Log ₁₀ (μM DC₅₀s in MV411 cells (FIG. 2B).

FIGS. 3A-3C. In vitro cellular potency (against MV411 cells) in human serum (HS) versus fetal bovine serum (FBS) for compounds 4.7b (FIG. 3A) 4.9c (FIG. 3B), and 4,8a (FIG. 3C).

FIGS. 4A-4B. Graphical plot of cytotoxicity (IC₅₀) against lipophilicity (log k′80) for the AT7519 click series (FIG. 4A) and the VIP152-based degrader series (FIG. 4B).

FIG. 5. Graphical plot of kinetic solubility against lipophilicity (10*log k′80) for VIP152-based degraders indicating degraders with above or below average KS and/or lipophilicity.

FIG. 6. Graphical plot of cytotoxicity (IC₅₀) against kinetic solubility (log S) for VIP152-based degraders indicating compounds with above or below average KS and/or in vitro potency.

FIG. 7. Graph of cellular cytotoxicity of all AT7519-inspired triazole containing degraders against predicted and chromatographic log D.

### DETAILED DESCRIPTION

Provided herein are bifunctional compounds that bind CDK9 and recruit an E3 ligase (e.g., Cereblon, VHL) to promote the degradation of CDK9. In one aspect, the disclosure provides compounds of Formula I, and pharmaceutically acceptable salts, solvates, hydrates, polymorphs, co-crystals, tautomers, stereoisomers, isotopically labeled derivatives, prodrugs, and pharmaceutical compositions thereof. In another aspect, the disclosure provides compounds of Formula II, and pharmaceutically acceptable salts, solvates, hydrates, polymorphs, co-crystals, tautomers, stereoisomers, isotopically labeled derivatives, prodrugs, and pharmaceutical compositions thereof. The compounds are useful for the treatment of diseases associated with CDK9 (e.g., cancer) in a subject in need thereof.

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

At various places in the present specification, divalent linking substituents are described. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to in carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cu-m alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “CG-m alkoxy”, employed alone or in combination with other terms, refers to a group of formula —O-alkyl, wherein the alkyl group has n to in carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to in carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “C-n-m alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to in carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NH—C(O)NH₂.

As used herein, the term “Cₙ₋ₘ alkylaminocarbonylamino” refers to a group of formula —NHC(O)NH(alkyl), wherein the alkyl group has n to in carbon atoms. In some embodiments, the alkyl group has 1 to 6, 1 to 4, or 1 to 3 carbon atoms.

As used herein, the term “di(CG-m alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to in carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O)NH₂.

As used herein, the term “carbonyl”, employed alone or in combination with other terms, refers to a —C(═O)— group, which may also be written as C(O).

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to in carbon atoms.

In some embodiments, each alkyl group independently has 1 to 6, 1 to 4, or 1 to 3 carbon atoms.

As used herein, the term “halo” refers to F, Cl, Br, or I. In some embodiments, a halo is F, Cl, or Br. In some embodiments, a halo is F or Cl.

As used herein, “Cₙ₋ₘ haloalkoxy” refers to a group of formula —O-haloalkyl having n to in carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to in carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C3-m). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms.

In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimnidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. AX heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

As used herein, the suffix “-ene” is used to describe a bivalent group with two radical points for forming two covalent bonds with two other moieties. In other words, any of the terms as defined above can be modified with the suffix “-ene” to describe a bivalent version of that moiety. For example, a bivalent aryl ring structure is “arylene,” a bivalent benzene ring structure is “phenylene,” a bivalent heteroaryl ring structure is “heteroarylene,” a bivalent cycloalkyl ring structure is a “cycloalkylene,” a bivalent heterocycloalkyl ring structure is “heterocycloalkylene,” a bivalent cycloalkenyl ring structure is “cycloalkenylene,” a bivalent alkenyl chain is “alkenylene,” and a bivalent alkynyl chain is “alkynylene.”

As used herein, the term “CG-m alkylene”, employed alone or in combination with other terms, refers to a divalent alkyl linking group having n to in carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

In some embodiments, the alkylene moiety contains 2 to 6, 2 to 4, 2 to 3, 1 to 6, 1 to 4, or 1 to 2 carbon atoms.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge.

Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description. The compounds described herein can also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present description. Unless otherwise mentioned or indicated, the chemical designation of a compound encompasses the mixture of all possible stereochemically isomeric forms of that compound.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H, et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p.268 (E. L. Eliel, Ed., Univ. of Notre Dame Press, Notre Dame, IN 1972), each of which is incorporated herein by reference in their entireties. It is also understood that the compounds described herein include all possible regioisomers, and mixtures thereof, which can be obtained in pure form by standard separation procedures known to those skilled in the art, and include, but are not limited to, column chromatography, thin-layer chromatography, and high-performance liquid chromatography.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutic ally acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

The term “prodrug” refers to a compound that have cleavable groups and become by solvolysis or under physiological conditions the compounds described herein, which are pharmaceutically active in vivo. Such examples include, but are not limited to, choline ester derivatives and the like, N-allylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H, Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodmgs. In some cases it is desirable to prepare double ester type prodmgs such as (acyloxy)alkyl esters or ((alkoxycarbonyl)oxy)alkylesters. C₁₋₈ alkyl, C₂₋₈ alkenyl, C₂₋₈ alkynyl, aryl, C7-12 substituted aryl, and C7-12 arylalkyl esters of the compounds described herein may be preferred.

The term “E3 ubiquitin ligase” or “13 ligase” refers to any protein that recruits an E2 ubiquitin-conjugating enzyme that has been loaded with ubiquitin, recognizes a protein substrate, and assists or directly catalyzes the transfer of ubiquitin from the E2 protein to the protein substrate.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for CDK binding and/or promoting the degradation of CDK9. In certain embodiments, a therapeutically effective amount is an amount sufficient for treating a cancer.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL) (e.g., B-cell ALL, T-cell ALL), acute myelocytic leukemia (AML) (e.g., B-cell AXIL, T-cell AML), chronic myelocytic leukemia (CML) (e.g., B-cell CML, T-cell CML), and chronic lymphocytic leukemia (CLL) (e.g., B-cell CLL, T-cell CLL)); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MAL T) lymphoma, nodal marginal zone B-cell lymphoma, splenic marginal zone B-cell lymphoma), primary mediastinal B-cell lymphoma, Burkitt lymphoma, Waldenstrom's macroglobulinemia (WM, lymphoplasmacytic lymphoma), hairy cell leukemia (HCL), immunoblastic large cell lymphoma, precursorB-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangio sarcoma, lymphangioendotheliosarcoma, hemangio sarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g. neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “hematological cancer” refers to cancer that begins in blood-forming tissue, such as the bone marrow, or in the cells of the immune system. Examples of hematologic cancer are leukemia, lymphoma, and multiple myeloma. Hematological cancer is also called blood cancer.

The term “leukemia” refers to broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

### Cyclin-Dependent Kinase 9 (CDK9) Degraders

Provided herein are compounds defined by Formula I below

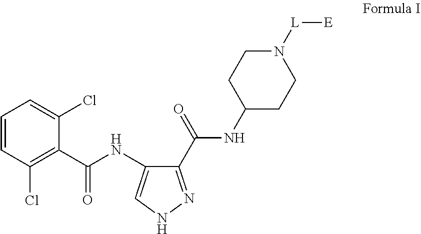

or a pharmaceutically acceptable salt, solvate, hydrate, cocrystal, or prodrug thereof wherein
L comprises one or more linking groups selected from optionally substituted —C₁₋₁₀ alkylene-, —O—C1-10 alkylene-, —C1-10 alkenylene-, —O—C1-10 alkenylene-, —C1-10 alkynylene-, —O-C1-10 alkynylene-, -arylene-, -heteroarylene-, -cycloalkylene-, -heterocycloalkylene-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR)N(R^{b})—, —N(R^{b})S(O)_{w}—, —S(O)_{w}N(R^{b})—, —S(O)_{w}—O—, —OS(O)_{w}—, —OS(O)_{w}—, —O(O)P(OR^{b})O—, (O)P(OR^{b})—, O—O(S)P(OR^{b})O—, and (S)P(OR^{b})₃, wherein w is 1 or 2, and R^{b} is independently hydrogen, optionally substituted alkyl, or optionally substituted aryl; and
E comprises an E3 ubiquitin ligase ligand moiety.

Also provided herein are compounds defined by Formula II below

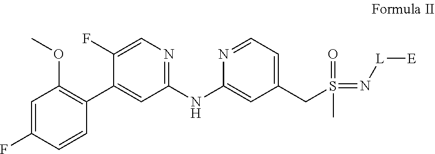

or a pharmaceutically acceptable salt, solvate, hydrate, cocrystal, or prodrug thereof, wherein
L comprises one or more linking groups selected from optionally substituted —C1-10 alkylene-, —O—C1-10 alkylene-, —C1-10 alkenylene-, —O—C1-10 alkenylene-, —C1-10 alkynylene-, —O—C1-10 alkynylene-, -arylene-, -heteroarylene-, -cycloalkylene-, -heterocycloalkylene-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(N(R^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)_{w}, —O(S)P(OR^{b})O—, and (S)P(O—)₃, wherein w is 1 or 2, and R^{b} is independently hydrogen, optionally substituted alkyl, or optionally substituted aryl; and
E comprises an E3 ubiquitin ligase ligand moiety.

In some embodiments of Formula I and Formula II, L comprises one or more linking groups independently selected from optionally substituted —C1-10 alkynylene-, —O—C1-10 alkynylene-, -arylene-, -heteroarylene-, -cycloalkylene-, and -heterocycloalkylene-. For example, in some embodiments of Formula I and Formula II, L can comprise one linking group, two linking groups, three linking groups, four linking groups, or five linking groups independently selected from optionally substituted —C1-10 alkynylene-, —O—C1-10 alkynylene-, -arylene-, -heteroarylene-, -cycloalkylene-, and -heterocycloalkylene-.

In some embodiments of Formula I and Formula II, L comprises at least two linking groups independently selected from optionally substituted —C₁₋₁₀ alkynylene-, -heteroarylene-, and -heterocycloalkylene-. For example, in some embodiments of Formula I and Formula II, L can comprise two linking groups, three linking groups, four linking groups, or five linking groups independently selected from optionally substituted —C1-10 alkynylene-, -heteroarylene-, and -heterocycloalkylene-.

In certain embodiments of Formula I and Formula II, L comprises at least two heterocycloalkylene groups. For example, in some embodiments of Formula I and Formula II, L can comprise two heterocycloalkylene groups, three heterocycloalkylene groups, four heterocycloalkylene groups, or five heterocycloalkylene groups.

In some embodiments of Formula I and Formula II, L comprises at least one piperidine moiety. For example, in some embodiments of Formula I and Formula II, L can comprise one piperidine moiety, two piperidine moieties, or three piperidine moieties.

In some embodiments of Formula I and Formula II, L comprises at least one piperazine moiety. For example, in some embodiments of Formula I and Formula II, L can comprise one piperidine moiety, two piperidine moieties, or three piperazine moieties.

In some embodiments of Formula I and Formula II, L comprises at least one triazole moiety, such as a 1,2,3-triazole moiety. For example, in some embodiments of Formula I and Formula II, L can comprise one piperidine moiety, two piperidine moieties, or three triazole moieties.

In some embodiments of Formula I and Formula II, L comprises at least one alkynyl moiety. For example, in some embodiments of Formula I and Formula II, L can comprise one piperidine moiety, two piperidine moieties, or three alkynyl moieties.

In some embodiments of Formula I and Formula II, L comprises at least one azetidine moiety. For example, in some embodiments of Formula I and Formula II, L can comprise one piperidine moiety, two piperidine moieties, or three azetidine moieties.

In some embodiments of Formula I and Formula II, L comprises at least two moieties independently selected from a piperidine moiety, a piperazine moiety, a triazole moiety, such as a 1,2,3-triazole moiety, an alkynyl moiety, and an azetidine moiety. For example, in some embodiments of Formula I and Formula II, L can comprise two moieties, three moieties, four moieties, or five moieties independently selected from a piperidine moiety, a piperazine moiety, a triazole moiety, such as a 1,2,3-triazole moiety, an alkynyl moiety, and an azetidine moiety.

In some embodiments of Formula I and Formula II, L comprises a piperidine moiety, L further comprises at least one additional linking group selected from optionally substituted —C1-10 alkynylene-, —O—C1-10 alkynylene-, -arylene-, -heteroarylene-, -cycloalkylene-, and -heterocycloalkylene.

In some embodiments of Formula I and Formula II, L has a length of at least 8 atoms or at least 10 atoms, such as a length of from 8 atoms to 25 atoms, from 10 atoms to atoms, from 8 atoms to 20 atoms, or from 10 atoms to 20 atoms.

In certain embodiments of Formula I and Formula II, L is not defined by the structure below

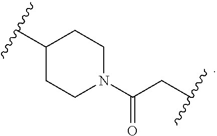 [SMILES: *CC(=O)N1CCC(*)CC1]

E can be any moiety that binds, or can bind, an E3 ubiquitin ligase. In certain embodiments, E is capable of binding an E3 ubiquitin ligase, such as Cereblon or von Hippel-Lindau tumor suppressor (VHL). In certain embodiments, E is capable of binding to multiple different E3 ubiquitin ligases. In certain embodiments, E binds to Cereblon. In certain embodiments, E binds to VHL Non-limiting examples CRBN ligands include thalidomide, lenalidomide, pomalidomide, and any substructure thereof. Any MDM2 ligand is contemplated by the present disclosure. Non-limiting examples MDM2 ligands include idasanutlin, RG7112, RG7388, MI 773/SAR 405838, AMG 232, DS-3032b, RO6839921, RO5045337, RO5503781, CGM-097, MK-8242, and any substructure thereof. Any VHL ligand is contemplated by the present disclosure. Non-limiting examples MDM2 ligands include VHL ligand 1 (VHL-1), VHL ligand 2 (VHL-2), VH032, and any substructure thereof. Examples of suitable E groups are discussed in more detail below.

Human Cereblon (CRBN) is a protein of 442 amino acids with an apparent molecular weight of ˜51 kDa (GenBank: AAH17419). (For the CRBN protein sequence see: Higgins et al, Neurology. 2004, 63, 1927-31. For additional information related to the CRBN structure see Hartmann et al, PLoS One. 2015, 10, e0128342.) Human CRBN contains the N-terminal part (237-amino acids from 81 to 317) of ATP-dependent Lon protease domain without the conserved Walker A and Walker B motifs, 11 casein kinase II phosphorylation sites, 4 protein kinase C phosphorylation sites, 1 N-linked glycosylation site, and 2 myristoylation sites. CRBN is widely expressed in testis, spleen, prostate, liver, pancreas, placenta, kidney, lung, skeletal muscle, ovary, small intestine, peripheral blood leukocyte, colon, brain, and retina. CRBN is located in the cytoplasm, nucleus, and peripheral membrane.

Cereblon is an E3 ubiquitin ligase, and it forms an E3 ubiquitin ligase complex with damaged DNA binding protein 1 (DDB 1), Cullin-4A (CUL4A), and regulator of cullins 1 (ROC₁). This complex ubiquitinates a number of other proteins. Through a mechanism which has not been completely elucidated, Cereblon ubiquitination of target proteins results in increased levels of fibroblast growth factor 8 (FGF8) and fibroblast growth factor 10 (FGF10). FGF8, in turn, regulates a number of developmental processes, such as limb and auditory vesicle formation.

In certain embodiments, E is a modulator, binder, inhibitor, or ligand of Cereblon. In certain embodiments, E is a modulator of Cereblon. In certain embodiments, E is a binder of Cereblon. In certain embodiments, E is an inhibitor of Cereblon. In certain embodiments, E is a ligand of Cereblon. In certain embodiments, E is any modulator, binder, inhibitor, or ligand of Cereblon disclosed in U.S. Patent Application, U.S. Ser. No. 16/523,219, filed Jul. 26, 2019; U.S. Patent Application, U.S. Ser. No. 16/502,529, filed Jul. 3, 2019; U.S. patent application U.S. Ser. No. 16/375,643, filed Apr. 4, 2019; U.S. Patent Application, U.S. Ser. No. 16/230,792, filed Dec. 21, 2018; U.S. patent application U.S. Ser. No. 15/840,950, filed Sep. 13, 2018; US. Patent Application, U.S. Ser. No. 15/996,151, filed Jun. 1, 2018; U.S. Patent Application, U.S. Ser. No. 15/953,108, filed Apr. 13, 2018; U.S. patent application U.S. Ser. No. 15/885,671, filed Jan. 31, 2018; U.S. patent application U.S. Ser. No. 15/881,318, filed Jan. 26, 2018; U.S. patent application U.S. Ser. No. 15/853,166, filed Dec. 22, 2017; U.S. patent application U.S. Ser. No. 15/852,854, filed Dec. 22, 2017; U.S. patent application U.S. Ser. No. 15/851,053, filed Dec. 21, 2017; U.S. patent application U.S. Ser. No. 15/829,541, filed Dec. 1, 2017; U.S. patent application U.S. Ser. No. 15/801,243, filed Nov. 1, 2017; U.S. Patent Application, U.S. Ser. No. 15/730,728, filed Oct. 11, 2017; U.S. patent application U.S. Ser. No. 15/706,064, filed Sep. 15, 2017; U.S. patent application U.S. Ser. No. 15/663,273, filed Jul. 28, 2017 U.S. patent application U.S. Ser. No. 15/230,354, filed Aug. 5, 2016; U.S. patent application U.S. Ser. No. 15/206,497, filed Jul. 11, 2016; U.S. Patent Application, U.S. Ser. No. 15/209,648, filed Jul. 13, 2016; U.S. patent application U.S. Ser. No. 14/822,309, filed Aug. 10, 2015; U.S. Patent Application, U.S. Ser. No. 14/792,414, filed Jul. 6, 2015; U.S. Patent Application, U.S. Ser. No. 14/707,930, filed May 8, 2015; International Patent Application PCT/US2019/040545, filed Jul. 3, 2019; International Patent Application, PCT/US2019/040520, filed Jul. 3, 2019; International Patent Application, PCT/US2019/013481, filed Jan. 14, 2019; International Patent Application, PCT/US2018/052181, filed Sep. 21, 2018; and International Patent Application, PCT/US2013/054663, filed Aug. 13, 2013, each of which is incorporated herein by reference. In certain embodiments, F is a modulator, binder, inhibitor, or ligand of a Cereblon variant. In certain embodiments, E is a modulator, binder, inhibitor, or ligand of a Cereblon isoform.

In certain embodiments, E is defined by Formula E-I:

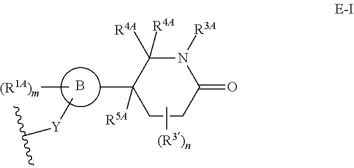

wherein
B is a substituted or unsubstituted monocyclic, bicyclic, or tricyclic fused ring system; Y is —(CH₂)ₖ—, —(CH₂)ₖ—O—, —O)CH₂)ₖ—, —NR^{B}(CH₂)ₖ—, —(CH₂)ₖ—NR^{B}—, —(CH₂)ₖ—(C═O)NR^{b}—, —O)CH₂)ₖ—(C═O)NR^{B}—, —O)CH₂)ₖ—NR^{B}(C═O)—, —NR_{B}(C═O)—(CH₂)ₖ—O—, —NR^{B}(CH₂)ₖ—NR^{B}(C═O)—, or —(CH₂)ₖ—NR(C═O)—
each R^{b} is, independently, hydrogen, or substituted or unsubstituted alkyl; each R^{1A} is, independently, halogen, OH, C₁-C₆ alkyl, or C₁-C₆alkoxy;
each R^{3A} is, independently, hydrogen or C₁-C₃ alkyl;
each R³ is, independently, C₁-C₃ alkyl;
each R^{4A} is, independently, hydrogen or C₁-C₃ alkyl; or two R^{4A}, together with the carbon atom to which they are attached, form a C(O), C₃-C₆ carbocycle, or a 4-6-membered heterocycle comprising 1 or 2 heteroatoms selected from N and O;
R^{5A} is hydrogen, C₁-C₃ alkyl, F, or Cl;
k is 0, 1, 2, 3, 4, 5, or 6;
m is 0, 1, 2 or 3; and
n is 0, 1, or 2.

In certain embodiments, E is defined by Formula E-I-a.

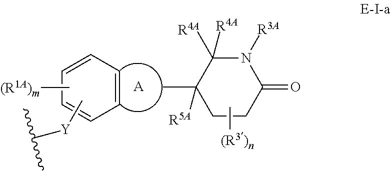

wherein
A is a substituted or unsubstituted heterocyclyl, or substituted or unsubstituted heteroaryl ring;
Y is —(CH₂)ₖ—, —(CH₂)k-O—, —O)CH₂)ₖ—, —NR^{B}(CH₂)ₖ—, —(CH₂)ₖ—NR^{B}—, —(CH₂)ₖ—(C═O)NR^{b}—, —O)CH₂)ₖ—(C═O)NR^{B}—, —O)CH₂)ₖ—NR^{B}(C═O)—, —NR^{B}(CO)—(CH₂)ₖ—O—, —NR^{B}(CH₂)ₖ—NR^{B}(C═O)—, or —(CH₂)ₖ—NR^{B}(C═O)—;
each R^{B} is, independently, hydrogen, or substituted or unsubstituted alkyl;
each R^{1A} is, independently, halogen, OH, C₁-C₆ alkyl, or C₁-C₆alkoxy;
each R^{3A} is, independently, hydrogen or C₁-C₃ alkyl;
each R³ is, independently, C₁-C₃ alkyl;
each R^{4A} is, independently, hydrogen or C₁-C₃ alkyl; or two R^{4A}, together with the carbon atom to which they are attached, form a C(O), C₃-C₆ carbocycle, or a 4-6-membered heterocycle comprising 1 or 2 heteroatoms selected from N and O;
R^{5A} is hydrogen, C₁-C₂ alkyl, F, or Cl;
k is 0, 1, 2, 3, 4, 5, or 6;
m is 0, 1, 2 or 3; and
n is 0, 1, or 2.

In certain embodiments, E is of Formula E-I-b:

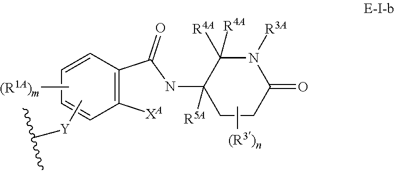

wherein:
Y is —(CH₂)ₖ—, —(CH₂)ₖ—O—, —O)CH₂)ₖ—, —NR^{B}(CH₂)ₖ—, —(CH₂)ₖ—NR^{B}—, —(CH₂)ₖ—(C═O)NR^{b}—, —O)CH₂)ₖ—(C═O)NR^{B}—, —O(CH₂)ₖ—NR^{B}(C═O)—, —NR(C═O)—(CH₂)ₖ—O—, —NR^{B}((CH₂)ₖ—NR^{B}(C═O)—, or —(CH₂)ₖ—NR^{B}(C═O)—;
X^{A} is C(O) or C(R^{3A})₂;
each R^{B} is, independently, hydrogen, or substituted or unsubstituted alkyl;
each R^{1A}, independently, halogen, OH, C₁-C₆ alkyl, or C₁-C₆alkoxy;
each R^{3A} is, independently, hydrogen, or C₁-C₃ alkyl;
each R³ is, independently, C₁-C₃ alkyl;
each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl;
k is 0, 1, 2, 3, 4, 5, or 6;
m is 0, 1, 2 or 3; and
n is 0, 1, or 2.

In certain embodiments of Formula E-I-b, Y is —(CH₂)ₖ—, —(CH₂)k-O—, —O)CH₂)ₖ—, —NR^{B}(CH₂)ₖ—, —(CH₂)ₖ—NR^{B}—, —(CH₂)ₖ— (C═O)NR^{b}—, —O)CH₂)ₖ—(C═O)NR^{B}—, —O)CH₂)ₖ—NR^{B}(C═O)—, —NR^{B}(CH₂)ₖ—NR^{B}(C═O)—, or —(CH₂)ₖ—NR^{B}(C═O)—; X^{A} is C(O) or C(R^{3A})₂; each R^{B} is, independently, hydrogen, or substituted or unsubstituted alkyl; each R^{1A} is, independently, halogen, OH, C₁-C₆ alkyl, or C₁-C₆alkoxy; each R^{4A} is, independently, hydrogen, or C₁-C₃ alkyl; each R³ is, independently, C₁-C₃ alkyl; each R^{4A} is, independently, hydrogen, or C₁-C₃ alkyl; or two R^{4A} together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; k is 0, 1, 2, 3, 4, 5, or 6; m is 0, 1, 2 or 3; and n is 0, 1, or 2.

In certain embodiments of Formula E-I-b, Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—; X^{A} is C(O) or C(R^{3A})₂; each R^{B} is, independently, hydrogen, or substituted or unsubstituted alkyl; each R^{1A} is, independently, halogen, OH, C₁-C₆ alkyl, or C₁-C₆alkoxy; each R^{3A} is, independently, hydrogen, or C₁-C₃ alkyl; each R³ is, independently, C₁-C₃ alkyl; each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; k is 0, 1, 2, 3, 4, 5, or 6; m is 0, 1, 2 or 3; and n is 0, 1, or 2.

In certain embodiments of Formula E-I-b, Y is —(CH₂)ₖ—NR^{B}—, —O)CH₂)ₖ—(C═)NR^{B}—, or —(CH)ₖ—NR^{B}(C═O)—; X^{A} is C(O) or C(R^{3A})₂; each R^{B} is, independently, hydrogen, or substituted or unsubstituted alkyl; each R^{1A} is, independently, halogen, OH, C₁-C₆ alkyl, or C₁-C₆alkoxy; each R^{3A} is, independently, H or C₁-C₃ alkyl; each R³ is, independently, C₁-C₃ alkyl; each R^{4A} is, independently, H or C₁-C₆ alkyl; or two R^{4A}, together with the carbon atom to which they are attached, form a C(O), C₅-C₆ carbocycle, or a 4-, 5-, or 6-membered heterocycle comprising 1 or 2 heteroatoms selected from N and O; R^{5A} is hydrogen, C₁-C₃ alkyl, F, or Cl; k is 0, 1, 2, 3, 4, 5, or 6; m is 0, 1, 2 or 3; and n is 0, 1, or 2.

In certain embodiments, E is defined by the formula below

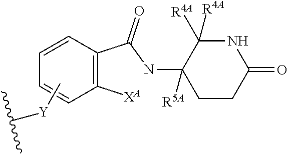

wherein Y, X^{A}, R^{5A}, and R^{4A} are as defined above.

In certain embodiments, F is of Formula E-I-d

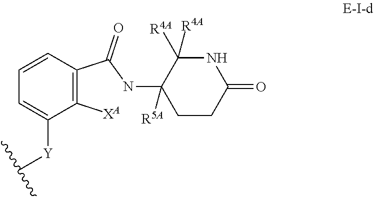

wherein
Y is —(CH₂)ₖ—NR^{B}—, —O)CH₂)ₖ—(C═O)NR^{B}—, —NR^{B}(C═O)—(CH₂)ₖ—O—, or —(CH₂)ₖ—NR^{B}(C═O)—;
X^{A} is C(O) or C(R^{3A})₂;
each R^{B} is, independently, hydrogen, or substituted or unsubstituted alkyl;
each R^{3A} is, independently, hydrogen or C₁-C₃ alkyl;
each R^{4A} is, independently, hydrogen or C₁-C₆ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl; and
k is 0, 1, 2, 3, 4, 5, or 6.

In certain embodiments of Formula E-I-d, Y is —(CH₂)ₖ—NR^{B}—, —O)CH₂)ₖ—(C═O)NR^{B}—, or —(CH₂)ₖ—, —NR^{B}(C═O)—; X^{A} is C(O) or C(R^{3A})₂; each R^{B} is, independently, hydrogen, or substituted or unsubstituted alkyl; each R^{3A}, independently, H or C₁-C₃ alkyl; each R^{4A} is, independently, H or C₁-C₆ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; and k is 0, 1, 2, 3, 4, 5, or 6.

In certain embodiments of Formula E-I-d, Y is —(CH₂)ₖ—NR^{B}—; X^{A} is C(O) or C(R^{3A})₂; each R^{B} is, independently, hydrogen, or substituted or unsubstituted alkyl; each R^{3A} is, independently, hydrogen or C₁-C₃ alkyl; each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; and k is 0, 1, 2, 3, 4, 5, or 6.

In certain embodiments, E is of Formula E-I-e

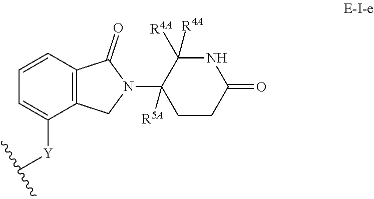

wherein:
Y is —(CH₂)ₖ—NR^{B}—, —O)CH₂)ₖ—(C═O)NR^{B}—, —NR^{B}(C═O)—(CH₂)ₖ—O—, or —(CH₂)ₖ—NR^{B}(C═O)—;
each R^{B} is, independently, hydrogen, or substituted or unsubstituted alkyl;
each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl; and
k is 0, 1, 2, 3, 4, 5, or 6.

In certain embodiments of Formula E-I-e, Y is —(CH₂)ₖ—NR^{B}—; each R^{B} is, independently, hydrogen, or substituted or unsubstituted alkyl; each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; and k is 0, 1, 2, 3, 4, 5, or 6.

In certain embodiments, E is of Formula E-I-f

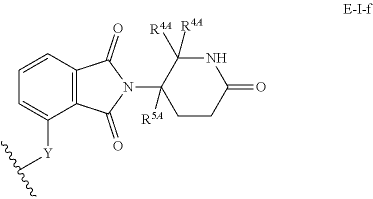

wherein:
Y is —(CH₂)ₖ—NR^{B}—, —O)CH₂)ₖ—(C═O)NR^{B}—, —NR^{B}(C═O)—(CH₂)ₖ—O—, or —(CH₂)ₖ—NR^{B}(C═O)—;
each R^{B} is, independently, hydrogen, or substituted or unsubstituted alkyl;
each R^{4A} is, independently, hydrogen, or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl; and
k is 0, 1, 2, 3, 4, 5, or 6.

In certain embodiments of Formula E-I-f, is —(CH₂)ₖ—NR^{B}—; each R^{B} is independently, hydrogen, or substituted or unsubstituted alkyl; each R^{4A} is, independently, hydrogen or C₁-C₃ alkyl; or two R^{4A}, together with the carbon atom to which they are attached, form a C(O), C₃-C₃ carbocycle, or a 4-, 5-, or 6-membered heterocycle comprising 1 or 2 heteroatoms selected from N and O; R^{5A} is hydrogen, C₁-C₃ alkyl, F, or Cl; and k is 0, 1, 2, 3, 4, 5, or 6.

In certain embodiments, E is of formula E-I-g

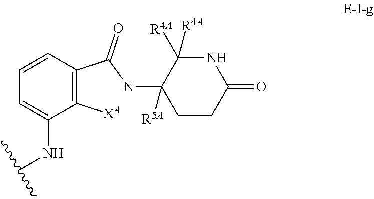

wherein:
X^{A} is C(O) or C(R^{3A})₂;
each R^{4A} is, independently, hydrogen, or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and 0;
each R^{3A} is, independently, hydrogen, or C₁-C₃ alkyl; and
R^{5A} is hydrogen, C₁-C₃ alkyl, F, or Cl.

In certain embodiments of compounds of Formula E-I-h, X^{A} is C(O).

In certain embodiments, E is of formula E-I-h

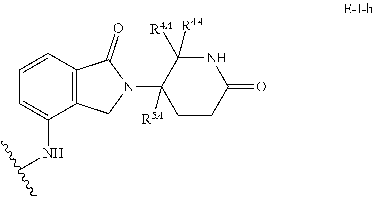

wherein each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and R^{5A} is hydrogen, C₁-C₃ alkyl, F, or Cl.

In certain embodiments, E is of formula E-I-i

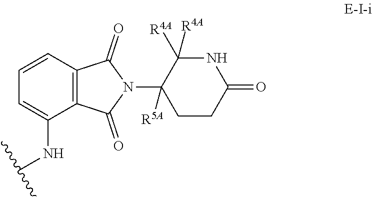

wherein each R^{4A} is, independently, H or C₁-C₃ alkyl; or two R^{4A} together with the carbon atom to which they are attached, form a C(O), C₃-C₆ carbocycle, or a 4-, 5-, or 6-membered heterocycle comprising 1 or 2 heteroatoms selected from N and O; and R^{5A} is H, C₁-C₃ alkyl, F, or Cl.

In certain embodiments, E is of Formula E-II

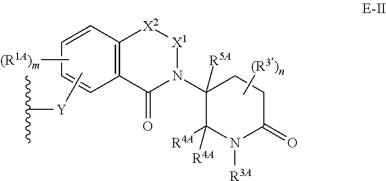

wherein:
Y is —(CH₂)ₖ—, —(CH₂)ₖ—O—, —O)CH₂)ₖ—, —NR^{B}(CH₂)ₖ—, —(CH₂)ₖ—NR^{B}—, —(CH₂)ₖ—(C═O)NR^{B}—, —O)CH₂)ₖ—(C═O)NR^{B}—, —O)CH₂)ₖ—NR^{B}(C═O)—, —NR^{B}(C═O)(CH₂)ₖ—O—, —NR^{B}(CH₂)ₖ—NR^{B}(C═O)—, or —(CH₂)ₖ—NR^{B}(C═O)—;
X¹-X² is C(R^{3A})N or C(R^{3A})₂—C(R^{3A})₂;
each R^{B} is, independently, hydrogen, or substituted or unsubstituted alkyl;
each R^{1A} is, independently, halogen, OH, C₁-C₆ alkyl, or C₁-C₆alkoxy;
each R^{3A} is, independently, hydrogen, or C₁-C₃ alkyl;
each R³ is, independently, C₁-C₃ alkyl;
each R^{4A} is, independently, hydrogen, or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl;
k is 0, 1, 2, 3, 4, 5, or 6;
m is 0, 1, 2 or 3; and
n is 0, 1, or 2.

In certain embodiments of Formula E-II, Y is —(CH₂)ₖ—, —(CH₂)ₖ—O—, —O)CH₂)ₖ—, —NR^{B}(CH₂)ₖ—, —(CH₂)ₖ—NR^{B}—, —(CH₂)ₖ—(C═O)NR^{B}—, —O)CH₂)ₖ—(C═O)NR^{B}—, —O)CH₂)ₖ—NR^{B}(C═O)—, —NR^{B}(CH₂)ₖ—NR^{B}(C═O)—, or —(CH₂)—NR^{B}(C═O)—; X¹-X² is C(R^{3A})═N or C(R^{3A})₂—C(R^{3A})₂; each R^{B} is, independently, hydrogen, or substituted or unsubstituted alkyl; each R^{1A} is, independently, halogen, OH, C₁-C₆ alkyl, or C₁-C₆ alkoxy; each R^{3A} is, independently, H or C₁-C₃ alkyl; each R³ is, independently, C₁-C₃ alkyl; each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; k is 0, 1, 2, 3, 4, 5, or 6; m is 0, 1, 2 or 3; and n is 0, 1, or 2.

In certain embodiments of Formula E-II, Y is —(CH₂)ₖ—NR^{B}—, —O)CH₂)ₖ— (C═O)NR^{B}—, —(CH₂)ₖ—O—, —NR^{B}(C═O)—(CH₂)ₖ—O—, or —(CH₂)ₖ—NR^{B}(C═O)—. In certain embodiments, Y is —(CH₂)ₖ—NR^{B}—, —O)CH₂)ₖ—(C═O)NR^{B}—, or —(CH₂)ₖ—NR^{B}(C═O)—. In certain embodiments, Y is —(CH₂)ₖ—NR^{B}—, —(CH₂)ₖ—O—, or —NR^{B}(C═O)—(CH₂)ₖ—O—. In certain embodiments, Y is —(CFhVNH—. In certain embodiments, Y is —NH—. In certain embodiments, Y is —(CH₂)ₖ—O—. In certain embodiments, Y is —O—. In certain embodiments, Y is —NH(C═O)—(CH₂)—O—. In certain embodiments, Y is —NH—, —O—, or —NH(C═O)—(CH₂)—O—.

In certain embodiments, E is of formula E-II-a

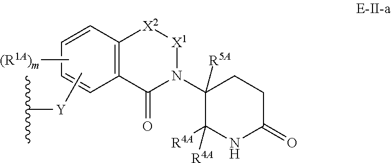

wherein
Y is —(CH₂)ₖ—, —(CH₂)ₖ—O—, —O)CH₂)ₖ—, —NR^{B}(CH₂)ₖ—, —(CH₂)ₖ—NR^{B}—, —(CH₂)ₖ—(C═O)NR^{b}—, —O)CH₂)ₖ—(C═O)NR^{B}—, —O)CH₂)ₖ—, —NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;
X¹—X² is C(R^{3A})═N or C(R^{3A})₂—C(R^{3A})₂;
each R^{B} is, independently, hydrogen, or substituted or unsubstituted alkyl;
each R^{1A} is, independently, halogen, OH, C₁-C₃ alkyl, or C₁-C₆alkoxy;
each R^{3A} is, independently, H or C₁-C₃ alkyl;
each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl;
k is 0, 1, 2, 3, 4, 5, or 6; and
m is 0, 1, 2, or 3.

In certain embodiments of formula E-II-a, Y is —(CH₂)ₖ—, —(CH₂)ₖ—O—, —O)CH₂)ₖ—, —NR^{B}(CH₂)ₖ—, —(CH₂)ₖ—NR^{b}—, —(CH₂)ₖ— (C═O)NR^{b}—, —O)CH₂)ₖ—(C═O)NR^{B}—, —O)CH₂)ₖ—NR^{B}(C═O)—, —NR^{B}(CH₂)ₖ—NR^{B}(C═O)—, or —(CH₂)ₖ—NR^{B}(C═O)—; X¹—X² is C(R^{3A})═N or C(R^{3A})₂—C(R^{3A})₂; each R^{B} is, independently, hydrogen, or substituted or unsubstituted alkyl; each R^{1A} is, independently, halogen, OH, C₁-C₆ alkyl, or C₁-C₆alkoxy; each R^{3A} is, independently, H or C₁-C₃ alkyl; each R^{4A}, independently, H or C₁-C₃ alkyl; or two R^{4A}, together with the carbon atom to which they are attached, form a C(O), (C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; k is 0, 1, 2, 3, 4, 5, or 6; and m is 0, 1, 2, or 3.

In certain embodiments of formula E-II-a, Y is —(CH₂)ₖ—NR^{B}—, —O)CH₂)ₖ—(C═O)NR^{B}—, —NR(C═O)—(CH₂)ₖ—O—, or —(CH₂)ₖ—NR^{B}(C═O)—; X¹-X² is (R^{3A})═N or C(R^{3A})₂—C(R^{3A})₂; each R^{B} is, independently, hydrogen, or substituted or unsubstituted alkyl; each R^{1A} is, independently, halogen, OH, C₁-C₆ alkyl, or C₁-C₆alkoxy; each R^{1A} is, independently, H or C₁-C₃ alkyl; each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{A} is hydrogen, C₁-C₃ alkyl, F, or Cl; k is 0, 1, 2, 3, 4, 5, or 6; and m is 0, 1, 2, or 3.

In certain embodiments of formula E-II-a, Y is —(CH₂)ₖ—NR^{B}—, —O)CH₂)ₖ—(C═O)NR^{B}—, or —(CH₂)ₖ—NR^{B}(C═O)—; X¹—X² is C(R^{3A})═N or C(R^{3A})₂—C(R^{3A})₂; each R^{B} is, independently, hydrogen, or substituted or unsubstituted alkyl; each R^{1A} is, independently, halogen, OH, C C alkyl, or C₁-C₆alkoxy; each R^{3A} is, independently, H or C₁-C₃ alkyl; each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or C1; k is 0, 1, 2, 3, 4, 5, or 6; and m is 0, 1, 2 or 3.

In certain embodiments, E is of formula E-II-b

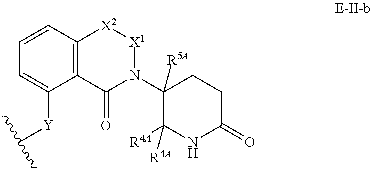

wherein:
Y is —(CH)ₖ—NR^{B}—, —O)CH₂)ₖ—(C═O)NR^{B}—, —N(C═O)—(CH₂)ₖ—O—, or —(CH₂)ₖ—NR^{B}(C═O)—;
X¹—X² is C(R^{3A})═N or C(R^{3A})₂C(R^{3A})₂;
each R^{B} is, independently, hydrogen, or substituted or unsubstituted alkyl;
each R^{3A} is, independently, H or C₁-C₃ alkyl;
each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl; and
k is 0, 1, 2, 3, 4, 5, or 6.

In certain embodiments of formula E-II-b, Y is —(CH₂)ₖ—NR^{B}—, —O)CH₂)ₖ—(C═O)NR^{B}—; or —(CH₂)ₖ—NR^{B}(C═O)—; X¹-X² is C(R^{3A})═N or C(R^{3A})₂—C(R^{3A})₂; each R^{B} is, independently, hydrogen, or substituted or unsubstituted alkyl; each R^{3A} is, independently, H or C₁-C₃ alkyl; each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{5A} is hydrogen, C₁-C₃ alkyl, F, or Cl; and k is 0, 1, 2, 3, 4, 5, or 6.

In certain embodiments, E is of formula E-II-c

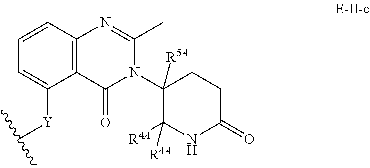

wherein:
Y is —(CH₂)ₖ—NR^{B}—, —O)CH₂)ₖ—(C═O)NR^{B}—NR^{B}(C═O)—(CH)ₖ—O—, or —(CH₂)ₖ—NR^{B}(C═O)—;
each R^{B} is, independently, hydrogen, or substituted or unsubstituted alkyl,
each R^{3A} is, independently. H or C₁-C₃ alkyl;
each R^{4A} is, independently, H or C₁-C₃ alkyl; or two R^{4A}, together with the carbon atom to which they are attached, form a C(O), C₁-C₆ carbocycle, or a 4-, 5-, or 6-membered heterocycle comprising 1 or 2 heteroatoms selected from N and O;
R^{5A} is hydrogen, C₁-C₃ alkyl, F, or Cl; and
k is 0, 1, 2, 3, 4, 5, or 6.

In certain embodiments of formula E-II-c, Y is —(CH₂)ₖ—NR^{B}—, —O)CH₂)ₖ—(C═O)NR^{B}—; or —(CH₂)ₖ—NR^{B}(C═O)—; X¹-X² is C(R^{3A})═N or C(R^{3A})₂—C(R^{3A})₂; each R^{B} is independently, hydrogen, or substituted or unsubstituted alkyl; each R^{3A} is, independently, H or C₁-C₃ alkyl, each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; R^{3A} is hydrogen, C₁-C₃ alkyl, F, or Cl; and k is 0, 1, 2, 3, 4, 5, or 6.

In certain embodiments, F is of formula E-II-d

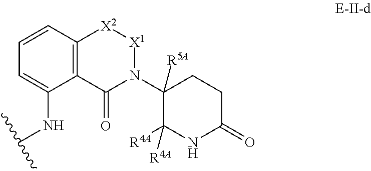

wherein:
X¹-X² is C(R^{3A})═N or C(R^{3A})₂—C(R^{3A})₂;
each R^{B} is, independently, hydrogen, or substituted or unsubstituted alkyl
each R^{3A} is, independently, H or C₁-C₃ alkyl;
each R^{4A} is, independently, H or C₁-C₃ alkyl; or two R^{4A} together with the carbon atom to which they are attached, form a C(O), C₃-C₆ carbocycle, or a 4-, 5-, or 6-membered heterocycle comprising 1 or 2 heteroatoms selected from N and O; and
R^{5A} is hydrogen, C₁-C₃ alkyl, F, or Cl.

In certain embodiments, E is defined by the formula below

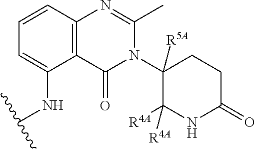

wherein each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and R^{5A} is hydrogen, C₁-C₃ alkyl, F, or Cl.

In certain embodiments, E is

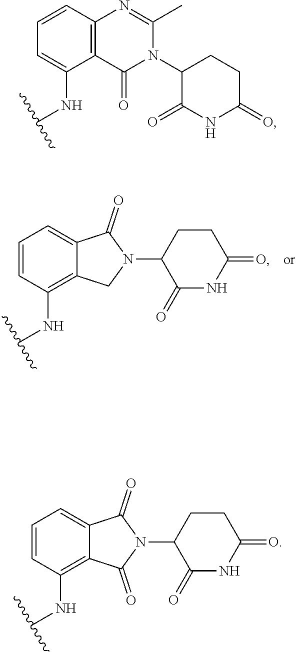 [SMILES: *Nc1cccc2nc(C)n(C3CCC(=O)NC3=O)c(=O)c12 ; *Nc1cccc2c1CN(C1CCC(=O)NC1=O)C2=O ; *Nc1cccc2c1C(=O)N(C1CCC(=O)NC1=O)C2=O]

In certain embodiments, E is

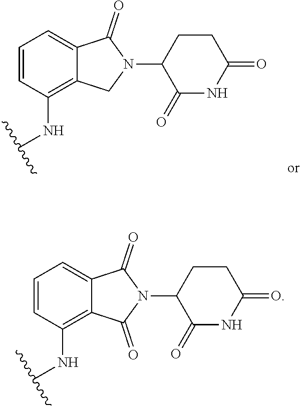 [SMILES: *Nc1cccc2c1CN(C1CCC(=O)NC1=O)C2=O ; *Nc1cccc2c1C(=O)N(C1CCC(=O)NC1=O)C2=O]

In certain embodiments, E is

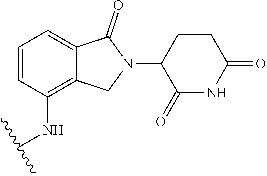 [SMILES: *Nc1cccc2c1CN(C1CCC(=O)NC1=O)C2=O]

In certain embodiments, E is

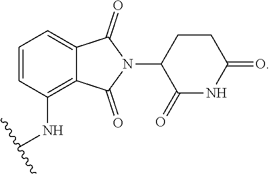 [SMILES: *Nc1cccc2c1C(=O)N(C1CCC(=O)NC1=O)C2=O]

The von Hippel-Lindau tumor suppressor (VHL) is an E3 ubiquitin ligase. VHL comprises the substrate recognition subunit/E3 ubiquitin ligase complex VCB, which includes elongins B and C, and a complex including Cullin-2 and Rbx1. The primary substrate of VHL is Hypoxia Inducible Factor 1a (HIF-1a), a transcription factor that upregulates genes, such as the pro-angiogenic growth factor VEGF, and the red blood cell-inducing cytokine, erythropoietin, in response to low oxygen levels. VCB is a known target in cancer, chronic anemia, and ischemia.

The full-length von Hippel-Lindau tumor suppressor protein (VHL) contains 213 amino acids. (For the VHL protein sequence see: Duan el al, Proc. Natl. Acad. Sci. U.S.A. 1995, 92, 6459-63. For additional information related to the VHL structure see Stebbins et al, Science 1999, 284, 455-61 and Minervini et al., Sci. Rep. 2015, 5, 12605) A second VHL-gene product arises by internal translation initiation from the codon 54 methionine, producing a 160 amino-acid protein (“pVHL19”). VHL has two main structural domains: an N-terminal domain composed mainly of b-sheets (b-domain) and a smaller C-terminal domain between amino acids 155-192 composed mainly of a helices (a-domain). The a-domain consists of three a helices that combines with a fourth a helix donated by elongin C. The b-domain is on the opposite side of the a domain and is free to contact other protein.

In certain embodiments, E is a modulator, binder, inhibitor, or ligand of VHL In certain embodiments, E is a modulator of VHL In certain embodiments, E is a binder of V-L. In certain embodiments, E is an inhibitor of VHL. In certain embodiments, E is a ligand of Cereblon. In certain embodiments, E is any ligand of VHL disclosed in U.S. patent application U.S. Ser. No. 16/523,219, filed Jul. 26, 2019; U.S. Patent Application, U.S. Ser. No. 16/375,643, filed Apr. 4, 2019, U.S. Patent Application, U.S. Ser. No. 16/230,792, filed Dec. 21, 2018; U.S. patent application U.S. Ser. No. 15/840,950, filed Sep. 13, 2018; U.S. patent application U.S. Ser. No. 15/996,151, filed Jun. 1, 2018; U.S. Patent Application, U.S. Ser. No. 15/953,108, filed Apr. 13, 2018; U.S. patent application U.S. Ser. No. 15/881,318, filed Jan. 26, 2018; U.S. patent application U.S. Ser. No. 15/853,166, filed Dec. 22, 2017; U.S. Patent Application, U.S. Ser. No. 15/852,854, filed Dec. 22, 2017; U.S. Patent Application, U.S. Ser. No. 15/851,053, filed Dec. 21, 2017; U.S. Patent Application, U.S. Ser. No. 15/829,541, filed Dec. 1, 2017; U.S. patent application U.S. Ser. No. 15/801,243, filed Nov. 1, 2017; U.S. Patent Application, U.S. Ser. No. 15/730,728, filed Oct. 11, 2017; U.S. patent application U.S. Ser. No. 15/706,064, filed Sep. 15, 2017; U.S. patent application U.S. Ser. No. 15/663,273, filed Jul. 28, 2017; U.S. patent application U.S. Ser. No. 15/230,354, filed Aug. 5, 2016; U.S. patent application U.S. Ser. No. 15/209,648, filed Jul. 13, 2016; U.S. Patent Application, U.S. Ser. No. 15/206,497, filed Jul. 11, 2016; U.S. patent application U.S. Ser. No. 15/574,770, filed Jun. 6, 2016; U.S. patent application U.S. Ser. No. 15/002,203, filed Jan. 20, 2016; U.S. patent application U.S. Ser. No. 14/822,309, filed Aug. 10, 2015; U.S. patent application U.S. Ser. No. 14/707,930, filed May 8, 2015; International Patent Application, PCT/US2019/040545, filed Jul. 3, 2.019; International Patent Application, PCT/US2019/040520, filed Jul. 3, 2019; International Patent Application, PCT/US2019/013481, filed Jan. 14, 2019; International Patent Application, PCT/US2018/052181, filed Sep. 21, 2018; International Patent Application, PCT/US2013/054663, filed Aug. 13, 2013; and Galdeano, C. et al. J. Med. Chem. 2014, 57, 8657, each of which is incorporated herein by reference. In certain embodiments, E is a modulator, binder, inhibitor, or ligand of a VHL variant. In certain embodiments, E is a modulator, binder, inhibitor, or ligand of a VHL isoform. In certain embodiments, E is a modulator, binder, inhibitor, or ligand of a VHL gene-product (e.g., pVHL19).

In certain embodiments, E is of Formula (E-III):

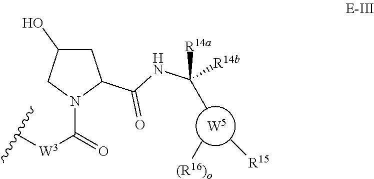

wherein:
W³ is substituted or unsubstituted arylene, substituted or unsubstituted heteroarylene, or

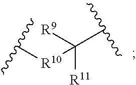

R⁹ and R¹¹ are independently hydrogen, substituted or unsubstituted alkyl, substituted or unsubstituted cycloalkyl, substituted or unsubstituted hydroxyalkyl, substituted or unsubstituted heteroaryl, or haloalkyl; or R⁹, R¹¹, and the carbon atom to which they are attached form a substituted or unsubstituted cycloalkyl;
R¹⁰ is —O—, —NH—, substituted or unsubstituted heterocyclylene, substituted or unsubstituted heteroarylene, or substituted or unsubstituted arylene;
R^{14a} and R^{14b} are each independently hydrogen, haloalkyl, or substituted or unsubstituted alkyl;
W⁵ is aryl or heteroaryl;
R¹⁵ is hydrogen, halogen, CN, OH, NO₂, —NR^{14a}R^{14b}, OR^{14a}, CONR^{14a}R^{14b}, NR^{14a}COR^{14b}, SO₂NR^{14a}R^{14b}, NR^{14a}SO₂R^{14b}, substituted or unsubstituted alkyl, haloalkyl, haloalkoxy, substituted or unsubstituted aryl, substituted or unsubstituted heteroaryl, substituted or unsubstituted cycloalkyl, or substituted or unsubstituted heterocyclyl;
each R¹⁶ is independently halo, substituted or unsubstituted alkyl, haloalkyl, hydroxy, haloalkoxy, or

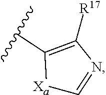

wherein R¹⁷ is hydrogen, halogen, substituted or unsubstituted C₃₋₆ cycloalkyl, substituted or unsubstituted C₁₋₆ alkyl, substituted or unsubstituted C₁₋₆ alkenyl, or C₁₋₆ haloalkyl;
Xₐ is S or O; and
o is 0, 1, 2, 3, or 4

In certain embodiments, E is of Formula E-III-a

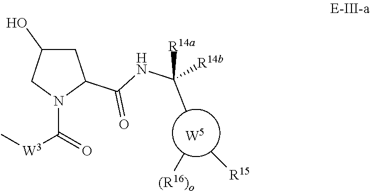

wherein W³, W⁵, R^{14a}, R^{14b}, R¹⁵, R¹⁶, and o are as defined herein.

In certain embodiments, E is of Formula E-III-a-1

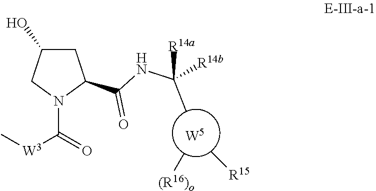

wherein W³, W⁵, R^{14a}, R^{14b}, R¹⁵, R¹⁶, and o are as defined herein.

In certain embodiments, E is of Formula E-III-b

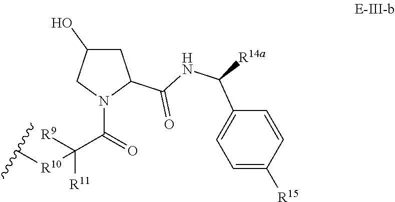

wherein W³, R⁹, R¹⁰, R¹¹, R^{14a}, and R¹⁵ are as defined herein.

In certain embodiments, E is of Formula E-III-b-1

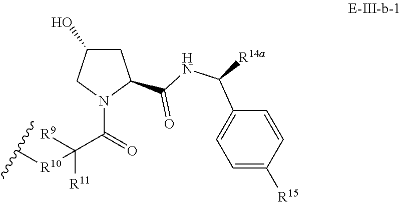

wherein W³, R⁹, R¹⁰, R¹¹, R^{14a}, and R¹⁵ are as defined herein.

In certain embodiments, E is of Formula E-III-c

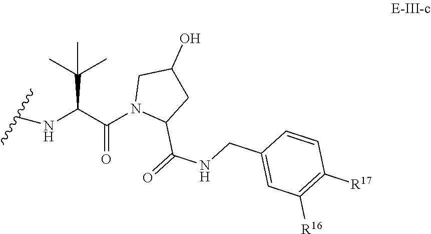

wherein R¹⁵ and R¹⁶ are as defined herein.

In certain embodiments, f is of Formula (E-III-c-1):

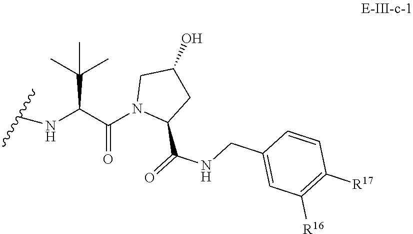

wherein R¹⁵ and R¹⁶ are as defined herein.

In certain embodiments, E is of Formula E-III-d

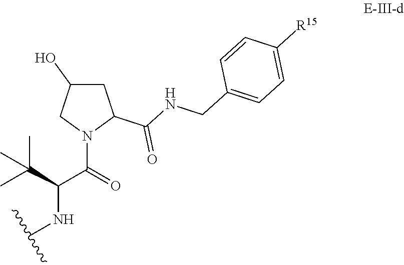

wherein R¹⁵ is as defined herein.

In certain embodiments, E is of Formula E-III-d-1

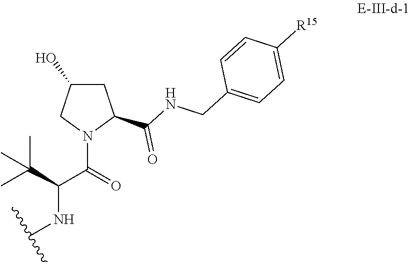

wherein R¹⁵ is as defined herein.

In certain embodiments, E is of the formula:

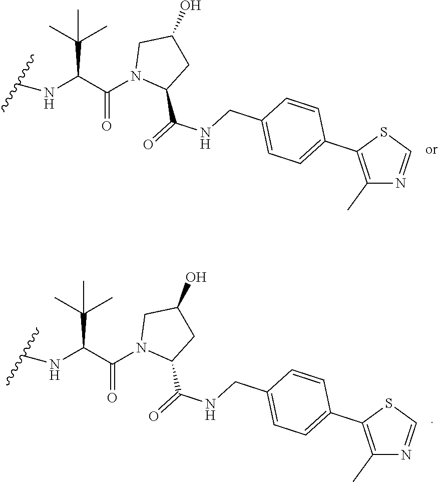 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@@H](O)C[C@@H]1C(=O)NCc1ccc(-c2scnc2C)cc1)C(C)(C)C]

In certain embodiments, E is of the formula:

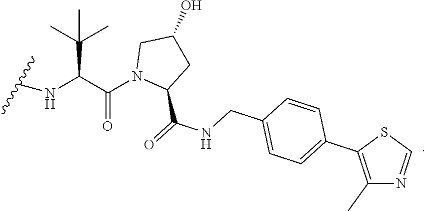 [SMILES: *N[C@H](C(=O)N1C[C@H](O)C[C@H]1C(=O)NCc1ccc(-c2scnc2C)cc1)C(C)(C)C]

In certain embodiments, E is of the formula:

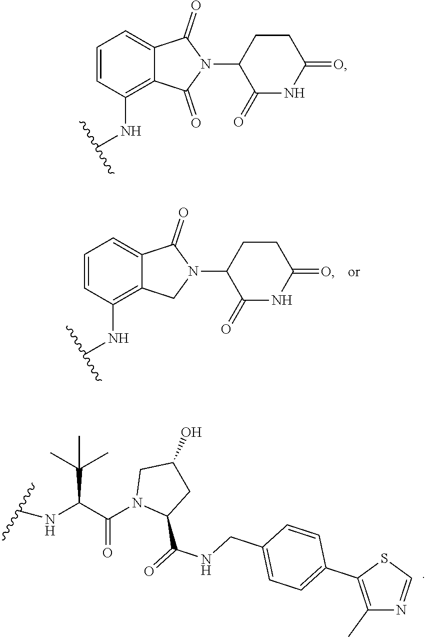 [SMILES: *Nc1cccc2c1C(=O)N(C1CCC(=O)NC1=O)C2=O ; *Nc1cccc2c1CN(C1CCC(=O)NC1=O)C2=O ; *N[C@H](C(=O)N1C[C@H](O)C[C@H]1C(=O)NCc1ccc(-c2scnc2C)cc1)C(C)(C)C]

In certain embodiments, E is of the formula:

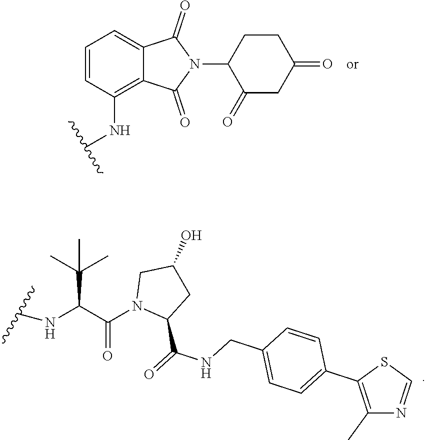 [SMILES: *Nc1cccc2c1C(=O)N(C1CCC(=O)CC1=O)C2=O ; *N[C@H](C(=O)N1C[C@H](O)C[C@H]1C(=O)NCc1ccc(-c2scnc2C)cc1)C(C)(C)C]

In some embodiments, the E3 ubiquitin ligase ligand moiety is selected from:

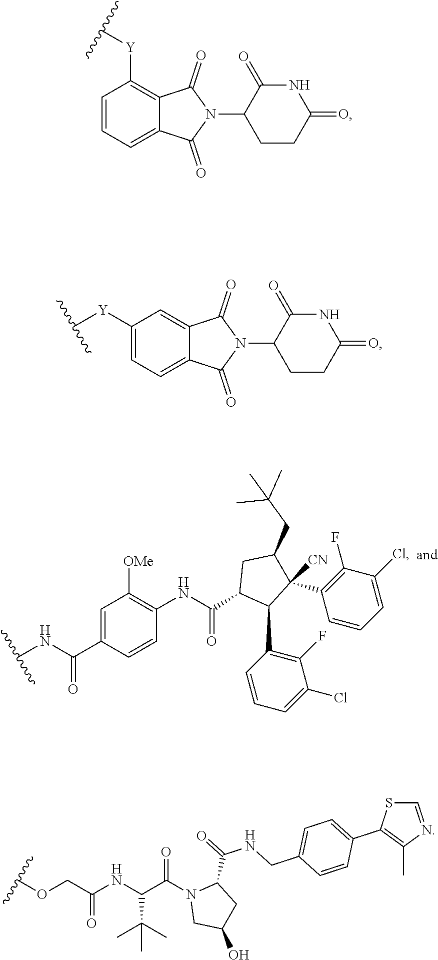

wherein Y is selected from —N(R)—, —N(H)—, and —O—, wherein R is optionally substituted alkyl.

In some embodiments, the E3 ubiquitin ligase ligand moiety is selected from.

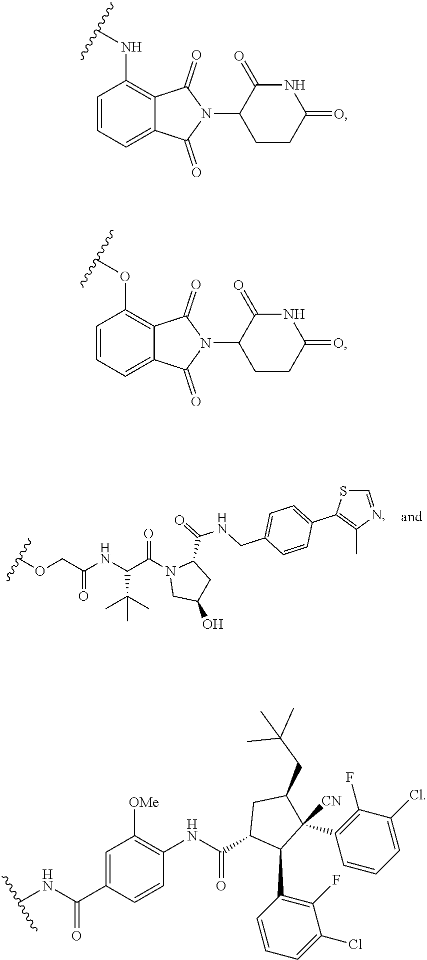

In certain embodiments, the E3 ligase ligand moiety binds an E3 ubiquitin ligase with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the E3 ligase ligand moiety binds Cereblon with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nm, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM. [00224] In certain embodiments, the E3 ligase binding moiety binds VHL with a K_{d} of less than 100,000 nM, less than 50,000 nM, less than 20,000 nM, less than 10,000 nM, less than 5,000 nM, less than 2,500 nM, less than 1,000 nM, less than 900 nM less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the E3 ligase ligand moiety selectively binds an E3 ubiquitin ligase as compared to another protein. In some embodiments, the E3 ligase ligand moiety selectively binds Cereblon over another protein. In some embodiments, the E3 ligase ligand moiety selectively binds Cereblon over another E3 ubiquitin ligase. In some embodiments, the E3 ligase ligand moiety selectively binds VHL over another protein. In some embodiments, the E3 ligase ligand moiety selectively binds VHL over another E3 ubiquitin ligase. In certain embodiments, the selectivity is between about 2-fold and about 5-fold. In certain embodiments, the selectivity is between about 5-fold and about 10-fold.

In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In some examples of Formula I, the compound can be defined by the formula below

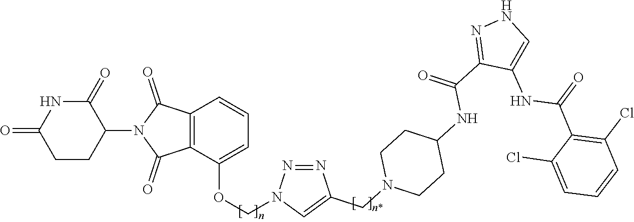

wherein n and n*are each independently an integer from 1 to 20, such as an integer from 1 to 15, an integer from 1 to 12, an integer from 1 to 10, an integer from 2 to 20, an integer from 2 to 15, an integer from 2 to 12, an integer from 2 to 10, an integer from 4 to 20, an integer from 4 to 15, an integer from 4 to 12, or an integer from 4 to 10. In some of these embodiments, the sum of n and n*can be from 8 to 14 (e.g., 8, 9, 10, 11, 12, 13, or 14).

In some examples of Formula II, the compound can be one of the following:

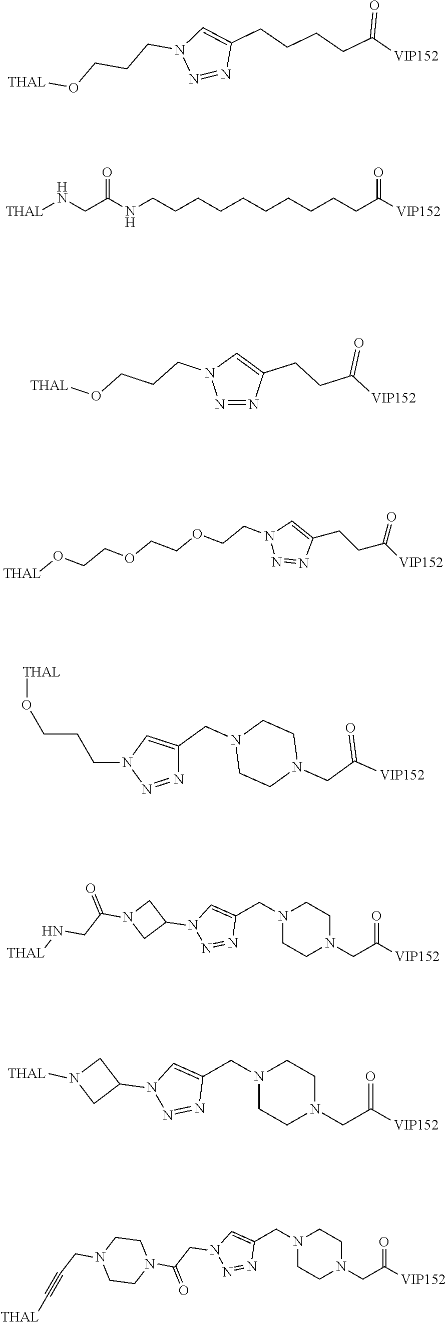

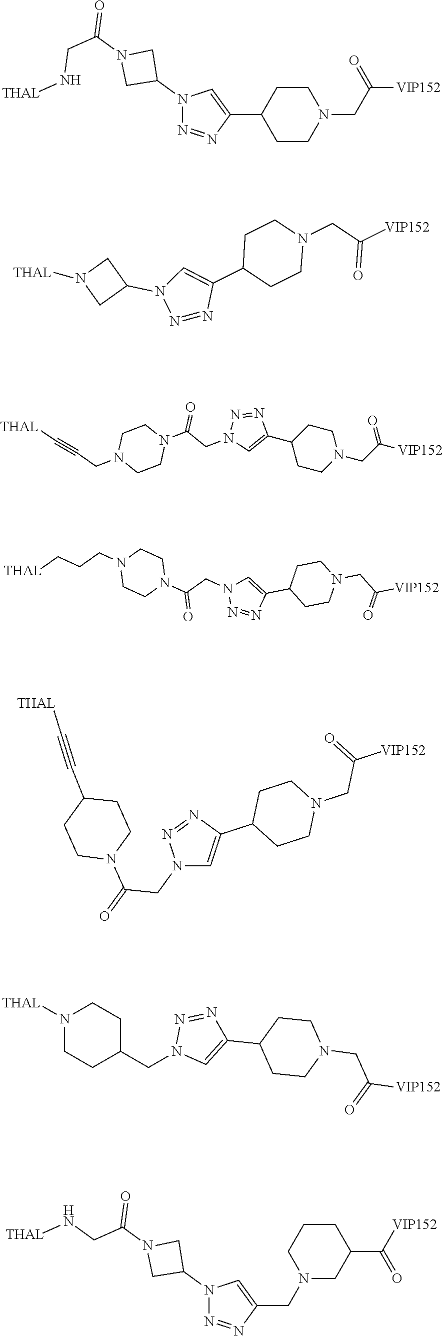

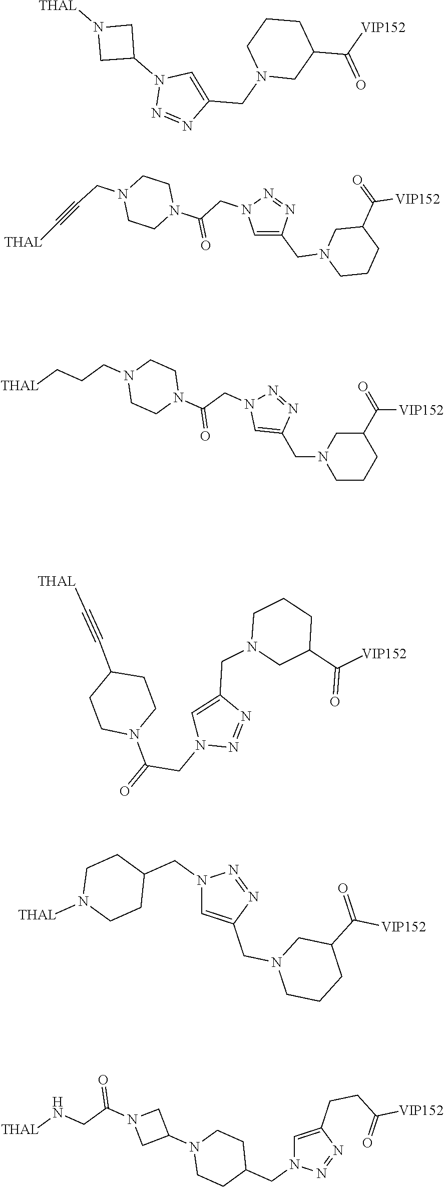

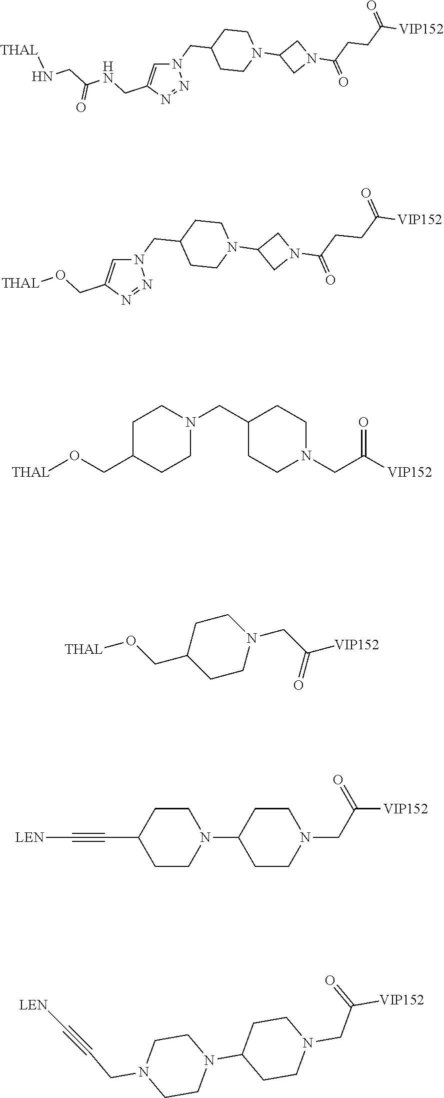

wherein THAL is

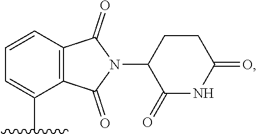 [SMILES: *c1cccc2c1C(=O)N(C1CCC(=O)NC1=O)C2=O]

### LEN is

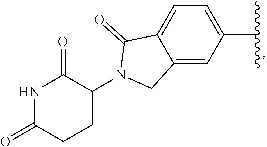 [SMILES: *c1ccc2c(c1)CN(C1CCC(=O)NC1=O)C2=O]

### and VIP152 is

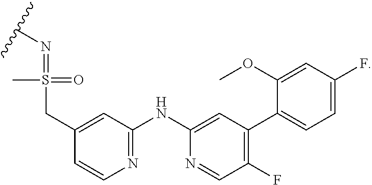 [SMILES: *N=S(C)(=O)Cc1ccnc(Nc2cc(-c3ccc(F)cc3OC)c(F)cn2)c1]

### Methods of Use

The compounds and compositions described herein can be used in methods for treating diseases and disorders. In some embodiments, the compounds and compositions described herein can be used in methods for treating diseases associated with the upregulation of myeloid cell leukemia-1 (Mcl-1) oncoprotein. In some embodiments, the compounds and compositions described herein can be used for the treatment of hyperproliferative disorders, including those hyperproliferative disorders associated with the upregulation of Mcl-1. The compounds and compositions described herein may also be used in treating other disorders as described herein and in the following paragraphs.

In one aspect, the disclosure provides a method of treating or preventing a disease or disorder alleviated by inhibiting Mcl-1 protein activity in a patient in need of said treatment or prevention. In some embodiments, the method comprises administering a therapeutically effective amount of one or more compounds of the disclosure, or a pharmaceutically acceptable salt thereof. In one aspect, the disclosure provides a method of treating or preventing a disease or disorder alleviated by indirectly inhibiting Mcl-1 protein activity in a patient in need of said treatment or prevention. In some embodiments, the method comprises administering a therapeutically effective amount of one or more compounds of the disclosure, or a pharmaceutically acceptable salt thereof. In some embodiments, the Mcl-1 protein activity is inhibited by the compounds of the disclosure binding to a target that downregulates and/or inhibits Mcl-1 protein activity.

In one aspect, the disclosure provides a method of treating or preventing a disease or disorder alleviated by inhibiting (CDK9 protein activity in a patient in need of said treatment or prevention. In some embodiments, the method comprises administering a therapeutically effective amount of one or more compounds of the disclosure, or a pharmaceutically acceptable salt thereof. In one aspect, the disclosure provides a method of treating or preventing a disease or disorder alleviated by indirectly inhibiting CDK9 protein activity in a patient in need of said treatment or prevention. In some embodiments, the method comprises administering a therapeutically effective amount of one or more compounds of the disclosure, or a pharmaceutically acceptable salt thereof.

In some embodiments, the disease or disorder is cancer. In some embodiments, the cancer is selected from the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like. In some embodiments, the cancer is acute myeloid leukemia (AML).

In some embodiments, the cancer is a blood cancer. In some embodiments, the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

In one aspect, the disclosure provides a method of treating or preventing acute myeloid leukemia (AML) in a patient in need of said treatment or prevention. In some embodiments, the method comprises administering a therapeutically effective amount of one or more compounds of the disclosure, or a pharmaceutically acceptable salt thereof.

In some embodiments, the hyperproliferative disorder treated by the compounds and compositions described herein includes cells having Mcl-1 protein and/or Mcl-1 related protein expression. In some embodiments, the disease treated by the compounds and compositions described herein is selected from the group consisting of myeloid leukemia, non-small cell lung cancer, pancreatic cancer, prostate cancer, and ovarian cancer.

In some embodiments, the compounds described herein may induce cell cycle arrest and/or apoptosis in cells containing functional Mcl-1 proteins. The compounds described herein may be used for sensitizing cells to additional agent(s), such as inducers of apoptosis and/or cell cycle arrest, and chemoprotection of normal cells through the induction of cell cycle arrest prior to treatment with chemotherapeutic agents.

In some embodiments, the compounds described herein may be useful for the treatment of disorders, such as those responsive to induction of apoptotic cell death, e.g., disorders characterized by dysregulation of apoptosis. In some embodiments, the compounds may be used to treat cancer that is characterized by resistance to cancer therapies (e.g., those cancer cells which are chemoresistant, radiation resistant, hormone resistant, and the like). In other embodiments, the compounds can be used to treat hyperproliferative diseases characterized by expression of functional Mcl-1 and/or Mcl-1 related proteins, which may or may not be resilient to Bcl-x_{L} inhibitors.

Efficacy of the compounds and combinations of compounds described herein treating the indicated diseases or disorders can be tested using various models known in the art, and described herein, which provide guidance for treatment of human disease.

In some embodiments, the methods provided herein further comprise administering one or more additional therapeutic agents to the subject. In some embodiments, each of the one or more additional therapeutic agents is independently selected from the group consisting of a steroid, an anti-allergic agent, an anesthetic (e.g., for use in combination with a surgical procedure), an immunosuppressant, an anti-microbial agent, an anti-inflammatory agent, and a chemotherapeutic agent Example steroids include, but are not limited to, corticosteroids such as cortisone, dexamethasone, hydrocortisone, methylprednisolone, prednisolone, and prednisone.

Example immunosuppressants include, but are not limited to, azathioprine, chlorambucil, cyclophosphamide, cyclosporine, daclizumab, infliximab, methotrexate, and tacrolimus.

Example anti-microbial agents include, but are not limited to, aminoglycosides (e.g., gentamicin, neomycin, and streptomycin), penicillins (e.g., amoxicillin and ampicillin), and macrolides (e.g., erythromycin).

Example anti-inflammatory agents include, but are not limited to, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example chemotherapeutics include, but are not limited to, proteosome inhibitors (e.g., bortezomib), thalidomide, revlimid, and DNA-damaging agents such as melphalan, doxorubicin, cyclophosphamide, vincristine, etoposide, carmustine, and the like. For example, one or more of the following agents may be used in combination with the compounds provided herein and are presented as a non-limiting list: a cytostatic agent, cisplatin, taxol, etoposide, innotecan, topotecan, paclitaxel, docetaxel, epothilones, tamoxifen, 5-fluorouracil, temozolomide, cyclophosphamide, gefitinib, erlotinib hydrochloride, imatinib mesylate, gemcitabine, uracil mustard, chlormethine, ifosfamide, chlorambucil, pipobroman, triethylenemelamine, triethylenethiophosphoramine, busulfan, lomustine, streptozocin dacarbazine, floxuridine, cytarabine, 6-mercaptopurine, 6-thioguanine, fludarabine phosphate, oxaliplatin, folinic acid, pentostatin, vinblastine, vindesine, bleomycin, dactinomycin, daunorubicin, doxorubicin epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methyltestosterone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, porfimer, erbitux, thiotepa, altretamine, trastuzumab, fulvestrant, exemestane, ifosfamide, rituximab, alemtuzumab, clofarabine, cladribine, aphidicolin, sunitinib, dasatinib, tezacitabine, triapine, trimidox, amidox, bendamustine, and ofatumumab.

### Pharmaceutical Compositions

In an embodiment, an active pharmaceutical ingredient or combination of active pharmaceutical ingredients, such as any of the CDK9 degraders described herein, is provided as a pharmaceutically acceptable composition.

In some embodiments, the concentration of each of the active pharmaceutical ingredients provided in the pharmaceutical compositions of the disclosure, such as any of the CDK9 degraders described herein, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0004%, 0.003%, 0.002%, 0.001%, 0.0009%, 0.0008%, 0.0007%, 00006%, 0.0005%, 0.0004%, 00003%, 0.0002% or 0.0001% w/w, w/v or v/v of the pharmaceutical composition.

In some embodiments, the concentration of each of the active pharmaceutical ingredients provided in the pharmaceutical compositions of the disclosure, such as any of the CDK9 degraders described herein, is greater than 90%, 80%, 70%, 60%, 50%, 40%, 30%, 20%, 19.75%, 19.50%, 19.25% 19%, 18, 75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25%, 10.75%, 10.50%, 10.25% 10% N), 9.75%, 9.50%, 9.25%, 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the CDK9 degraders described herein, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 13% to about 10% w/w, w/v or v/v of the pharmaceutical composition.

In some embodiments, the concentration of each of the active pharmaceutical ingredients provided in the pharmaceutical compositions of the disclosure, such as any of the CDK9 degraders described herein, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of each of the active pharmaceutical ingredients provided in the pharmaceutical compositions of the disclosure, such as any of the CDK9 degraders described herein, is equal to or less than 10 g, 9.5 g, 9.0 g, 8.5 g, 8.0 g, 75 g, 7.0 g, 6.5 g, 6.0 g, 55 g, 5.0 g, 4.5 g, 4.0 g, 3.5 g, 3.0 g, 2.5 g, 2.0 g, 15 g, 1.0 g 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 03 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the disclosure, such as any of the CDK9 degraders described herein, is more than 0.0001 g, 0.0002 g, 0.0003 g, 0.0004 g, 0.0005 g, 0.0006 g, 0.0007 g, 00008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g 0.70 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The active pharmaceutical ingredients described herein can be effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. Clinically-established dosages of the CDK9 degraders described herein may also be used if appropriate.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1.2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In one aspect, the disclosure provides a pharmaceutical composition comprising one or more of the CDK9 degraders described herein, or a pharmaceutically acceptable salt thereof, and a physiologically compatible carrier medium (also referred to as an excipient). In one aspect, the disclosure provides a pharmaceutical composition for treating or preventing a disease or disorder alleviated by inhibiting CDK9 protein activity, the pharmaceutical composition comprising one or more CDK9 degraders described herein, or a pharmaceutically acceptable salt thereof and a physiologically compatible carrier medium. In some embodiments, the disease or disorder is cancer.

In one aspect, the disclosure provides a pharmaceutical composition for treating or preventing a disease or disorder alleviated by indirectly inhibiting CDK9 protein activity, the pharmaceutical composition comprising one or more CDK9 degraders described herein, or a pharmaceutically acceptable salt thereof, and a physiologically compatible carrier medium.

In some embodiments, the disease or disorder is cancer. In some embodiments, the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is acute myeloid leukemia (AML).

In some embodiments, the cancer is a blood cancer. In some embodiments, the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma. Furthermore, the described methods of treatment may normally include medical follow-up to determine the therapeutic or prophylactic effect brought about in the subject undergoing treatment with the compound(s) and/or composition(s) described herein. In one aspect, the disclosure provides a pharmaceutical composition for treating or preventing from acute myeloid leukemia (AML), the pharmaceutical composition comprising one or more CDK9 degraders described herein, or a pharmaceutically acceptable salt thereof, and a physiologically compatible carrier medium. Described below are non-limiting pharmaceutical compositions.

When employed as pharmaceuticals, the CDK9 degraders described herein can be administered in the form of pharmaceutical compositions. These compositions can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral Parenteral administration includes intravenous, intraarterial, subcutaneous, intraperitoneal intramuscular or injection or infusion; or intracranial, (e.g., intrathecal or intraventricular, administration), Parenteral administration can be in the form of a single bolus dose, or may be, for example, by a continuous perfusion pump. In some embodiments, the compounds provided herein, or a pharmaceutically acceptable salt thereof, are suitable for parenteral administration. In some embodiments, the compounds provided herein are suitable for intravenous administration. In some embodiments, the compounds provided herein are suitable for oral administration. In some embodiments, the compounds provided herein are suitable for topical administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the pharmaceutical compositions provided herein are suitable for intravenous administration. In some embodiments, the pharmaceutical compositions provided herein are suitable for oral administration. In some embodiments, the pharmaceutical compositions provided herein are suitable for topical administration.

Also provided are pharmaceutical compositions which contain, as the active ingredient, a compound provided herein in combination with one or more pharmaceutically acceptable carriers (e.g. excipients). In making the pharmaceutical compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be, for example, in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as tale, magnesium stearate, and mineral oil; wetting agents; emulsifying and suspending agents; preserving agents such as methyl- and propylhydroxy-benzoates; sweetening agents; flavoring agents, or combinations thereof.

The active compound can be effective over a wide dosage range and is generally administered in an effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The compositions provided herein can be administered one from one or more times per day to one or more times per week; including once every other day. The skilled artisan will appreciate that certain factors can influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a compound described herein can include a single treatment or a series of treatments.

Dosage, toxicity and therapeutic efficacy of the compounds provided herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/IED₅₀. Compounds exhibiting high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: Design and Synthesis of CDK9-Targeting Protein Degraders for the Achievement of Improvement of Potency and Physicochemical Properties

Acute Myeloid Leukemia (AML) is a rare, but deadly hematological cancer with <30% 5-year survival rate. Current therapies for AML have low efficacy and/or high toxicity. Proteolysis Targeting Chimeras (PROTACs), also called degraders, are heterobifunctional molecules that contain a ligand for a target protein tethered through a linker to another ligand for an E3 Ligase complex within cells. The E3 ligase complex tags the target protein through proximity-induced ubiquitination, and this is followed by degradation by a proteasome. This mechanism of action allows a single PROTAC molecule to degrade multiple proteins, affording a catalytic process. The catalytic nature of PROTAC activity is an opportunity for lower dosing in AML patients, reducing the toxicity observed in current chemotherapies. Moreover, PROTAC technology has been shown to be less vulnerable to resistance development, a current and significant hurdle in the development of cancer therapeutics. This is because of the ability to completely degrade a target protein, without the limitation of the reliance on strong binding that drives the activity of traditional inhibitors.

The Cyclin Dependent Kinase (CDK9) pathway is dysregulated in AML cells, causing the upregulation of pro-survival genes, MCL-1 and MYC, among others. The inhibition of CDK9 in AML cells can be detrimental to their survival and this observation has led to the development of several CDK9 inhibitors over the years. However, no CDK9 inhibitor has been approved for the treatment of AML. To address the toxicity of CDK9 inhibitors and develop a degrader with potential as a clinical candidate for AML therapy, we incorporated AT-7519, a potent (K₁<10 nM) CDK9 inhibitor, into degraders with thalidomide (THAL) as Cereblon (CRBN) E3 ligase recruiters. A combinatorial approach was employed that involves click chemistry, a cycloaddition between an alkyne and an azide, that tethers CDK9-recruiter precursors to CRBN recruiter precursors. A significant advantage of this approach is the ability to use fragments from each compound library to generate PROTACs containing a variety of warheads for different targets and ultimately, different cancers. Multiple AT-7519-inspired CDK9 degraders were synthesized utilizing this chemistry, most of which demonstrated low nanomolar activity against AML cells. The availability of fragments from both compound libraries allowed a seamless transition to a new warhead, VIP152 (also referred to as BAY1251152 or B2), a potent (Kᵢ=3 nM) and selective inhibitor of CDK9 (minimum selectivity over other CDKs=120), This transition is important because AT-7519 is a pan-CDK inhibitor and its lack of selectivity was observed in its degraders. Alternately, the potency and selectivity of VIP152 can be conferred onto its degrader form.

Atuveciclib, another potent and selective CDK9 inhibitor, can be incorporated into a CRBN-recruiting degrader, B303. B03 exhibits high potency and selectivity, along with in vivo efficacy. However, the high lipophilicity of B03 (Log D=3.73) presents a disadvantage for future developability into a drug for human application. This is because of the high plasma protein binding can be associated with high lipophilicity, resulting in lower doses available for pharmacodynamic effects. Solvents like PEG400 and DMSO are sometimes used in formulations to solubilize highly lipophilic compounds during in vivo testing. However, these compounds present some toxicity concerns. Conversely, simple aqueous solutions, like phosphate buffered saline (PBS), as IV vehicles have been shown to be less toxic and to be isotonic relative to human plasma, a property that is important to the safety of IV drugs.

Therefore, for the development of VIP152-PROTAC series, a diverse library of chemical moieties are incorporated into the linkers to lower lipophilicity, thereby improving aqueous solubility. These degraders can exhibit in vivo activity suggesting that they are suitable as clinical candidates for AML.

### Employing Click Chemistry for the Facile Generation of AT7519-Based Degraders

In this Example, efficacious protein degraders of CDK9 are identified. AT7519 was chosen as the first warhead to incorporate into a degrader due to its potency, synthetic feasibility, and structural data that confirmed the presence of a synthetically useful linker attachment handle. Notably, the piperidine ring in AT7519 extends outside of the ATP-binding pocket within CDK2, a homolog of CDK9 within which the gene sequence encoding the binding pocket is conserved across CDKs. Therefore, the piperidine nitrogen can be used as an attachment point for the assembly of AT7519-based degraders. An initial approach involved the synthesis of a homologous series of AT7519-based degraders using acetic acid-derivatives of 4-hydroxythalidomide and pomalidomide, 4.1 and 4.2, respectively. This series utilized amino acid alkyl linkers to tether E3 ligase ligands to AT7519 through amide coupling. An illustration of this synthetic approach is shown in Scheme 1.

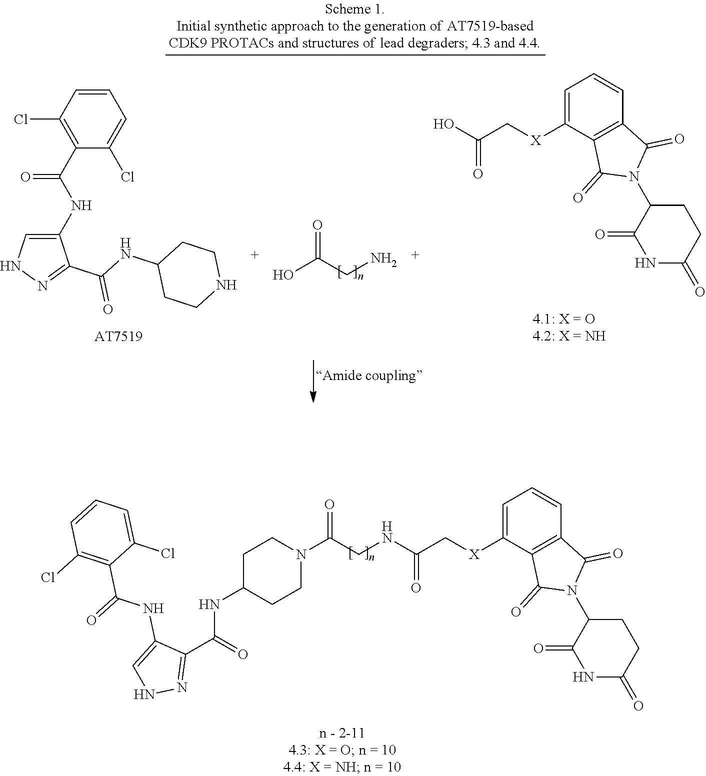

The generation of this degrader series led to the identification of 4.3 and 4.4 as two of the lead compounds that demonstrated the highest in vitro potency in cellular AML models; MV411 and MOLM13 cell lines. Both degraders were able to degrade CDK9 at low nanomolar concentrations. The PROTACs also demonstrated fold improvement in potency over AT7519 with regard to cytotoxicity against MV411 cells (Table 1). However, both molecules were tested in mice and failed to demonstrate CDK9 degradation. Moreover, dissolution of the test compounds in the 10 mM phosphate buffered saline (PBS) media was very difficult. Solvents, such as dimethylsulfoxide (DMSO), polyethylene glycol and Tween 80©, had to be added in generous quantities to the PBS vehicle to facilitate solubility of the test compounds and allow for the preliminary in vivo assessment that led to the conclusion of inactivity in mice. Significant toxicity, in the form of death and inflammation at the injection site, was also observed due to the inclusion of these additives in the PBS vehicle. These observations were attributed to the high lipophilicity of 4.3 and 4.4, as indicated by their predicted log D values (Table 1).

TABLE 1
Biological and physicochemical properties of lead AT7519-
based degraders bearing simple alkyl linkers.

| Compound | EC₅₀ | DC₅₀ | | |
|---|---|---|---|---|
| ID | (MV411) | (Molm-13) | | LogD |
| 4.3 | 0.4 μM | 0.1 | μM | 3.57 |
| 4.4 | 0.2 μM | 0.04 | μM | 3.85 |

| | | | |
|---|---|---|---|
| AT7519 | 0.2 μM | NA | −0.67 |

Given these data, it was concluded that there is a need for intentional linker design strategies that would facilitate an investigation into the impact of linker structure on overall activity, particularly on physicochemical properties. This led to the selection of Click chemistry as a modular approach to the generation of a target series of AT7519-based degraders. This next approach would then involve the generation of a series of thalidomide-linked alkyl azides (4.5a-d) and AT7519-bearing alkynes (4.6a-f), as shown in Scheme 2. The ability to synthesize the azides and alkynes separately afforded the opportunity to modify linker length more easily. This led to synthetic access to several combinations of precursors within each group that could have afforded 24 unique PROTAC molecules. Of the 24 degraders that could have been accessed using the synthesized AT7519- and IMiD-derived compound library, ten compounds were targeted for synthesis and characterization: 4.7a-c-4.10. The “N” and “N*” notations are used to indicate the number of linker atoms on the IMiD and AT7519 sides, respectively. These compounds were chosen to investigate the impact on overall properties by varying the position of the rigid triazole relative to each ligand within the PROTAC molecule. Therefore, this triazole-bearing series contained three sets of degraders with identical/similar linker lengths, but with varying positions of the triazole ring. The purpose of the outlier, 4.10, was to synthesize a degrader that was comparable, based on number of methylene units within the linker, to degrader 3.

Scheme 2. Alternate modular synthetic approach to the generation of AT7519-based CDK9 degraders using click chemistry.

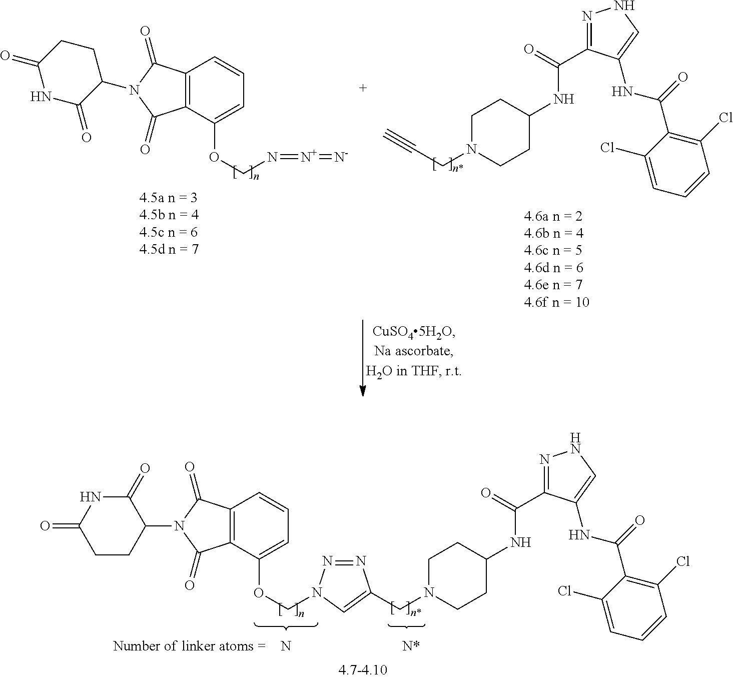

| ID | N:N* |
|---|---|
| 4.7a | 5:4 |
| 4.8a | 8:2 |
| 4.9a | 8:6 |
| 4.7b | 4:4 |
| 4.8b | 5:5 |
| 4.9b | 7:7 |
| 4.7c | 4:5 |
| 4.8c | 4:6 |
| 4.9c | 4:10 |
| ID | N:N |
| 4.10 | 7:5 |

This study was designed to evaluate the impact of the triazole position on degrader efficacy and drug properties. It was hypothesized that the position of the rigid triazole would primarily impact ternary complex formation due to changes in the binding pose and/or affinity within the ligand in closest proximity. This effect on the ternary complex by the triazole position was expected to consequently lead to differences in potency within a set of degraders bearing identical/similar linker lengths. The emphasis on the investigation of the impact of the triazole position was the motivation for the selection of simple alkyl precursors for the generation of the azide and alkyne intermediates. This was due to the ability to mitigate influence from additional factors such as hydrogen bond and polar interactions that could have resulted from the incorporation of linkers with added functionality, such as PEG linkers.

The synthesis of the azide-linked IMiD library is represented in Scheme 3. A series of commercially available diols was tosylated using p-toluene sulfonyl chloride in dichloromethane (DCM) under basic conditions, through the addition of triethylamine to produce 4.11a-d. The derived mono-tosylated products were then heated with sodium azide in dimethylformamide (DMF) to afford azidoalcohols (4.12a-d) which were afforded cleanly, in quantitative yield. The free alcohols were then taken forward to a second tosylation using the same reaction conditions described earlier. This led to the synthesis of the tosylated alkylazide 4.13a-d which were then reacted with 4-hydroxy thalidomide (4.14) in a nucleophilic substitution (S_{N}²) at high temperatures in DMF with diisopropylethylamine (DIPEA). This led to regioselective conversion to the desired thalidomide-linked azides: 4.5a-d. The synthesis of 4.14 is also shown, as the IMiD derivative is efficiently afforded by the reaction of 3-aminopiperidine-2,6-dione with 4-hydroxyisobenzofuran-1,3-dione under acidic conditions at a high temperature.

Scheme 4.3. Synthesis of the IMID-azide library; alkylazide derivatives of 4-OH thalidomide (4.14).

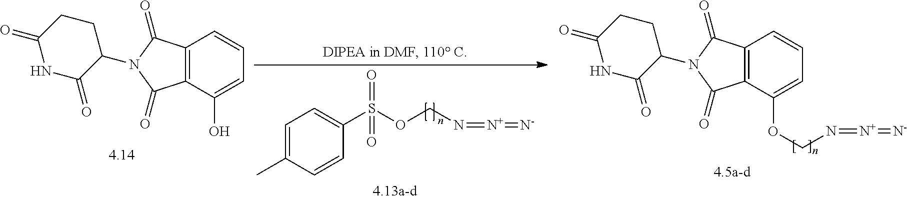

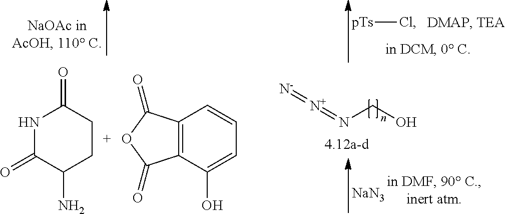

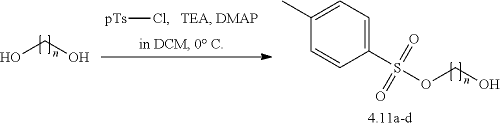

| ID | n | N |
|---|---|---|
| 4.5a | 3 | 4 |
| 4.5b | 4 | 5 |
| 4.5c | 6 | 7 |
| 4.5d | 7 | 8 |

The set of A7519-linked alkynes was obtained more easily compared to the IMiD-azides, due to the commercial availability of certain alkynyl halides (4.15a-b) and alkynols. Using appel conditions, four alkynols were converted to their iodo-analogues using triphenylphosphine, diiodide and imidazole in DCM. The reactions were effective in providing the desired iodoalkynes (4.15c-f) within an hour of setup. All alkynyl halides were then taken forward into an S_{N}² reaction with AT7519 under basic conditions using carbonate bases in acetonitrile (ACN). These reactions successfully resulted in the desired alkyne-linked AT7519 intermediates 4.6a-f.

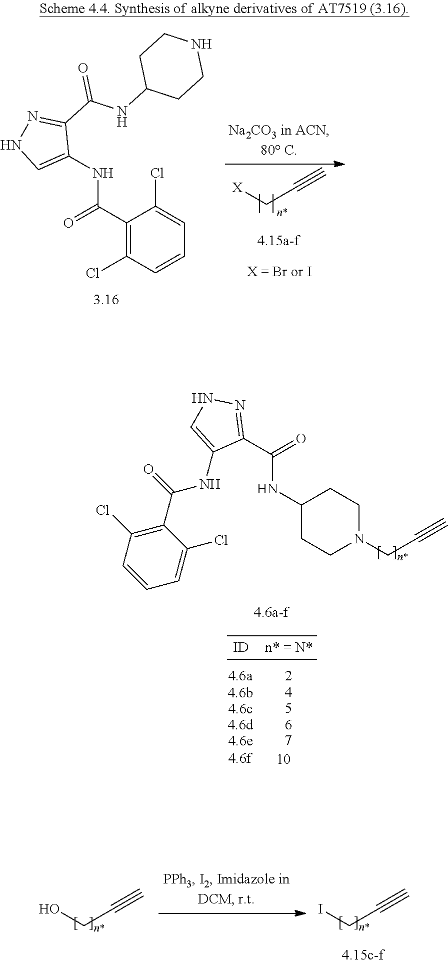

The generation of the azide and alkyne intermediates would then facilitate the Huigsen cycloaddition between select pairs to afford the assembly of the triazole-containing CDK9 degrader series (4.7-4.10). Click chemistry was performed using copper sulfate pentahydrate and sodium ascorbate. The two salts would be added to a stirring mixture of the azide and alkyne in tetrahydrofuran (THF), after which water was added to facilitate dissolution of the salts in the reaction mixture. DMF was also added, as needed, to improve the solubility of each ligand derivative so that a homogenous reaction mixture could be obtained. These reaction conditions successfully provided each of the ten degraders in the target series (4.7-4.10).

### Characterization of Triazole-Containing AT7519 Degrader Series; Understanding Impact of Linker Structure on Overall Properties

The obtained compounds were tested in biological experiments to measure their potency in inhibiting the growth of AML cells and in their degradation of CDK9. These measurements are reported as half-maximum growth inhibitory and half-maximum degradation concentrations, IC₅₀s and DC₅₀s, respectively. Maximum percent degradation (Dₘₐₓ) values were also obtained during the CDK9 degradation assessments. The common MTS assay was used to acquire the IC₅₀ values, while the DC₅₀ values were obtained using an automated western blot experiment. These values are presented in Table 2, along with pIC₅₀ (log(nM IC₅₀)) to more accurately reflect the differences/lack thereof in in vitro cytotoxicity. The notation “N:N*” represents the number of linker atoms on each side of the rigid triazole ring for a given degrader. Predicted log D values, calculated using the ChemAxon logarithm, are also provided for each degrader. To characterize the physicochemical properties of the new degrader series, two additional assays were performed: a kinetic aqueous solubility assay and the chromatographic log D experiment described earlier. Aqueous solubility is shown in μM while experimentally determined log D is shown as log k′80. Details regarding the execution of each experiment are provided below.

The values derived from these analyses for each degrader are also shown in Table 2.

TABLE 4.2
Physicochemical properties and in vitro activity of
triazole-containing degraders

| | | | | Solubility | DC₅₀ | Dₘₐₓ | IC₅₀ | |
|---|---|---|---|---|---|---|---|---|
| ID | N:N* | LogD | logk′80 | (μM) | (nM) | (%) | (nM) | pIC₅₀ |
| 4.7a | 5:4 | 1.81 | 0.100 | 12.4 | 130.4 | 67.5 | 240.8 | 2.4 |
| 4.7b | 4:4 | 1.29 | 0.084 | 40.9 | 69.2 | 79.1 | 190.1 | 2.3 |
| 4.7c | 4:5 | 1.57 | 0.106 | 31.3 | 403.5 | 58.6 | 193.5 | 2.3 |
| 4.8a | 8:2 | 3.21 | 0.146 | 5.65 | 60.1 | 88.3 | 46.9 | 1.7 |
| 4.8b | 5:5 | 2.08 | 0.112 | 13.2 | 950.8 | 68.5 | 130.1 | 2.1 |
| 4.8c | 4:6 | 1.93 | 0.116 | 12.9 | 98.9 | 75.3 | 71.3 | 1.9 |
| 4.9a | 8:6 | 3.78 | 0.184 | 2.38 | 74.6 | 86.2 | 72.8 | 1.9 |
| 4.9b | 7:7 | 3.78 | 0.176 | 4.70 | 61.4 | 78.0 | 63.3 | 1.8 |
| 4.9c | 4:10 | 3.70 | 0.197 | 3.42 | 44.8 | 88.6 | 8.8 | 0.9 |
| 4.10 | 7:5 | 2.97 | 0.140 | 5.46 | 99.9 | 70.1 | 46.6 | 1.7 |
| 4.3 | N/A^{a} | 3.57 | 0.154 | 3.40 | ND^{b} | ND | 200.0 | 2.3 |
| AT- | — | −0.61 | ND | ND | ND | ND | 118.6 | 2.1 |
| 7519 | | | | | | | | |

(^{a}N/A = not applicable,
^{b}ND = not determined).

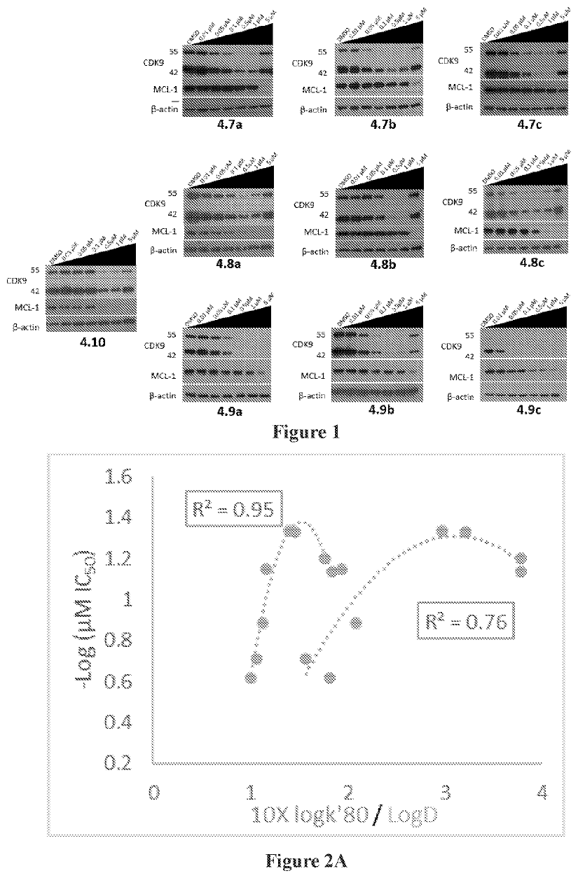

The cytotoxicity data showed that a significant portion of the degraders in the click series (4.8, 4.9a-e and 4.10) were able to achieve low nanomolar potency against MV411 cells, outperforming AT7519 in the same cell line. Most importantly, certain degraders demonstrated downstream repression of the anti-apoptotic gene MCL-1, as determined by western blots obtained from drugged MOLM13 cells (FIG. 1). To better evaluate the catalytic mechanism of action of this degrader series, a drug washout (WO) experiment was performed. This experiment involved the drugging of cells for six hours after which the cell media was replaced with a drug-free batch. After a total drug incubation time of 48 hours, cytotoxicity (Post-WO IC₅₀) was evaluated and compared to the values derived from cells treated under the same conditions but did not undergo a washout procedure (No-WO IC₅₀).

The results showed that degraders 4.9b, c and 4.10 were able to retain their potency after the drug washout. The potency of each of these degraders after drug washout was at least 5-fold the observed activity of the parent warhead, AT7519, under the same experimental conditions. These data may allude to a benefit of a degrader approach to target engagement as opposed to traditional inhibition, i.e., the need for a lower dose of a PROTAC to exert the desired pharmacological effect.

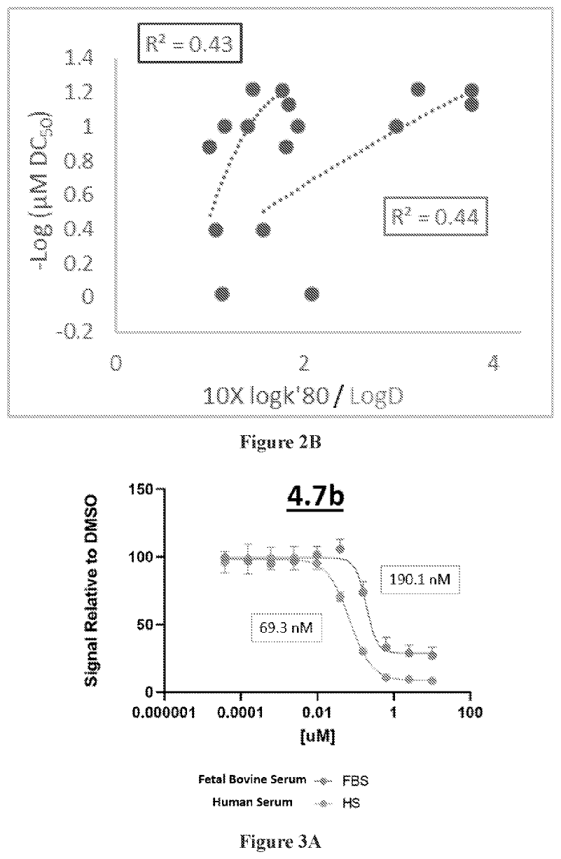

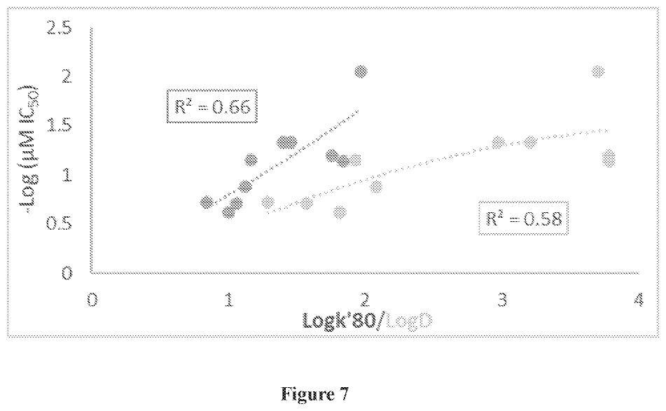

Additionally, across the three sets of isomers, an increase in potency, hydrophobicity (according to aqueous solubility) and lipophilicity with increase in linker length is observed. A curve fit analysis shows that within the click series, IC₅₀ and predicted log D were moderately correlated (R²=0.76), when 4.7b and 4.9c are excluded, given that both degraders show higher activity than expected relative to their predicted and measured lipophilicities (see FIG. 7). The same curve fit analysis performed with log k′80 shows that chromatographic log D has a stronger relationship to cellular potency (R²=0.95) than predicted log D. These data (shown in FIG. 2A) demonstrate that the use of bRo5-relevant methods to assess physicochemnical properties of PROTACs can better facilitate understanding of degrader SAR relationships, particularly for linker design. Interestingly, as shown in FIG. 2B, DC₅₀ is weakly correlated to both predicted log D and measured log k′80. The lack of a strong relationship in either case suggests that degradation potency is more closely related to the unique mechanism of action of degraders, specifically, the catalytic mode of action. This is because the correlation between overall potency (IC₅₀) and log k′80 is likely based on permeability, which affects both traditional inhibition and degradation activity. However, with PROTACs, the impact of permeability is attenuated due to the ability of a single PROTAC molecule to degrade multiple units of a POI.

Therefore, while log k′80 may correlate to the number of PROTAC molecules that successfully cross the cell membrane, the value does not dictate the efficiency of each molecule in degrading the target protein. This may explain the weak correlation between lipophilicity and the measured DC₅₀S.

### Examining the Impact of the Embedded Triazole on Biological Activity and Physicochemical Properties

Comparing the performances of 4.10 and 4.3 demonstrates the value of the structural design of the click series. First, removing the amide linkages that were present in 4.3, results in over 5-fold improvement in cytotoxicity in 4.10. This is interesting given the fact that both degraders have the same overall linker length and identical number of methylene units (11). The observed differences in activity could be attributed to differences in binding affinity caused by changing the nature of attachment of the linkers to the constituent ligands. This is further supported by the moderately higher observed lipophilicity of 4.3 (log k′80=0.14), relative to 4.10 (log k′80=0.15). Given that higher lipophilicity is generally found to result in higher potency, the improved activity of 4.10 over 4.3 is likely due to the specific design of the click series.

Within the 4.8 and 4.9 sets of constitutional isomers, some significant changes in potency upon varying the position of the triazole are observed, particularly with regard to cytotoxicity. It is also important to note that the compounds within each set that possess the highest observed log k′80 demonstrated the highest cellular potency. This could conceivably have resulted in differences in permeability that would ultimately translate to higher potency. However, the minimum difference in log k′80 between 4.9c and its constitutional isomers is 0.013, while the corresponding value for 4.8a is 0.030. Despite this, 4.9c outperforms its isomeric counterparts in cytotoxicity by almost 10-fold while 4.8a had a modest 1.5 to 2.7-fold improvement in activity over 4.8b and 4.8c. These results suggest that the exceptional performance of 4.9c is likely due to factors beyond its lipophilicity or even its linker length. The data therefore support the proposed hypothesis, that the movement of the triazole position can impact the potency of these AT7519-based degraders. The impact of the triazole is also notable when examining the aqueous solubility values within the 4.7 set. A notable difference in aqueous solubility is observed between 4.7a and 4.7c from simply moving the rigid triazole by one methylene unit closer to one ligand over the other. The presented data also show that beyond the 4.8 set, the aqueous solubility of the degraders begin to stay below 5 μM. By contrast, 4.7b is able to demonstrate the highest aqueous solubility within the series, 40 μM, likely due to its low predicted log D and low measured log k′80.

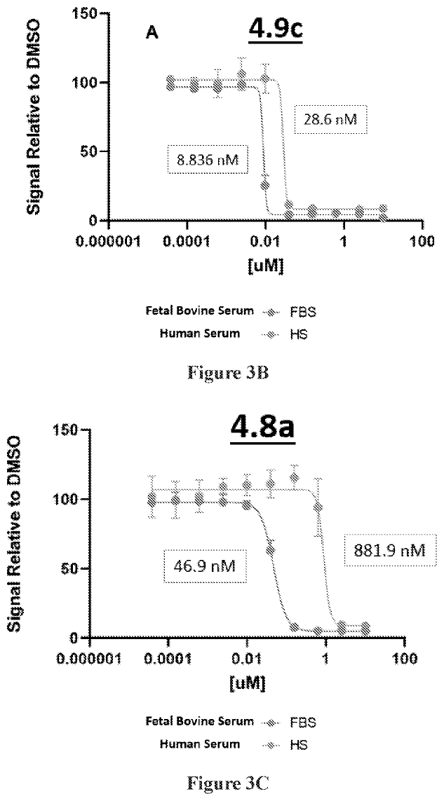

The impact of the improved aqueous solubility of 4.7b over the other analogues within the click series is seen when tested in human serum (HS), as opposed to fetal bovine serum (FBS). FIG. 3A shows a retention of potency in human serum, with 4.7b actually demonstrating near 3-fold improvement in cytotoxicity against MV411 cells. This result is peculiar because the most commonly observed effect when compounds are tested in HS compared to FBS is that there is either a retention of potency or a loss thereof, rarely is an improvement observed. Nevertheless, these results highlight the importance of balancing lipophilicity and polarity in degrader development. This is due to the fact that the difference in activity in HS vs. FBS (as known as “a protein shift”) is usually associated with the binding of drugs to the plasma proteins that are present to a significantly higher degree in HS, as opposed to FBS. Therefore, the low lipophilicity (according to log k′80) and higher aqueous solubility of 4.7b is likely the reason for the lack of a protein shift for the degrader. This contrasts with 4.8a, which loses its potency by almost 20-fold in human serum (FIG. 3C). Interestingly, 4.9c only loses its potency in human serum by 3-fold, while still maintaining superior potency over other analogues within both FBS and HS (FIG. 3B).

Within this series, the ability to tune physicochemical properties while managing potency and molecular weight is demonstrated through the use of click chemistry. The discovery of 4.7b was also quite encouraging, based on its duality of potency in HS and its higher aqueous solubility Unfortunately, in a preliminary study in AML-xenografted mice, in which 4.7b was administered, in vivo CDK9 degradation was not observed. This led to the decision to rethink the design of these degraders: first, by incorporating a more selective warhead and second, by exploring alternative linker building blocks to improve aqueous solubility.

Improving Degrader Potency, Selectivity and Aqueous Solubility with a New Warhead and Alternative Linker Chemistry

VIP152 (3.19) is one of the most potent and selective CDK9 inhibitors reported to date. The small molecule has been shown to be effective in repressing MCL-1 and BCL-2 anti-apoptotic genes in different hematological disorders, including in AML and chronic lymphocytic leukemia (CLL). Therefore, VIP152 was chosen for the next phase of the CDK9 degrader development project.

Racemic VIP152 (4.16) would first be synthesized using the route presented in Scheme 5. This scheme follows the synthetic approach that was used in the original patent in which the discovery of VIP152 was first reported. The route began with a Suzuki coupling between the aryl iodide 4.17 and boronic acid 4.18, using [1,1′-Bis(diphenylphosphino)ferrocene]dichloropalladium(II) as the catalyst. The reaction cleanly provided intermediate 4.19 in high yield. In a nucleophilic aromatic substitution, 4.19, was then reacted with the amine 4.20, at high temperature with sodium tert-butoxide to provide the alcohol 4.21. Using thionyl chloride, 4.21 was converted into its corresponding chloride 4.22, which was obtained in quantitative yield. The intermediate 4.23 was then obtained by stirring 4.22 with sodium thiomethoxide at room temperature. Following purification, 4.23 was then treated very slowly with a solution of brominated trifluoroacetamide, while carefully monitoring reaction temperature. These reaction conditions provided 4.24 in good yield. The trifluoroacetate 4.24, was then hydrolyzed using potassium hydroxide (KOH) and oxidized to the desired sulfoximine 4.16 using oxone.

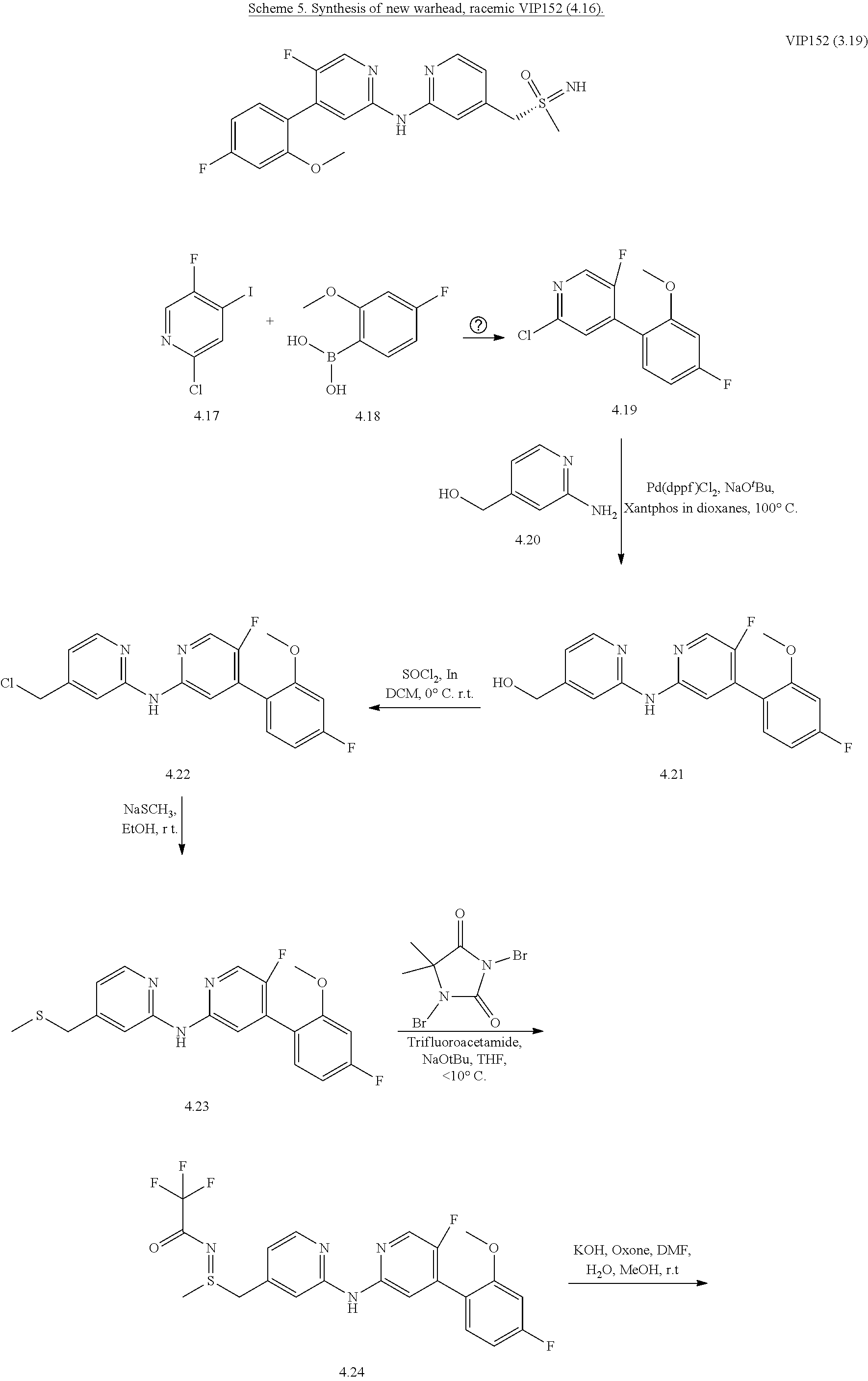

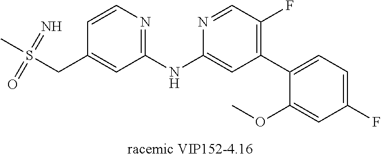 [SMILES: COc1cc(F)ccc1-c1cc(Nc2cc(CS(C)(=N)=O)ccn2)ncc1F]

The synthesis of multi-gram quantities of 4.16 would then facilitate the generation of a novel series of VIP152-based degraders. Along with the choice to switch from the pan-CDK inhibitor, AT7519, to VIP152, it was also decided that other chemical moieties for the linker would be explored, beyond alkyl and PEG groups. However, in order to determine the level of potency that VIP152 could offer to this new series, a short series of VIP152-inspired degraders bearing simple alkyl and PEG linkers had to be synthesized and biologically characterized. To this end, using the intermediates 4.13a and 4.25, the alkyl azide derivatives of 4-hydroxy thalidomide 4.5a and 4.26 were synthesized. In order to properly compare the impact of the new warhead on degrader potency, relative to the AT7519-based degrader 4.3, the intermediate 4.27 was used to synthesize the IMiD derivative 4.28.

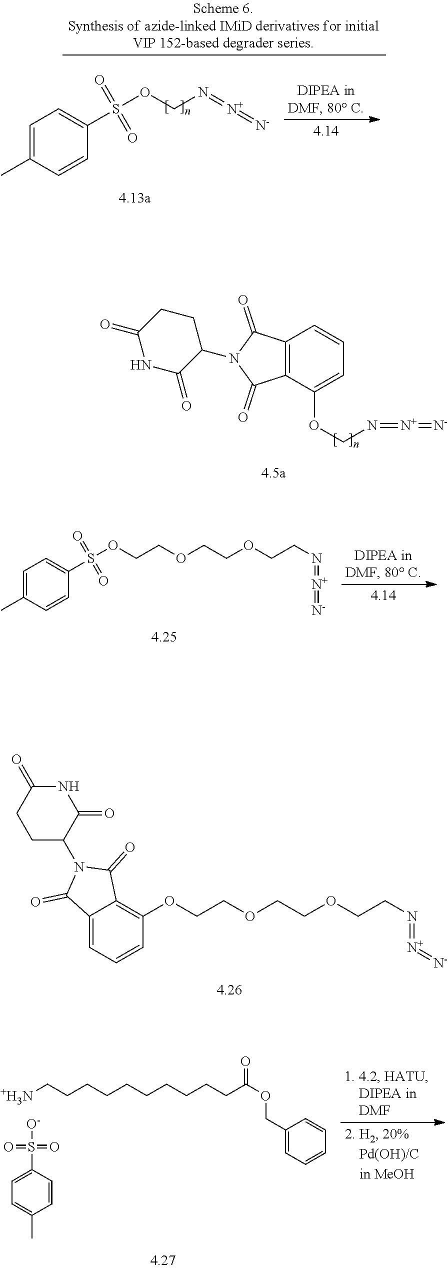

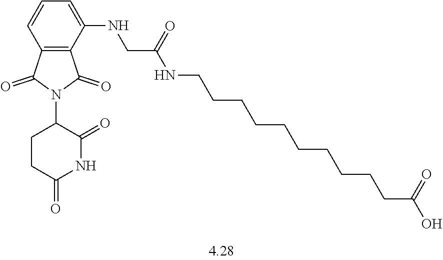 [SMILES: O=C(O)CCCCCCCCCCNC(=O)CNc1cccc2c1C(=O)N(C1CCC(=O)NC1=O)C2=O]

Through amide coupling using I-ethyl-3-carbodiimide hydrochloride (EDC.HCl), the alkynyl derivatives of 4.16, 4.29 and 4.30 were synthesized. Using these VIP152-derivatives, degraders 4.31, 4.33 and 4.34 were synthesized using click chemistry. Degrader 4.32 was also synthesized using amide coupling conditions with (1-[Bis(diethylamino)methylene]-1H-1,2,3-triazolo[4,5-b]pyridinium 3-oxide hexafluorophosphate (HATU) and the carboxylic acid 4.28. Scheme 7 illustrates the synthesis of this initial series. The series was evaluated for cytotoxicity against MOLM13 cells (IC₅₀) and for their CDK9 degradation potency (DC₅₀). The results are provided in Table 3.

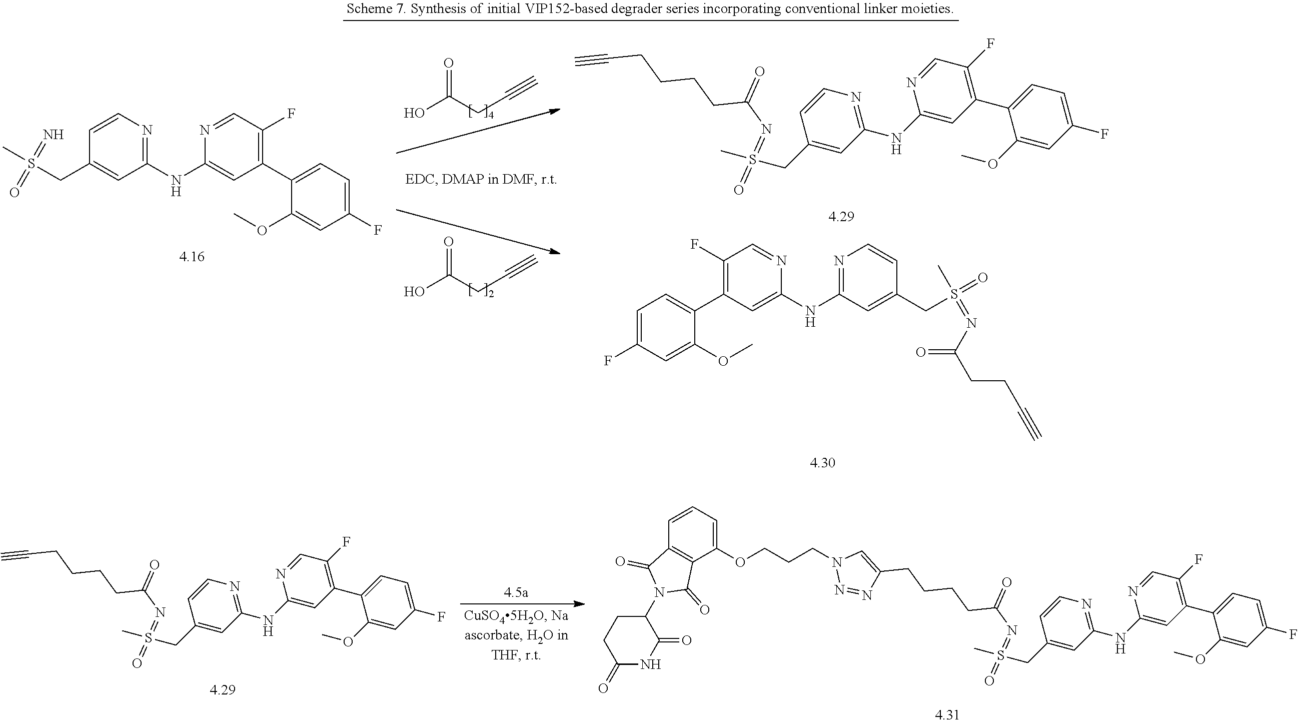

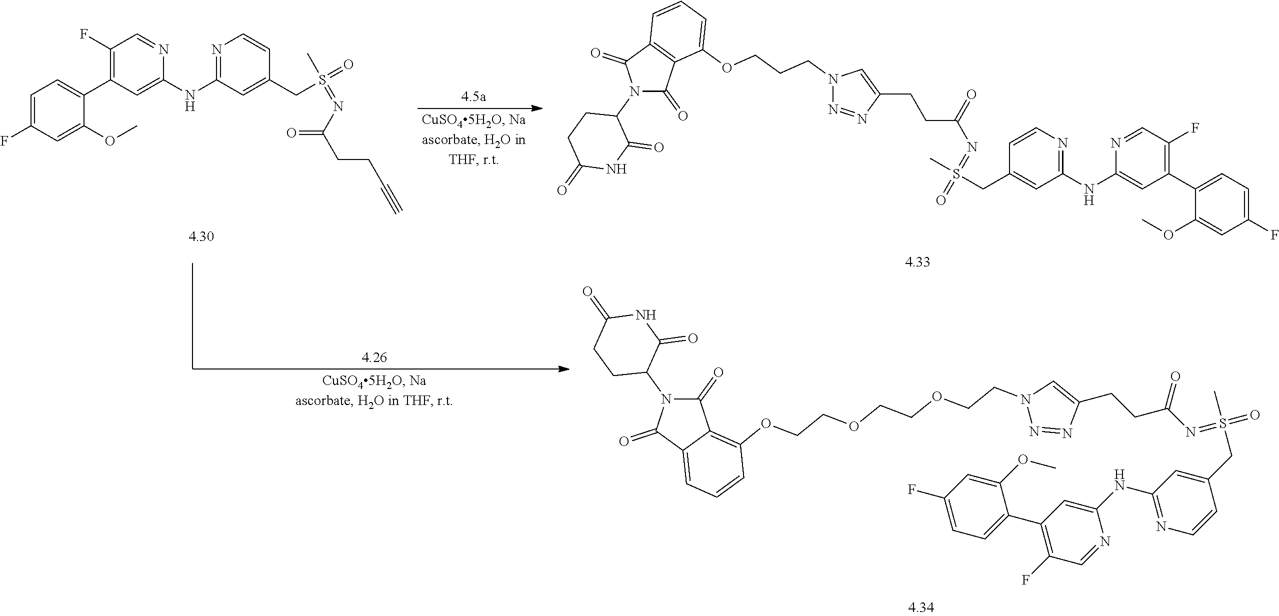 [SMILES: C#CCCC(=O)N=S(C)(=O)Cc1ccnc(Nc2cc(-c3ccc(F)cc3OC)c(F)cn2)c1 ; COc1cc(F)ccc1-c1cc(Nc2cc(CS(C)(=O)=NC(=O)CCc3cn(CCCOc4cccc5c4C(=O)N(C4CCC(=O)NC4=O)C5=O)nn3)ccn2)ncc1F ; COc1cc(F)ccc1-c1cc(Nc2cc(CS(C)(=O)=NC(=O)CCc3cn(CCOCCOCCOc4cccc5c4C(=O)N(C4CCC(=O)NC4=O)C5=O)nn3)ccn2)ncc1F]

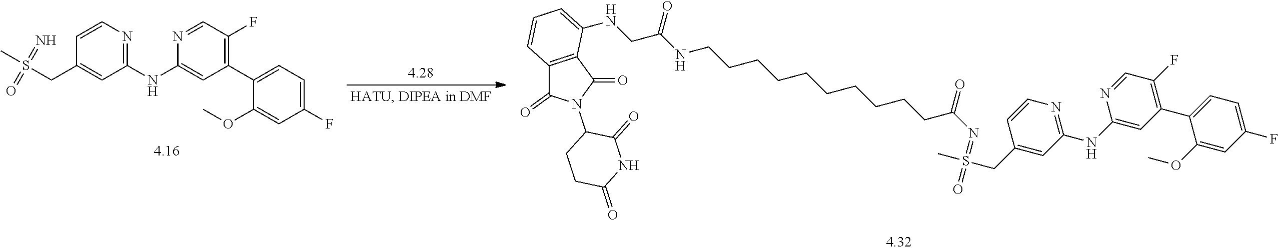 [SMILES: COc1cc(F)ccc1-c1cc(Nc2cc(CS(C)(=N)=O)ccn2)ncc1F ; COc1cc(F)ccc1-c1cc(Nc2cc(CS(C)(=O)=NC(=O)CCCCCCCCCCNC(=O)CNc3cccc4c3C(=O)N(C3CCC(=O)NC3=O)C4=O)ccn2)ncc1F]

TABLE 3
Biological activity, kinetic solubility and chromatographic logD of initial VIP152-based degraders.

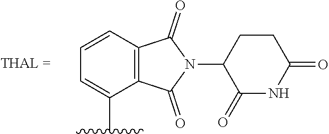

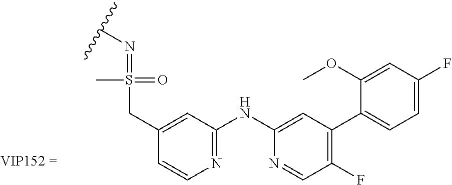

| | | | Solubility | DC₅₀ | IC₅₀ |
|---|---|---|---|---|---|
| Structure | ID | logk′80 | (μM) | (μM) | (μM) |
| 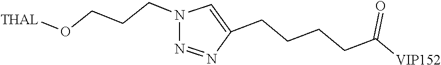 | 4.31 | 0.17 | <5.0 | ND | 0.17 |
| 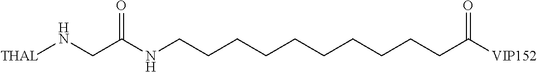 | 4.32 | 0.28 | ND | 0.12 | 0.14 |
| 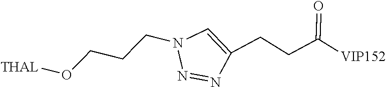 | 4.33 | 0.15 | ND | 0.17 | 0.19 |
| 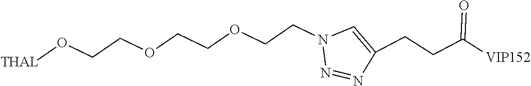 | 4.34 | 0.13 | 5.2 | 0.07 | 0.06 |
| 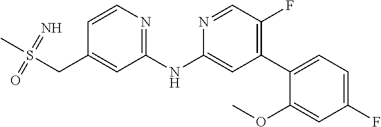 [SMILES: COc1cc(F)ccc1-c1cc(Nc2cc(CS(C)(=N)=O)ccn2)ncc1F] | 4.16 | 0.15 | 402.9 | N/A | 0.06 |

The degraders within the initial series were able to demonstrate low nanomolar cytotoxicity against MOLM13 cells, with degrader 4.34 showing comparable potency to the parent warhead, 4.16. However, none of the degraders in the series showed significant aqueous solubility. At first, the lack of good aqueous solubility was surprising, especially for degrader 4.31 which shares the same structural design as its AT7519 analogue, 4.7b. However, the poor aqueous solubility of 4.31 can be rationalized by the considerably higher predicted log D of 4.16 (log D=2.22), relative to AT7519 (log D=−0.61). The high lipophilicity of 4.31 and 4.34 set the stage for the challenges that would follow in the design of this new degrader series. It was clear that, starting with the new, more lipophilic warhead, the achievement of good aqueous solubility would be more challenging within this series. The characterization of this initial series with classical linkers had certainly validated the decision to move towards a different set of linker moieties for the next generation of CDK9 degraders.

As the first step towards incorporating nitrogen-containing heterocycles within the VIP152-based degraders, the VIP152 alkyne derivative 4.35 was synthesized according to Scheme 8. This alkyne intermediate would incorporate a piperazine ring, a modification that was expected to increase aqueous solubility based on the ability of the piperazine to ionize at physiological pH (pKa=10 Compound 4.35 was synthesized by reacting Boc-protected piperazine 4.36 with propargyl bromide in an S_{N}² reaction to produce 4.37a which was treated with trifluoroacetic acid (TFA) to obtain 4.37b Tinder the same basic conditions, 4.37b was reacted with tert-butyl 2-bromoacetate to produce the pivoyl ester of 4.38. Using the same TEA conditions for the tert-butyl deprotection, the carboxylic acid, 4.38 was obtained and reacted under EDC coupling conditions with 4.16 to obtain 4.35. The alkyne was then reacted under click chemistry conditions to produce the first VIP152-based degrader hearing a saturated heterocyclic linker, 4.39.

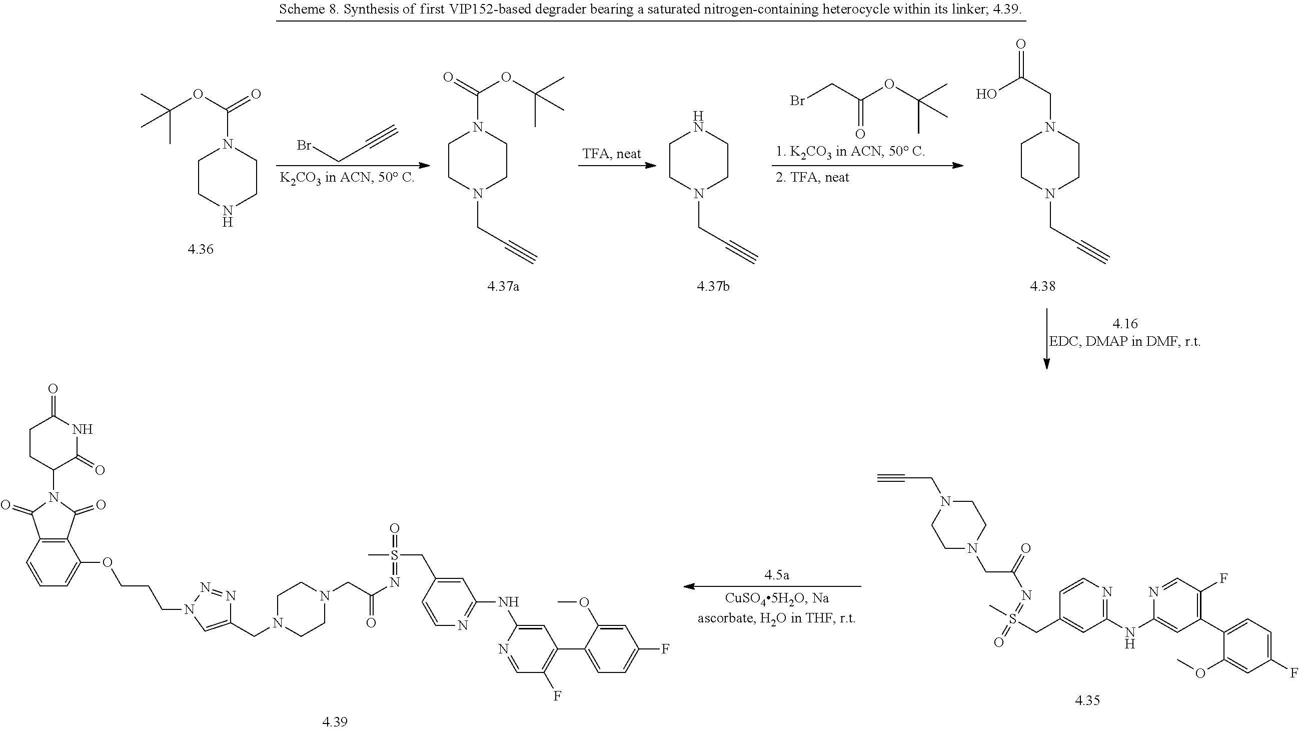

The incorporation of the piperazine moiety, in 4.39, did deliver an improvement in aqueous solubility (26.5 μM). However, this was accompanied by a drop in potency (0.83 μM). Despite the loss in poency, the increased kinetic solubility of 4.39 indicated that there was value in exploring the new chemical space for the degrader design, particularly with regard to managing physicochemical properties. To further explore the opportunities for tuning properties that the piperazine could offer, a series of ImiD derivatives incorporating azetidine and piperazine was synthesized: 4.40-4.42.

The synthesis of the three new IMiD derivatives is illustrated in Scheme 9. Using sodium azide at high temperature conditions in DMF, and following TFA deprotection, the bromide 4.43 was converted to the azidoazetidine 4.44. The azide intermediate was then reacted with 4.2 using HATU coupling conditions to afford 4.40. The intermediate 4.44 was also reacted in a nucleophilic aromatic substitution with 4-fluoro thalidomide 4.45 to produce the IMiD azide 4.41. Through a Sonogashira coupling with 4-bromo thalidomide 4.46 and the propargyl derivative 4.37, the piperazine-linked thalidomide derivative 4.47a was synthesized. Following TFA deprotection to 4.47b, the resulting amine was coupled to azidoacetic acid 4.48, to obtain the IMiD azide 4.42

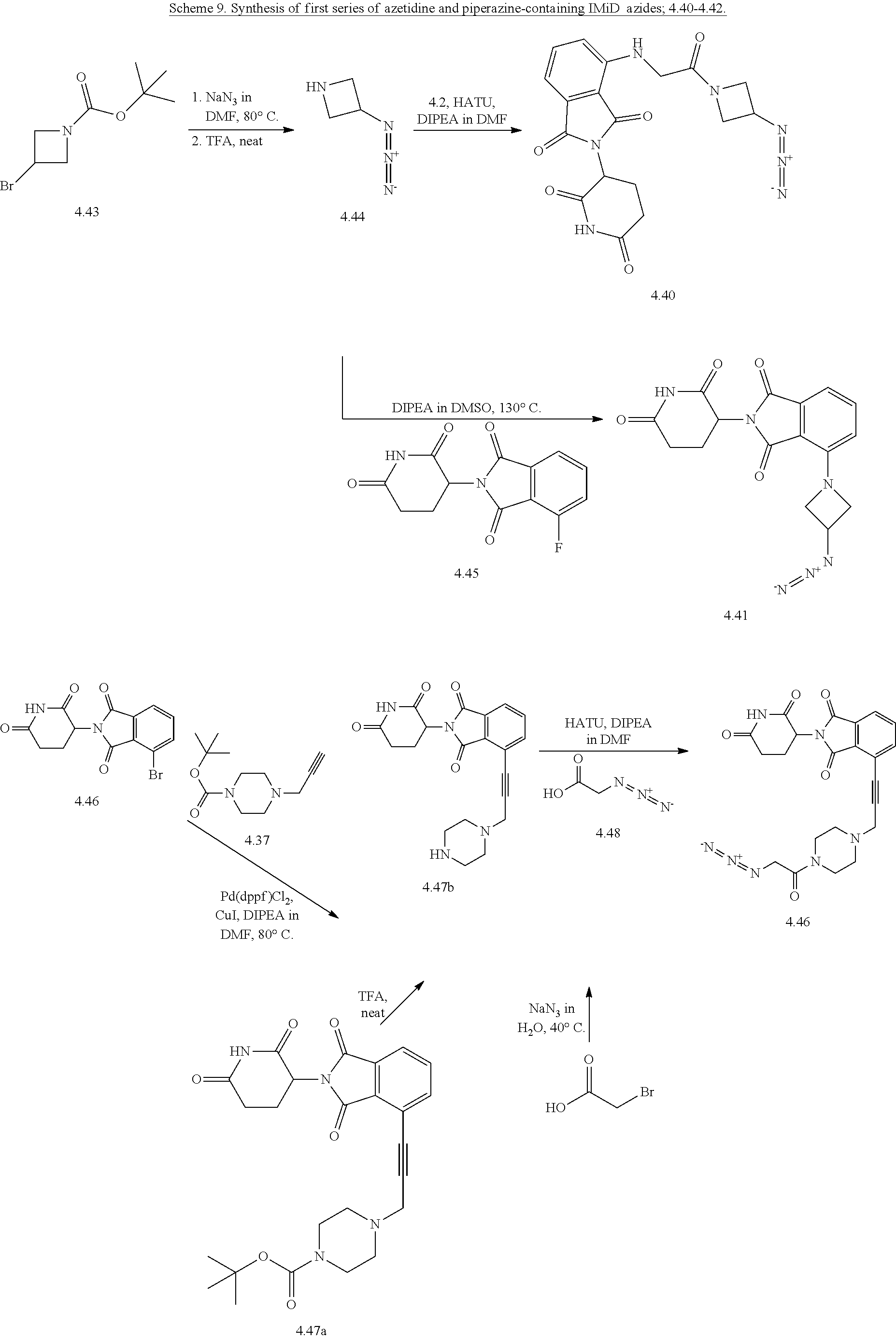

Scheme 10 presents the structures of the first series of the heterocyclic click series that was generated using the VIP152-based alkyne 4.35 and the three IMiD azides 4.40, 4.41 and 4.42. The evaluation of 4.49, 4.50 and 4.51 would then reveal some opportunities for improving physicochemical properties while setting realistic expectations with regard to the effect on potency from reducing lipophilicity (Table 4). This is seen from the results of characterizing degrader 4.49 compared to 4.50 and 4.51. This compound is able to achieve near 60 μM aqueous solubility. However, the compound shows only modest potency (420 nM). Despite this, 4.49 still represented the top molecule within the series, based on its balance between good aqueous solubility and modest potency. The other two degraders, 4.50 and 4.51 demonstrate low aqueous solubility (˜15 μM), while also showing high micromolar IC₅₀s against MOLM13 cells.

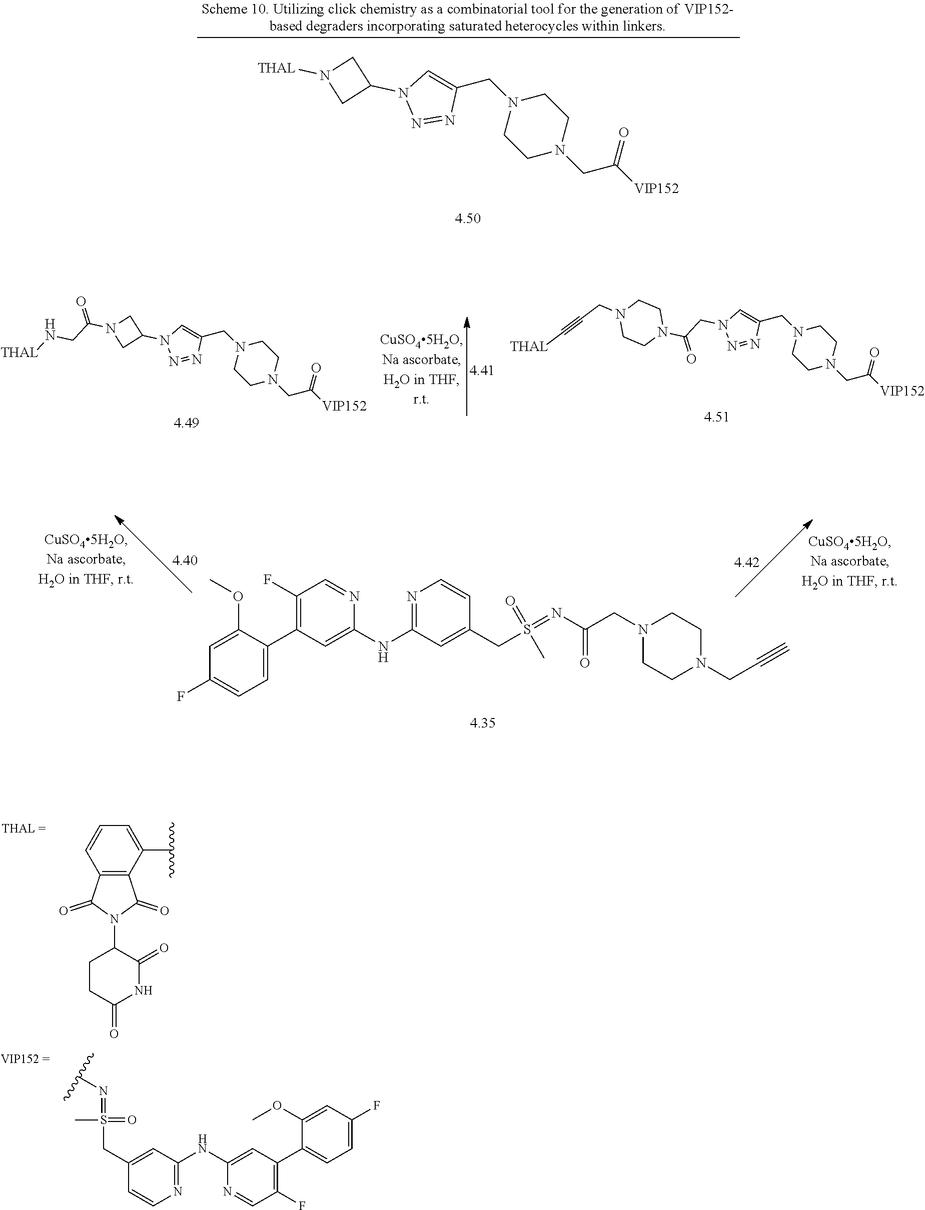

TABLE 4.4
Biological activity, kinetic solubility and chromatographic logD of first
heterocyclic click series of VIP152-based degraders.

| | | | Solubility | DC₅₀ | IC₅₀ |
|---|---|---|---|---|---|
| Structure | ID | logk′80 | (μM) | (μM) | (μM) |
| 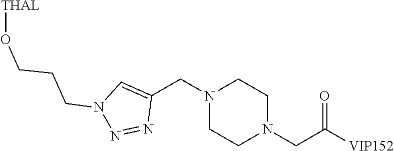 | 4.39 | 0.10 | 26.5 | 0.64 | 0.83 |
| 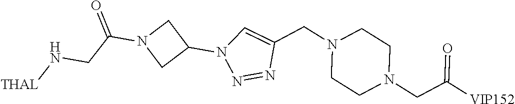 | 4.49 | 0.07 | 58.2 | 0.22 | 0.42 |
| 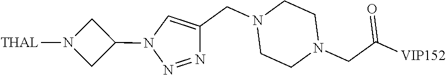 | 4.50 | 0.11 | 13.9 | 0.93 | 1.54 |
| 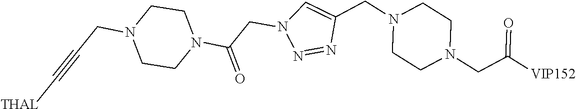 | 4.51 | 0.09 | 14.8 | 0.37 | 1.54 |

Given the loss of potency that was observed with the initial heterocyclic click series, other heterocycle-containing VIP152-based alkynes were synthesized: 4.52 and 4.53. The purpose of synthesizing these alkyne intermediates was to vary the linker structure on the 4.16 side, in order to explore the potential for improving potency, as was done with 4.40-4.42. The synthesis of the two piperidine-containing alkynes is illustrated in Scheme 11. Boc-protected ethynyl piperidine 4.54 was treated with TFA and subjected to reductive amination with sodium triacetoxyborohydride to produce the ester 4.56. Following hydrolysis of the ester tinder basic conditions, the resulting acid was coupled using EDC.HCl to 4.16, to obtain 4.52. Alkylation of the nipecotate ester 4.57 afforded the propargyl derivative 4.58. The hydrolysis afforded the carboxylic acid derivative which was used in the same EDC.HCl amide coupling procedure to produce 4.53.

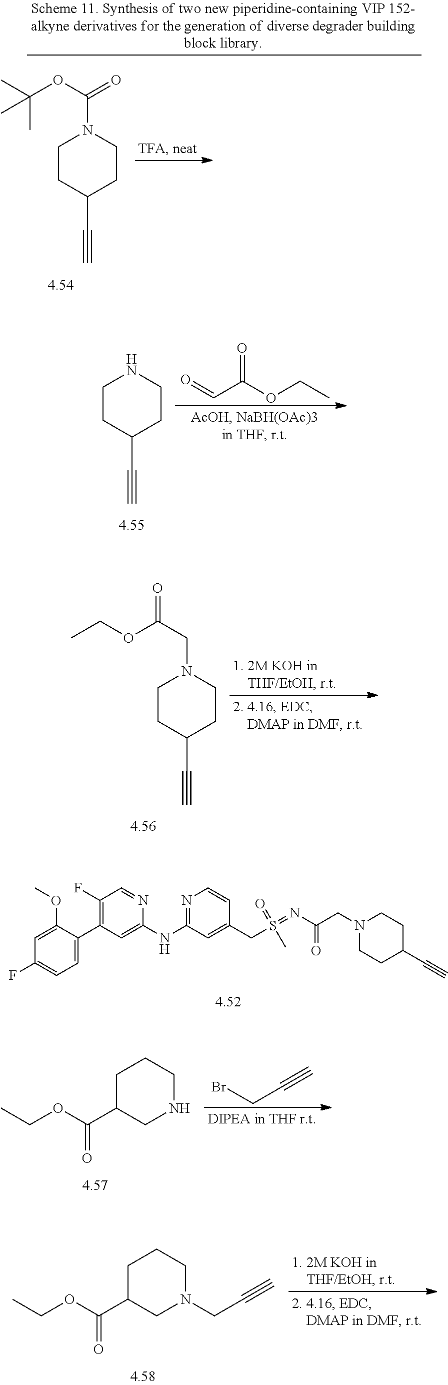

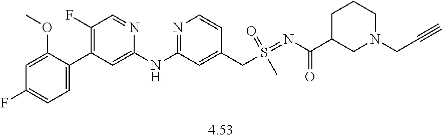 [SMILES: C#CCN1CCCC(C(=O)N=S(C)(=O)Cc2ccnc(Nc3cc(-c4ccc(F)cc4OC)c(F)cn3)c2)C1]

The synthesis of the two new alkynes would also contribute to the generation of valuable intermediates that could be used in a combinatorial manner along with synthesized IMiD azides to generate a diverse degrader library. This allowed for the rapid assembly of a second series of heterocyclic triazole-containing PROTACs: 4.59-4.64, The IMiD azides, 4.40-4.42 were used in the synthesis of this next series, as shown in Scheme 12. The results of the characterization of these PROTACs are shown in Table 5. The data show that the use of the two new piperidine-containing alkyne intermediates favored potency. However, in some cases, aqueous solubility was reduced. IMiD derivatives 4.41 and 4.42 resulted in degraders with overall lower potency, particularly cytotoxicity. This is based on the loss of activity observed with degraders 4.60 and 4.64. By contrast, the amide-containing azetidine analogue 4.59 was able to recover the potency lost in its piperazine analogue 4.49.

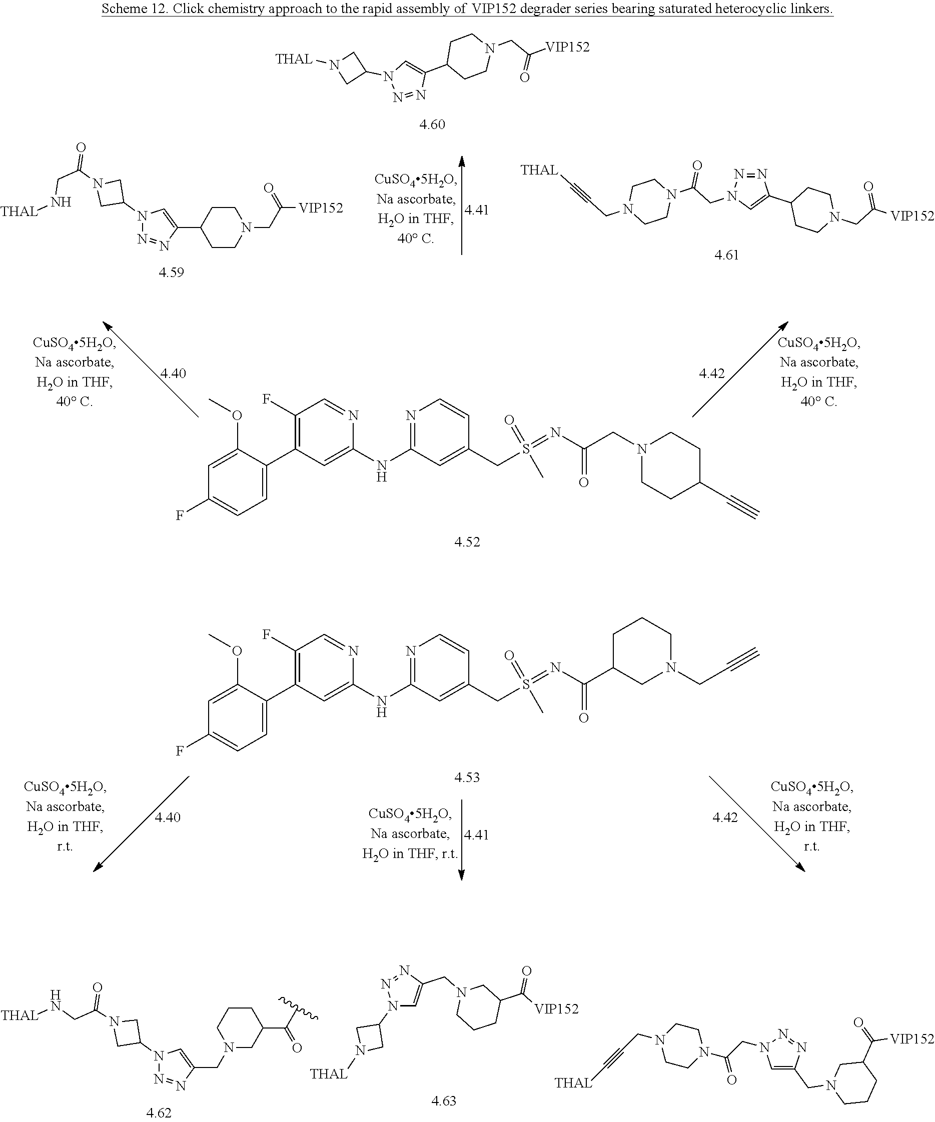

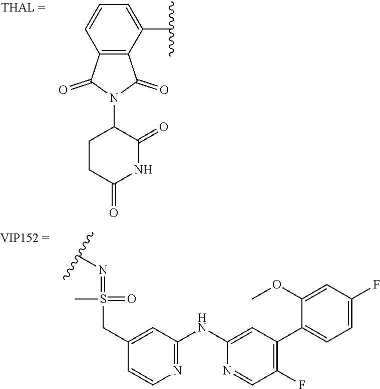 [SMILES: *c1cccc2c1C(=O)N(C1CCC(=O)NC1=O)C2=O ; *N=S(C)(=O)Cc1ccnc(Nc2cc(-c3ccc(F)cc3OC)c(F)cn2)c1]

TABLE 5
Biological activity, kinetic solubility and chromatographic logD of second series of
heterocyclic VIP152-based degraders 4.59-4.64.

| | | | Solubility | DC₅₀ | IC₅₀ |
|---|---|---|---|---|---|
| Structure | ID | logk′80 | (μM) | (μM) | (μM) |
| 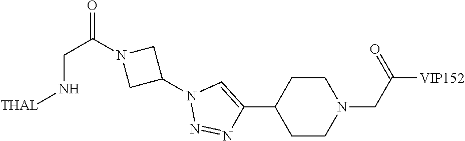 | 4.59 | 0.09 | 5.0 | 0.11 | 0.21 |
| 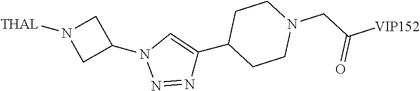 | 4.60 | 0.16 | 4.1 | 1.66 | 1.91 |
| 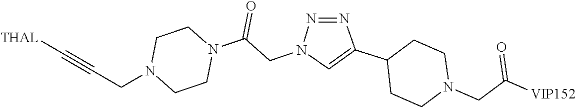 | 4.61 | 0.11 | 1.8 | 0.17 | 0.34 |
| 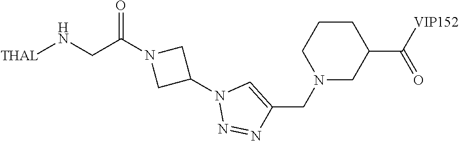 | 4.62 | 0.11 | 13.8 | 0.15 | 0.36 |
| 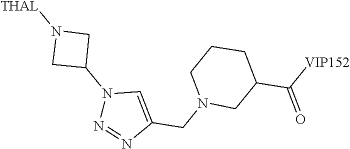 | 4.63 | 0.19 | 10.8 | 0.46 | 0.55 |
| 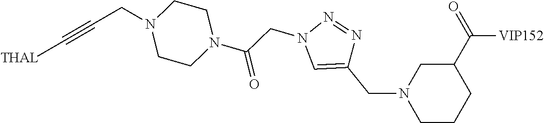 | 4.64 | 0.14 | 33.0 | 0.78 | 0.85 |

Interestingly, even though the two sets of degraders, 4.59-4.61 and 4.62-4.64 possess the same overall chemical structure, and are simply constitutional isomers of one another, notable differences can be observed from their physicochemical property assessments. With regard to aqueous solubility, 4.59-4.61 experienced a significant reduction in the solubility parameter relative to their corresponding isomers. This was interesting, given that the 4.59-4.61 set possessed the lower lipophilicity on average, according to the chromatographic log D determination. These data emphasized the complexity of structure-to-property translation for degraders and the value of the subtle, but impactful structural changes that were made between the two degrader sets. This is also due to the significant differences in potency that were observed between isomers across the two sets.

Due to the pattern of low potency that was associated with the incorporation of the two IMiD azides 4.41 and 4.42, a new set of IMiD azides was generated: 4.65-4.67. These would serve to facilitate the investigation of SAR for this set of PROTACs and to allow the assembly of a third series of VIP152-based degraders incorporating saturated heterocyclic linkers, with improved properties. The synthesis of this new IMiD azide series is illustrated in Scheme 13. The palladium-catalyzed reduction of intermediate 4.47a led to the facile synthesis of the propyl derivative 4.68 which was treated with TFA and subsequently coupled to acetic acid 4.48 using HATU to afford the IMiD azide 4.65. Using the same Sonogashira coupling conditions discussed earlier, the intermediate 4.69a, was synthesized from ethynyl piperidine 4.54 and subsequently deprotected using TFA. The resulting amine 4.69b was then coupled to azidoacetic acid 4.48 as was performed with 4.65, to obtain IMiD azide 4.66. For the synthesis of 4.67, the iodomethyl piperidine 4.70 was reacted with sodium azide with high heating to produce 4.71a, in good yield. The Boc-protected intermediate was then treated with TFA to obtain the piperidine azide 4.71b. The amine was then subjected to a nucleophilic aromatic substitution with 4.45 to obtain the desired IMiD azide product 4.67.

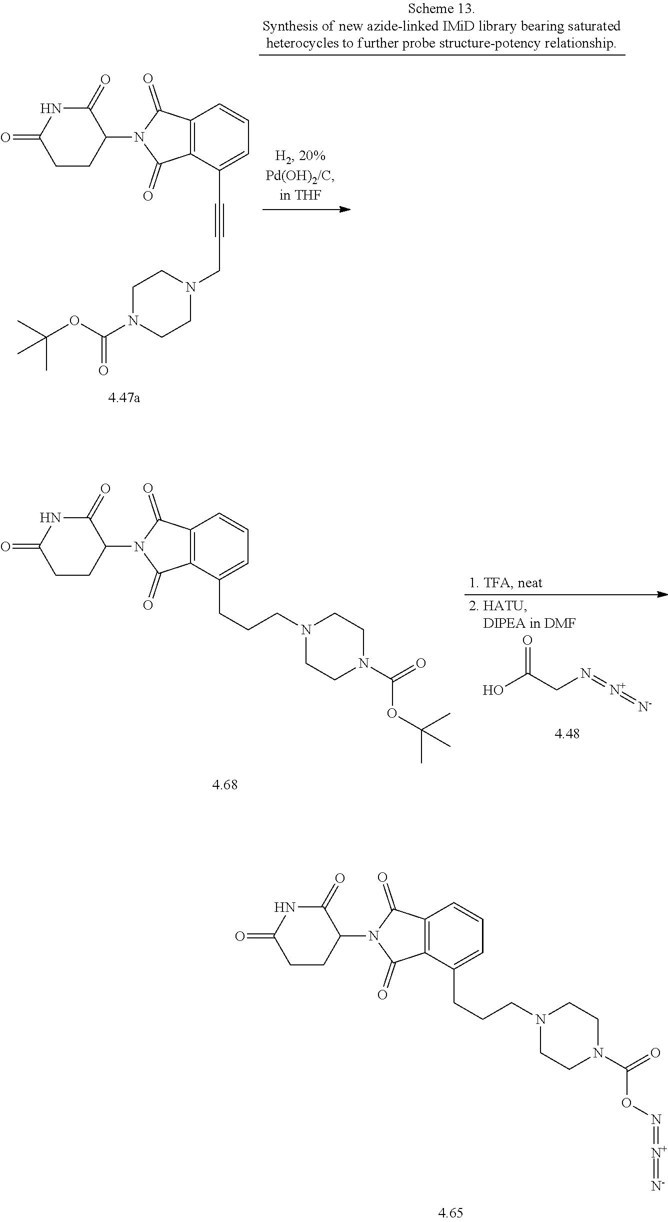 [SMILES: CC(C)(C)OC(=O)N1CCN(CC#Cc2cccc3c2C(=O)N(C2CCC(=O)NC2=O)C3=O)CC1 ; CC(C)(C)OC(=O)N1CCN(CCCc2cccc3c2C(=O)N(C2CCC(=O)NC2=O)C3=O)CC1 ; [N-]=[N+]=NCC(=O)O ; [N-]=[N+]=NOC(=O)N1CCN(CCCc2cccc3c2C(=O)N(C2CCC(=O)NC2=O)C3=O)CC1]

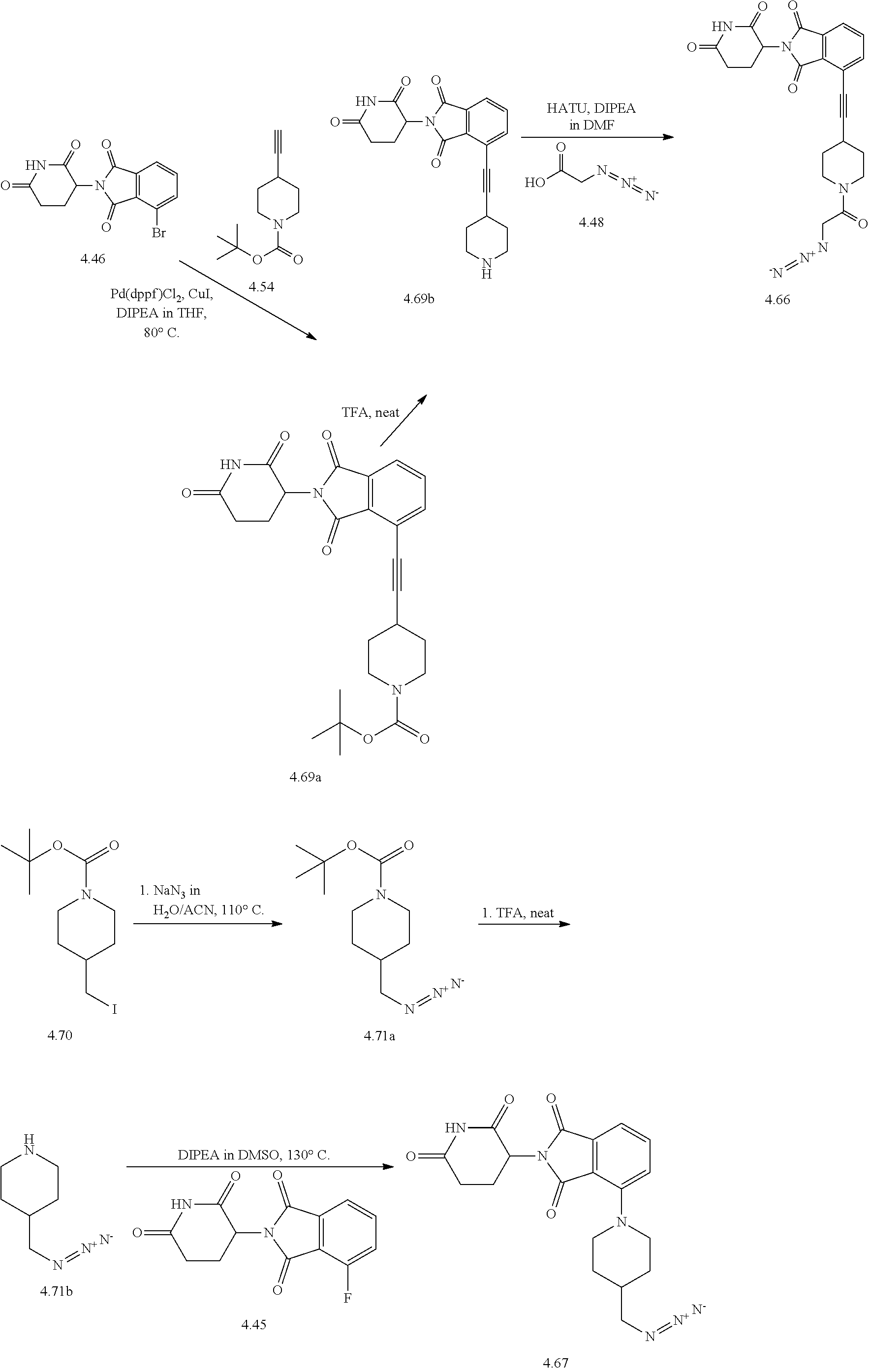

After obtaining the three new IMiD azides, the previously synthesized VIP152 alkyne derivatives, 4.52 and 4.53, were then reacted with the azides to afford a third heterocyclic click series 4.72-4.77. The degraders were synthesized according to Scheme and rapidly provided the desired PROTACs. The reduction of the alkynyl linkage in the imide azide 4.42 was performed to add a level of conformational flexibility that was expected to favor binding to the CRBN E3 ligase, while retaining the modest aqueous solubility that was observed in degrader 4.64. As presented in Table 6, this goal was somewhat attained by the degrader 4.72, with one of the highest potencies (150 nM) observed within the VIP152 series while achieving higher than average kinetic solubility 16 μM). However, while on average, the potency of this third series is higher than the previous 4.59-4.64 degrader set, aqueous solubility still suffers within the new series. It is also important to note the repetition of the pattern observed in the previous degrader series, i.e., the difference in measured kinetic solubility and lipophilicity between constitutional isomers.

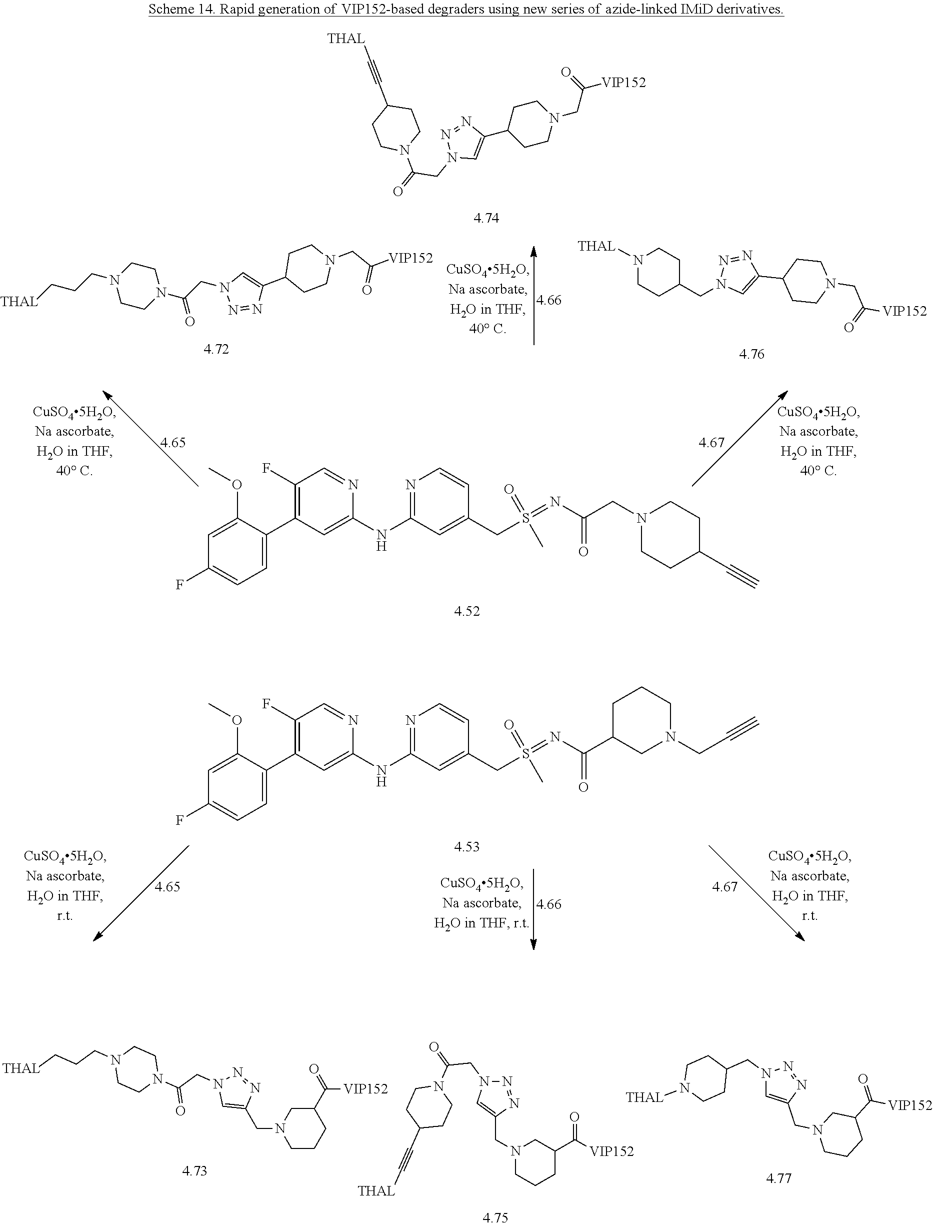

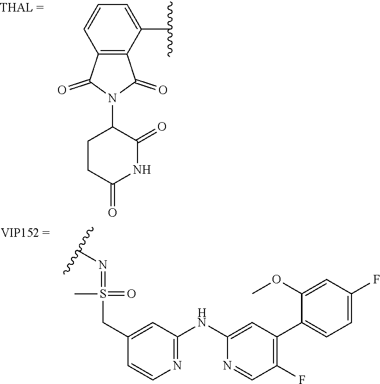 [SMILES: *c1cccc2c1C(=O)N(C1CCC(=O)NC1=O)C2=O ; *N=S(C)(=O)Cc1ccnc(Nc2cc(-c3ccc(F)cc3OC)c(F)cn2)c1]

TABLE 4.6
Cytotoxicity, degradation potency and physicochemical properties of third
VIP152-based heterocyclic click series.

| | | | Solubility | DC₅₀ | IC₅₀ |
|---|---|---|---|---|---|
| Structure | ID | logk′80 | (μM) | (μM) | (μM) |
| 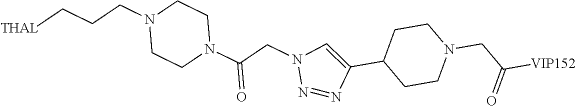 | 4.72 | 0.11 | 15.7 | 0.21 | 0.18 |
| 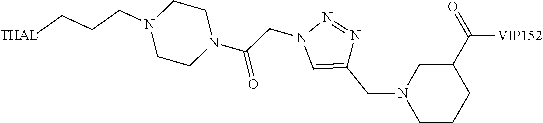 | 4.73 | 0.13 | 3.5 | 2.63 | 0.70 |
| 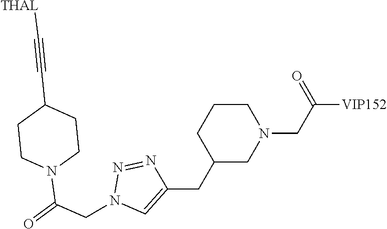 | 4.74 | 0.15 | 4.7 | 0.21 | 0.43 |
| 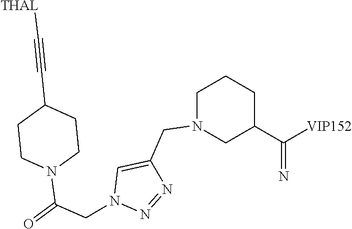 | 4.75 | 0.18 | 4.8 | 1.62 | 1.48 |
| 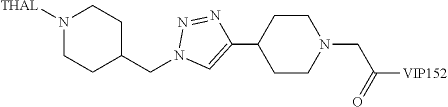 | 4.76 | 0.19 | 4.7 | 0.24 | 0.30 |
| 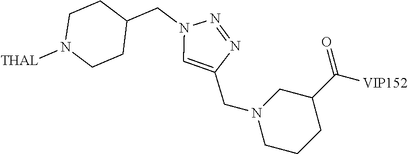 | 4.77 | 0.22 | 8.9 | 0.26 | 0.64 |

After this third series, the IMiD azide and VIP-152 alkyne derivative libraries had been explored for the generation of a diverse set of triazole-containing PROTACs. However, the set goals of a minimum 0.06 mM kinetic solubility and a maximum IC₅₀ of 60 nM in MOLM13 had not been attained. The one degrader that was closest to meeting these criteria was 4.49, but the molecule was still several fold less potent than the parent warhead. Given these data, it was clear that a redesign of the PROTACs was needed to further improve both potency and kinetic solubility. Therefore, the simplification of the linkers was performed by removal of the embedded triazole. This resulted in the design and synthesis of 4.78 and 4.79. By using simple piperidine rings to construct the linker, contribution to aqueous solubility by the ionizable nitrogens was expected. In addition, the removal of the triazole was expected to allow better permeability, especially given the reduction in molecular weight that would result. Finally, the higher flexibility of the new linkers relative to that of the triazole-containing analogues was expected to increase the potency of the new degraders. Both 4.78 and 4.79 were synthesized according to the route presented in Scheme 15. In a nucleophilic substitution, 4-hydroxy thalidomide 4.14 was reacted with the iodo-methyl piperidine 4.70. The product was treated with TFA to afford intermediate 4.80. In another nucleophilic substitution with 4.70 and following TFA treatment, the bipiperidine derivative 4.81 was obtained. The product was reacted with bromoacetic acid to afford the acid derivative 4.82 which was coupled to 4.16 using EDC.HCl. Similarly, to obtain the degrader 4.79, 4.80 was reacted with bromoacetic acid, resulting in the acetic acid derivative 4.83b. The obtained product was then coupled to 4.16 under the same amide coupling conditions as 4.78 to afford degrader 4.79.

Neither degrader 4.78 nor 4.79 demonstrated exceptional potency, specifically potency comparable to VIP52 (Table 7), However, 4.78 did show higher than average aqueous solubility while demonstrating modest potency.

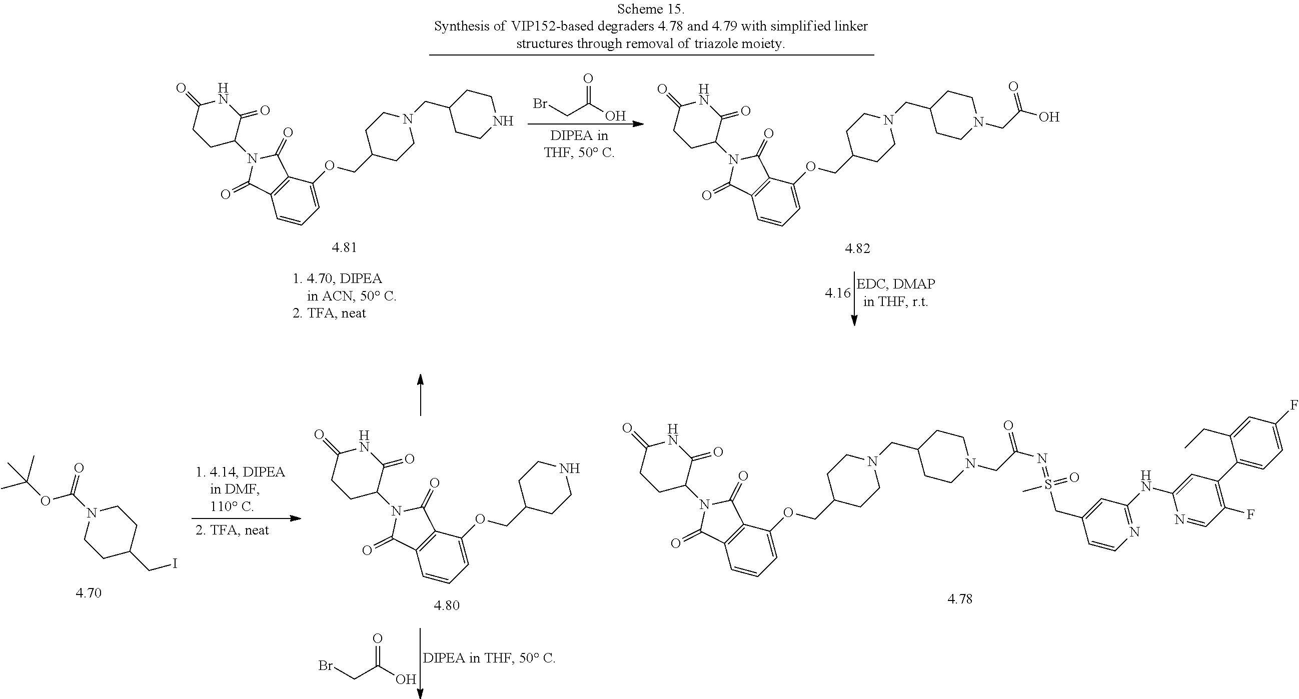

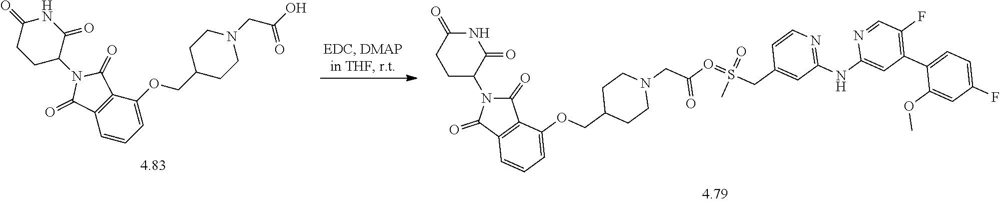

TABLE 4.7
Characterization of simplified degraders 4.78 and 4.79.

| | | | Solubility | DC₅₀ | IC₅₀ |
|---|---|---|---|---|---|
| Structure | ID | logk′80 | (μM) | (μM) | (μM) |
| 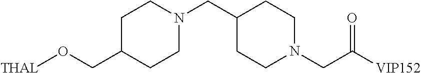 | 4.78 | 0.18 | 28.9 | 0.82 | 0.41 |
| 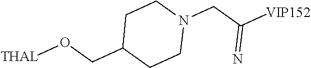 | 4.79 | 0.20 | 3.1 | 0.36 | 0.19 |

Due to the unexceptional potency demonstrated by these simplified degraders, the click chemistry strategy was chosen, yet again, for the assembly of the next set of degraders. The purpose of choosing the synthetic strategy a second time was to explore the potential to augment potency by varying the position of the rigid triazole across a relatively, more flexible linker. This is based on the influence on cytotoxicity that was observed in the AT7519 click series. This led to the synthesis of the azide derivatives 4.84 and 4.85 (illustrated in Scheme 16). The purpose of these IMiD and VIP152 derivatives, with their long heterocyclic linker precursors, was to perform click chemistry with shorter, simpler alkyne derivatives, such that, as with the AT7519 click series, the number of linker atoms could be varied on the side of each ligand. This strategy was expected to improve potency while allowing an increase in TPSA that would favor aqueous solubility. This is based on a study that was performed on degraders wherein it was determined that good aqueous solubility could be attained for compounds with higher TPSA, despite high apparent lipophilicity. Therefore, to obtain 4.84, the oxoazetidine 4.86, was reacted in a reductive amination with the piperazine azide 4.71b. The acquired product was treated with TFA to obtain the free azetidine 4.87b which was reacted with 4.2 tinder amide coupling conditions to afford 4.84. To obtain the desired VIP152 azide analogue 4.85, the carboxylic acid derivative 4.88, would first be synthesized through amide coupling with succinic acid. The product would then be reacted with 4.87b using EDC.HCl as the coupling reagent to afford the desired azide-linked precursor, 4.85.

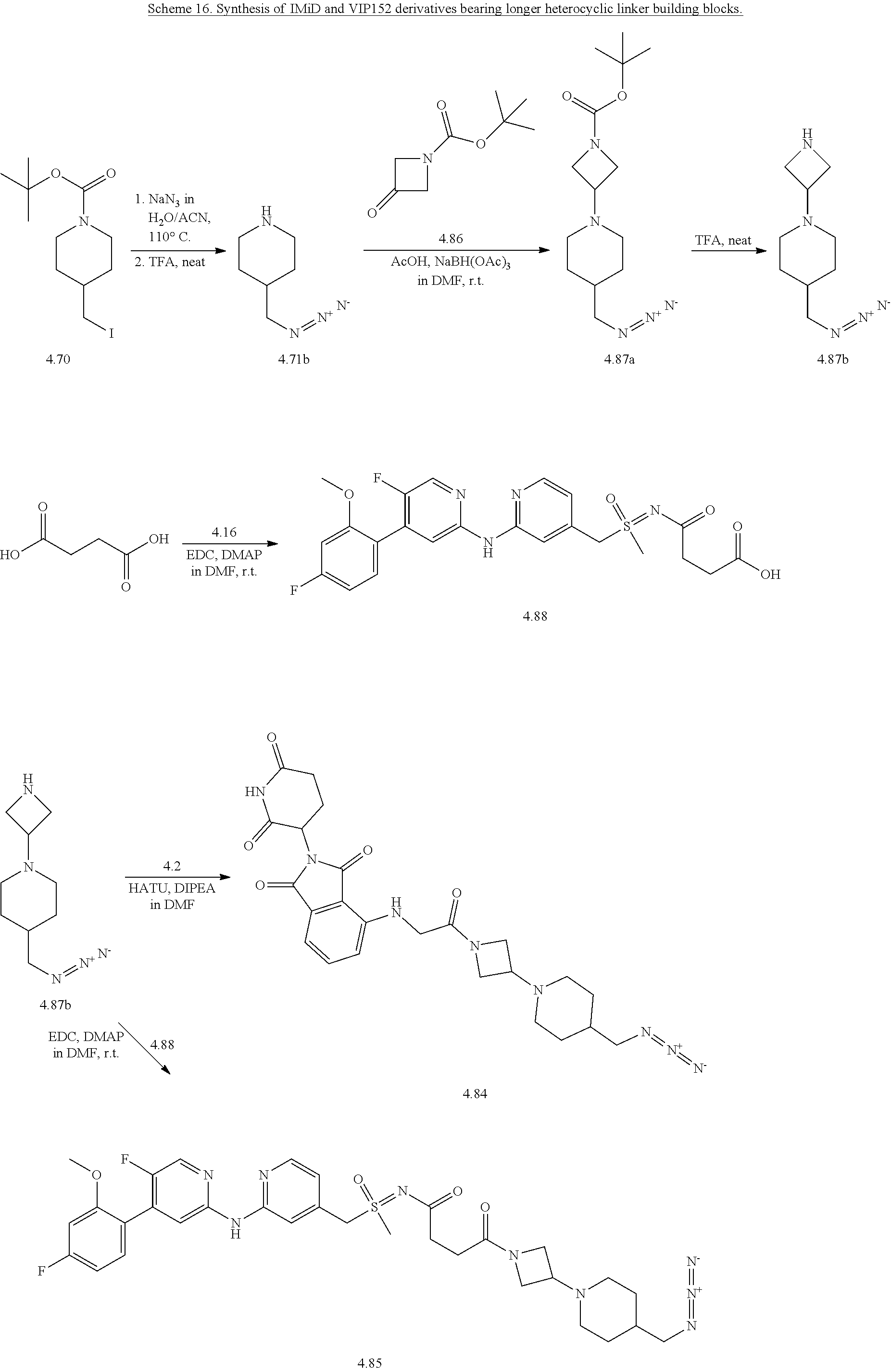

Using the previously discussed butyne derivative of VIP152 4.30, degrader 4.89 could be afforded using standard click chemistry conditions with azide 4.84. The simple alkyne derivative of pomalidomide 4.92 was synthesized by coupling 4.2 to propargylamine using HAT coupling conditions. The propargyl derivative of 4-hydroxy thalidomide 4.93 was obtained through a nucleophilic substitution reaction with propargyl bromide. With these alkyne intermediates in hand, the two degraders 4.90 and 4.91 could be rapidly generated from the azide intermediate 4.85.

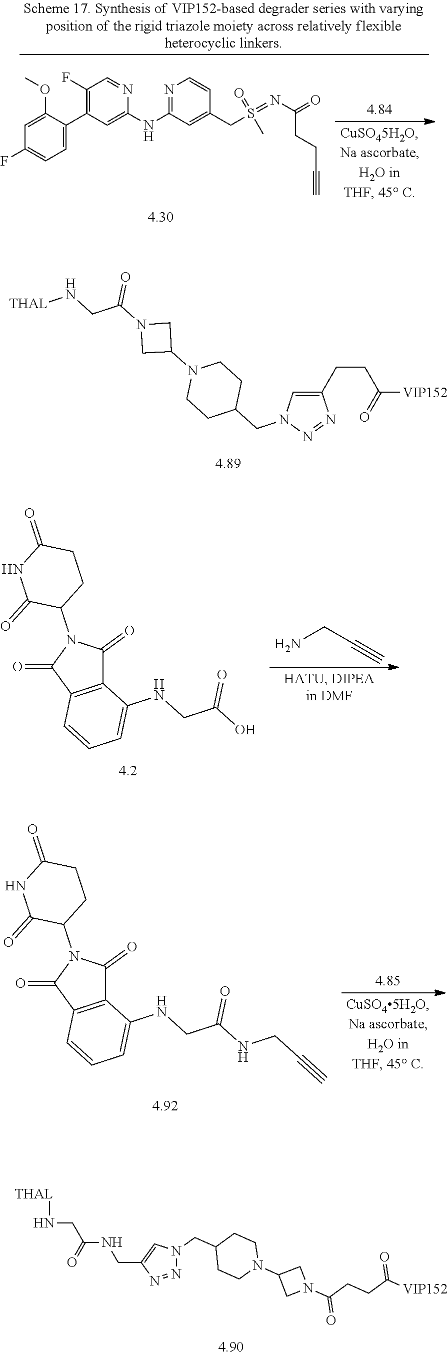

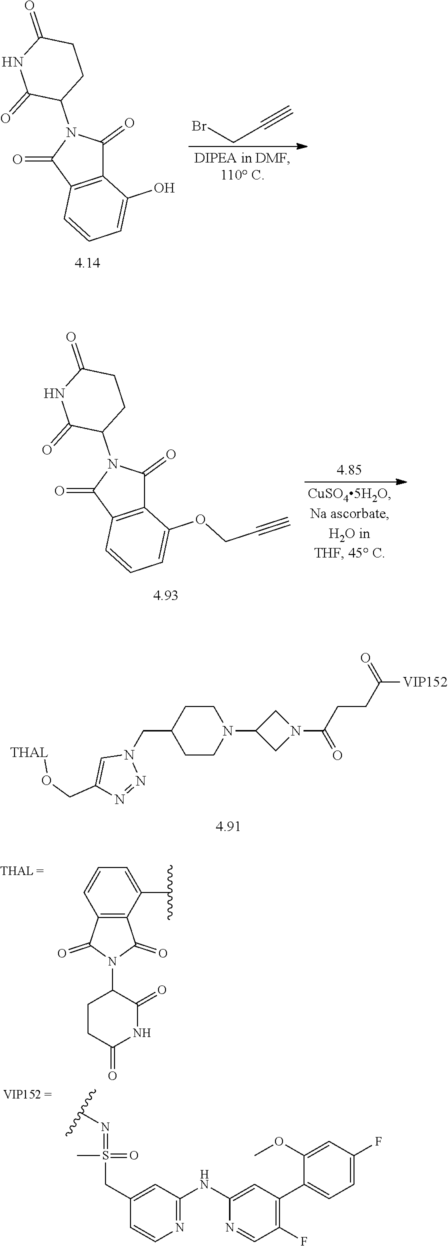

The results of biological and physicochemical determinations for the three degraders 4.89-4.91 are presented in Table 8. The data show that neither aqueous solubility nor potency is significantly improved within this degrader set. However, the degradation potency of 4.89 was one of the highest observed within the VIP152 series. With regard to DC₅₀, the potency of 4.89 is similar to that of 4.34, the most potent degrader within the VIP152 degrader series. Despite the above average biological activity of 4.89, the molecule fails to demonstrate significant aqueous solubility, an unanticipated outcome, given its low log k′80.

TABLE 4.8
Characterization of the final triazole-containing VIP152-based degrader series.

| | | | Solubility | DC₅₀ | IC₅₀ |
|---|---|---|---|---|---|
| Structure | ID | logk′80 | (μM) | (μM) | (μM) |
| 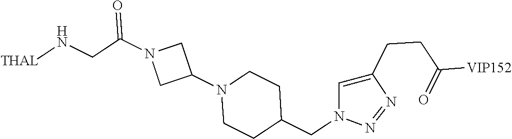 | 4.89 | 0.09 | 1.8 | 0.08 | 0.17 |
| 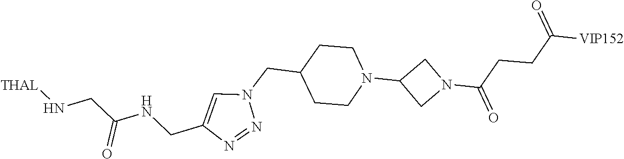 | 4.90 | 0.07 | 8.3 | 0.90 | 1.38 |
| 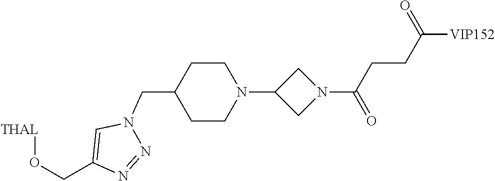 | 4.91 | 0.09 | 3.8 | 0.26 | 0.59 |

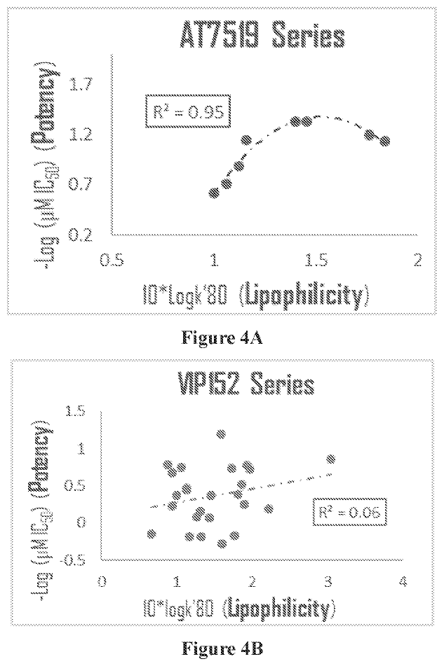

Given the number and diversity of the VIP152-inspired degraders that had now been generated, it was important to examine structure-physicochemical property relationships. First, a plot of cytotoxicity against measured log k′80 was created and is presented in FIG. 4.4A, for the AT7519 click series and in 4.43, for the VIP152-based degraders. The curve fit analysis shows that the correlation between potency and cytotoxicity is significantly reduced in the VIP152 series, relative to the AT7519 click series. This is attributed to the redesign of the linker building blocks that make up the diverse VIP152 degrader set.

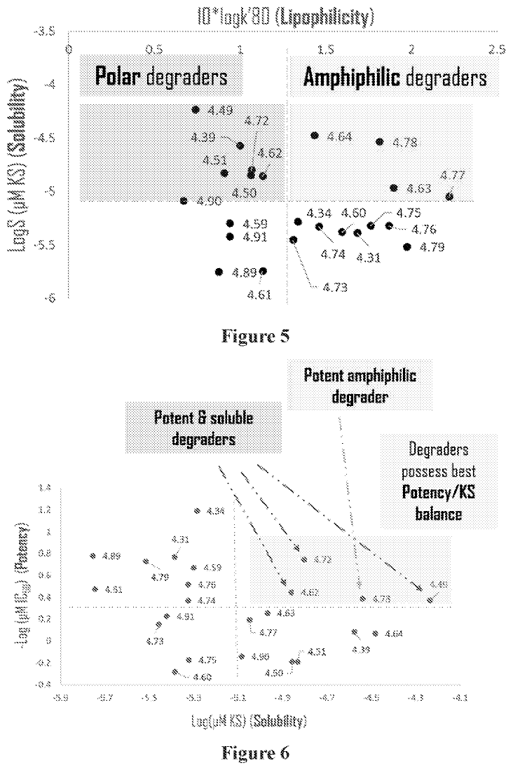

Also, a plot of kinetic solubility against measured chromatographic log D (log k′80) was created and is presented in FIG. 5. Interestingly, there was no correlation (R²═0.00) between these two measured parameters for the VIP152 series. Moreover, three degraders were found to possess higher than average aqueous solubility while demonstrating higher log k′80s. These degraders demonstrate amphiphilicity, a physicochemical property that is known to favor good oral absorption. A higher number of degraders demonstrated the expected pattern of low aqueous solubility and high log k′80, and vice versa. However, the discovery of the compounds that defy the more common pattern is noteworthy. FIG. 5 provides a labeled graphical plot of measured solubility against lipophilicity that indicates the compound corresponding to a particular data point. For the polar degraders in the upper-left quadrant of the plot, it was found that they possess, on average, higher TPSA and molecular weight. The same review of calculated properties for the amphiphilic degraders reveals that the compounds possess lower molecular weight and TPSA than average and do not contain any hydrogen bond donors within their linkers.

Along with an analysis of the structure-physicochemical property relationships within the VIP152 degrader series, lead compounds also needed to be selected. This was achieved by identifying the compounds that demonstrated both higher than average aqueous solubility and cytotoxicity. FIG. 6 illustrates these efforts using a graphical plot of cytotoxicity in MOLM13 cells against kinetic solubility. In the upper-right quadrant of the plot, there are four degraders that meet the criteria for lead compound selection. Three of the four compounds are polar overall, based on their low log k′80s and higher than average kinetic solubility. However, there is one compound, 4.78, which is an amphiphilic molecule, that is able to demonstrate decent potency and aqueous solubility.

In order to determine the suitability of the lead compounds for advanced biological studies, the four degraders (4.49, 4.62, 4.72 and 4.78) were selected for a protein-shift analysis due to their higher potency and solubility. The most potent degrader 4.34, was also selected for this analysis to determine if its high potency would be retained in human serum. The data are provided in Table 9. The data show that despite the high potency of 4.34 in fetal bovine serum, the degrader was outperformed by every other degrader when tested in human serum in particular, 4.78 and 4.72 were able to retain their cytotoxic activity against the MOLM13 cells in human serum Interestingly, the other two polar degraders, 4.49 and 4.62, lose their potency significantly.

TABLE 9
Results of protein shift experiment on lead compounds and 4.34 to determine cytotoxicity in human serum.

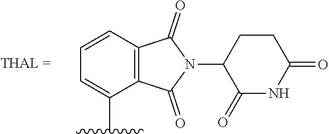

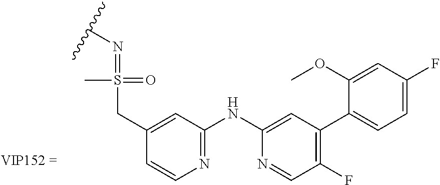

| | | | | IC₅₀ | IC₅₀ |
|---|---|---|---|---|---|
| | | | Solubility | (μM) | (μM) |
| Structure | ID | logk′80 | (μM) | FBS | HS |
| 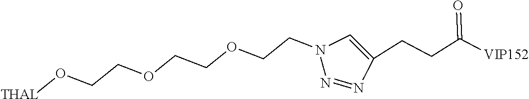 | 4.34 | 0.16 | 5.2 | 0.29 | 1.52 |
| 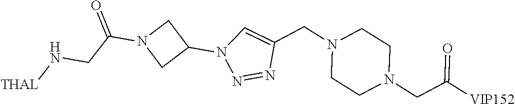 | 4.49 | 0.10 | 58.2 | 0.67 | 1.24 |
| 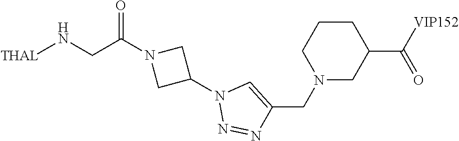 | 4.62 | 0.11 | 13.8 | 0.49 | 1.34 |
| 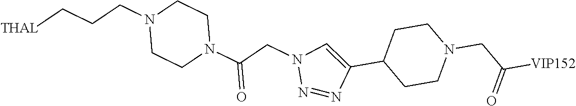 | 4.72 | 0.11 | 15.7 | 0.47 | 0.55 |
| 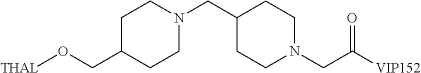 | 4.78 | 0.18 | 28.9 | 0.52 | 0.49 |

Upon reviewing the data from the protein shift experiment, it was clear that the most favorable linker structure was that of 4.78. This is because despite its similarity to 4.72, based on their activity in the protein shift assessment, 4.78 still delivers the higher aqueous solubility. Given this conclusion, the next set of degraders would then be designed such that the triazole moiety would be removed and the molecules would possess the piperazine and piperidine moieties, with free, ionizable tertiary amines. The linker structure would then be rigidified and a more potent IMiD, lenalidomide, would be incorporated to improve potency. Scheme 18 illustrates the synthesis of two key linker building blocks for the next degraders: 4.94 and 4.95. The synthesis of the degraders was facilitated by the gram-scale synthesis of the piperidine- and piperazine-containing alkynes 4.96 and 4.97. The building blocks were obtained through reductive amination with the alkynes 4.55 and 4.37b. The propargyl derivative 4.98 was then deprotected with TFA and reacted with tert-butyl bromoacetate to obtain the ester 4.97. The derived products 4.96 and 4.97 were reacted with bromno-lenalidomide 4.99 through sonogashira coupling to produce 4.100 and 4.101. The bipiperidine intermediate 4.100 was then treated with TFA to obtain 4.102 which was reacted in a nucleophilic substitution with bromoacetic acid to obtain the intermediate 4.94. Similarly, Boc-protected piperazine-containing 4.101 was treated with TFA and neatly provided the desired carboxylic acid 4.95. With the carboxylic acids 4.94 and 4.95 in hand, both degraders 4.103 and 4.104 were obtained through HATU amide coupling with 4.16. The synthesis of the final two VIP152-based degraders is presented in Scheme 19.

The results from analyses of these two degraders are provided in Table 10. The characterization of 4.104 shows that while the piperazine-containing degrader is able to attain a relatively high kinetic solubility of over 50 nM, the compound demonstrates poor potency against AML cells (>1 μM). By contrast, 4.103 attained significant potency while demonstrating a modest kinetic solubility of 29 μM. Therefore, within this VIP152-inspired series, 4.103 would represent the degrader that offered the best of the two prioritized parameters: aqueous solubility and potency.

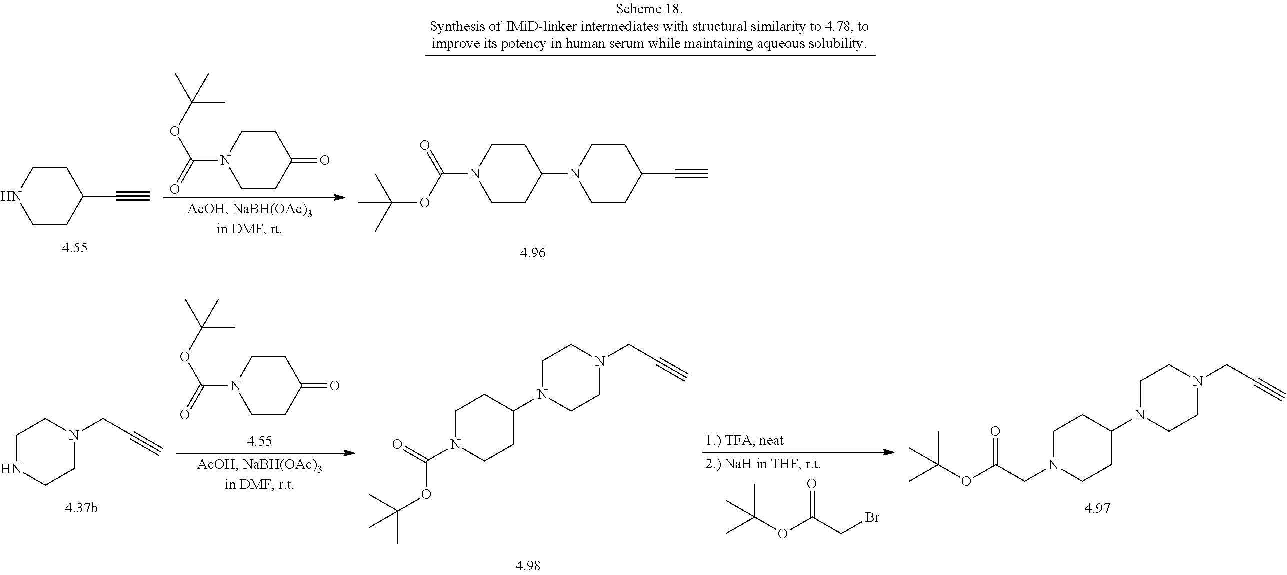

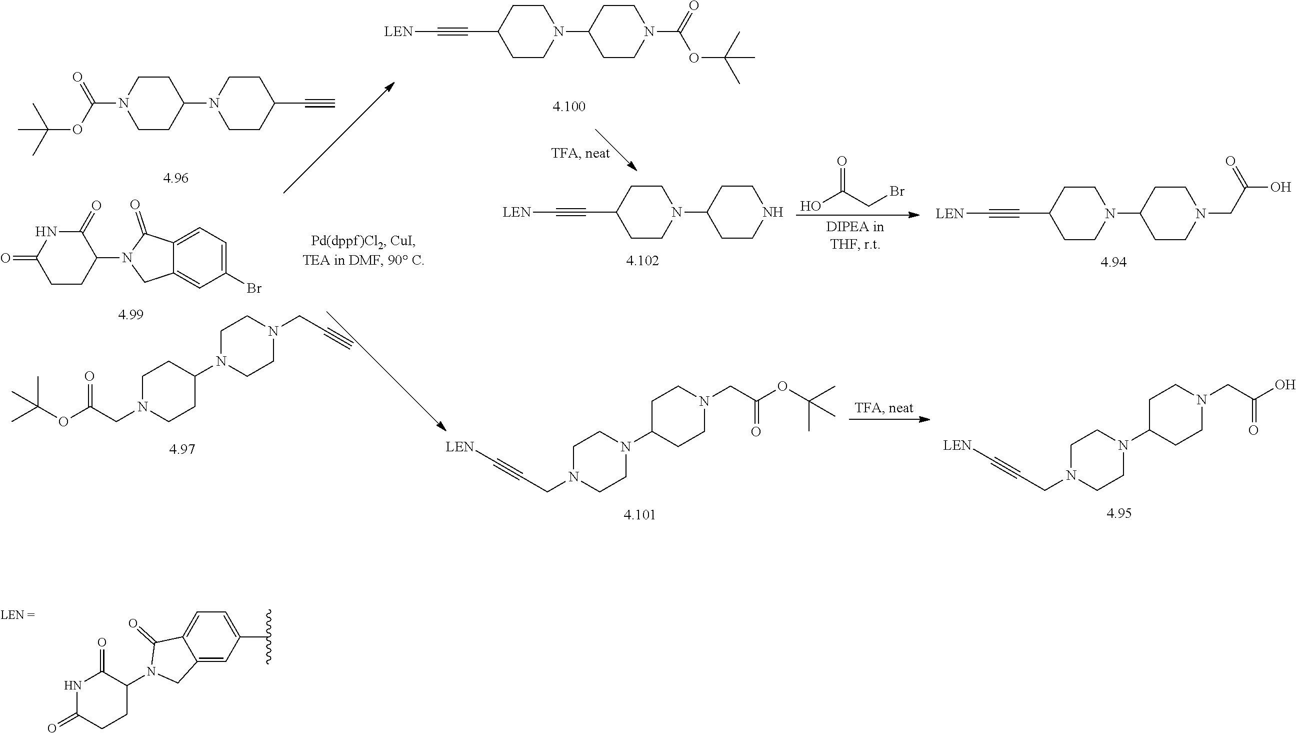

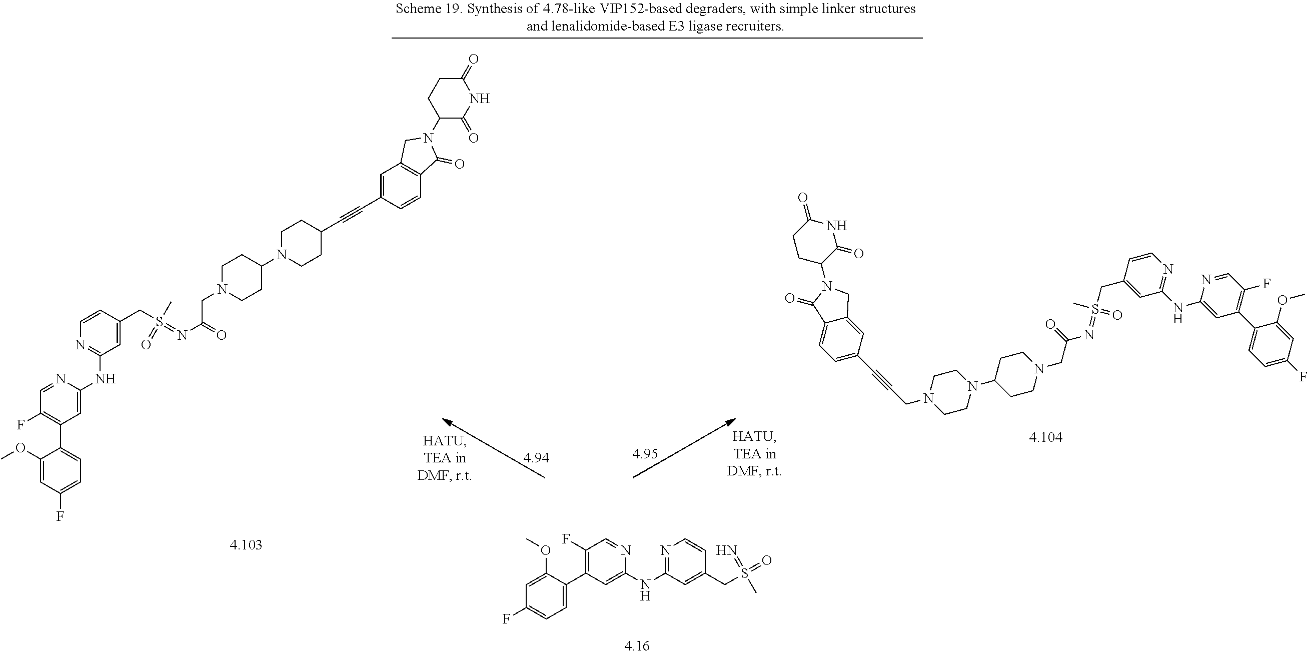 [SMILES: COc1cc(F)ccc1-c1cc(Nc2cc(CS(C)(=O)=NC(=O)CN3CCC(N4CCC(C#Cc5ccc6c(c5)CN(C5CCC(=O)NC5=O)C6=O)CC4)CC3)ccn2)ncc1F ; COc1cc(F)ccc1-c1cc(Nc2cc(CS(C)(=O)=NC(=O)CN3CCC(N4CCN(CC#Cc5ccc6c(c5)CN(C5CCC(=O)NC5=O)C6=O)CC4)CC3)ccn2)ncc1F ; COc1cc(F)ccc1-c1cc(Nc2cc(CS(C)(=N)=O)ccn2)ncc1F]

TABLE 10
Results of characterization of the final set of VIP152-based degraders.

| | | | Solubility | DC₅₀ | IC₅₀ |
|---|---|---|---|---|---|
| Structure | ID | logk′80 | (μM) | (μM) | (μM) |
| 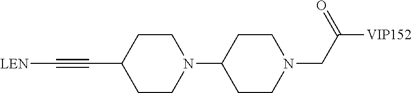 | 4.103 | 0.15 | 29.1 | ND | 0.12 |
| 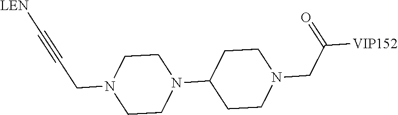 | 4.104 | 0.11 | 51.6 | ND | 1.10 |

### Conclusion

Overall, the design and characterization of this VIP152-based CDK9 degrader series has demonstrated the complexity of SAR with heterocyclic linkers. There was no significant correlation found between individual predicted physicochemical properties and observed aqueous solubility or lipophilicity. Moreover, there was no clear relationship between linker structure and potency. Despite this, some lessons regarding linker design are evident upon analyzing this degrader series and its observed properties. First, incorporation of the ionizable tertiary amines within the embedded heterocycles results in improvements in aqueous solubility and attenuation of lipophilicity. This is seen from the results of transforming classical alkyl and PEG groups within 4.31-4.34 (Table 3) into piperazine-bearing 4.39 (Table 4). However, when linkers are overwhelmed with groups that significantly increase molecular weight with a disproportional increase in TPSA, the desired improvement in kinetic solubility is not observed. Examples of this phenomenon are seen in 4.61, 4.73, 4.74, 4.89 and 4.91, which can all be found in the lower left quadrant of plot in FIG. 5. These compounds were unable to exhibit above average kinetic solubility despite possessing ionizable amines, relatively high TPSA and low log k′80s. A closer look at these compounds shows that they all have higher than average molecular weight. Conversely, 4.63, 4.77 and 4.78 are compounds with lower TPSA and lower molecular weight which are found in the upper right quadrant of the plot. These compounds are able to attain higher than average kinetic solubility, despite their relatively high lipophilicity. Together, these observations show that when incorporating these ionizable heterocyclic groups into linkers, minimization of molecular weight is the best strategy for improving aqueous solubility Additionally, the choice of the saturated heterocycles often proved critical as all six nipecotate and ethynyl piperidine-derived isomeric pairs demonstrated differences in kinetic solubility and/or observed lipophilicity (Tables 5 and 6). Lastly, the potency and kinetic solubility exhibited by 4.103 positions the molecule as a good candidate for further investigation. These would comprise in vitro assays such as the serum shift experiment, permeability and metabolic analyses. These experiments would then be followed by in vivo evaluations in AML engrafted mice to determine the potential of the molecule as an AML therapy for human applications. In summary, the linker design efforts described herein demonstrate that a heterocyclic linker library can be a useful tool for obtaining degraders with potency and physicochemical properties that are advantageous for good bioavailability.

### Materials and Methods

General Materials and Methods. Reagents used were purchased from commercial sources, as reagent grade quality chemicals, and were used without further purification. AT7519 was synthesized as the TFA salt as previously described. 4-hydroxy thalidomide was also synthesized using a previously reported synthetic scheme. Reactions that required inert conditions were performed under argon atmosphere. Reactions were monitored using Thin-Layer Chromatography (TLC) on silica gel plates with aluminum backing. Mass spectrometry was also used to monitor reaction progress as needed. Normal phase (NP) chromatography was run manually using silica gel while reversed phase (RP) purifications were run on C18 RediSep columns on a Teledyne Isco CombiFlash system. Crude materials to be purified were adsorbed onto either silica gel or Celite®545 for NP or RP chromatography, respectively. Purity was determined on a Shimadzu High-Performance Liquid Chromatography (HPLC) system on a Phenomenex Luna 5-micron C18 column 150×4.60 mm) where UV absorbance was monitored under 254 and 337 nm. Separations were run with a water/methanol eluent solvent system (with 0.1% formic acid) and flow rate was 1 mL/min. High resolution accurate mass (HRAM) data was acquired on a Thermo Scientific Q-Exactive Orbitrap mass spectrometer using external calibration. Samples were introduced into the mass spectrometer by flow injection using an Agilent 1100 HPLC operating at a 700 μL/min flow rate using 99.9% LC/MS grade Methanol (Fisher Optima) 0.1% LC/MS grade Formic Acid (Fisher Optima) as eluent. ESI ionization was performed using default parameters given by Q-Exactive Tune software (version 2.11) for the 500 μL/min flow rate without further optimization. Characterization by ¹Proton NMR (400 MHz or 700 MHz) and ¹³CNMR (100 MHz or 175 MHz) was performed on a Bruker Advanced III NMR spectrometer where the selected deuterated solvent was used as a standard for internal deuterium lock. Chemical shifts for signals are given as “6 chemical shift (multiplicity, coupling constant (J value in Hz), integration).” Chemical shifts are reported in parts per million (ppm) relative to tetramethylsilane (TMS) which has δH=0.00 ppm Multiplicity is denoted by “s,” “d,” “t” “q,” “p,” and “m” to indicate singlet, doublet, triplet, quartet, quintet and multiplet signals, respectively. Solvent signals were identified based on the chemical shifts.

Kinetic Solubility Assay. Kinetic solubility (KS) was measured according to Millipore protocol PC2445 EN00. Test compounds were weighed and diluted with DMSO to 10 mM. From the stock solutions as well as blank (100% DMSO) was taken 17.5 μL and diluted to 500 μM with 332.5 μL of 100 mM phosphate Buffered Saline (PPBS) at pH 7.4 in a 96-well quartz plate. From the 500 μM solutions was taken 150 μL and dispensed into a 96-well Millipore filter plate (Product number: MSSLBPC10). In the quartz plate, serial dilutions were performed down to 0.5 μM using the 100 mM PBS solution, leaving 100 μL of each test sample at 11 different concentrations, representing standards. These steps were performed in triplicate, at minimum, for each test compound. Both plates were shaken on an orbital shaker at 150 rpm for 1.5 hours. The contents of the filter plate were filtered and 100 μL of the filtrate from each well was dispensed into the quartz plate containing standards. The quartz plate was then analyzed using a UV plate reader where absorbance was measured at the relevant wavelength for each compound. After blank subtraction, for each test sample, solubility was determined based on the optical density of filtered samples and the equations derived from the corresponding linear standard curve.

Chromatographic Log D Determination. Test samples were prepared from 10 mM stock solutions in DMSO, to make 122 μM solutions in acetonitrile. Retention times were determined on a Shimadzu High Performance Liquid Chromatography (HPLC) system with Agilent PLRP-S column, 100 Å, 5 um particle size, 50×4.6 mm (part no.: PL1512-1500) with 1 mL/min flow rate. A 65 mM aqueous solution of ammonium acetate was prepared in water and diluted with acetonitrile to give 0.1% ammonium acetate 20:80 acetonitrile solution with pH 7.72. Eluents were detected with UV spectrophotometry at 337 nm. As a standard set, the retention times of β-estradiol, nifedipine, quinoline, phenol and 2-nitrophenol were determined (log k′80) and the dead time was extrapolated based on the reported log k′80 values originally reported by Caron et. al (log k′80*). The average value of the dead time for the standard set was 0.3001 minutes. Therefore, for consistency, 0.3 minutes was used as the dead time for all test samples. Experiments were performed in triplicate with a maximum percent standard deviation of 6.85%.

General procedure A (amide coupling with VIP152). To a solution of carboxylic acid in DMF (0.2 M) was added N,N-dimethylpyridin-4-amine-DMAP and N′-(3-dimethylaminopropyl)-N-ethylcarbodimide, hydrochloride salt-EDC·HCl. The mixture was stirred for ten minutes before addition of VIP152. The reaction mixture was stirred for a minimum of two hours after which the reaction was concentrated under reduced pressure and taken directly onto purification. The crude mixture was purified by reversed phase chromatography (C18, ACN:H₂O eluent).

General procedure B (click chemistry). Sodium ascorbate and CuSO₄·5H₂O were completely dissolved in water (0.07 M). The mixture was then added to a solution of alkyne and azide in THF (0.05 M). DMF was added to facilitate dissolution as needed. The reaction was allowed to proceed at room temperature or 45° C. overnight. The reaction mixture was concentrated under reduced pressure and the crude mixture was purified by reversed phase chromatography (C18, ACN:H₂O eluent).

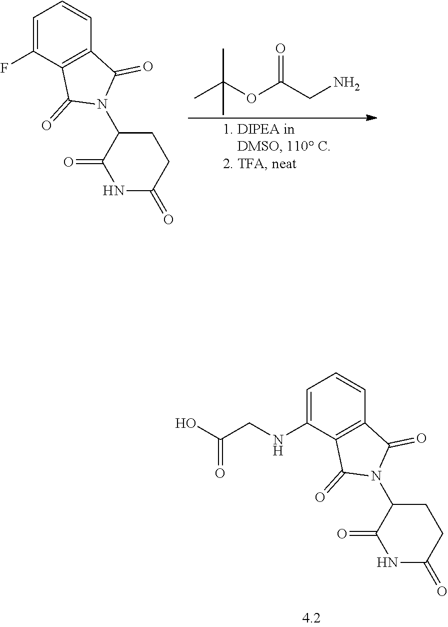

### (2-(2,6-dioxopiperidin-3-yl)-1,3-dioxoisoindolin-4-yl)glycine (4.2)

2-(2,6-dioxopiperidin-3-yl)-4-fluoroisoindoline-1,3-dione (2.0 g, 7.24 mmol) and tert-butyl glycinate (2.8 g, 16.7 mmol) were dissolved in DMSO (0.13 M) and heated to 110° C. overnight. The reaction mixture was concentrated under reduced pressure and purified using reversed phase chromatography (C18, H₂O:ACN eluent) to provide the t-butyl ester of 4.2 (700 mg, 26% yield) as a yellow solid.

The purified product was treated with TFA (1.6 M) until complete hydrolysis of the ester as determined by normal phase TLC. The resulting mixture was concentrated under reduced pressure and carried onto the next step without further purification. Spectroscopic data are consistent with those reported previously in the literature.^{229 1}H NMR (400 MHz, DMSO) δ 11.10 (s, 1H), 7.59 (dd, J=8.5, 7.1 Hz, 1H), 7.09 (d, J=7.0 Hz, 1H), 7.00 (d, J=8.6 Hz, 1H), 6.86 (s, 1H), 5.08 (dd, J=12.9, 5.4 Hz, 1H), 4.15-4.09 (m, 2H), 2.90 (ddd, J=17.4, 14.0, 5.4 Hz, 1H), 2.66-2.53 (m, 2H), 2.11-1.97 (m, 1H)

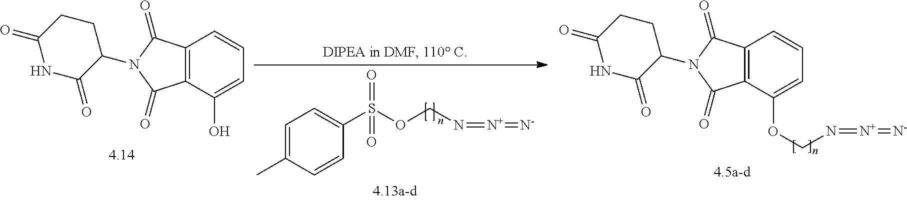

| ID | n | N |
|---|---|---|
| 4.5a | 3 | 4 |
| 4.5b | 4 | 5 |
| 4.5c | 6 | 7 |
| 4.5d | 7 | 8 |

### 4-(3-azidopropoxy)-2-(2,6-dioxopiperidin-3-yl)isoindoline-1,3-dione (4.5a)

4-hydroxy-thalidomide 4.14 (350 mg, 1.28 mmol) was dissolved in DMF (0.1 M) with DIPEA (496 mg, 3.84 mmol) and heated at 110° C., under argon and stirred for 5 minutes before stirring in 3-azidopropyl 4-methylbenzenesulfonate 4.13a (475.7 mg, 1.6 mmol). The reaction was allowed to proceed for 17 hours. The reaction mixture was concentrated under reduced pressure, then purified by reversed phase chromatography (C18, 10-100% ACN in water) to provide 4.5a as a white powder (268.5 mg, 59% yield). ¹H NMR (400 MHz, CDCl₃) δ 8.33 (s, 1H), 7.61 (dd, J=8.5, 7.3 Hz, 1H), 7.40 (d, J=7.3 Hz, 1H), 7.16 (d, J=8.4 Hz, 1H), 4.94-4.84 (m, 1H), 4.19 (t, 1=5.9 Hz, 2H), 3.55 (t, J=6.4 Hz, 2H), 2.86-2.59 (m, 3H), 2.06 (p, J=6.4 Hz, 3H). ¹³C NMR (101 MHz, CDCl₃) δ 171.13, 168.21, 165.62, 156.22, 136.59, 133.84, 119.05, 117.42, 116.21, 65.89, 49.15, 47.80, 31.38, 28.54, 22.62. HRMS m/z 358.11396 [M+H]⁺ (calcd. for C₁₆H₁₆N₅O₅, 358.11460).

### 4-(4-azidobutoxy)-2-(2,6-dioxopiperidin-3-yl)isoindoline-1,3-dione (4.5b)

4-hydroxy-thalidomide (200 mg, 0.73 mmol) was dissolved in DMF (7 mL, 0.1 M) with DIPEA (0.4 mL, 2.19 mmol) and heated at 110° C., under argon and stirred for 5 minutes before stirring in 4-azidobutyl 4-methylbenzenesulfonate 4.13b (236 mg, 0.875 mmol). The reaction was allowed to proceed for 20 hours. The reaction mixture was concentrated under reduced pressure, then purified by reversed phase chromatography (C18, 10-100% ACN in water) to provide 4.5b as a white powder (91.1 mg, 34% yield). ¹H NMR (400 MHz, CDCl₃) δ 8.17 (s, 1H), 7.61 (t, J=7.3 Hz, 1H), 7.39 (d, J=7.3 Hz, 1H), 7.14 (d, J=8.5 Hz, 1H), 4.93-4.84 (m, 1H), 4.14 (t, 1=6.0 Hz, 2H), 3.36 (t, J=6.6 Hz, 2H), 2.87-2.59 (m, 3H), 2.12-2.00 (m, 1H), 1.91 (tt, J=8.2, 5.8 Hz, 2), 1.79 (dtd, J=8.9, 6.6, 5.2 Hz, 2H). ¹³C NMR (101 MHz, CDCl₃) δ 171.00, 168.12, 167.01, 165.63, 156.40, 136.56, 133.86, 118.93, 117.30, 116.01, 68.79, 51.11, 49.13, 31.40, 26.21, 25.61, 22.62. HRMS m/z 372.12946 [M+H]⁺ (calcd. for C₁₇H₁₈N₅O₅, 372.13025).

### 4-((6-azidohexyl)oxy)-2-(2,6-dioxopiperidin-3-yl)isoindoline-1,3-dione (4.5c)

4-hydroxy-thalidomide (350 mg, 1.28 mmol) was dissolved in DMF (0.1 M) with DIPEA (496 mg, 3.84 mmol) and heated at 110° C. under argon and stirred for 5 minutes before stirring in 6-azidohexyl 4-methylbenzenesulfonate 4.13c (255.3 mg, 1.6 mmol). The reaction was allowed to proceed overnight. The reaction mixture was concentrated under reduced pressure, then purified by reversed phase chromatography (C18, 10-100% ACN in water) to provide 4.5c as a white powder (139.4 mg, 27% yield). ¹H NMR (400 MHz, CDCl₃) δ 8.26 (s, 1H), 7.68 (dd, J=8.4, 7.3 Hz, 1H), 7.46 (dd, J=7.3, 0.7 Hz, 1H), 7.22 (d, J=8.4 Hz, 1H), 5.02-4.92 (m, 1H), 4.20 (t, J=6.4 Hz, 2H), 3.31 (t, J=6.9 Hz, 2H), 2.96-2.68 (m, 3H), 2.21-2.09 (m, 1H), 1.97-1.86 (m, 1H), 1.72-1.42 (m, 6H). ¹³C NMR (101 MHz, CDCl₃) δ 171.02, 168.14, 167.07, 165.67, 156.64, 136.50, 133.84, 118.93, 117.19, 69.23, 51.35, 49.11, 31.40, 28.74 (d, J=3.0 Hz), 26.36, 25.46, 22.63. HRMS m/z 422.14173 [M+H]⁺ (calcd. for C₁₉H₂₁N₅O₅, 422.14349).

### 4-((7-azidoheptyl)oxy)-2-(2,6-dioxopiperidin-3-yl)isoindoline-1,3-dione (4.5d)

4-hydroxy-thalidomide (350 mg, 1.28 mmol) was dissolved in DMF (0.1 M) with DIPEA (0.4 mL, 3.83 mmol) and heated at 110° C., under argon and stirred for 5 minutes before stirring in 7-azidoheptyl 4-methylbenzenesulfonate 4.13d (477 mg, 1.53 mmol). The reaction was allowed to proceed overnight. The reaction mixture was concentrated under reduced pressure, then purified by reversed phase chromatography (C18, 10-100% ACN in water) to provide 4.5d as a white powder (364.3 mg, 84% yield). ¹H NMR (400 MHz, CDCl₃) δ 8.18 (s, 1H), 7.60 (dd, =8.5, 7.3 Hz, 1H), 7.37 (d, J=7.1 Hz, 1H), 7.14 (d, J=8.4 Hz, 1H), 4.93-4.84 (m, 1H), 4.10 (t, 1.65 Hz, 2H), 3.20 (t, J 6.9 Hz, 2H), 2.87-2.59 (m, 3H), 2.12-2.00 (m, 1H), 1.81 (d, J=8.6, 6.5 Hz, 2H), 1.62-1.41 (m, 4H), 1.41-1.27 (m, 4H). ¹³C NMR (101 MHz, CDCl₃) δ 171.04, 168.15, 167.08, 165.68, 156.68, 136.48, 133.84, 118.93, 117.17, 115.75, 69.37, 51.42, 49.10, 31.39, 28.76 (d, J=5.6 Hz), 26.58, 25.75, 22.64. HRMS m/z 436.15772 [M+Na]⁺ (calcd. for C₂₀H₂₃N₅NaO₅, 436.15914).

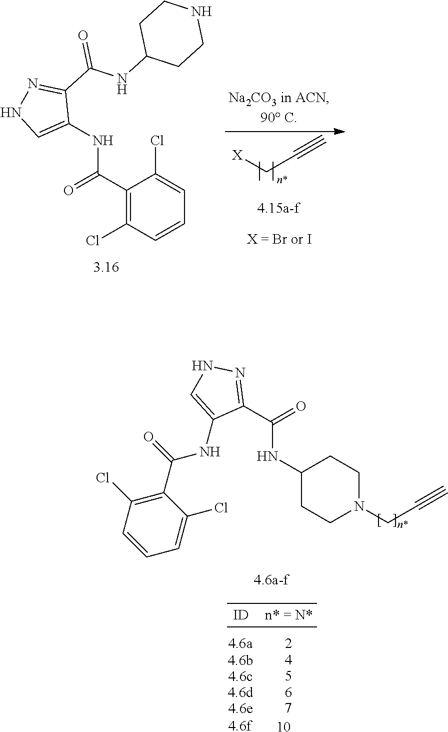

### N-(1-(but-3-yn-1-yl)piperidin-4-yl)-4-(2,6-dichlorobenzamido)-1H-pyrazole-3-carboxamide (4.6a)

4-(2,6-dichlorobenzamido)-N-(piperidin-4-yl)-1H-pyrazole-3-carboxamide (136.4 mg, 0.275 mmol) was added to a solution of 4-bromo butyne (48.4 mg, 0.364 mmol) and disodium carbonate (111.1 mg, 0.275 mmol) in acetonitrile (0.08M). The resulting mixture was heated at 90° C. for 8 hrs. After cooling, brine was added, and the mixture was extracted three times with 10% MeOH in DCM. The combined organic layers were dried over sodium sulfate, filtered, then concentrated under reduced pressure. The crude mixture was purified by column chromatography (SiO₂, 3-5% MeOH in DCM) to provide 4.6a as a white powder (39.3 mg, 33% yield). ¹H NMR (400 MHz, CDCl₃) δ 13.14 (s, 1H), 9.87 (s, 1H), 8.52 (s, 1H), 7.40-7.24 (m, 3H), 6.88 (d, J=8.9 Hz, 1H), 4.05 (td, J=9.7, 4.9 Hz, 1H), 3.07 (d, J=11.3 Hz, 2H), 2.72 (t, 1=7.2 Hz, 2H), 2.51 (td, J=7.3, 3.9 Hz, 2H), 2.22 (td, J=11.8, 3.4 Hz, 2H), 2.13-190 (m, 5H). ¹³C NMR (101 MHz, CDCl₃) δ 163.13, 161.69, 135.53, 133.15, 132.66, 131.09, 128.24, 122.48, 121.82, 81.92, 77.36, 70.44, 7039, 56.87, 52.51, 45.30, 31.32, 29.79, 16.88. Exact Mass (EM): Chemical Formula: C₂₀H₂₆Cl₂N₅O₂, Exact Mass: 433.10723. Found [M+H]⁺ HRMS m/z 434.11382 [M+H]⁺ (calcd. for C₂₂H₂₆Cl₂N₅O₂, 434.11451).

### 4-(2,6-dichlorobenzamido)-N-(1-(hex-5-yn-1-yl)piperidin-4-yl)-1H-pyrazole-3-carboxamide (4.6b)

4(2,6-dichlorobenzamido)-N-(piperidin-4-yl)-1H-pyrazole-3-carboxamide (200 mg, 0.403 mmol, 1 equiv.) was dissolved in acetonitrile (0.1 M) with 6-iodohex-1-yne (83.80 mg, 0.403 mmol) and disodium carbonate (123 mg, 1.16 mmol). The reaction mixture was heated at 90° C. for 16.5 hrs. After cooling, brine was added, and extraction was performed with 2% EtOH in DCM. The combined organic layers were dried over sodium sulfate, filtered, then concentrated under reduced pressure. The crude mixture was purified by column chromatography (SiO₂, 5-10% MeOH in DCM) to provide 4.6b as an orange powder (132.4 mg, 71% yield). Chemical Formula: C₂₂H₂₅Cl₂N₅O₂, Exact Mass: 461.13853. Found 462.14581 [M+H]⁺ ¹H NMR (400 MHz, CDCl₃) δ 13.85 (s, 1H), 9.84 (s, 1H), 8.53 (s, 1H), 7.42-7.21 (m, 3H), 6.89 (d, J=9.2 Hz, 1H), 4.09 (q, J=7.1 Hz, 1H), 3.08 (dd, J=9.2, 5.6 Hz, 2H), 2.53 (t, J=8.1 Hz, 2H), 2.29 (td, J=6.8, 2.7 Hz, 21-1), 2.25-2.14 (m, 3H), 2.13-1.97 (m, 4H), 1.87-1.73 (m, 2H), 1.68-1.51 (m, 2H). ¹³C NMR (101 MHz, CDCl₃) δ 163.02, 161.45, 135.54, 132.60, 130.93, 128.13, 122.42, 121.54, 83.65, 69.70, 57.95, 52.64, 44.98, 31.30, 29.70, 26.20, 18.20. HRMS m/z 462.14508 [M+H]⁺ (calcd. for C₂₁H₂₆Cl₂N₅O₂, 462.14581).

### 4-(2,6-dichlorobenzamido)-N-(1-(hept-6-yn-1-yl)piperidin-4-yl)-1H-pyrazole-3-carboxamide (4.6c)

4-(2,6-dichlorobenzamido)-N-(piperidin-4-yl)-1H-pyrazole-3-carboxamide (200 mg, 0.403 mmol) was dissolved in acetonitrile (0.1 M) with 7-iodohept-1-yne 4.15c (89.50 mg, 0.403 mmol) and disodium carbonate (123 mg, 1.16 mmol). The reaction mixture was heated at 90° C. for 18 hrs. After cooling, brine was added, and extraction was performed with 2% EtOH in DCM. The combined organic layers were dried over sodium sulfate, filtered, then concentrated under reduced pressure. The crude mixture was purified by column chromatography (SiO₂, 5-10% MeOH in DCM) to provide 4.6c as an orange powder (80.0 mg, 42% yield), ¹H NMR (400 MHz, CDCl₃) δ 13.14 (br s, 14), 9.85 (s, 1H), 8.48 (s, 1H), 7.38-7.27 (m, 3H), 6.89 (d, J=8.8 Hz, 1H), 4.06 (h, J=8.3 Hz, 1H), 3.10 (t, J=7.2 Hz, 2H), 2.54 (s, 2H), 2.23 (td, J=6.8, 2.7 Hz, 4H), 2.12-1.91 (m, 5H), 1.76-1.55 (m, 4H), 1.54-1.43 (m, 2H). ¹³C NMR (101 MHz, CDCl₃) δ 163.08, 161.55, 135.50, 132.59, 130.95, 128.14, 122.45, 121.49, 84.07, 77.25, 68.73, 58.37, 52.50, 45.05, 31.08, 28.08, 26.64, 26.18, 18.34. HRMS m/z 476.16151 [M+H]⁺ (calcd. for C₂₃H₂₈Cl₂N₅O₂, 476.16146).

### 4-(2,6-dichlorobenzamido)-N-(1-(oct-7-yn-1-yl)piperidin-4-yl)-1H-pyrazole-3-carboxamide (4.6d)

4-(2,6-dichlorobenzamido)-N-(piperidin-4-yl)-1H-pyrazole-3-carboxamide (200 mg, 0.403 mmol) was dissolved in acetonitrile (0.1M) with 8-iodooct-I-yne 4.16d (95.10 mg, 0.403 mmol) and disodium carbonate (123 mg, 1.16 mmol). The reaction mixture was heated at 90° C. for 19 hrs. After cooling, brine was added, and extraction was performed with 2% EtOH in DCM. The combined organic layers were dried over sodium sulfate, filtered, then concentrated tinder reduced pressure. The crude mixture was purified by column chromatography (SiO₂, 5-10% MeOH in DCM) to provide 4.6d as an orange powder (65.2 mg, 33% yield). ¹H NMR (400 MHz, CDCl₃) δ 13.26 (br s, 1H), 9.84 (s, 1H), 8.46 (s, 1H), 7.37-7.28 (m, 3H), 6.96 (s, 1H), 4.07 (h, J=8.3 Hz, 1H), 3.29-3.00 (m, 2H), 2.56 (t, J=8.1 Hz, 2H), 2.20 (td, J=6.9, 2.7 Hz, 4H), 2.14-2.00 (m, 4H), 1.96 (t, J=2.6 Hz, 1H), 1.80-1.62 (m, 2H), 1.58-1.33 (m, 6H). ¹C NMR (101 MHz, CDCl₃) δ 163.09, 161.53, 135.52, 132.59, 130.95, 128.14, 122.49, 121.58, 84.40, 68.42, 58.39, 5245, 45.02, 30.93, 28.43, 28.30, 27.08, 26.35, 18.30. FIRMS t/Z 490.17689 [M+H]⁺ (calcd. for C₂₄H₃₀Cl₂N₅O₂, 490.1771).

### 4-(2,6-di chlorobenzamido)-N-(1-(non-8-yn-1-yl)piperidin-4-yl)-1-pyrazole-3-carboxamide (4.6e)

4-(2,6-dichlorobenzamido)-N-(piperidin-4-yl)-1H-pyrazole-3-carboxamide (378.9 mg, 0.76 mmol) was dissolved in acetonitrile (0.1M) with 9-iodonon-1-yne 4.15e (252.89 mg, 1.01 mmol) and disodium carbonate (302.58 tug, 2.85 mmol). The reaction mixture was heated at 90° C. for 19 hrs. After cooling, brine was added, and extraction was performed with 90:10 DCM:MeOH. The combined organic layers were dried over sodium sulfate, filtered, then concentrated under reduced pressure. The crude mixture was purified by column chromatography (SiO₂, 2-10% MeOH in DCM) to provide 4.6e as an orange powder (140.3 mg, 37% yield). ¹H NMR (400 MHz, CDCl₃) δ 12.00 (br s, 1H), 9.78 (s, 1H), 8.49 (s, 1H), 7.39-7.27 (m, 3H), 4.10 (s, 1H), 3.35 (s, 2H), 2.73 (s, 4H), 2.27-2.05 (m, 5H), 1.95 (t, J=2.6 Hz, 1H), 1.53 (p, J=6.7 Hz, 2H), 1.46-1.32 (m, 7H). ¹³C NMR (101 μHz, CDCl₃) δ 161.50, 135.45, 132.54, 130.99, 128.17, 84.51, 77.24, 68.34, 57.84, 52.09, 44.57, 29.70, 28.62, 28.55, 28.45, 28.39, 28.23, 26.97, 24.86, 18.32. HRMS m/z 504.19215 [M+H]⁺ (calcd. for C₂₅H₃₂C₂N₅O₂, 504.19276).

### 4-(2,6-dichlorobenzamido)-N-(1-(dodec-11-yn-1-yl)piperidin-4-yl)-1H-pyrazole-3-carboxamide (4.6f)

4-(2,6-dichlorobenzamido)-N-(piperidin-4-yl)-1H-pyrazole-3-carboxamide (398 mg, 0.801 mmol) was dissolved in acetonitrile (0.1 M) with 12-iodododec-1-yne 4.15f (234 mg, 0.801 mmol) and disodium carbonate (245 mg, 2.31 mmol). The reaction mixture was heated at 90° C. for 14 hrs. After cooling, brine was added, and extraction was performed with 2% EtOH in DCM. The combined organic layers were dried over sodium sulfate, filtered, then concentrated tinder reduced pressure. The crude mixture was purified by column chromatography (SiO₂, 1-3% MeOH in DCM) to provide 4.6f as an orange powder (253 mg, 58% yield). ¹H NMR (400 MHz, CDCl₃) δ 13.45 (s, 1H), 9.83 (s, 1H), 8.47 (s, 1H), 7.38-7.28 (m, 3H), 6.98 (s, 1H), 4.16-3.98 (m, 1H), 3.36-3.01 (m, 2H), 2.60 (s, 2H), 2.31 (s, 2H), 2.18 (td, J=7.1, 2.7 Hz, 3H), 2.08 (s, 3H), 1.93 (d, J=2.6 Hz, 1H), 1.57-1.46 (m, 2H), 1.42-1.27 (m, 12H). ¹³C NMR (101 MHz, CDCl₃) δ 163.17, 161.51, 135.52, 132.60, 130.94, 128.14, 122.49, 84.80, 68.06, 58.43, 52.45, 44.96, 29.71, 29.49, 29.38, 29.35, 29.04, 28.72, 28.47, 27.50, 18.39. HRMS m/z 546.23971 [M+H]⁺ (calcd, for C₂₈H₃₈Cl₂N₅O₂, 546.23971).

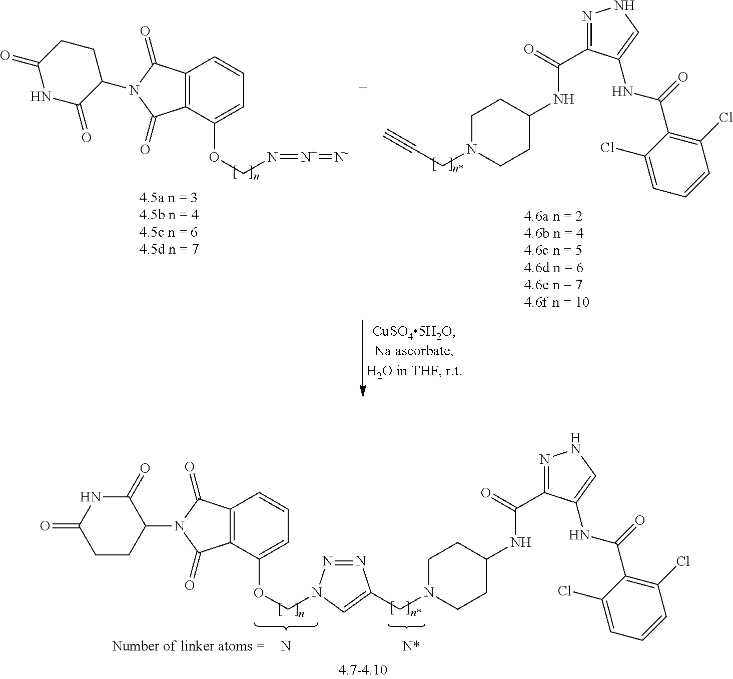

| ID | N:N* |
|---|---|
| 4.7a | 5:4 |
| 4.8a | 8:2 |
| 4.9a | 8:6 |
| 4.7b | 4:4 |
| 4.8b | 5:5 |
| 4.9b | 7:7 |
| 4.7c | 4:5 |
| 4.8c | 4:6 |
| 4.9c | 4:10 |
| ID | N:N |
| 4.10 | 7:5 |

### 4-(2,6-dichlorobenzamido)-N-(1-(4-(1-(4-((2-(2,6-dioxopiperidin-3-yl)-1,3-dioxoisoindolin-4-yl)oxy)buty)-1H-1,2,3-triazol-4-yl)butyl)piperidin-4-yl)-1H-pyrazole-3-carboxamide (4.7a)

Sodium ascorbate (16.1 mg, 0.081 mmol) and CuSO₄·5H₂O (26.9 mg, 0.108 mmol) were completely dissolved in water (0.07 M). The mixture was then added to a solution of 4-(2,6-dichlorobenzamido)-N-(1 (hex-5-yn-1-yl)piperidin-4-yl)-1H-pyrazole-3-carboxamide 4.6b (25.0 mg, 0.0541 mmol) and 4-(4-azidobutoxy)-2-(2,6-dioxopiperidin-3-yl)isoindoline-1,3-dione 4.5b (25.1 mg, 0.068 mmol) in THF (0.04 M). The reaction was allowed to proceed at room temperature for 26 hours under argon. The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 7-10% MeOH in DCM) to provide 4.7a as a white solid (13 mg, 30% yield). ¹H NMR (400 MHz, DMSO) δ 13.43 (s, 1H), 11.10 (s, 1H), 10.14 (s, 1H), 8.37 (s, 1H), 7.92-7.73 (m, 2H), 7.64-7.37 (m, 6H), 5.08 (dd, J=12.8, 5.4 Hz, 1H), 4.43 (t, J=6.9 Hz, 3H), 4.23 (t, J=6.1 Hz, 2H), 3.39 (t, J=6.7 Hz, 2H), 2.95-2.80 (m, 1H), 2.65-2.55 (m, 3H), 2.24 (t, J=7.5 Hz, 2H), 2.01 (q, J=7.3 Hz, 31H), 1.80-1.45 (m, 8H), 1.24 (s, 2H). ¹³C NMR (101 MHz, CDCl₃) δ 172.33, 169.32, 167.14, 166.00, 161.72, 156.31, 136.93, 135.60, 133.86, 132.71, 131.10, 128.28, 127.92, 122.97, 122.05, 118.90, 117.20, 116.15, 68.81, 62.58, 56.82, 49.90, 49.27, 32.07, 31.59, 31.08, 30.31, 29.51, 27.88, 26.91, 26.57, 25.70, 25.03, 23185, 22.92, 22.84, 14.27. HRMS m/z 833.26877 [M+H]⁺ (calcd. for C₃₉H₄₃Cl₂N₁₀O₇, 833.26878).

### 4-(2,6-dichlorobenzamido)-N-(1-(4-(1-(3-((2-(2,6-dioxopiperidin-3-yl)-1,3-dioxoisoindolin-4-yl)oxy)propyl)-1H-1,2,3-triazol-4-yl)butyl)piperidin-4-yl)-1H-pyrazole-3-carboxamide (4.7b)

Sodium ascorbate (25.7 mg, 0.130 mmol) and CuSO₄·5H₂O (43.03 mg, 0.173 mmol) were completely dissolved in water (0.07 M). The mixture was then added to a solution of 4-(2,6-dichlorobenzamido)-N-(1-(hex-5-yn-1-yl)piperidin-4-yl)-1H-pyrazole-3-carboxamide 4.6b (40 mg, 0.086 mmol) and 4-(3-azidopropoxy)-2-(2,6-dioxopiperidin-3-yl)isoindoline-1,3-dione 4.5a (38.6 mg, 0.108 mmol) in THE (0.04 M). The reaction was allowed to proceed at room temperature for 21 hours under argon. The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 6-8% MeOH in DCM) to provide 4.7b as a white solid (29.4 mg, 42% yield). ¹H NMR (400 MHz, DMSO) δ 13.44 (s, 1H), 11.11 (s, 1H), 10.14 (s, 1H), 8.37 (s, 1H), 7.90 (s, 1H), 7.82 (dd, J=8.6, 7.2 Hz, 1H), 7.63-7.42 (m, 6H), 5.10 (dd, J=12.9, 5.4 Hz, 1H), 4.54 (t, J=6.9 Hz, 2H), 4.22 (q, J=6.3 Hz, 2H), 3.14-2.73 (m, 3H), 2.67-2.54 (m, 4H), 2.38-2.25 (M, 3H), 2.09-2.00 (m, 1H), 1.86 (s, 3H), 1.75-1.36 (m, 7H), 1.24 (s, 2H). ¹³C NMR (101 MHz, MeOD) δ 174.57, 171.50, 168.53, 167.52, 163.31, 157.43, 148.46, 138.07, 136.68, 135.13, 133.46, 132.82, 129.49, 124.06, 123.02, 120.74, 118.54, 116.92, 66.96, 58.36, 52.96, 50.49, 47.84, 46.33, 32.74, 32.17, 31.03, 30.74, 30.49, 27.89, 25.83, 25.69, 23.69, 14.41. HRMS m/z 819.25174 [M+H]⁺ (calcd. for C₃₈H₄₁Cl₂N₁₀O₇, 819.25313).

### 4-(2,6-dichlorobenzamido)-N-(1-(5-(1-(3-((2-(2,6-dioxopiperidin-3-yl)-1,3-dioxoisoindolin-4-yl)oxy)propyl)-11H-1,2,3-triazol-4-yl)pentyl)piperidin-4-yl)-1H-pyrazole-3-carboxamide (4.7c)

Sodium ascorbate (31.2 mg, 0.157 mmol) and CuSO₄·5H₂O (52.2 mg, 0.21 mmol) were completely dissolved in water (0.14 M). The mixture was then added to a solution of 4-(2,6-dichlorobenzamido)-N-(1-(hept-6-yn-1-yl)piperidin-4-yl)-1H-pyrazole-3-carboxamide 4.6c (50.0 mg, 0.105 mmol) and 4-(3-azidopropoxy)₂-(2,6-dioxopiperidin-3-yl)isoindoline-1,3-dione 4.5a (46.9 mg, 0.131 mmol) in THF (0.04 M). The reaction was allowed to proceed at room temperature for 40.5 hours under argon. The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 8% MeOH in DCM) to provide 4.7c as a white solid (37 mg, 42% yield). Desired product was recovered in 41.9% yield as white solid (37 mg, 0.044 mmol). ¹H NMR (400 MHz, DMSO) δ 13.42 (s, 1H), 11.11 (s, 1H), 10.15 (s, 1H), 8.36 (s, 3H), 7.88 (s, 1H), 7.68-7.41 (m, 4H), 4.54 (t, J=6.9 Hz, 2H), 4.20 (t, J=5.9 Hz, 2H), 3.52 (s, 1H), 2.90 (s, 2H), 2.65-2.54 (m, 4H), 2.38-2.27 (m, 3H), 1.70-1.51 (m, 4H), 1.35 (s, 2H), 1.24 (s, 6H). ¹³C NMR (101 MHz, CDCl₃) δ 171.71, 168.77, 166.94, 165.82, 163.07, 161.47, 155.89, 147.96, 136.70, 135.56, 133.79, 132.66, 132.53, 130.90, 128.09, 122.49, 122.04, 121.59, 119.21, 117.50, 116.39, 77.29, 65.49, 58.15, 53.45, 52.27, 49.25, 46.16, 31.53, 3143, 29.67, 29.41, 28.94, 26.84, 26.34, 25.32, 22.71. HRMS m/z 833.26681 [M+H]⁺ (calcd. for C₃₉H₄₃Cl₂N₁₀O₇, 833.26878).

### 4-(2,6-dichlorobenzamido)-N-(1-(2-(1-(7-((2-(2,6-dioxopiperidin-3-yl)-1,3-dioxoisoindolin-4-yl)oxy)heptyl)-1H-1,2,3-triazol-4-yl)ethyl)piperidin-4-yl)-1H-pyrazole-3-carboxamide (4.8a)

Sodium ascorbate (17.1 mg, 0.086 mmol) and CuSO₄·5H₂O (28.6 mg, 0.115 mmol) were completely dissolved in water (0.44 M). The mixture was then added to a solution of N-(1-(but-3-yn-1-yl)piperidin-4-yl)-4-(2,6-dichlorobenzamido)-1H-pyrazole-3-carboxamide 4.6a (25.0 mg, 0.058 mmol) and 4-((7-azidoheptyl)oxy)-2-(2,6-dioxopiperidin-3-yl)isoindoline-1,3-dione 4.5d (29.7 mg, 0.072 mmol) in THF (0.04 M). The reaction was allowed to proceed at room temperature for 19.5 hours under argon. The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 7% MeOH in DCM) to provide 4.8a as a white solid (19.3 mg, 40% yield). ¹H NMR (400 MHz, DMSO) δ 13.40 (s, 1H), 11.10 (s, 1H), 10.17 (s, 1H), 8.36 (s, 2H), 7.94-7.76 (i, 2H), 7.66-734 (m, 5H), 5.08 (dd, J=12.8, 5.4 Hz, 1H), 4.30 (t, J=7.0 Hz, 2H), 4.19 (t, J=6.4 Hz, 2H), 3.24 (d, J=9.1 Hz, 1H), 2.88 (ddd, J=16.5, 13.7, 6.4 Hz, 2H), 2.76 (s, 1H), 2.65-2.54 (m, 2H), 2.03 (ddd, J=11.6, 6.2, 3.8 Hz, 2H), 1.87-1.54 (i, 71-1), 1.53-1.32 (m, 4H), 1.31-1.08 (m, 7H), 0.94-0.80 (m, 1H). ¹³C NMR (176 MHz, DMSO) δ 173.26, 170.43, 167.32, 165.80, 160.75, 156.47, 137.50, 135.84, 133.72, 133.38, 132.37, 131.72, 128.91, 121.95, 120.24, 116.69, 115.61, 69.21, 52.66, 49.61, 49.20, 31.70, 31.43, 30.11, 29.50, 28.73, 28.45, 26.24, 25.59, 22.47. HRMS m/z 847.28136 [M+H]⁺ (calcd. for C₄₀H₄₅Cl₂N₁₀O₇, 847.28443).

### 4-(2,6-dichlorobenzamido)-N-(1-(5-(1-(4-((2-(2,6-dioxopiperidin-3-yl)-1,3-dioxoisoindolin-4-yl)oxy)butyl)-1H-1,2,3-triazol-4-yl)pentyl)piperidin-4-yl)-1H-pyrazole-3-carboxanmide (4.8b)

Sodium ascorbate (18.7 mg, 0.094 mmol, 1.5 equiv.) and CUSO₄·5H₂O (31.3 mg, 0.126 mmol) were completely dissolved in water (0.08 M). The mixture was then added to a solution of 4-(2,6-dichlorobenzamido)-N-(1-(hept-6-yn-1-yb)piperidin-4-yl)-1-H-pyrazole-3-carboxamide 4.6c (30.0 tmg, 0.063 innol) and 4-(4-azidobutoxy)-2-(2,6-dioxopiperidin-3-yb)isoindoline-1,3-dione 4.5b (29.2 mg, 0.079 innol) in THF (0.04 M). The reaction was allowed to proceed at room temperature for 26 hours under argon). The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 8-9% MeOH in DCM) to provide 4.8b as a white solid (27.1 mg, 51% yield). ¹H NMR (400 MHz, DMSO) δ 13.39 (s, 1H), 11.10 (s, 1H), 10.16 (s, 1H), 8.35 (s, 2H), 7.95-7.72 (m, 2H), 7.63-7.35 (m, 5H), 5.15-4.96 (m, 1H), 4.42 (t, J=6.9 Hz, 2H), 4.22 (t, J=6.1 Hz, 2H), 3.71 (s, 1H), 3.51 (s, 1H), 2.94-2.77 (m, 2H), 2.58 (dd, J=12.5, 5.1 Hz, 3H), 2.30-2.10 (m, 2H), 2.01 (t, J=7.0 Hz, 3H), 1.76-1.54 (m, 8H), 1.44 (d, J=6.6 Hz, 2H), 1.37-1.21 (m, 5H). —C NMR (101 MHz, MeOD) δ 174.58, 171.55, 168.59, 167.50, 164.99, 163.32, 157.69, 148.78, 138.04, 136.68, 135.11, 133.46, 132.83, 129.50, 123.46, 123.04, 120.48, 118.25, 116.59, 69.93, 58.30, 52.77, 50.99, 50.44, 45.86, 32.18, 30.75, 30.57, 28.05, 27.29, 26.73, 25.87, 25.69, 2368. HRMS m/z 847.28241 [M+H]⁺ (calcd. for C₄₀H₄₅Cl₂N₁₀O₇, 847.28443).

### 4-(2,6-dichlorobenzamido)-N-(1-(6-(1-(3-((2(2,6-dioxopiperidin-3-yl)-1,3-dioxoisoindolin-4-yl)oxy)propyl)-1H-1,2,3-triazol-4-yl)hexyl)piperidin-4-yl)-1H-pyrazole-3-carboxamide (4.8c)

Sodium ascorbate (18.2 mg, 0.092 mmol) and CuSO₄·5H₂O (30.4 mg, 0.122 mmol) were completely dissolved in water (0.44 M). The mixture was then added to a solution of 4-(2,6-dichlorobenzamido)-N-(1-(oct-7-yn-1-yl)piperidin-4-yl)-1H-pyrazole-3-carboxamide 4.6d (30.0 mg, 0.061 mmol) and 4-(3-azidopropoxy)-2-(2,6-dioxopiperidin-3-yl)isoindoline-1,3-dione 4.5a (27.3 mg, 0.076 mmol) in THF (0.04 M). The reaction was allowed to proceed at room temperature for 40.5 hours under argon). The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 6% MeOH in DCM) to provide 4.8c as a white solid (53.4 mg, 103% yield). ¹H NMR (400 MHz, MeOD) δ 8.36 (s, 1H), 7.80-7.73 (m, 2H), 7.57-7.43 (m, 41H), 7.40 (d, J=8.5 Hz, 1H), 5.15-5.09 (m, 1H), 4.67 (t, J=6.7 Hz, 2H), 4.19 (tq, J=8.6, 4.3 Hz, 2H), 4.02 (q, J=8.5 Hz, 1H), 3.35 (s, 1H), 2.88 (ddd, J=17.8, 14.3, 5.1 Hz, 1H), 2.82-2.63 (m, 8H), 2.51-2.38 (m, 2H) 2.19-2.02 (m, 3H), 1-83 (q, J=12.0 Hz, 2H), 1.63 (q, J=7.7 Hz, 4H), 1.40-1.32 (m, 5H). Did not observe 4 Hs; 2 amides, pyrazole and glutarimide. ¹³C NMR (101 MHz, CDCl₃) δ 171.83, 168.77, 166.95, 165.82, 163.16, 161.46, 155.84, 148.16, 136.64, 135.61, 133.84, 132.77, 132.55, 130.86, 128.07, 122.48, 121.92, 121.52, 119.06, 117.54, 116.34, 77.28, 65.22, 58.45, 53.43, 52.41, 52.34, 49.27, 46.05, 45.90, 31.57, 29.67, 29.36, 29.05, 28.94, 27.27, 26.70, 25.50, 22.69. HRMS m/z 847.28192 [M+H]⁺ (calcd. for C₄₀H₄₅Cl₂N₁₀O₇, 847.28443).

### 4-(2,6-dichlorobenzamido)-N-(1-(6-(1-(7-((2-(2,6-dioxopiperidine-3-yl)-1,3-dioxoisoindolin-4-yl)oxy)heptyl)-1H-1,2,3-triazol-4-yl)hexyl)piperidin-4-yl)-1H-pyrazole-3-carboxamide (4.9a)

Sodium ascorbate (18.2 mg, 0.092 mmol) and CuSO₄·5H₂O (300.4 mg, 0.122 mmol) were completely dissolved in water (0.44 M). The mixture was then added to a solution of 4-(2,6-dichlorobenzamido)-N-(1-(oct-7-yn-1-yl)piperidin-4-yl)-1H-pyrazole-3-carboxamide 4.6f (30.0 mg, 0.061 mmol) and 4-((7-azidoheptyl)oxy)-2-(2,6-dioxopiperidin-3-yl)isoindoline-1,3-dione 4.5d (31.6 mg, 0.076 mmol) in THF (0.04 M). The reaction was allowed to proceed at room temperature for 26.5 hours under argon). The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 7% MeOH in DCM) to provide 4.9a as a white solid (42.3 mg, 77% yield). ¹H NMR (400 MHz, DMSO) δ 13.43 (s, 11H), 11.09 (s, 1H), 10.14 (s, 1H), 8.36 (s, 1H), 7.83-7.77 (m, 2H), 7.59 (d, J=2.1 Hz, 1H), 7.57 (d, J=0.7 Hz, 2H), 7.55-7.48 (m, 2H), 7.43 (d, J=7.2 Hz, 1H), 5.12-5.01 (m, 1H), 4.28 (t, J=7.0 Hz, 2H), 4.19 (t, J=6.4 Hz, 2H), 2.88 (ddd, J=16.7, 13.6, 5.3 Hz, 2H), 2.64-2.54 (m, 4H), 2.02 (dp, J=11.0, 3.5 Hz, 2H), 1.83-1.68 (m, 7H), 1.56 (dt, J=14.0, 6.2 Hz, 4H), 1.49-1.21 (m, 16H). ¹³C NMR (176 MHz, DMSO) δ 173.26, 170.43, 167.32, 165.80, 160.78, 156.47, 147.19, 137.51, 135.83, 133.72, 13332, 132.38, 131.72, 128.91, 122.04, 121.96, 120.25, 116.68, 115.62, 69.21, 49.56, 49.19, 31.43, 30.11, 29.29, 28.73, 28.44, 26.23, 25.59, 25.39, 22.47. HRMS m/z 903.34425 [M+H]⁺ (calcd, for C₄₄H₅₃Cl₂N₁₀O₇, 903.34703).

### 4-(2,6-dichlorobenzamido)-N-(1-(7-(1-(6(2-(2,6-dioxopiperidin-3-yl)-1,3-dioxoisoindolin-4-yl)oxy)hexyl)-1H-1,2,3-triazol-4-yl)heptyl)piperidin-4-yl)-1H-pyrazole-3-carboxamide (4.9b)

Sodium ascorbate (21.8 mg, 0.11 mmol) and CuSO₄·5H₂O (36.5 mg, 0.147 mmol) were completely dissolved in water (0.46 M). The mixture was then added to a solution of 4-(2,6-dichlorobenzamido)-N-(1-(non-8-yn-1-yl)piperidin-4-yl)-1H-pyrazole-3-carboxamide 4.6e (37.1 mg, 0.074 mmol) and 4-((6-azidohexyl)oxy)-2-(2,6-dioxopiperidin-3-yl)isoindoline-1,3-dione 4.5c (36.7 mg, 0.092 mmol) in THF (0.04 M). The reaction was allowed to proceed at room temperature for 20 hours). The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 6% MeOH in DCM) to provide 4.9b as a white solid (11.8 mg, 18% yield). ¹H NMR (400 M/Hz, DMSO) δ 13.42 (s, 1H), 11.09 (s, 1H), 10.14 (s, 1H), 8.36 (s, 1H), 7.84-7.76 (m, 2H), 7.66-7.32 (m, 6H), 5.07 (dd, J=12.8, 5.4 Hz, 1H), 4.29 (t, J=7.0 Hz, 2H), 4.18 (t, J=6.3 Hz, 2H), 2.88 (ddd, J=17.4, 14.0, 5.4 Hz, 2H), 2.64-2.52 (m, 4H), 2.07-1.96 (m, 2H), 1.86-1.68 (m, 7H), 1.61-1.42 (m, 7H), 1.33-1.13 (m, 13H). ¹³C NMR (176 MHz, DMSO) δ 172.78, 169.95, 166.84, 165.34, 155.97, 146.77, 137.05, 135.36, 133.25, 131.91, 131.24, 128.43, 121.60, 121.48, 119.77, 116.22, 115.17, 68.65, 49.03, 48.73, 30.95, 29.61, 28.92, 28.42, 28.16, 25.44, 24.97, 24.67, 22.00. HRMS m/z 903.34403 [M+H]⁺ (calcd, for C₄₄H ₅₃Cl₂N₁₀O₇, 903.34703).

### 4-(2,6-dichlorobenzamido)-N-(1-(10-(1-(3-((2-(2,6-dioxopiperidin-3-yl)-1,3-dioxoisoindolin-4-yl)oxy)propyl)-1H-1,2,3-triazol-4-yl)decyl)piperidin-4-yl)-1H-pyrazole-3-carboxamide (4.9c)

Sodium ascorbate (17.9 mg, 0.091 mmol) and CuSO₄·5H₂O (30.0 mg, 0.121 mmol) were completely dissolved in water (0.46 M). The mixture was then added to a solution of 4-(2,6-dichlorobenzamido)-N-(1-(dodec-11-yn-1-yl)piperidin-4-yl)-1H-pyrazole-3-carboxamide 4.6f (33.0 mg, 0.060 mmol) and 4-(3-azidopropoxy)-2-(2,6-dioxopiperidin-3-yl)isoindoline-1,3-dione 4.5a (27.1 mg, 0.076 mmol) in THF (0.04 M). The reaction was allowed to proceed at room temperature for 20.5 hours. The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 4-8% MeOH in DCM) to provide 4.9c as a white solid (11.9 mg, 22% yield). ¹H NMR (400 MHz, DMSO) δ 13.46 (s, 1H), 11.11 (s, 1H), 10.13 (s, 1H), 8.37 (s, 1H), 7.89-7.77 (m, 2H), 7.65-7.42 (m, 6H), 5.10 (dd, J=12.9, 5.4 Hz, 1H), 4.54 (t, J=6.9 Hz, 2H), 4.20 (t, J=6.0 Hz, 2H), 3.11-3.03 (m, 3H), 2.90 (ddd, J=17.4, 14.1, 5.4 Hz, 2H), 2.57 (q, J=6.6 Hz, 3H), 2.31 (q, J=6.4 Hz, 3H), 2.08-2.01 (m, 1H), 1.64-1.49 (m, 4H), 1.28-1.22 (m, 14H), 1.19 (t, J=7.3 Hz, 5H). ¹³C NMR (176 MHz, DMSO) δ 172.77, 169.91, 166.80, 165.39, 160.31, 155.60, 146.96, 137.10, 135.35, 133.21, 132.83, 139.1, 131.24, 128.43, 121.90, 121.49, 119.91, 116.50, 115.54, 69.77, 65.73, 48.77, 45.89, 45.60, 30.95, 29.22, 28.92, 28.85, 28.72, 28.54, 24.97, 22.01. HRMS m/z 903.34401 [M+H]⁺ (calcd. for C₄₄H₅₃Cl₂N₁₀O₇, 903.34703).

### 4-(2,6-dichlorobenzamido)-N-(1-(5-(1-(6-((2-(2,6-dioxopiperidin-3-yl)-1,3-dioxoisoindolin-4-yl)oxy)hexyl)-1H-1,2,3-triazol-4-yl)pentyl)piperidin-4-yl)-1H-pyrazole-3-carboxamide (4.10)

Sodium ascorbate (21.8 mg, 0.110 mmol) and CuSO₄·5H₂O (36.5 mg, 0.147 mmol) were completely dissolved in water (0.46 M). The mixture was then added to a solution of 4-(2,6-dichlorobenzamido)-N-(1-(hept-6-yn-1-yl)piperidin-4-yl)-1H-pyrazole-3-carboxamide 4.6e (35.0 mg, 0.073 mmol) and 4-((6-azidohexyl)oxy)-2-(2,6-dioxopiperidin-3-yl)isoindoline-1,3-dione 4.5d (36.7 mg, 0.092 mmol) in THF (0.04 M). The reaction was allowed to proceed at room temperature for 19.5 hours. The reaction mixture was then dissolved in brine and extraction was performed with 20% MeOH in DCM. The combined organic layers were dried over sodium sulfate, filtered, then concentrated under reduced pressure. The residue was purified by column chromatography (SiO₂, 6% MeOH in DCM) to provide 4.10 as a white solid (19.3 mg, 30% yield), ¹H NMR (400 MHz, DMSO) δ 13.39 (s, 1H), 11.10 (s, 1H), 10.16 (s, 1H), 8.42-8.13 (m, 2H), 7.86-7.75 (m, 2H), 7.64-7.34 (m, 5H), 5.07 (dd, J=12.9, 5.4 Hz, 1H), 4.29 (t, J=7.0 Hz, 2H), 4.18 (t, J=6.3 Hz, 2H), 3.68 (s, 1H), 2.93-2.78 (m, 2H), 2.61-2.54 (m, 3H), 2.20 (t, J=7.3 Hz, 2H), 2.02 (d, J=12.5 Hz, 1H), 1.82 (dd, J=14.2, 7.2 Hz, 5H), 1.74 (t, J=7.2 Hz, 2H), 1.67-1.53 (m, 5H), 1.50-1.36 (m, 4H), 1.31-1.22 (m, 6H); ¹³C NMR (176 MHz, DMSO) δ 172.77, 169.95, 166.84, 165.32, 160.30, 155.97, 146.76, 137.03, 135.38, 133.24, 131.87, 131.25, 128.42, 121.61, 121.45, 119.75, 116.22, 115.15, 68.64, 57.83, 52.43, 49.02, 48.73, 46.29, 31.26, 30.95, 29.61, 28.93, 28.69, 28.15, 26.55, 26.35, 25.43, 24.99, 24.66, 21.99; HRMS m/z 875.31479 [M+H]⁺ (calcd. for C₄₂H₄₉Cl₂N₁₀O₇, 875.31573).

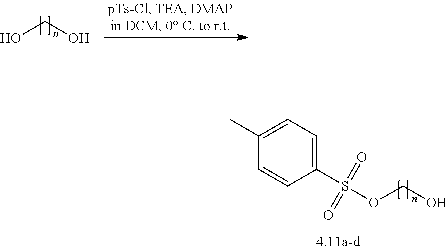

### 3-hydroxypropyl 4-methylbenzenesulfonate (4.11a)

Propane-1,3-diol (5.00 g, 65.7 mmol), TEA (3.66 g, 36.1 mmol), DMAP (803 mg (0.80 g, 6.57 mmol) were stirred in DCM (0.2M) at 0° C. p-Toluenesulfonyl chloride (6.26 g, 32.9 mmol) was added to the stirring mixture portionwise and allowed to stir at room temperature for 9 hrs. The mixture was dissolved in ammonium chloride solution and extracted with DCM. The combined organic layers were washed with brine and dried over sodium sulfate, then concentrated under reduced pressure. The crude mixture was purified by column chromatography (SiO₂, 35-60% EtOAc in Hexanes) to provide 4.11a as a colorless liquid (5.31 g, 70% yield). Spectroscopic data are consistent with those reported previously.^{230 1}H NMR (400 MHz, CDCl₃) δ 7.86-7.73 (m, 2H), 7.43-7.30 (m, 2H), 4.19 (t, J=6.0 Hz, 2H), 3.72 (t, J 5.9 Hz, 2H), 2.45 (s, 3H), 1.89 (p, J=6.0 Hz, 2H), 1.56 (s, 1H).

### 4-hydroxybutyl-4-methylbenzenesulfonate (4.11b)

Butane-1,4-diol (10.00 g, 111 mmol), TEA (6.18 g, 61.0 mmol), DMAP (1.36 g, 11.1 mmol) were stirred in DCM (0.2M) at 0° C. p-toluenesulfonyl chloride (10.6 g, 55.5 mmol) was added to the stirring mixture portionwise and allowed to stir at room temperature for 3 hrs. The mixture was dissolved in ammonium chloride solution and extracted with DCM. The combined organic layers were washed with brine and dried over sodium sulfate, then concentrated under reduced pressure. The crude mixture was purified by column chromatography (SiO₂, 35-60% EtOAc in Hexanes) to provide 4.11b as a colorless liquid (7.76 g, 57% yield). Spectroscopic data are consistent with those previously reported in the literature.^{230 1}H NMR (400 MHz, CDCl₃) δ 7.83-7.71 (m, 2H), 7.35 (d, J=8.0 Hz, 2H), 4.07 (t, J=6.3 Hz, 2H), 3.62 (t, J=6.3 Hz, 2H), 2.45 (s, 3H), 1.76 (tt, J=8.5, 6.1 Hz, 2H), 1.63-1.57 (m, 3H).

### 6-hydroxyhexyl 4-methylbenzenesulfonate (4.11c)

Hexane-1,4-diol (0.73 g, 6.18 mmol), TEA (0.69 g, 6.80 mmcl), DMAP (0.15 g, 1.24 mmol) were stirred in DCM (0.13M) at 0° C. p-toluenesulfonyl chloride (10.6 g, 55.5 mmol) was added to the stirring mixture portionwise and allowed to stir at room temperature overnight. The mixture was dissolved in ammonium chloride solution and extracted with ethyl acetate. The combined organic layers were washed with brine and dried over sodium sulfate, then concentrated under reduced pressure. The crude mixture was purified by column chromatography (SiO₂, 35% EtOAc in Hexanes) to provide 4.11c as a colorless gel (1.22 g, 49% yield). Spectroscopic data are consistent with those previously reported in the literature.^{230 1}H NMR (400 MHz, CDCl₃) δ 7.85-7.73 (m, 2H), 7.34 (d, J=8.0 Hz, 2H), 4.03 (t, J=6.4 Hz, 2H), 3.61 (t, 1=6.5 Hz, 2H), 2.45 (s, 3H), 1.66 (dq, J=8.2, 6.6 Hz, 2H), 1.59-1.45 (m, 2H), 1.40-1.23 (m, 5H).

### 7-hydroxyheptyl 4-methylbenzenesulfonate (4.11d)

p-toluenesulfonyl chloride (6.49 g, 34.0 mmol), TEA (4.21 g, 41.6 mmol), DMAP (0.92 g, 7.56 mmol) were stirred in DCM (0.13M) at 0° C. Heptane-1,7-diol (5.00 g, 37.8 mmol) was added to the stirring mixture portionwise and allowed to stir at room temperature overnight. The mixture was dissolved in ammonium chloride solution and extracted with DCM. The combined organic layers were washed with brine and dried over sodium sulfate, then concentrated under reduced pressure. The crude mixture was purified by column chromatography (SiO₂, 35-100% EtOAc in Hexanes) to provide 4.11d as a colorless liquid (5.09 g, 47% yield). Spectroscopic data are consistent with those previously reported in the literature.^{231 1}H NMR (400 MHz, CDCl₃) δ 7.88-7.76 (m, 2H), 7.37 (d, J=8.0 Hz, 2H), 4.04 (t, J=6.5 Hz, 2H), 3.64 (t, J=6.6 Hz, 2H), 2.47 (s, 3H), 1.75-1.48 (m, 4H), 1.45 (s, 2H), 1.36-1.24 (m, 5H).

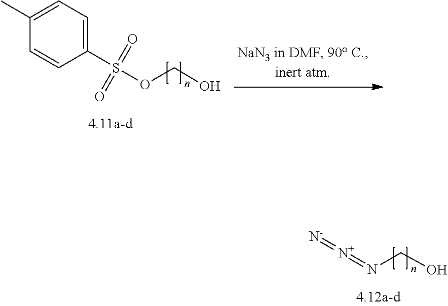

### 3-azidopropan-1-ol (4.12a)

3-[(4-methylbenzenesulfonyl)oxy]propan-1-ol 4.11a (5.31 g, 23.1 mmol), Sodium Azide (3.00 g, 46.1 mmol) were stirred in DMF (0.75M) at 90° C. for 12 hrs. The mixture was dissolved in water and extracted with ethyl acetate. The combined organic layers were washed with brine and dried over sodium sulfate, then concentrated under reduced pressure. The crude yellow liquid was carried onto the next step without further purification. Spectroscopic data are consistent with those previously reported in the literature.^{232 1}H NMR (400 MHz, CDCl₃) δ 3.75 (t, J=6.0 Hz, 2H), 3.45 (t, J=6.6 Hz, 2H), 2.24 (s, 1H), 1.87-1.78 (m, 2H).

### 4-azidobutan-1-ol (4.12b)

4-[(4-methylbenzenesulfonyl)oxy]butan-1-ol 4.11b (7.45 g, 30.5 mmol), Sodium Azide (3.97 g, 61.1 mmol) were stirred in DMF (0.75M) at 90° C. overnight. The mixture was dissolved in water and extracted with ethyl acetate. The combined organic layers were washed with brine and dried over sodium sulfate, then concentrated under reduced pressure. The crude yellow liquid was carried onto the next step without further purification. Spectroscopic data are consistent with those previously reported in the literature.^{233 1}H NMR (400 MHz, CDCl₃) δ 3.70 (t, J=6.1 Hz, 2H), 3.34 (t, J=6.5 Hz, 2H), 1.79-1.61 (m, 5H).

### 6-azidohexan-1-ol (4.12c)

6-[(4-methylbenzenesulfonyl)oxy]hexan-1-ol 4.11c (0.76 g, 2.80 mmol), Sodium Azide (0.36 g, 5.61 mmol) were stirred in DMF (0.75M) at 90° C. overnight. The mixture was dissolved in water and extracted with ethyl acetate. The combined organic layers were washed with brine and dried over sodium sulfate, then concentrated under reduced pressure. The crude liquid was carried onto the next step without further purification. Spectroscopic data are consistent with those previously reported in the literature.^{10 1}H NMR (400 MHz, CDCl₃) δ 3.59 (t, J=6.5 Hz, 2H), 3.20 (t, J=6.9 Hz, 2H), 1.53 (dtd, J=13.5, 6.9, 3.8 Hz, 3H), 1.40 (s, 2H), 1.34 (p, J=3.6 Hz, 4H).

### 7-azidoheptane-1-ol (4.12d)

4-[(4-methylbenzenesulfonyl)oxy]heptan-1-ol 4.11d (5.09 g, 17.8 mmol), Sodium Azide (2.31 g, 35.6 mmol) were stirred in DMF (0.75M) at 90° C. overnight. The mixture was dissolved in water and extracted with ethyl acetate. The combined organic layers were washed with brine and dried over sodium sulfate, then concentrated under reduced pressure. The crude liquid was carried onto the next step without further purification. Spectroscopic data are consistent with those previously reported in the literature.^{234 1}H NMR (400 MHz, CDCl₃) δ 3.66 (t, J=6.6 Hz, 2H), 3.28 (t, J=6.9 Hz, 2H), 1.68 (s, 1H), 1.61 (dt, J=12.4, 7.3 Hz, 4H), 1.48-1.29 (m, 6H).

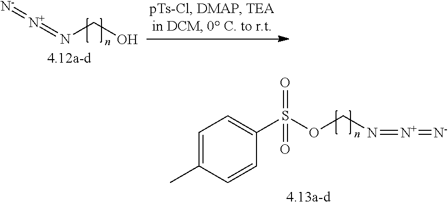

### 3-azidopropyl 4-methylbenzenesulfonate (4.13a)

3-azidopropan-1-cl 4.12a (2.32 g, 22.9 mmcl), TEA (6.97 g, 68.8 mmol), DMAP (294 mg, 2.29 mmol) were stirred in DCM (0.67M) at 0° C. p-toluenesulfonyl chloride (6.56 g, 34.4 mmol) was added to the stirring mixture portionwise and allowed to stir at room temperature overnight. The mixture was dissolved in ammonium chloride solution and extracted with DCM. The combined organic layers were washed with brine and dried over sodium sulfate, then concentrated under reduced pressure. The crude mixture was purified by column chromatography (SiO₂, 5-15% EtOAc in Hexanes) to provide 4.13a as a yellow oil (1.86 g, 35% yield). Spectroscopic data are consistent with those previously reported in the literature.^{235 1}H NMR (400 MHz, CDCl₃) δ 7.86-7.79 (dd, 2H), 7.39 (d, J=8.1 Hz, 2H), 4.13 (t, J=5.9 Hz, 2H), 3.40 (t, J=6.5 Hz, 2H), 2.48 (s, 3H), 1.92 (p, J=6.2 Hz, 2H).

### 4-azidobutyl 4-methylbenzenesulfonate (4.13b)

4-azidobutan-1-ol 4.12b (3.66 g, 31.8 mmol), TEA (9.65 g, 95.4 mmol, 3 equiv.), DMAP (407.7 mg, 3.18 mmol) were stirred in DCM (0.67M) at 0° C., p-toluenesulfonyl chloride (9.09 g, 32.9 mmol) was added to the stirring mixture portionwise and allowed to stir at room temperature overnight. The mixture was dissolved in ammonium chloride solution and extracted with DCM. The combined organic layers were washed with brine and dried over sodium sulfate, then concentrated under reduced pressure. The crude mixture was purified by column chromatography (SiO₂, 5-17% EtOAc in Hexanes) to provide 4.13b as a yellow oil (5.31 g, 70% yield). Spectroscopic data are consistent with those previously reported in the literature.^{236 1}H NMR (400 MHz, CDCl₃) δ 7.82-7.72 (m, 2H), 7.35 (d, J=8.1 Hz, 2H), 4.05 (t, J=6.1 Hz, 2H), 3.25 (t, J=6.6 Hz, 2H), 2.45 (s, 3H), 1.79-1.68 (m, 21H) 1.67-1.54 (m, 2H).

### 6-azidohexyl 4-m ethylbenzenesulfonate (4.13c

6-azidohexan-1-ol 4.12e (4.4 g, 31 mmol), TEA (9.4 g, 93 mmol), were stirred in DCM (0.67M) at 0° C. p-toluenesulfonyl chloride (8.86 g, 47 mmol) was added to the stirring mixture portionwise and allowed to stir at room temperature overnight. The mixture was dissolved in ammonium chloride solution and extracted with DCM. The combined organic layers were washed with brine and dried over sodium sulfate, then concentrated under reduced pressure. The crude mixture was purified by column chromatography (SiO₂, 8-15% EtOAc in Hexanes) to provide 4.13c as a yellow oil (5.78 g, 63% yield). Spectroscopic data are consistent with those previously reported in the literature.^{237 1}H NMR (400 MHz, CDCl₃) δ 7.83-7.75 (m, 21-1), 7.39-7.31 (m, 2H), 4.03 (t, J=6.4 Hz, 2H), 3.23 (t, J=6.8 Hz, 2H), 2.45 (s, 3H), 1.71-1.49 (m, 4H), 1.34 (ddt, J=7.0, 5.6, 3.4 Hz, 41).

### 7-azidoheptyl 4-methylbenzenesulfonate (4.13d)

7-azidoheptan-1-ol 4.12d (3.31 g, 21.1 mmol), TEA (6.4 g, 63.2 mmcl) were stirred in DCM (0.67M) at 0° C. p-toluenesulfonyl chloride (6.02 g, 31.6 mmol) was added to the stirring mixture portionwise and allowed to stir at room temperature overnight. The mixture was dissolved in ammonium chloride solution and extracted with DCM. The combined organic layers were washed with brine and dried over sodium sulfate, then concentrated under reduced pressure. The crude mixture was purified by column chromatography (SiO₂, 5-100% EtOAc in Hexanes) to provide 4.13d as a yellow oil (2.33 g, 36% yield). Spectroscopic data are consistent with those previously reported in the literature.^{238 1}H NMR (400 MHz, CDCl₃) δ 7.84-7.75 (m, 2H), 7.35 (d, J=8.0 Hz, 2H), 4.02 (t, J=6.4 Hz, 2H), 3.24 (t, J=6.9 Hz, 2H), 2.45 (s, 3H), 1.71-1.49 (m, 4H), 1.42-1.18 (m, 6H).

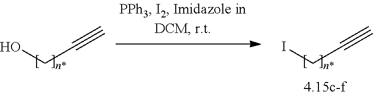

| ID | n* = N* |
|---|---|
| 4.6a | 2 |
| 4.6b | 4 |
| 4.6c | 5 |
| 4.6d | 6 |
| 4.6e | 7 |
| 4.6f | 10 |

### 7-iodohept-1-yne (415c)

Imidazole (0.85 g, 12.5 mmol.) and PPh₃ (3.28 g, 12.5 mmol) were dissolved in DCM (0.21) and stirred for 10 minutes at room temperature. Iodine (3.18 g, 12.5 mmol) was then added to the reaction mixture. After stirring for 10 minutes, 6-heptyn-1-ol (1 g, 7.92 mmol) in DCM (6 M) was then added and the reaction was stirred for 30 minutes. The reaction was concentrated under reduced pressure and saturated sodium sulfite solution was added. The mixture was extracted with hexanes and the combined organic layers were concentrated under reduced pressure. The residue was purified by column chromatography (SiO₂, hexanes) to provide 4.15c as a colorless liquid (0.838 g, 42% yield). Spectroscopic data are consistent with those previously reported in the literature.^{239 1}H NMR (400 MHz, CDCl₃) δ 3.19 (t, J=7.0 Hz, 2H), 2.21 (td, J=6.7, 2.6 Hz, 2H), 1.95 (d, J=5.3 Hz, 1H), 1.91-1.80 (m, 2H), 1.61-1.45 (m, 4H).

### 8-iodooct-1-yne (4.15d)

Imidazole (0.76 g, 11.1 mmol) and PPh₃ (2.91 g, 11.1 mmol) were dissolved in DCM (0.2M) and stirred for 10 minutes at room temperature. Iodine (2.82 g, 11.1 mmol.) was then added to the reaction mixture. After stirring for 10 minutes, 7-octyn-1-ol (1 g, 7.92 mmol) dissolved in DCM (1 mL) was added and stirred for 30 minutes. The reaction was concentrated tinder reduced pressure and saturated sodium sulfite solution was added. The mixture was extracted with hexanes and the combined organic layers were concentrated under reduced pressure. The residue was purified by column chromatography (SiO₂, hexanes) to provide 4.15d as a colorless liquid (1.276 g, 68% yield). Spectroscopic data are consistent with those previously reported in the literature.^{4 1}H NMR (400 MHz, CDCl₃) δ 3.19 (t, J=7.0 Hz, 2H), 2.20 (td, J=7.0, 2.7 Hz, 2H), 1.94 (t, J=2.7 Hz, 1H), 1.84 (p, J=7.2 Hz, 2H), 1.52 (m, J=13.7 Hz, 2H), 1.48-1.37 (m, 4H).

### 9-iodnon-1-yne (4.15e)

Imidazole (0.34 g, 9.98 mmol) and PPh₃ (1.3 g, 9.98 mmol) were dissolved in DCM (0.2M) and stirred for 10 minutes at room temperature. Iodine (1.27 g, 9.98 mmol) was then stirred into the reaction mixture for another 10 minutes. 8-nonyn-1-ol (0.5 g, 7.13 mmol) dissolved in DCM (6 M) was added and allowed to stir for 30 minutes. The reaction was concentrated under reduced pressure and saturated sodium sulfite solution was added. The mixture was extracted with hexanes and the combined organic layers were concentrated under reduced pressure. The residue was purified by column chromatography (SiO₂, hexanes) to provide 4.15e as a colorless liquid (0.337 g, 38% yield). Spectroscopic data are consistent with those previously reported in the literature.^{239 1}H NMR (400 MHz, CDCl₃) δ 3.19 (t, J=7.0 Hz, 2H), 2.24-2.17 (td, 2H), 1.94 (t, J=2.7 Hz, 1H), 1.83 (p, J=7.1 Hz, 2H), 1.59-1.47 (m, 2H), 1.46-1.27 (m, 6H).

### 12-ioddodec-1-yne (4.15f)

Imidazole (0.5 g, 2.74 mmol) and PPh₃ (1.01 g, 3.84 mmol) were dissolved in DCM (0.2M) and stirred for 10 minutes at room temperature. Iodine (0.98 g, 3.84 mmol) was then stirred into the reaction mixture for another 10 minutes. 8-dodecyn-1-ol (0.5 g, 2.74 mmol) in DCM (6 M) and added and allowed to stir for 30 minutes. The reaction was concentrated under reduced pressure and saturated sodium sulfite solution was added. The mixture was extracted with hexanes and the combined organic layers were concentrated under reduced pressure. The residue was purified by column chromatography (SiO₂ hexanes) to provide 4.15f as a colorless liquid (0.684 g, 85% yield). Spectroscopic data are consistent with those previously reported in the literature.^{241 1}H NMR (400 MHz, CDCl₃) δ 3.19 (t, J=7.0 Hz, 2H), 2.18 (td, J=7.1, 2.6 Hz, 2H), 1.94 (t, J=5.3 Hz, 1H), 1.82 (p, J=7.1 Hz, 2H), 1.58-1.47 (p, 2H), 1.45-1.34 (m, 4H), 1.29 (s, 8H).

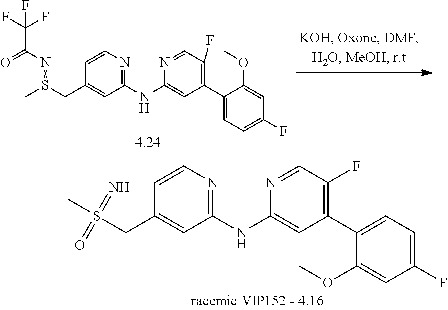 [SMILES: COc1cc(F)ccc1-c1cc(Nc2cc(CS(C)=NC(=O)C(F)(F)F)ccn2)ncc1F ; COc1cc(F)ccc1-c1cc(Nc2cc(CS(C)(=N)=O)ccn2)ncc1F]

### ((2-((5-fluoro-4-(4-fluoro-2-methoxyphenyl)pyridin-2-yl)amino)pyridin-4-yl)methyl)(imino)(methyl)₄-sulfa-none (4.16)

2,2,2-trifluoro-N-(((2-((5-fluoro-4-(4-fluoro-2-methoxyphenyl)pyridin-2-yl)amino)pyridin-4-yl)methyl)(methyl)-λ⁴-sulfaneylidene)acetamide 4.24 (600 mg, 1.24 mmcl) was dissolved in a mixture of DMF (0.03 M), H₂O (0.1 M) and MeOH (0.1 M). The pH of the mixture was adjusted to >10 by dropwise addition of 2 M KOH. Oxone (648 mg, 1.05 mmol) was then added to the stirring mixture and the reaction proceeded for 3 hours, during which the pH was kept above 10. The reaction mixture was filtered through a Whatman filter which was rinsed with plenty of DCM, The filtrate was then washed with brine followed by an aqueous solution of sodium thiosulfate. The recovered organic layer was concentrated under reduced pressure and the desired product 4.16 was isolated as an off-white powder (322 mg, 64% yield) from the mixture through suction filtration while rinsing with DCM and diisopropyl ether. Spectroscopic data are consistent with those reported previously.^{219 1}H NMR (400 MHz, DMSO) δ 9.82 (s, 1H), 8.24-8.15 (m, 2H), 7.80 (d, J=5.4 Hz, 1H), 7.61 (s, 1H), 7.36 (dd, J=8.4, 6.7 Hz, 1H), 7.11 (dd, J=11.5, 2.5 Hz, 1H), 6.97-6.88 (m, 2H), 4.44-4.31 (m, 2H), 3.81 (s, 3H), 3.74 (s, 1H), 2.89 (d, J=1.0 Hz, 3H).

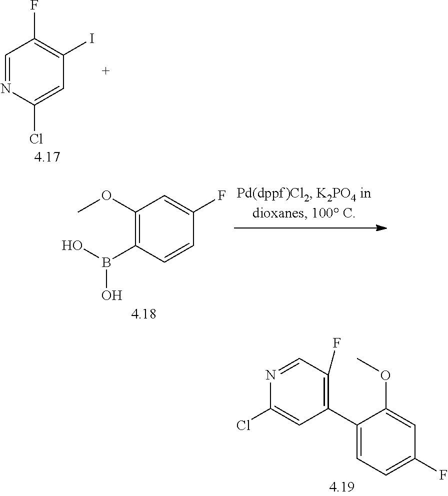

### 2-chloro-5-fluoro-4-(4-fluoro-2-methoxyphenyl)pyridine (4.19)

2-chloro-5-fluoro-4-iodopyridine (7.0 g, 27.2 mmol), (4-fluoro-2-methoxyphenyl)boronic acid (4.62 g, 27.2 mol), 1,1′-Bis(diphenylphosphino)ferrocene]dichloropalladium(II) (2.0 g, 2.72 mmol) and tripotassium phosphate (11.5 g, 54.4 mmol) were dissolved in DIE (0.4 M) and the mixture was purged with argon. The reaction mixture was then subjected to heat at 100° C. and allowed to stir overnight. The reaction was then concentrated under reduced pressure, dissolved in water and extracted multiple times with DCM. The combined organic layers were washed with brine and dried over Na₂SO₄, then filtered. The crude mixture was then loaded onto SiO₂ and rinsed with ethyl acetate. The resulting filtrate was purified by column chromatography (SiO₂, 0-10% ethyl acetate in hexanes) to provide 4.19 as a colorless liquid (5.34 g, 77% yield). Spectroscopic data are consistent with those previously reported.^{219 1}H NMR (400 MHz, CDCl₃) δ 8.27 (d, J=1.5 Hz, 1H), 7.32 (s, 1H), 7.28-7.19 (m, 1H), 6.82-6.70 (m, 2H), 3.82 (s, 3H).

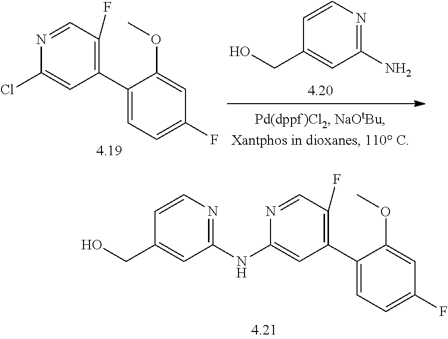 [SMILES: COc1cc(F)ccc1-c1cc(Cl)ncc1F ; Nc1cc(CO)ccn1 ; COc1cc(F)ccc1-c1cc(Nc2cc(CO)ccn2)ncc1F]

### 2-chloro-5-fluoro-4-(4-fluoro-2-methoxyphenyl)pyridine (4.19)

2-chloro-5-fluoro-4-(4-fluoro-2-methoxyphenyl)pyridine 4.19 (3 g, 11.7 mmol), (2-aminopyridin-4-yl)methanol 4.20 (2.01 g, 16.2 mol), 1,1′-Bis(diphenylphosphino)ferrocene]dichloropalladium(II) (6.87 mg, 0.94 mmol), [5-(diphenylphosphanyl)-9,9-dimethyl-9H-xanthen-4-yl]diphenylphosphane (543 mg, 0.94 mmol) and sodium tert-butoxide (1.69 g, 17.6 mmol) were dissolved in dioxanes (0.2 M) and the mixture was purged with argon. The reaction mixture was then subjected to heat at 110° C. and allowed to stir overnight. The reaction was then concentrated under reduced pressure, dissolved in water and extracted multiple times with DCM. The combined organic layers were washed with brine and dried over Na₂SO₄, then filtered. The crude mixture purified by column chromatography (SiO₂, 5-50% ethyl acetate in hexanes) to provide 4.21 as a colorless liquid (1.75 g, 44% yield). Spectroscopic data are consistent with those previously reported in the literature. ¹H NMR (400 MH, DMSO) δ 8.17 (d, J=5.2 Hz, 1H), 8.13 (d, J=1.8 Hz, 1H), 7.60 (d, J=52 Hz, 1H), 7.45 (s, 1H) 7.35 (s, 1H), 7.30-7.26 (m, 1H), 6.85-6.70 (m, 3H), 4.72 (s, 2H), 3.82 (s, 3H), 1.64-1.58 (m, 4H).

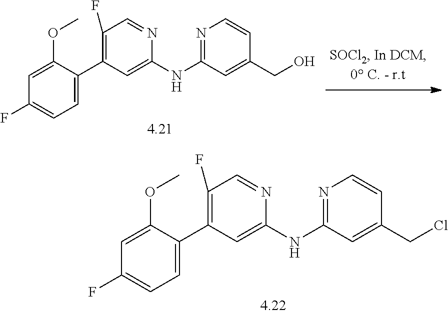 [SMILES: COc1cc(F)ccc1-c1cc(Nc2cc(CO)ccn2)ncc1F ; COc1cc(F)ccc1-c1cc(Nc2cc(CCl)ccn2)ncc1F]

### N-(4-(chloromethyl)pyridin-2-yl)-5-fluoro-4-(4-fluoro-2-methoxyphenyl)pyridin-2-amine 4.22

(2-((5-fluoro-4-(4-fluoro-2-methoxyphenyl)pyridin-2-yl)amino)pyridin-4-yl)methanol 4.21 (1.75 g, 5.1 mmol) was dissolved in DCM (0.7 M) with 1-methylpyrrolidin-2-one (1.92 g, 19.3 mmol) and cooled to 0° C. SOC1₂ 1.53 g, 12.8 mmol) was then added portionwise. The reaction mixture was stirred at room temperature overnight then quenched with NaHCO₃ and brine, then extracted with DCM. The combined organic layers were washed with brine and dried over Na₂SO₄, then filtered. The crude mixture was then loaded onto SiO₂ and rinsed with ethyl acetate. The resulting filtrate was concentrated under reduced pressure to provide 4.22 an orange solid in quantitative yield. The product was taken onto the next step without further purification. Spectroscopic data are consistent with those previously reported in the literature.^{223 1}H NMR (400 MHz, CDCl₃) δ 8.15-8.06 (m, 2H), 7.54 (s, 1H), 7.47 (d, J=5.2 Hz, 1H), 7.25-7.17 (m, 2H), 6.81 (dd, J=5.5, 1.5 Hz, 1H), 6.75-6.63 (m, 2H), 4.46 (s, 2H), 3.76 (s, 3H).

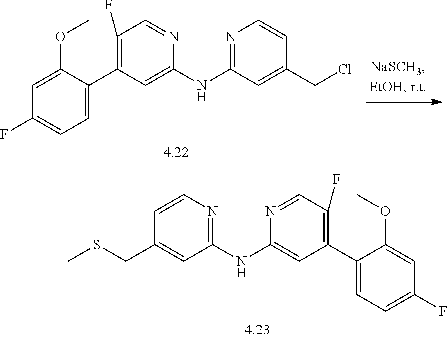 [SMILES: COc1cc(F)ccc1-c1cc(Nc2cc(CCl)ccn2)ncc1F ; COc1cc(F)ccc1-c1cc(Nc2cc(CSC)ccn2)ncc1F]

### 5-fluoro-4-(4-fluoro-2-methoxyphenyl)-N-(4-((methylthio)methyl)pyridin-2-yl)pyridin-2-amine (4.23)

N-(4-(chloromethyl)pyridin-2-yl)-5-fluoro-4-(4-fluoro-2-methoxyphenyl)pyridin-2-amine 4.22 (300 mg, 0.83 mmol) was dissolved in EtOH (0.08M) and the solution was cooled to 0° C. Sodium thiomethoxide (131 mg, 1.87 mmol) was then added portionwise after which the reaction mixture was stirred at room temperature overnight. The reaction mixture was dissolved in brine then extracted with DCM. The combined organic layers were concentrated under reduced pressure to provide 4.22 an orange solid in quantitative yield. The product was taken onto the next step without further purification, Spectroscopic data are consistent with those previously reported in the literature.^{223 1}H NMR (400 MHz, CDCl₃) δ 8.07 (d, J=1.9 Hz, 2H), 7.56 (d, J=5.2 Hz, 1H), 7.37 (s, 1H), 7.21 (ddd, J=8.4, 6.6, 0.8 Hz, 2H), 6.77 (d, J=5.3 Hz, 1H), 6.75-6.63 (m, 2H), 3.75 (s, 3H), 3.55 (s, 2H), 1.96 (s, 3H).

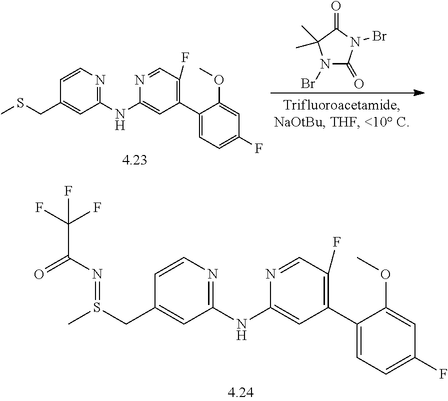 [SMILES: COc1cc(F)ccc1-c1cc(Nc2cc(CSC)ccn2)ncc1F ; CC1(C)C(=O)N(Br)C(=O)N1Br ; COc1cc(F)ccc1-c1cc(Nc2cc(C/S(C)=N/C(=O)C(F)(F)F)ccn2)ncc1F]

### 2,2,2-trifluoro-N-(((2-((5-fluoro-4-(4-fluoro-2-methoxyphenyl)pyridin-2-yl)amino)pyridin-4-yl)methyl)(methyl)-14-sulfaneylidene)acetamide (4.24)

Trifluoroacetamide (254 mg, 2.25 mmol) and sodium tert-butoxide (1.89 mg, 1.97 mmol) were dissolved in anhydrous THF (2 M) under inert atmosphere. The resulting mixture was cooled to 0° C. after which a solution of 1,3-dibromo-5,5-dimethylimidazolidine-2,4-dione (268 mg, 0.94 mmol) in THF (0.9 M) was added slowly. The reaction mixture was then added slowly to a solution of 5-fluoro-4-(4-fluoro-2-methoxyphenyl)-N-(4-((methylthio)methyl)pyridin-2-yl)pyridin-2-amine 4.23 (700 mg, 1.87 mmol) in THF (1.2 M) at 0° C. The reaction temperature was kept below 10° C. for 2.5 hours, after which the reaction was quenched with an aqueous solution of sodium sulfite and toluene. The resulting mixture was then exteracted multiple times with ethyl acetate. The combined organic layers were washed with brine and filtered through a Whatman filter. The filtrate was concentrated under reduced pressure and the crude mixture was purified by column chromatography (SiO₂, 0-5% EtOH in DCM) to provide 4.24 as an orange solid (620 mg, 68% yield). Spectroscopic data are consistent with those previously reported in the literature.^{223 1}H NMR (400 MHz, CDCl₃) δ 8.21-8.16 (m, 1H), 8.08 (d, J=1.7 Hz, 1H), 7.74 (s, 1H), 7.26 (d, J=5.1 Hz, 1H), 7.20 (ddd, J=8.3, 6.6, 0.9 Hz, 2H), 6.76-6.63 (m, 3H), 4.42 (d, J=12.9 Hz, 1H), 4.17-4.04 (m, 1H), 3.76 (s, 3H), 2.62 (s, 3H).

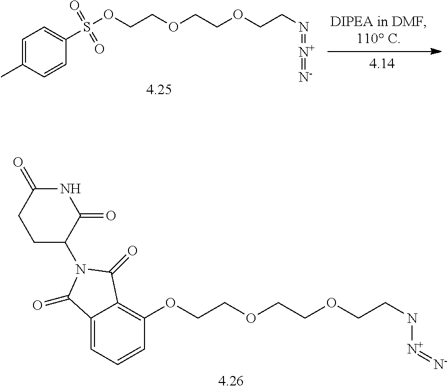 [SMILES: Cc1ccc(S(=O)(=O)OCCOCCOCCN=[N+]=[N-])cc1 ; [N-]=[NH+]NCCOCCOCCOc1cccc2c1C(=O)N(C1CCC(=O)NC1=O)C2=O]

### 4-(2-(2-(2-azidoethoxy)ethoxy)ethoxy)-2-(2,6-dioxopiperidin-3-yl)isoindoline-1,3-dione (4.26)

4-hydroxy-thalidomide 4.14 (300 mg, 1.09 mmol) was dissolved in DMF (0.1M) with DIPEA (424 mg, 3.28 mmol) and heated at 110° C., in a sealed tube and stirred for 5 minutes before stirring in 2-(2-(2-azidoethoxy)ethoxy)ethyl 4-methylbenzenesulfonate 4.25 (360 mg, 1.09 mmol). The reaction was allowed to proceed overnight. The reaction mixture was concentrated under reduced pressure, then purified by reversed phase chromatography (C18, 10-100% ACN in water) to provide 4.26 as a yellow gel (143 mg, 30% yield). ¹H NMR (400 MHz, CDCl₃) δ 8.62 (s, 1H), 7.59 (t, J=7.9 Hz, 1 h), 7.38 (d, J=7.3 Hz, 1H), 7.19 (d, J=8.5 Hz, 1H), 4.89 (dt, J=9.3, 4.5 Hz, 1H), 4.28 (t, J=4.7 Hz, 2H), 3.88 (t, J=4.7 Hz, 2H), 3.72 (dd, J=5.8, 3.4 Hz, 2H), 3.60 (td, J=5.2, 3.2 Hz, 4H), 3.30 (t, J=5.1 Hz, 2H), 2.73 (dddd, J=29.7, 18.7, 9.6, 5.6 Hz, 3H), 2.11-1.99 (m, 1H). ¹³C NMR (101 MHz, CDCl₃) δ 171.42, 168.42, 167.04, 165.60, 156.41, 136.46, 133.75, 119.50, 117.29, 116.11, 77.32, 71.13, 70.67, 70.10, 70.01, 69.97, 69.41, 69.28, 50.67, 49.11, 31.37, 22.59. HRMS in m/z 432.15059 [M+]⁺ (calcd. for C₁₉H₂₂N₅O₇, 432.15137).

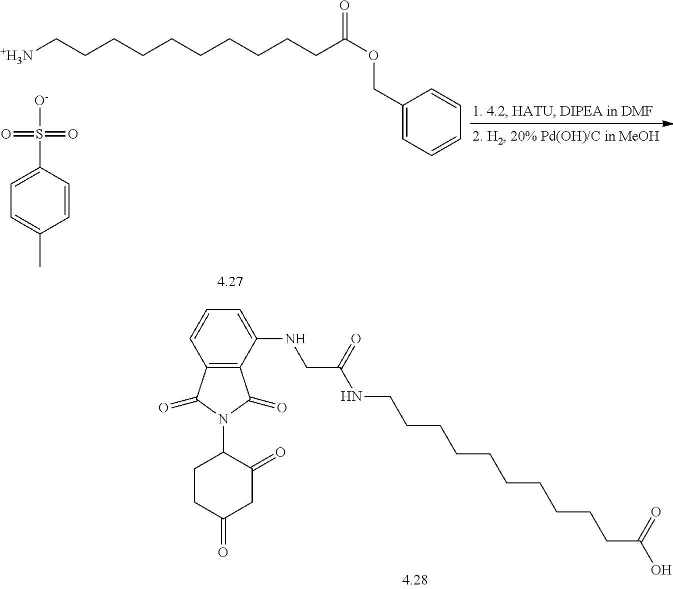 [SMILES: Cc1ccc(S(=O)(=O)[O-])cc1 ; [NH3+]CCCCCCCCCCC(=O)OCc1ccccc1 ; O=C(O)CCCCCCCCCCNC(=O)CNc1cccc2c1C(=O)N(C1CCC(=O)CC1=O)C2=O]

### 11-(2-((2-(2,6-dioxopiperidin-3-yl)-1,3-dioxoisoindolin-4-yl)amino)acetamido)undecanoic acid (4.28)

11-(benzyloxy)-11-oxoundecan-1-aminium 4-methylbenzene-1-sulfonate (4.27) (163 mg, 0.351 mmol, 1 equiv.), 2-{[2-(2,6-dioxopiperidin-3-yl)-1,3-dioxo-2,3-dihydro-1H-isoindol-4-yl]amino}acetic acid (I) (116 mg, 0.351 mmol, 1 equiv.), HATU (200 mg, 0.527 mmol, 1.5 equiv.) and DIPEA (54.5 mg, 0.422 mmol, 1.2 equiv.) were dissolved in DMF (2.64 mL, 0.2 M). The mixture was stirred overnight at room temperature. The reaction was quenched with water extracted multiple times with ethyl acetate (EtOAc). The combined organic layers were washed with brine and dried over Na₂SO₄, then concentrated under reduced pressure. The crude mixture was purified by column chromatography (SiO₂, 60-95% EtOAc in Hexanes) to provide benzoate ester of 4.28 as a green solid (138 mg, 65% yield).

In a solution of benzyl 11-(2-((2-(2,6-dioxopiperidin-3-yl)-1,3-dioxoisoindolin-4-yl)amino)acetamido)undecanoate (138 mg, 0.234 mmol, 1 equiv.) in methanol (5.84 mL, 0.04 M) was added Palladium hydroxide on carbon (13.1 mg, 0.094 mmol, 0.4 equiv.) under argon protection. The reaction vessel was purged with hydrogen gas and the reaction mixture was stirred at room temperature under hydrogen atmosphere overnight. The reaction was filtered through celite and concentrated under reduced pressure. The crude mixture was purified by column chromatography (SiO₂, 0-10% MeOH in EtOAc) to provide 4.28 as a green solid (50.6 mg, 42% yield). Spectroscopic data are consistent with those reported previously.^{214 1}H NMR (300 MHz, CDCl₃) δ 8.47 (s, 1H), 7.56 (dd, J=8.5, 7.2 Hz, 1H), 7.24 (s, 2H), 6.84 (d, J=8.5 Hz, 1H), 6.67 (s, 1H), 6.48 (s, 1H), 4.96 (dt, J=11.7, 5.7 Hz, 1H), 3.97 (s, 1H), 3.49 (s, 4H), 3.24 (tt, J=14.2, 7.3 Hz, 2H), 2.96-2.68 (m, 3H), 2.34 (t, J=7.2 Hz, 2H), 2.16 (dd, J=8.2, 4.5 Hz, 1H), 1.63 (t, J=7.2 Hz, 2H), 1.48 (ddd, J=16.5, 9.2, 7.0 Hz, 4H), 1.29 (s, 2H), 1.24 (s, 5H).

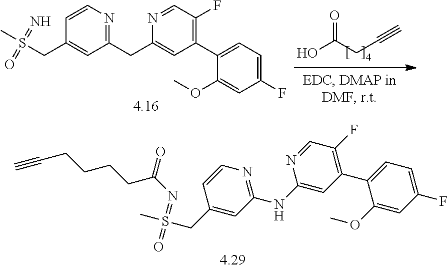

### N-((2-((5-fluoro-4-(4-fluoro-2-methoxyphenyl)pyridin-2-yl)amino)pyridin-4-yl) methyl) (methyl) (oxo)-16-sulfaneylidene) hept-6-ynamide (4.29)

Hept-6-ynoic acid (43.4 mg, 0.727 mmol, 1.1 equiv.), DMAP (66.5 mg, 0.544 mmol, 2.2 equiv.), EDC.HCI (94.8 mg, 0.495 mmol, 2 equiv.) and VIP152 (4.16) (100 mg, 0.247 mmol, 1 equiv.) were reacted according to general procedure A to provide 4.29 as a white powder (104 mg, 82% yield). 1H NMR (400 MHZ, CDC13) δ 8.28 (d, J=5.1 Hz, 1H), 8.17 (d, J=1.7 Hz, 1H), 7.79-7.74 (m, 1H), 7.67 (s, 1H), 7.44 (d, J=5.0 Hz, 1H), 7.30 (dd, J=8.3, 6.5 Hz, 1H), 6.91 (dd, J=5.2, 1.5 Hz, 1H), 6.84-6.72 (m, 2H), 4.75 (d, J=13,6 Hz, 1H), 4.66 (d, J=13.6 Hz, 1H), 3.85 (s, 3H), 3.13 (s, 3H), 2.40 (t, J=7.5 Hz, 2H), 2.21 (td, J=7.1, 2.6 Hz, 2H), 1.97 (t, J=2.6 Hz, 1H), 1.82-1.70 (m, 3H), 1.64-1.52 (m, 2H), 13C NMR (101 MHZ, CDC13) 8 182.57, 165.54, 163.07, 158.18, 158.07, 154.59, 154.19, 151.71, 149.71, 149.69, 148.70, 137.58, 135.86, 135.71, 134.93, 134.65, 131.79, 131.69, 118.58, 118.54, 117.95, 113.97, 112.88, 107.47, 107.25, 99.81, 99.55, 84.26, 77.24, 68.49, 58.71, 55.97, 39.08, 38.51, 28.05, 24.70, 18.25. HRMS m/z 513.17612 [M+H]⁺ (calcd. for C26H27F2N4O3S, 513.17664).

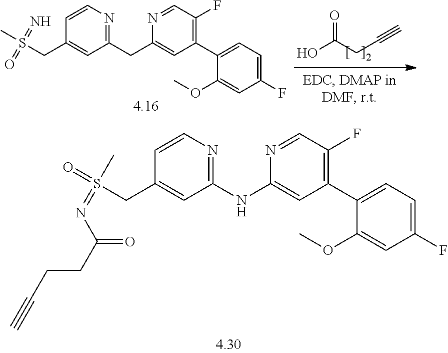

### N-(((2-((5-fluoro-4-(4-fluoro-2-melhoxyphenyl)pyridin-2-yl)amino)pyridin-4-yl)methyl)(methyl)(oxo)-16-sulfaneylidene)pent-4-ynamide (4.30)

Pent-4-ynoic acid (24.3 mg, 0.247 mmol, 1 equiv.), DMAP (66.5 mg, 0.544 mmol, 2.2 equiv.), EDC·HCl (94.8 mg, 0.495 mmol, 2 equiv.) and VIP152 (100 mg, 0.247 mmol, equiv.) were reacted according to general procedure A to provide 4.30 as a white powder (114.5 mg, 95% yield). ¹H NMR (400 MHz, CDCl₁) δ 8.18 (d, J=5.2 Hz, 1H), 8.07 (d, J=1.6 Hz, 1H), 7.94 (s, 1H), 7.65 (d, J=1.4 Hz, 1H), 7.39 (d, J=5.1 Hz, 1H), 7.24-7.15 (m, 1H), 6.81 (dd, J=5.2, 1.5 Hz, 1H), 6.73-6.62 (m, 2H), 4.67 (d, J=13.6 Hz, 1H), 4.58 (d, J=13.6 Hz, 1H), 3.74 (s, 3H), 3.41 (s, 1H), 3.05 (s, 3H), 2.52 (td, J=7.1, 1.4 Hz, 2H), 2.47-2.38 (m, 2H), 1.91 (t, J=2.5 Hz, 11H). ³C NMR (101 MHz, CDCl₃) δ 180.48, 165.53, 163.06, 158.177, 158.07, 154.72, 154.16, 151.68, 149.80, 148.67, 137.43, 135.84, 135.69, 134.91, 134.64, 131.80, 131.70, 131.69, 118.60, 118.57, 117.89, 114.03, 112.94, 107.46, 107.24, 99.81, 99.55, 83.52, 77.26, 68.70, 58.67, 55.97, 50.77, 38.52, 38.23, 14.80. HRMS m/z 485.14506 [M+H]⁺ (calcd. for C₂₄H₂₃F₂N₄O₃S, 485.14534).

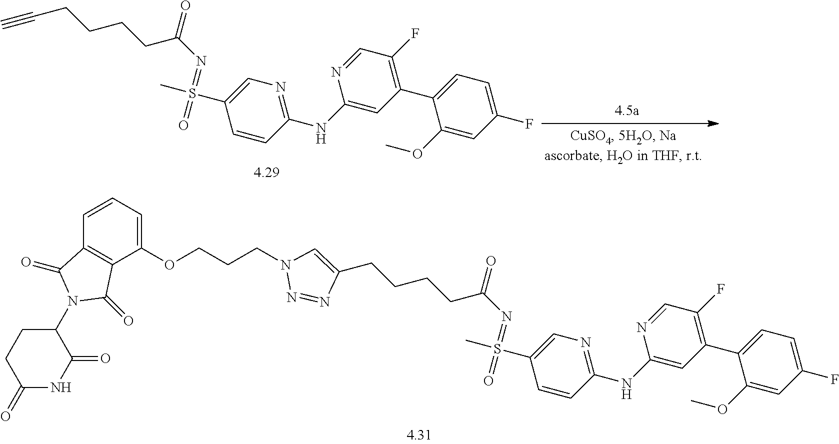 [SMILES: C#CCCCCC(=O)N=S(C)(=O)c1ccc(Nc2cc(-c3ccc(F)cc3OC)c(F)cn2)nc1 ; COc1cc(F)ccc1-c1cc(Nc2ccc(S(C)(=O)=NC(=O)CCCCc3cn(CCCOc4cccc5c4C(=O)N(C4CCC(=O)NC4=O)C5=O)nn3)cn2)ncc1F]

### 5-(1-(3-((2-(2,6-dioxopiperidin-3-yl)-1,3-dioxoisoindolin-4-yl)oxy)propyl)-1H-1,2,3-triazol-4-yl)-N-(((2-((5-fluoro-4-(4-fluoro-2-methoxyphenyl)pyridin-2-yl)amino)pyridin-4-yl)methyl)(methyl)(oxo)-16-sulfaneylidene)pentanamide (4.31)

Sodium ascorbate (34.6 mg, 0.175 mmol, 1.5 equiv.), CuSO₄·H₂O (58.0 mg, 0.233 mmol, 2 equiv.), N-(((2-((5-fluoro-4-(4-fluoro-2-methoxyphenyl)pyridin-2-yl)amino)pyridin-4-yl)methyl)(methyl)(oxo)-16-sulfaneylidene)hept-6-ynamide (4.29) (60.0 mg, 0.117 mmol, 1 equiv.) and 4-(3-azidopropoxy)-2-(2,6-dioxopiperidin-3-yl)isoindoline-1,3-dione (4.5a) (41.7 mg, 0.117 mmol, 1 equiv.) were reacted according to general procedure B to provide 4.31 as a white powder (7 mg, 7% yield), ¹H NMR (400 MHz, CDCl₃) δ 11.16 (d, J=66.1 Hz, 1H), 8.49 (d, J=27.9 Hz, 1H), 8.27 (d, J=4.9 Hz, 1H), 8.16 (d, J=1.7 MHz, 1H), 7.79 (d, J=21.6 Hz, 1H), 7.68 (dd, J=8.5, 7.3 Hz, 1H), 7.50 (d, J=7.3 Hz, 2H), 7.41 (d, =4.5 Hz, 1H), 7.17 (d, 1=8.4 Hz, 2H), 6.88 (s, 1H), 6.74 (ddt, J=15.1, 10.3, 2.2 Hz, 2H), 4.99 (dt, J=12.4, 4.9 Hz, 1H), 4.76-4.56 (m, 4H), 4.17 (d, J=7.6 m, 1H), 3.97 (s, 1H), 3.80 (d, J=1.7 Hz, 3H), 3.10 (s, 3H), 297-2.72 (m, 4H), 2.63 (dd, J=13.9, 7.0 Hz, 2H), 2.49 (p, J=5.9 Hz, 2H), 2.31 (td, J=7.1, 4.8 Hz, 2H), 2.22-2.13 (m, 1H), 1.61 (d, J=6.2 Hz, 3H). ¹³C NMR (176 MHz, CDCl₃) δ 182.83, 167.01, 166.12, 166.09, 158.18, 158.12, 155.96, 149.87, 147.70, 136.75, 133.82, 131.78, 131.72, 122.47, 119.52, 117.87, 117.69, 116.64, 116.62, 107.41, 107.29, 99.71, 99.56, 65.33, 58.55, 55.96, 49.38, 45.87, 39.38, 39.34, 31.56, 29.71, 29.16, 28.73, 25.25, 24.94, 21.89, 22.79. HRMS m/z 870.28156 [M+H]⁺ (calcd. for C₄₂H₄₂F₂N₉O₈S, 870.28396).

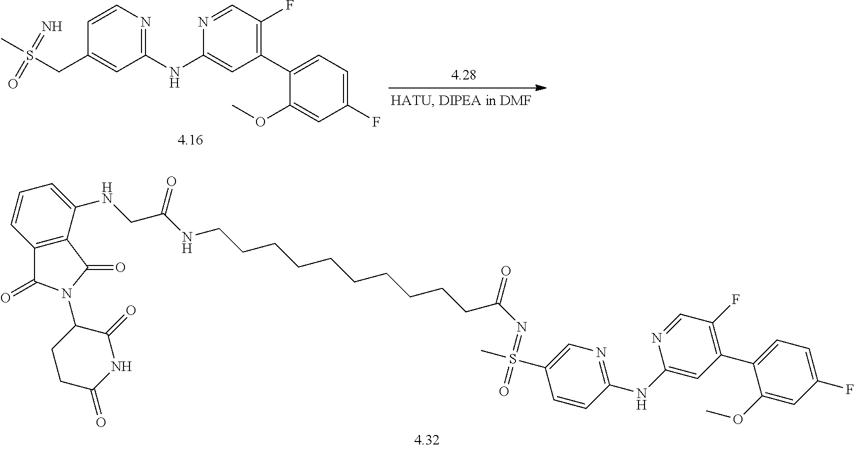 [SMILES: COc1cc(F)ccc1-c1cc(Nc2cc(CS(C)(=N)=O)ccn2)ncc1F ; COc1cc(F)ccc1-c1cc(Nc2ccc(S(C)(=O)=NC(=O)CCCCCCCCCCNC(=O)CNc3cccc4c3C(=O)N(C3CCC(=O)NC3=O)C4=O)cn2)ncc1F]

### 11-(2-((2-(2,6-dioxopiperidin-3-yl)-1,3-dioxoisoindolin-4-yl)amino)acetamido)-N-(((2-((5-fluoro-4-(4-fluoro-2-methoxyphenyl)pyridin-2-yl)amino)pyridin-4-yl)methyl)(methyl)(oxo)-16-sulfaneylidene)undecanamide (4.32)

1-(2-((2-(2,6-dioxopiperidin-3-yl)-1,3-dioxoisoindolin-4-yl)amino)acetamido)undecanoic acid (4.28) (70 mg, 0.136 mmol, 1 equiv.) was reacted with ((2-((5-fluoro-4-(4-fluoro-2-methoxyphenyl)pyridin-2-yl)amino)pyridin-4-yl)methyl)(imino)(methyl)-16-sulfenone (4.16) (55 mg, 0.135 mmol, 1 equiv.), EDC·HCl (52.2 mg, 0.272 mmol, 2 equiv.) and DMAP (36.6 mg, 0.299 mmol, 2.2 equiv.) according to general procedure B to provide 4.32 as a green powder (103 mg 84% yield). ¹H NMR (400 MHz, CDCl₃) δ 11.11 (d, J=82.2 Hz, 1H), 8.55 (d, J=36.3 Hz, 1H), 8.36-8.21 (m, 1H), 8.15 (s, 1H), 7.96 (s, 1H), 7.56 (t, J=7.8 Hz, 1H), 7.39 (s, 1H), 6.96-6.54 (m, 6H), 6.39 (d, J=6.2 Hz, 1H), 4.97 (dt, 1=12.1, 5.2 Hz, 1H), 4.81 (dd, =20.6, 13.4 Hz, 1H), 4.60 (t, J=14.3 Hz, 1H), 3.96 (d, J=6.0 Hz, 2H), 3.82 (s, 3H), 3.27 (ddq, J=38.9, 20.7, 6.6 Hz, 2H), 3.1 (d, J=2.0 Hz, 3H), 2.98-2.66 (m, 4H), 2.31 (dd, J=8.8, 6.7 Hz, 2H), 2.16 (d, J=7.9 Hz, 1H), 1.52-1.35 (m, 3H), 1.33-1.12 (m, 13H). ¹³C NMR (101 MHz, CDC₃) δ 183.24, 172.78, 169.43, 168.77, 167.41, 163.03, 154.64, 150.02, 148.05, 145.93, 136.50, 134.45, 132.40, 131.77, 11869, 117.72, 117.15, 114.17, 113.32, 111.68, 107.41, 107.19, 99.77, 99.52, 58.62, 55.95, 49.14, 47.28, 3970, 39.47, 38.62, 31.54, 29.18, 29.00, 26.69, 25.58, 22.76. HRMS m/z 408.29999 [M+H]⁺ (calcd. for C₄₅H₅₁F₂N₈O₈S, 901.34918).

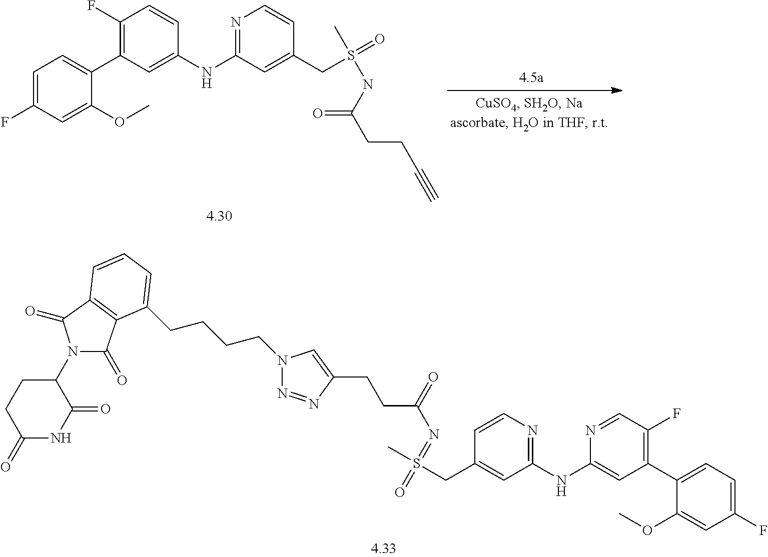

### 3-(1-(3-((2-(2,6-dioxopiperidin-3-yl)-1,3-dioxoisoindolin-4-yl)oxy)propyl)-1H-1,2,3-triazol-4-yl)-N-(((2-((5-fluoro-4-(4-fluoro-2-methoxyphenyl)pyridin-2-yl)amino)pyridin-4-yl)methyl)(methyl)(oxo)-16-sulfaneylidene)propenamide (4.33)

Sodium ascorbate (18.3 mg, 0.092 mmol, 1.5 equiv.), CuSO₄·5H₂O (30.7 mg, 0.123 mmol, 2 equiv.), N-(((2-((5-fluoro-4-(4-fluoro-2-methoxyphenyl)pyridin-2-yl)amino)pyridin-4-yl)methyl)(methyl)(oxo)-16-sulfaneylidene)pent-4-ynamide (4.30) (30.0 mg, 0.062 mmol, 1 equiv.) and 4-(3-azidopropoxy)-2-(2,6-dioxopiperidin-3-yl)isoindoline-1,3-dione (4.5a) (22 mg, 0.062 mmol, 1 equiv.) were reacted according to general procedure B to provide 4.33 as a white powder (15.2 mg, 29% yield). ¹H NMR (400 MHz, CDCl₃) δ 10.74 (d, J=63.9 Hz, 1H), 8.55 (s, 1H), 8.26-8.20 (m, 1H), 8.17 (d, J=1.7 Hz, 1H), 7.80 (dd, J=5.5, 1.9 Hz, 1H), 7.67 (ddd, J=8.5, 7.3, 1.2 Hz, 1H), 7.55 (s, 1H), 7.52-7.45 (m, 1H), 7.41 (d, J=2.1 Hz, 1H), 7.30-7.26 (m, 11H), 7.20-7.13 (m, 1H), 6.84-6.68 (m, 3H), 5.00 (ddd, J=12.2, 5.4, 3.0 Hz, 1H), 4.73-4.62 (m, 2H), 4.61 (d, J=7.0 Hz, 1H), 4.50 (d, J=13.6 Hz, 1H), 4.10 (ddd, J=15.3, 9.6, 6.2 Hz, 1H), 4.03 (d, J=6.3 Hz, 1H), 3.81 (d, J=1.3 Hz, 3H), 3.06-2.60 (m, 9H), 2.45 (p, J=6.1 Hz, 2H), 2.21-2.12 (m, 1H), 1.26 (s, 1H). ¹C NMR (176 MHz, CDCl₃) δ 181.59, 181.53, 171.85, 171.71, 169.66, 166.98, 165.93, 164.99, 163.58, 158.19, 158.13, 155.99, 155.96, 154.72, 153.63, 152.21, 149.88, 148.21, 146.94, 136.72, 136.68, 134.40, 133.82, 131.78, 131.73, 122.65, 119.58, 119.53, 118.80, 117.73, 117.65, 116.51, 114.31, 113.24, 107.37, 107.24, 99.69, 99.55, 65.36, 65.32, 58.68, 58.46, 55.95, 49.25, 46.01, 45.99, 38.76, 38.68, 38.55, 31.46, 29.71, 29.37, 22.83, 22.71, 21.57. HRMS m/z 842.25047 [M+H]⁺ (calcd. for C₄₀H₃₈F₂N₉O₈S, 842.25266).

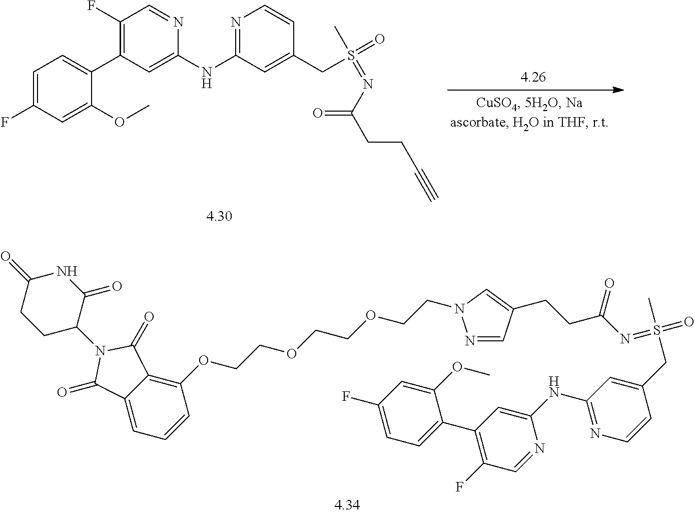 [SMILES: C#CCCC(=O)N=S(C)(=O)Cc1ccnc(Nc2cc(-c3ccc(F)cc3OC)c(F)cn2)c1 ; COc1cc(F)ccc1-c1cc(Nc2cc(CS(C)(=O)=NC(=O)CCc3cnn(CCOCCOCCOc4cccc5c4C(=O)N(C4CCC(=O)NC4=O)C5=O)c3)ccn2)ncc1F]

### 3-(1-(2-(2-(2-((2-(2,6-dioxopiperidin-3-yl)-1,3-dioxoisoindolin-4-yl)oxy)ethoxy)ethoxy)ethyl)-1H-1,2,3-triazol-4-yl)-N-(((2-((5-fluoro-4-(4-fluoro-2-methoxyphenyl)pyridin-2-yl)amino)pyridin-4-yl)methyl)(methyl)(oxo)-16-sulfaneylidene)propenamide (4,34)

Sodium ascorbate (18.3 mg, 0.092 mmol, 1.5 equiv.), CuSO₄·5H₂O (30.7 mg, 0.123 mmol, 2 equiv.), N-(((2-((5-fluoro-4-(4-fluoro-2-methoxyphenyl)pyridin-2-yl)amino)pyridin-4-yl)methyl)(methyl)(oxo)-16-sulfaneylidene)pent-4-ynamide (4.30) (30.0 mg, 0.062 mmol, 1 equiv.) and 4-(2-(2-(2-azidoethoxy)ethoxy)ethoxy)-2-(2,6-dioxopiperidin-3-yl)isoindoline-1,3-dione (4.26) (26.7 mg, 0.062 mmol, 1 equiv.) were reacted according to general procedure B to provide 4.34 as a white powder (15.2 mg, 27% yield). ¹H NMR (400 MHz, CDCl₃) δ 11.11 (s, 1H), 8.81 (s, 1H), 8.19 (s, 2H), 7.83 (t, J=6.6 Hz, 1H), 7.75-7.67 (m, 1H), 7.61 (s, 1H), 7.56-7.47 (m, 2H), 7.32 (d, J=7.5 Hz, 1H), 7.26 (s, 1H), 6.83-6.71 (m, 3H), 5.00 (dd, J=11.3, 4.9 Hz, 1H), 4.75 (dd, J=21.8, 13.9 Hz, 1H), 4.57 (s, 1H), 4.53-4.47 (m, 2H), 4.35 (qd, J=10.6, 5.5 Hz, 2H), 3.96-3.85 (i, 4H), 3.84 (s, 3H), 3.76 (d, J=4.4 Hz, 2H), 3.61 (q, J=5.1 Hz, 2H), 3.05 (t, J=8.8 Hz, 41), 2.95-2.71 (m, 5H), 2.17 (dd, J=12.7, 6.0 Hz, 1H), 1.28 (s, 1H). ¹C NMR (176 MHz, CDCl₃) δ 181.72, 169.66, 167.06, 165.75, 165.05, 163.63, 158.19, 158.14, 156.32, 149.92, 146.86, 136.64, 133.85, 133.83, 131.82, 131.76, 122.44, 119.18, 119.16, 117.51, 117.21, 117.17, 116.18, 116.16, 107.40, 107.28, 99.70, 99.55, 71.08, 71.03, 70.47, 70.44, 69.58, 69.22, 69.20, 69.16, 69.10, 58.49, 55.97, 50.23, 50.19, 49.24, 38.52, 31.54, 29.71, 22.78, 22.75, 21.51. HRMS m/z 916.28769 [M+H]⁺ (calcd. for C₄₃H₄₄F₂N₉O₁₀S, 916.28944).

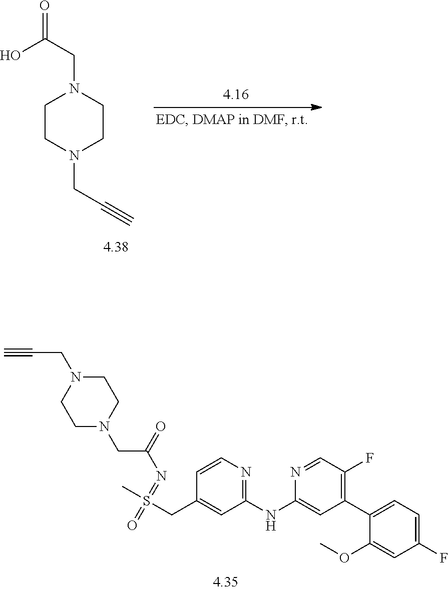 [SMILES: C#CCN1CCN(CC(=O)O)CC1 ; C#CCN1CCN(CC(=O)N=S(C)(=O)Cc2ccnc(Nc3cc(-c4ccc(F)cc4OC)c(F)cn3)c2)CC1]

### N-(((2-((5-fluoro-4-(4-fluoro-2-methoxyphenyl)pyridin-2-yl)amino)pyridin-4-yl)menthyl)(methyl)(oxo)-16-sulfaneylidene)-2-(4-(prop-2-yn-1-yl)piperazin-1-yl)acetamide (4.35)

2-(4-(prop-2-yn-1-yl)piperazin-1-yl)acetic acid (4.38) (225 mg, 1.24 mmol, 5 equiv.), DMAP (66.5 mg, 0.544 mmol, 2.2 equiv.), EDC HCl (94.8 mg, 0.495 mmol, 2 equiv.) and VIP152 (100 mg, 0.247 mmol, 1 equiv.) were reacted according to general procedure A to provide 4.35 (75.4 mg, 54% yield) as a white powder. ¹H NMR (400 MHz, CDCl₃) δ 8.25 (d, J=5.1 Hz, 1H), 8.14 (d, J=1.7 Hz, 1H), 7.74 (s, 1H), 7.65 (s, 1H), 7.42 (d, J=5.0 Hz, 1H), 7.31-7.26 (m, 11H), 6.88 (dd, J=5.2, 1.5 Hz, 1H), 6.81-6.70 (m, 2H), 4.73 (d, J=13.6 Hz, 1H), 4.65 (d, J=13.6 Hz, 1H), 3.82 (s, 3H), 3.28 (d, J=2.5 Hz, 2H), 3.23 (d, J=1.0 Hz, 2H), 3.13 (s, 3H), 2.65 (s, 8H), 2.23 (d, J=4.8 Hz, 1H). ¹³C NMR (101 MHz, CDCl₃) δ 178.94, 165.54, 163.07, 158.18, 158.07, 154.62, 154.18, 151.70, 149.68, 149.66, 148.71, 137.35, 135.86, 135.71, 134.94, 134.67, 131.79, 131.69, 118.58, 118.55, 117.85, 113.97, 112.93, 107.46, 107.25, 99.80, 99.54, 78.85, 77.24, 73.21, 63.61, 58.90, 55.97, 53.03, 51.69, 50.80, 46.83, 38.65. HRMS m/z 569.21356 [M+H]⁺ (calcd. for C₂₈H₃₁F₂N₆O₃S, 569.21409).

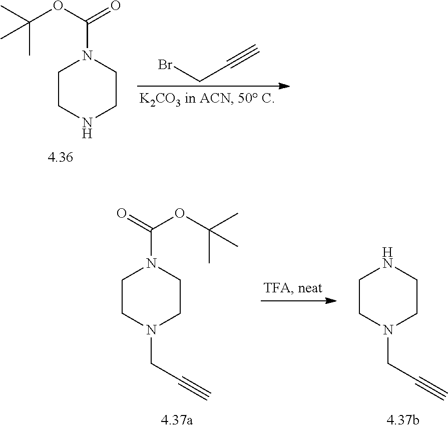

### 1-(prop-2-yn-1-yl)piperazine (4.37b)

tert-butyl piperazine-1-carboxylate 4.36 (1 g, 5.37 mmol, 1 equiv.), dipotassium carbonate (1.48 g, 10.7 mmol, 2 equiv.) and 3-bromnoprop-1-yne (639 mg, 5.37 mmol, 1 equiv.) were dissolved in ACN (13.4 mL 0.4 M) and heated at 50° C. overnight. The reaction mixture was filtered the filtrate was concentrated under reduced pressure. The crude mixture was purified by column chromatography (SiO₂, 30% EtOAc in Hexanes) to provide Boc-protected 4.37a as a yellow gel (1.28 g, 68% yield). ¹H NMR (400 MHz, CDCl₃) δ 3.40 (t, J=5.1 Hz, 4H), 3.25 (t, J=1.6 Hz, 2H), 2.44 (t, J=5.1 Hz, 4H), 2.19 (t, J=2.5 Hz, 1H), 1.39 (s, 9H).

The purified material was dissolved in TFA (1.6 M), stirred for thirty minutes and concentrated under reduced pressure to afford 4.37b, and taken directly onto the next step.

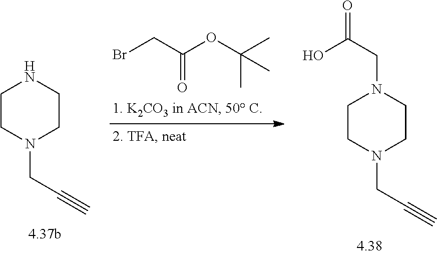

### 2-(4-(prop-2-yn-1-yl)piperazin-1-yl)acetic acid (4.38)

1-(prop-2-yn-1-yl)piperazine (4.37b) (1.28 g, 3.63 mmol, 1 equiv.), dipotassium carbonate (2.01 g, 14.5 mmol, 4 equiv.) and tert-butylbrooacetate (708 mg, 3.63 mmol, 1 equiv.) were dissolved in ACN (9.08 mL, 0.4 M) and heated at 50° C. overnight. The reaction mixture was filtered and the filtrate was concentrated under reduced pressure. The crude mixture was purified by column chromatography (SiO₂, 3% MeOH in DCM) to provide the t-butyl ester of 4.38 as a yellow gel (479 mg, 32% yield). Spectroscopic data are consistent with those previously reported in the literature.^{242 1}H NMR (400 MHz, CDCl₃) δ 3.40 (t, J=5.1 Hz, 4H), 3125 (t, J=1.6 Hz, 2H), 2.44 (t, J 5.1 Hz, 4H), 2.19 (t, J=2.5 Hz, 1H), 1.39 (s, 9H).

The purified material was dissolved in 1.3 mL of TFA, stirred for twenty minutes and concentrated under reduced pressure to afford O, and taken directly onto the next step. 0 was recovered as a brown solid and taken directly onto the next step. ¹H NMR (400 MHz, DMSO) δ 3.89 (d, J=9.4 Hz, 2H), 3.62 (d, J=4.6 Hz, 2H), 3.49 (s, 1H), 3.17 (s, 8H).

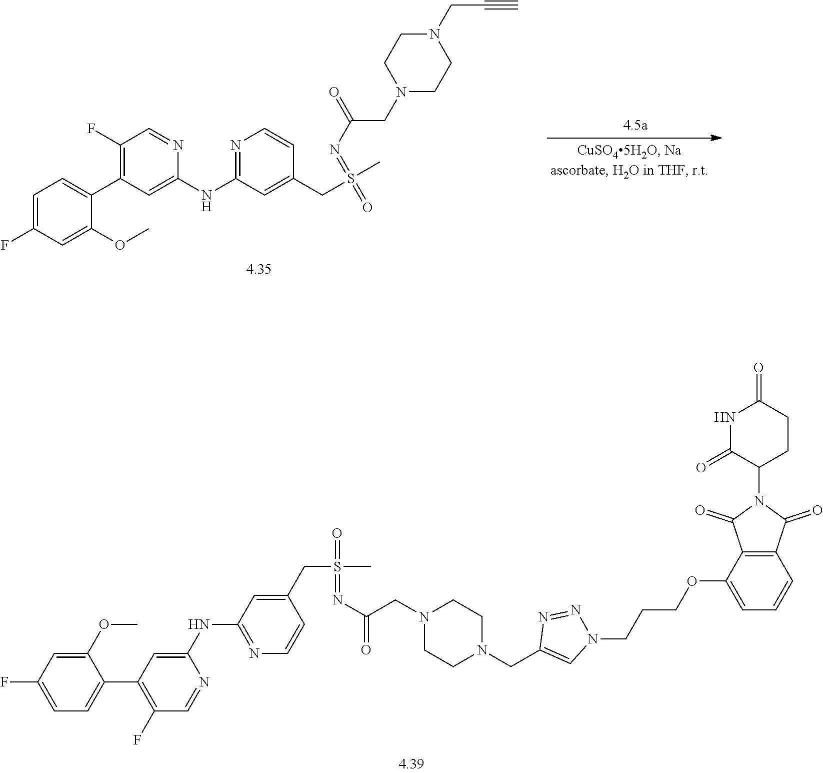 [SMILES: C#CCN1CCN(CC(=O)N=S(C)(=O)Cc2ccnc(Nc3cc(-c4ccc(F)cc4OC)c(F)cn3)c2)CC1 ; COc1cc(F)ccc1-c1cc(Nc2cc(CS(C)(=O)=NC(=O)CN3CCN(Cc4cn(CCCOc5cccc6c5C(=O)N(C5CCC(=O)NC5=O)C6=O)nn4)CC3)ccn2)ncc1F]

### 2-(4-((1-(3-((2-(2,6-dioxopiperidin-3-yl)-1,3-dioxoisoindolin-4-yl)oxy)propyl)-1H-1,2,3-triazol-4-yl)methyl)piperazin-1-yl)-N-(((2-((5-fluoro-4-(4-fluoro-2-methoxyphenyl)pyridin-2-yl)amino)pyridin-4-yl)methyl)(methyl)(oxo)-16-sulfaneylidene)acetamide (4.39)

Sodium ascorbate (3.6 mg, 0.018 mmol, 0.4 equiv.), CuSO₄·5H₂O) (4.5 mg, 0.018 mmol, 0.4 equiv.), N-(((2-((5-fluoro-4-(4-fluoro-2-methoxyphenyl)pyridin-2-yl)amino)pyridin-4-yl)methyl)(methyl)(oxo)-16-sulfaneylidene)-2-(4-(prop-2-yn-1-yl)piperazin-1-yl)acetamide (4.35) (25.5 mg, 0.045 mmol, 1 equiv.) and 4-(3-azidopropoxy)-2-(2,6-dioxopiperidin-3-yl)isoindoline-1,3-dione (4.5a) (20 mag, 0.045 mmol, 1 equiv.) were reacted according to general procedure B to provide 4.39 (11.3 mg, 26% yield) as a brown powder. ¹H NMR (400 MHz, CDCl₃) δ 11.19 (s, 1H), 8.44 (s, 1H), 8.29 (s, 1H), 8.16 (s, 1H), 7.88 (s, 1H), 7.69 (t, J=7.8 Hz, 1H), 7.51 (dd, J=7.3, 3.0 Hz, 1H), 7.47 (s, 1H), 7.29 (d, J=8.6 Hz, 2H), 7.18 (d, J=8.4 Hz, 1H), 6.93 (s, 1H), 6.80-6.70 (m, 2H), 4.99 (dd, J=12.0, 5.4 Hz, 1H), 4.76 (dd, J=12.2, 5.8 Hz, 1H), 4.63 (dd, J=16.0, 9.9 Hz, 2H), 4.22 (s, 1H), 4.04 (d, J=8.0 Hz, 1H), 3.81 (d, J=2.0 Hz, 3H), 3.75 (s, 2H), 3.19 (s, 5H), 3.01-2.73 (m, 5H), 2.62 (s, 4H), 2.50 (d, J=7.0 Hz, 4H), 2.18 (d, J=8.8 Hz, 1H). ¹C NMR (176 MHz, CDCl₃) δ 172.60, 169.16, 166.97, 166.94, 166.07, 165.00, 163.59, 158.19, 158.13, 155.87, 154.68, 153.66, 152.25, 149.93, 148.10, 137.57, 136.79, 135.94, 135.86, 134.56, 134.40, 133.82, 131.80, 131.74, 119.79, 119.72, 118.70, 117.80, 116.83, 116.79, 114.20, 113.30, 107.39, 107.27, 99.69, 99.55, 65.98, 59.03, 55.97, 52.99, 51.84, 49.42, 46.35, 38.99, 31.57, 29.72, 29.02, 22.80, 1.03. HRMS m/z 926.32042 [M+H]⁺ (calcd. for C₄₄H₄₆F₂N₁₁O₈S, 926.32141).

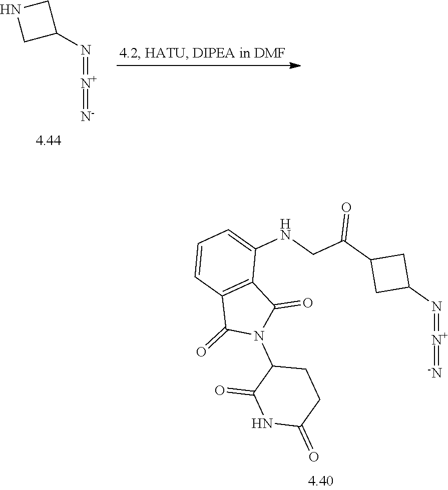

### 4-((2-(3-azidoazetidine-1-yl)-2-oxoethyl)amino)-2-(2,6-dioxopiperidin-3-yl)isoindoline-1,3-dione (4.40)

3-azidoazetidine (4.44) (226 mg, 1.75 mmol, 1 equiv.), 2-{[2-(2,6-dioxopiperidin-3-yl)-1,3-dioxo-2,3-dihydro-1H-isoindol-4-yl]amino}acetic acid (4.2) (331 mg, 0.875 mmol, equiv.), HATU (399 mg, 1.05 mmol, 1.2 equiv.) and DIPEA (226 mg, 1.75 mmol, 2 equiv.) were dissolved in DMF (21.9 mL, 0.04 M). The mixture was stirred overnight at room temperature. The reaction mixture was concentrated under reduced pressure and purified by reversed phase chromatography (C18, H₂O:ACN eluent) to provide 4.40 as a green solid (216 mg, 68% yield). ¹H NMR (400 MHz, DMSO) 11.10 (d, J=8.5 Hz, 1H), 7.66-7.53 (m, 1H), 7.07 (d, J=7.1 Hz, 1H), 6.99 (d, J=8.6 Hz, 1H), 6.84 (t, J=5.3 Hz, 1H), 5.07 (dt, J=13.0, 5.0 Hz, 1H), 4.63-4.46 (m, 2H), 4.28-4.18 (m, 1H), 4.17-4.07 (m, 1H), 4.00 (dd, J=5.3, 2.6 Hz, 2H), 3.81 (dt, J=10.4, 5.9 Hz, 1 h), 2.88 (ddd, J=173, 14.0, 5.3 Hz, 1H), 2.68-2.54 (m, 2H), 2.13-1.98 (m, 1H). ¹³C NMR (101 MHz, DMSO) δ 173.27, 170.59, 170.52, 170.46, 169.25, 169.22, 168.76, 168.55, 167.77, 1670.68, 146.10, 144.14, 136.62, 136.45, 132.50, 132.39, 118.38, 111.47, 111.30, 110.13, 56.33, 55.38, 55.25, 55.11, 50.19, 50.13, 49.14, 4907, 48.97, 42.69, 40.68, 40.47, 31.46, 22.61 HRMS m/z 412.13548 [M+H]⁺ (calcd. for C₁₈H₁₈N₇O₅, 412.13639).

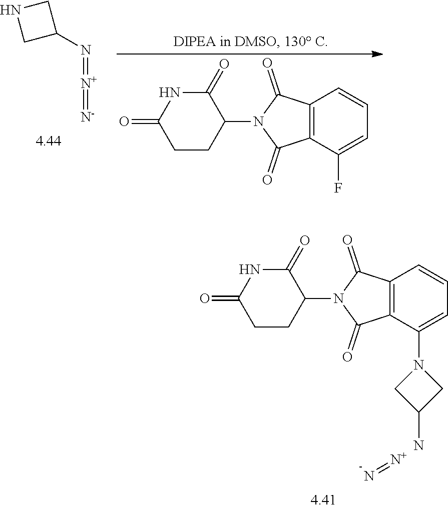

### 4-(3-azidoazetidine-1-yl)-2-(2,6-dioxopiperidin-3-yl)isoindoline-1,3-dione (4.41)

2-(2,6-dioxopiperidin-3-yl)-4-fluoroisoindoline-1,3′-dione (300 mg, 1.09 mmol, 1 equiv.), 3-azidoazetidine (4.45) (245 mg, 1.4 mmol, 1.3 equiv.) and DIPEA (281 mg, 2.2 mmol, 2 equiv.) were dissolved in DMSO. The mixture was heated to 130° C. in a scaled tube and stirred overnight. The reaction mixture was filtered, the filtrate was concentrated under reduced pressure and the crude mixture was purified by reversed phase chromatography (C18, 0-100% ACN in water) to afford 4.41 as a green powder (240 mg, 63% yield). ¹H NMR (400 MHz, CDCl₃) δ 8.03 (s, 1H), 7.49 (dd, J=8.5, 7.1 Hz, 1H), 7.24 (d, J=7.0 Hz, 1H), 6.60 (d, J=8.4 Hz, 1H), 4.97-4.88 (m, 1H), 4.57 (ddt, J=9.5, 7.2, 1.3 Hz, 2H), 4.40 (tt, J=7.1, 4.7 Hz, 1H), 4.18 (ddt, J=9.4, 4.7, 1.3 Hz, 2H), 2.94-2.65 (m, 3H), 2.18-2.07 (m, 1H). ¹C NMR (101 MHz, DMSO) δ 173.25, 170.45, 167.61, 166.94, 147.77, 135.54, 133.67, 120.69, 112.92, 111.47, 60.42, 55.38, 50.42, 49.17, 40.68, 40.48, 31.43, 22.56. HRMS m/z 355.11400 [M+H]⁺ (calcd. for C₁₆H₁₅N₆O₄, 355.11493).

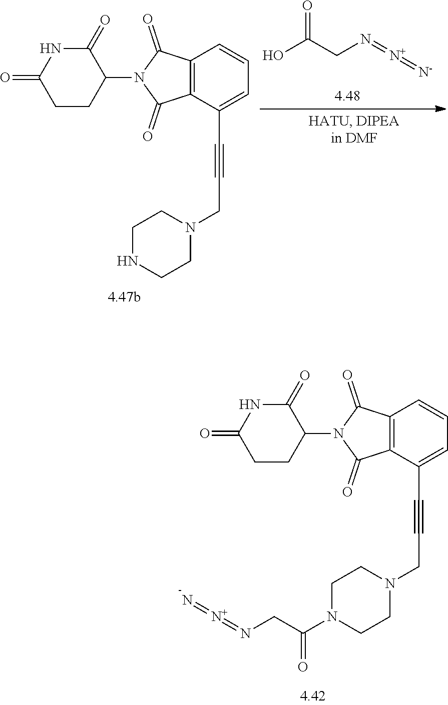

### 4-(3-(4-(2-azidoacetyl)piperazin-1-yl)prop-1-yn-1-yl)˜2-(2,6-dioxopiperidin-3-yl)isoindoline-1,3-dione (4.42)

2-(2,6-dioxopiperidin-3-yl)-4-(3-(piperazin-1-yl)prop-1-yn-1-yl)isoindoline-1,3-dione (4.47b) (174 mg, 0.457 mmol, 1 equiv.), 2-azidoacetic acid (4.48) (60.3 mg, 0.686 mmol, 1.5 equiv.), HATU (348 mg, 0.915 mmol, 1.2 equiv.) and DIPEA (177 mg, 1.37 mmol, 3 equiv.) were dissolved in DMF (11.4 mL, 0.04 M). The mixture was stirred overnight at room temperature. The reaction mixture was concentrated under reduced pressure and purified by reversed phase chromatography (C18, H₂O:ACN eluent) to provide 4.42 (96.3 mg, 45% yield) as a brown solid. ¹H NMR (400 MHz, CDCl₃) δ 8.20 (s, 1H), 7.82 (dd, J=6.7, 1.8 Hz, 1H), 7.75-7.65 (m, 2H), 5.00 (dd, J=12.0, 5.4 Hz, 1H), 3.97 (s, 2H), 3.72 (d, J=16.7 Hz, 4H), 3.47 (d, J=5.5 Hz, 2H), 2.99-2.70 (m, 7H), 2.21-2.11 (m, 1H). ¹³C NMR (101 MHz, CDCl₃) δ 171.10, 168.18, 166.36, 165.84, 165.52, 138.16, 134.02, 132.33, 130.98, 123.20, 120.25, 9224, 81.18, 77.25, 53.45, 51.22, 51.06, 50.68, 49.38, 47.63, 44.95, 42.05, 31.39, 22.61. HRMS m/z 464.16694 [M+H]⁺ (calcd. for C₂₂H₂₂N₇O₅, 464.16769).

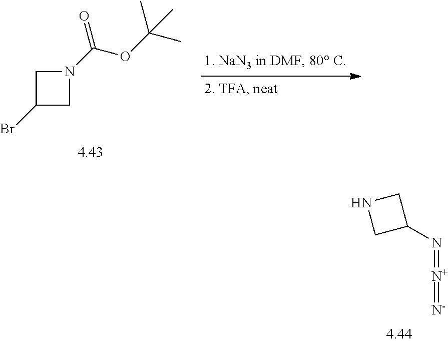

### 3-azidoazetidine (4.44)

Sodium azide and tert-butyl 3-bromo azetidine-1-carboxylate 4.43 were dissolved in DMF (21.2 mL, 0.2 M) and heated to 80° C. The reaction mixture was concentrated under reduced pressure and dissolved in DCM. The solution was washed with water and the aqueous layer was extracted multiple times with DCM. The combined organic layers were dried over sodium sulfate, filtered and concentrated under reduced pressure to afford tert-butyl 3-azidoazetidine-1-carboxylate (700 mg, 83% yield). Spectroscopic data are consistent with those previously reported in the literature.^{243 1}H NMR (400 MHz, CDCl₃) δ 4.20-4.04 (m, 3H), 3.82 (dtt, J=6.6, 2.4, 1.1 Hz, 2H), 1.37 (s, 9H). ³C NMR (101 MHz, CDCl₃) δ 155.92, 80.06, 55.55, 49.37, 28.30. HRMS m/z 199.11930 [M+H]⁺ (calcd. for C₈H₁₅N₄O₂, 199.11895).

Tert-butyl 3-azidoazetidine-1-carboxylate was dissolved in 2.6 mL of TFA and stirred for thirty minutes. The mixture was then concentrated under reduced pressure to afford 4.44 which was taken onto the next step without further purification.

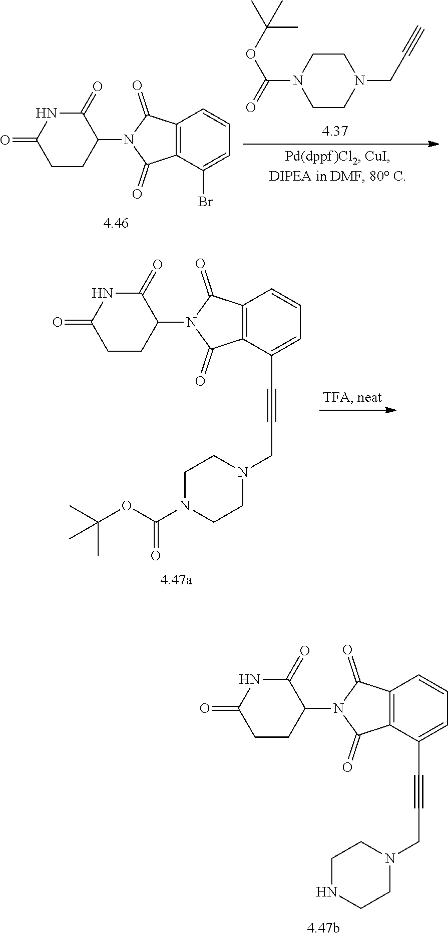

### 2-(2,6-dioxopiperidin-3-yl)-4-(3-(piperazin-1-yl)prop-1-yn-1-yl)isoindoline-1,3-dione (4.47b)

4-bromo-2-(2,6-dioxopiperidin-3-yl)isoindoline-1,3-dione (4.46) (250 mg, 0.742 mmol, 1 equiv.), 1-(prop-2-yn-1-yl)piperazine (4.37) (250 mg, 1.11 mmol, 1.5 equiv.), [1,1′-Bis(diphenylphosphino)ferrocene]dichloropalladium(II) (54.3 mg, 0.074 mmol, 0.1 equiv.), diiodocopper (47.1 mg, 0.148 mmol, 0.2 equiv.) and DIPEA (288 mg, 2.22 mmol, 3 equiv.) were dissolved in THF (3.71 mL, 0.2 M) and heated at 80° C. in a sealed tube under argon overnight. The reaction mixture was concentrated under reduced pressure and the crude mixture was purified by reversed phase chromatography (C18, 0-100% ACN in water) to afford tert-butyl 4-(3-(2-(2,6-dioxopiperidin-3-yl)-1,3-dioxoisoindolin-4-yl)prop-2-yn-1-yl)piperazine-1-carboxylate 4.47a as a brown crystalline solid (380 mg, 107% yield). ¹H NMR (400 MHz, CDCl₃) δ 8.30 (s, 1H), 7.81 (dd, J=7.2, 1.2 Hz, 1H), 7.73 (dd, J=7.9, 1.2 Hz, 11H), 7.70-7.64 (m, 1H), 5.03-4.94 (m, 1H), 3.65 (s, 2H), 3.52 (t, J=5.1 Hz, 4H), 2.95-2.62 (m, 7H), 2.14 (ddd, J=10.5, 5.1, 2.9 Hz, 1H), 1.46 (s, 9H). ¹³C NMR (101 MHz, CDCl₃) δ 170.96, 167.99, 166.40, 165.80, 154.74, 138.45, 133.95, 132.29, 130.90, 123.13, 120.50, 92.94, 80.54, 79.72, 77.24, 53.43, 51.82, 49.35, 47.88, 31.42, 28.43, 22.59. HRMS m/z 481.20736 [M+H]⁺ (calcd. for C₂₅H₂₉N₄O₆, 481.20816).

Tert-butyl 4-(3-(2-(2,6-dioxopiperidin-3-yl)-1,3-dioxoisoindolin-4-yl)prop-2-yn-1-yl)piperazine-1-carboxylate 4.47a was dissolved in TFA and stirred at room temperature until completion as determined by LCMS. The solution was concentrated in vacuo to afford 4.47b, which was taken onto the next step without further purification.

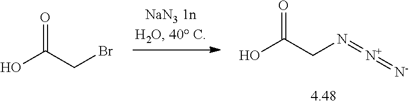 [SMILES: O=C(O)CBr ; [N-]=[N+]=NCC(=O)O]

### 2-azidoacetic acid (4.48)

2-bromoacetic acid (5 g, 36 mmol, 1 equiv.) and sodium azide (4.68 g, 72 mmol, 2 equiv.) were dissolved in water (36 mL, 1 M) and heated under inert atmosphere to 40° C. overnight. The pH of the reaction mixture was adjusted to 3 with 6 N HCl and extracted with 5% EtOH in DCM three times. The combined organic layers were dried over Na₂SO₄, filtered and concentrated under reduced pressure to provide 4.48 as a colorless oil which was used without further purification.

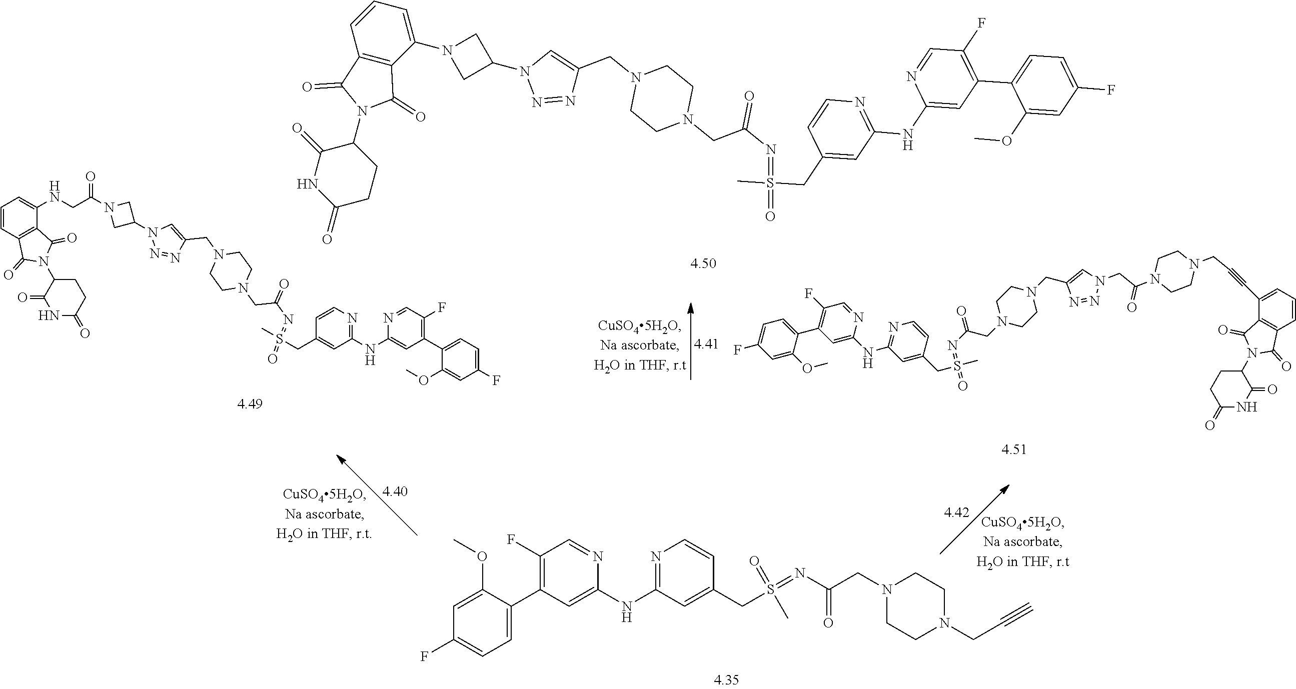 [SMILES: COc1cc(F)ccc1-c1cc(Nc2cc(CS(C)(=O)=NC(=O)CN3CCN(Cc4cn(C5CN(C(=O)CNc6cccc7c6C(=O)N(C6CCC(=O)NC6=O)C7=O)C5)nn4)CC3)ccn2)ncc1F ; COc1cc(F)ccc1-c1cc(Nc2cc(CS(C)(=O)=NC(=O)CN3CCN(Cc4cn(C5CN(c6cccc7c6C(=O)N(C6CCC(=O)NC6=O)C7=O)C5)nn4)CC3)ccn2)ncc1F ; COc1cc(F)ccc1-c1cc(Nc2cc(CS(C)(=O)=NC(=O)CN3CCN(Cc4cn(CC(=O)N5CCN(CC#Cc6cccc7c6C(=O)N(C6CCC(=O)NC6=O)C7=O)CC5)nn4)CC3)ccn2)ncc1F ; C#CCN1CCN(CC(=O)N=S(C)(=O)Cc2ccnc(Nc3cc(-c4ccc(F)cc4OC)c(F)cn3)c2)CC1]

### 2-(4-((1-(1-((2-(2,6-dioxopiperidin-3-yl)-1,3-dioxoisoindolin-4-yl)glycyl) azetidin-3-yl)-1H-1,2,3-triazol-4-yl)methyl) piperazin-1-yl)-N-(((2-((5-fluoro-4-(4-fluoro-2-methoxyphenyl)pyridin-2-yl)amino)pyridin-4-yl)methyl)(methyl) (oxo)-16-sulfaneylidene) acetamide (4.49)

Sodium ascorbate (3.5 mg, 0.018 mmol, 0.4 equiv.), CuSO₄·5H₂O (4.4 mg, 0.018 mmol, 0.4 equiv.), N-(((2-((5-fluoro-4-(4-fluoro-2-methoxyphenyl)pyridin-2-yl)amino)pyridin-4-yl)methyl)(methyl) (oxo)-16-sulfaneylidene)-2-(4-(prop-2-yn-1-yl) piperazin-1-yl) acetamide (4.35) (25 mg, 0.044 mmol, 1 equiv.) and 4-((2-(3-azidoazetidine-1-yl)-2-oxoethyl)amino)-2-(2,6-dioxopiperidin-3-yl) isoindoline-1,3-dione (4.40) (18.1 mg, 0.044 mmol, 1 equiv.) were reacted according to general procedure B to provide 4.49 (10 mg, 23% yield) as a green powder. ¹H NMR (700 MHz, Acetone) & 9.98-9.91 (m, 1H), 8.89 (s, 1H), 8.21 (d, J=5.1 Hz, 1H), 8.16 (d, J=1.5 Hz, 1H), 8.07 (s, 1H), 7.89 (d, J=5.8 Hz, 1H), 7.85 (s, 1H), 7.59 (dd, J=8.5, 7.1 Hz, 1H), 7.39 (dd, J=8.4, 6.7 Hz, 1H), 7.08 (d, J=7.1 Hz, 1H), 7.04 (d, J=8.5 Hz, 1H), 7.02-6.97 (m, 2H), 6.87 (td, J=8.3, 2.4 Hz, 1H), 5.67 (tt, J=8.1, 5.2 Hz, 1H), 5.12-5.05 (m, 1H), 4.94 (t, J=8.7 Hz, 1H), 4.85 (s, 1H), 4.75 (dd, J=9.6, 5.2 Hz, 1H), 4.61 (t, J=9.2 Hz, 1H), 4.41 (dd, J=10.3, 5.3 Hz, 1H), 4.19-4.10 (m, 2H), 3.87 (s, 3H), 3.60 (s, 2H), 3.26 (s, 3H), 3.06 (s, 2H), 3.01-2.93 (m, 1H), 2.78 (s, 3H), 2.53 (s, 4H), 2.44 (s, 4H), 2.21 (tt, J=12.0, 4.7 Hz, 1H), 2.09 (s, 1H). ¹³C NMR (176 MHz, Acetone) δ 178.35, 171.79, 169.32, 169.24, 169.12, 168.16, 167.39, 164.96, 163.56, 158.50, 158.44, 154.92, 154.85, 153.36, 151.96, 150.79, 150.72, 147.94, 145.88, 145.80, 144.85, 138.45, 135.99, 135.33, 135.25, 134.33, 134.18, 132.75, 131.79, 131.73, 122.40, 119.24, 118.10, 117.38, 117.35, 113.93, 113.88, 113.51, 113.47, 110.94, 110.72, 110.69, 107.01, 106.89, 99.78, 99.63, 63.47, 58.00, 56.94, 55.68, 53.14, 52.77, 52.54, 49.09, 49.04, 49.02, 42.51, 42.43, 38.42, 38.40, 31.14, 31.11, 29.69, 29.41, 22.53. HRMS m/z 980.34095 [M+H]⁺ (calcd. for C₄₆H₄₈F₂N₁₃O₈S, 980.34321).

### 2-(4-((1-(1-(2-(2,6-dioxopiperidin-3-yl)-1,3-dioxoisoindolin-4-yl)azetidin-3-yl)-11H-1,2,3-triazol-4-yl)methyl)piperazin-1-yl)-N-(((2-((5-fluoro-4-(4-fluoro-2-methoxyphenyl)pyridin-2-yl)amino)pyridin-4-yl)methyl)(methyl)(oxo)-16-sulfaneylidene)acetamide (4.50)

Sodium ascorbate (4.2 mg, 0.021 mmol, 0.4 equiv.), CuSO₄·5H₂O (5.2 mg, 0.021 mmol, 0.4 equiv.), N-(((2-((5-fluoro-4-(4-fluoro-2-methoxyphenyl)pyridin-2-yl)amino)pyridin-4-yl)methyl)(methyl)(oxo)-16-sulfaneylidene)-2-(4-(prop-2-yn-1-yl)piperazin-1-yl)acetamide (4.35) (30 mg, 0.053 mmol, 1 equiv.) and 4-(3-azidoazetidin-1-yl)-2-(2,6-dioxopiperidin-3-yl)isoindoline-1,3-dione (4.41) (18.7 mg, 0.053 mmol, 1 equiv.) were reacted according to general procedure B to provide 4.50 (18 mg, 37% yield) as a green powder. ¹H NMR (700 Hz, CDCl₃) δ 10.69 (d, J=156.5 Hz, 1H), 8.38 (d, J=83.0 Hz, 1H), 8.21 (dd, J=14.4, 5.2 Hz, 1H), 8.08 (d, J=4.0 Hz, 1H), 7.87 (d, J=65.9 Hz, 1H), 7.80-7.66 (m, 1H), 7.48 (t, J=7.8 Hz, 1H), 7.29 (d, J=33.9 Hz, 1H), 7.23 (dd, J=7.1, 3.1 Hz, 1H), 7.18-7.15 (m, 1H), 6.80 (dd, J=12.9, 5.2 Hz, 1H), 6.70-6.59 (m, 3H), 5.43 (dddd, J=20.2, 12.6, 8.0, 5.2 Hz, 1H), 4.88 (dt, J=12.6, 6.4 Hz, 1H), 4.80 (td, J=8.7, 4.7 Hz, 1H), 4.77-4.69 (m, 2H), 4.63 (s, 1H), 4.56-4.45 (m, 2H), 3.72 (d, J=9.3 Hz, 4H), 3.21-3.10 (m, 2H), 3.09 (s, 1H), 3.06 (s, 1H), 2.84-2.77 (m, 1H), 2.69 (tdd, J=18.6, 15.5, 9.2 Hz, 3H), 2.56 (s, 6H), 2.11-2.05 (m, 1H), 1.21, 1.17 (m, 3H). ¹³C NMR (176 MHz, CDCl₃) δ 178.78, 172.53, 172.17, 169.32, 169.21, 167.38, 166.96, 166.93, 164.98, 164.95, 163.57, 163.54, 158.15, 15813, 158.10, 158.07, 154.66, 154.62, 153.62, 153.58, 152.20, 152.17, 149.97, 149.91, 148.14, 147.13, 147.08, 137.98, 137.36, 135.95, 135.87, 135.78, 135.19, 134.56, 134.50, 134.40, 134.34, 133.84, 131.79, 131.73, 129.50, 119.62, 118.57, 1170.71, 117.62, 114.17, 11414, 113.99, 113.96, 113.18, 113.14, 112.26, 107.41, 107.28, 99.71, 99.57, 67.79, 63.64, 63.53, 61.66, 61.31, 60.14, 59.21, 58.74, 55.95, 55.93, 53.24, 53.12, 52.71, 52.47, 52.45, 49.71, 49.63, 49.17, 49.15, 49.06, 38.93, 38.73, 31.44, 30.58, 29.72, 29.38, 29.00, 24.00, 22.87, 22.70. HRMS m/z 923.32175 [M+H]⁺ (calcd. for C₄₄H₄₅F₂N₁₂O₇S, 923.32175).

### 2-(4-((1-(2-(4-(3-(2-(2,6-dioxopiperidin-3-yl)-1,3-dioxoisoindolin-4-yl)prop-2-yn-1-yl)piperazin-1-yl)-2-oxoethyl)-1H-1,2,3-triazol-4-yl)methyl)piperazin-1-yl)-N-(((2-((5-fluoro-4-(4-fluoro-2-methoxyphenyl)pyridin-2-yl)amino)pyridin-4-yl)methyl)(methyl)(oxo)-16-sulfaneylidene)acetamide (4.51)

Sodium ascorbate (4.2 mg, 0.021 mmol, 0.4 equiv.), CuSO₄·5H₂O (5.2 mg, 0.021 mmol, 0.4 equiv.), N-(((2-((5-fluoro-4-(4-fluoro-2-methoxyphenyl)pyridin-2-yl)amino)pyridin-4-yl)methyl)(methyl)(oxo)-16-sulfaneylidene)-2-(4-(prop-2-yn-1-yl)piperazin-1-yl)acetamide (4.35) (30 mug, 0.053 mmol, 1 equiv.) and 4-(3-(4-(2-azidoacetyl)piperazin-1-yl)prop-1-yn-1-yl)-2-(2,6-dioxopiperidin-3-yl)isoindoline-1,3-dione (4.42) (24.5 mg, 0.053 mmol, 1 equiv.) were reacted according to general procedure B to provide 4.51 (26.8 mg, 49% yield as an off-white powder. ¹H NMR (700 MHz, CDCl₃) δ 11.32 (d, J=327.3 Hz, 1H), 8.45 (d, J=166.7 Hz, 1H), 8.19 (dd, J=17.9, 4.3 Hz, 1H), 8.09 (d, J=11.8 Hz, 1H), 7.72 (dd, J=7.2, 4.0 Hz, 1H), 7.67 (s, 1H), 7.62 (ddd, J=14.8, 7.5, 3.4 Hz, 2H), 7.48 (d, J=21.0 Hz, 1H), 7.21 (d, J=7.5 Hz, 2H), 6.80 (dd, J=18.0, 5.1 Hz, 1H), 6.73-6.59 (m, 2H), 5.32 (d, J=16.3 Hz, 1H), 5.22 (d, J=7.5 Hz, 1H), 5.19 (d, J=15.6 Hz, 1H), 4.74 (dd, J=14.2, 5.7 Hz, 1H), 4.54 (dd, J=13.6, 8.4 Hz, 1H), 3.85 (d, J=14.1 Hz, 1H), 3.74 (s, 3H), 3.72-3.44 (m, 7H), 3.16 (d, 1′=18.1 Hz, 3H), 3.12-3.03 (m, 3H), 2.98 (d, J=26.9 Hz, 1H), 2.84 (tdd, J=23.8, 16.3, 9.7 Hz, 4H), 2.73 (d, J=7.1 Hz, 1H), 2.71-2.65 (m, 2H), 2.53 (d, J=70.5 Hz, 6H), 2.11 (q, J=14.0 Hz, 1H). ¹³C NMR (176 MHz, CDCl₃) δ 178.94, 172.97, 169.66, 169.47, 166.73, 166.60, 166.05, 165.97, 164.96, 163.55, 163.17, 163.10, 158.18, 158.13, 154.77, 154.74, 153.60, 152.18, 149.86, 148.33, 148.15, 138.05, 137.97, 137.61, 136.86, 135.93, 135.85, 135.78, 134.54, 134.38, 134.22, 133.92, 133.88, 132.51, 132.44, 131.81, 131.79, 131.75, 131.73, 131.00, 123.05, 123.00, 120.16, 118.87, 118.76, 117.62, 114.42, 114.24, 113.24, 113.16, 1070.35, 107.33, 107.23, 107.21, 99.68, 99.65, 99.53, 99.51, 92.02, 91.91, 81.93, 81.74, 63.48, 59.11, 58.49, 55.95, 52.47, 52.22, 50.86, 50.64, 50.42, 49.52, 49.49, 47.66, 47.64, 45.29, 45.25, 42.81, 42.70, 39.38, 38.94, 31.58, 31.51, 29.71, 22.58, 22.48. HRMS m/z 1032.37244 [M+H]⁺ (calcd. for C₅₀H₅₂F₂N₁₃O₈S, 1032.37451).

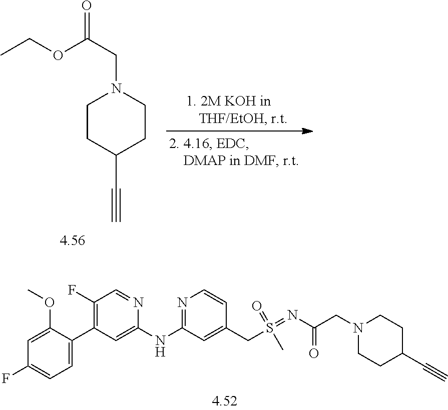 [SMILES: C#CC1CCN(CC(=O)OCC)CC1 ; C#CC1CCN(CC(=O)N=S(C)(=O)Cc2ccnc(Nc3cc(-c4ccc(F)cc4OC)c(F)cn3)c2)CC1]

### 2-(4-ethynylpiperidin-1-yl)-N-(((2-((5-fluoro-4-(4-fluoro-2-methoxyphenyl)pyridin-2-yl)amino)pyridin-4-yl)methyl)(methyl)(ox)-16-sulfaneylidene)acetamide (4.52)

Ethyl 2-(4-ethynylpiperidin-1-yl)acetate 4.56 was dissolved in ethanol (10.2 mL 0.15 M) and THF (10.2 mL, 0.15 M). A 2 N potassium hydroxide solution (3.11 mL 4 equiv.) was added, and the reaction mixture was stirred for 3 hours. The resulting mixture was cooled over ice and neutralized with 6 N HCL. The aqueous portion was then removed, leaving the carboxylic acid of 4.56 as a white solid which was taken to the next step without further purification.

2-(4-ethynylpiperidin-1-yl)acetic acid (4.56) (147 mg, 0.618 mmol, 1 equiv.), DMAP (166 mg, 1.36 mmol, 2.2 equiv.), EDC·HCl (237 mg, 1.24 mmol, 2 equiv.) and VIP152 4.16 (250 mg, 0.618 mmol, 1 equiv.) were reacted according to general procedure A to provide 4.52 (218 mg, 64% yield) as a white powder. ¹H NMR (700 MHz, DMSO) δ 9.91 (s, 1H), 8.24-8.20 (m, 2H), 7.73-7.70 (m, 2H), 7.35 (dd, J=8.4, 6.7 Hz, 1H), 7.11 (dd, J=11.4, 2.5 Hz, 1H), 6.96-6.89 (m, 2H), 4.91-4.83 (m, 2H), 3.81 (s, 3H), 3.27 (s, 3H), 3.18 (d, J=5.2 Hz, 1H), 3.01 (s, 2H), 2.87 (d, J=2.4 Hz, 1H), 2.65 (h, J=3.6 Hz, 2H), 2.22 (d, J=10.9 Hz, 2H), 1.73-1.67 (m, 2H), 1.50-1.42 (m, 2H). ¹³C NMR (176 MHz, DMSO) δ 178.63, 164.83, 163.44, 158.41, 158.35, 154.98, 153.12, 151.72, 151.16, 148.16, 138.48, 135.29, 135.20, 134.65, 134.50, 132.08, 132.03, 119.20, 119.18, 118.51, 114.18, 113.88, 107.67, 107.55, 100.70, 100.55, 88.04, 72.03, 63.96, 5783, 56.70, 51.59, 39.18, 31.91, 26.38. HRMS m/z 554.20269 [M+H]+ (calcd. for C₂₈H₃₀F₂N₅O₃S 554.20319).

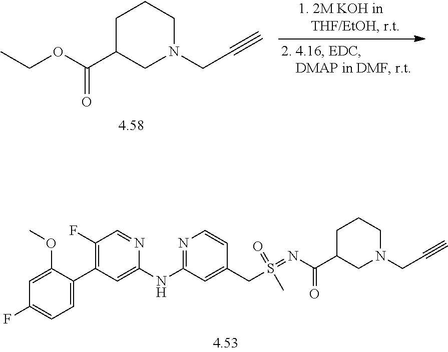 [SMILES: C#CCN1CCCC(C(=O)OCC)C1 ; C#CCN1CCCC(C(=O)N=S(C)(=O)Cc2ccnc(Nc3cc(-c4ccc(F)cc4OC)c(F)cn3)c2)C1]

### N-(((2-((5-fluoro-4-(4-fluoro-2-methoxyphenyl)pyridin-2-yl)amino)pyridin-4-yl)methyl)(methyl)(oxo)-16-sulfaneylidene)-1-(prop-2-yn-1-yl)piperidine-3-carboxamide (4.53)

Ethyl 1-(prop-2-yn-1-yl)piperidine-3-carboxylate 4.58 was dissolved in ethanol (0.15 M) and THE (0.15 M). A 2 N potassium hydroxide solution (0.5 M) was added, and the reaction mixture was stirred for 3 hours. The resulting mixture was cooled over ice and neutralized with 6 N HCl. The aqueous portion was then removed, leaving the carboxylic acid of 4.58 as a white solid which was taken to the next step without further purification. 1-(prop-2-yn-1-yl)piperidine-3-carboxylic acid (135 mg, 0.569 mmcl, 1 equiv.), DMAP (153 mg, 1.25 mmcl, 2.2 equiv.), EDC·HCl (218 mg, 1.14 mmcl, 2 equiv.) and VIP152 4.16 (230 mg, 0.569 mmol, 1 equiv.) were reacted according to general procedure A to provide 4.53 (191 mg, 61% yield) as a white powder. ¹H NMR (700 MHz, DMSO) δ 9.91 (s, 1H), 8.24-8.19 (m, 2H), 7.73 (d, 1=4.3 Hz, 1H), 7.69 (dd, J=5.4, 1.8 Hz, 1H), 7.35 (dd, J=8.4, 6.7 Hz, 1H), 7.11 (dd, J=11.4, 2.5 Hz, 1H), 6.96-6.88 (m, 2H), 4.90-4.83 (m, 2H), 3.81 (s, 3H), 3.26 (s, 3H), 3.21-3.16 (M, 2H), 3.10 (q, J=2.3 Hz, 1H), 2.87-2.82 (m, 1H), 2.63 (d, J=10.6 Hz, 1H), 2.30 (tq, J=11.0, 3.8 Hz, 1H), 2.09 (ddd, J=12.9-7.3, 2.8 Hz, 1H), 2.01-1.94 (m, 1H), 1.77 (ddd, J=12.7, 8.5, 4.1 Hz, 1H), 1.58 (dt, J=12.9, 3.6 Hz, 1H), 1.43-1.34 (m, 1H), 1.22-1.14 (m, 1H). ¹³C NMR (176 MHz, DMSO) δ 182.17, 164.83, 163.44, 158.41, 158.35, 154.94, 1530.12, 151.72, 151.19, 148.15, 138.50, 135.31, 135.22, 134.61, 134.46, 132.07, 132.01, 119.19, 119.17, 118.63, 114.22, 113.86, 107.68, 107.55, 100.70, 100.55, 76.02, 60.23, 57.63, 56.70, 55.03, 55.02, 52.08, 46.99, 45.87, 39.00, 27.29, 27.26, 24.74. HRMS m/z 554.20257 [M+H]⁺ (calcd. for C₂₈H₃₀F₂N₅O₃S, 554.20319).

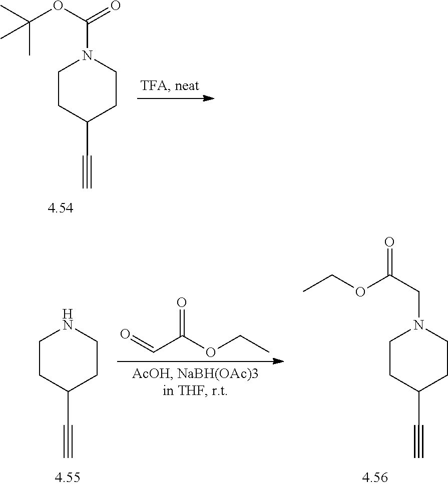

### Ethyl 2-(4-ethynylpiperidine-1-yl)acetate (4.56)

Tert-butyl 4-ethynylpiperidine-1-carboxylate (2.0 g, 9.65 mmol) was dissolved in TEA (1.6 M) and the mixture was stirred at room temperature for thirty minutes. The reaction was then concentrated under reduced pressure to provide 4-ethynylpiperidine 4.55 which was taken forward without further purification.

4-ethynylpiperidine 4.55 (1 g, 9.16 mmol, 1 equiv.) and ethyl-2-oxoacetate (935 mg, 9.16 mmol, 1 equiv.) were dissolved in THF (22.9 mL, 0.4 M) and stirred for ten minutes before addition of sodium (bis(acetoxy)boranuidyl acetate (3.88 g, 18.3 mmol, 2 equiv.). Reaction was allowed to proceed overnight after which the mixture was concentrated under reduced pressure, then dissolved in H₂O. The aqueous mixture was extracted with 5% EtOH in DCM. The combined organic layers were dried over Na₂SO₄ and filtered. The crude mixture was purified by column chromatography (SiO₂, 60% EtOAc in Hexanes) to provide ethyl 2-(4-ethynylpiperidin-1-yl)acetate 4.56 as a colorless oil (468 mg, 26% yield). ¹H NMR (400 MHz, CDCl₃) 4.18 (q, J=7.1 Hz, 2H), 3.20 (s, 2H), 2.80 (ddd, J=10.6, 6.2, 3.8 Hz, 2H), 2.38 (ddd, J=11.8, 9.1, 3.0 Hz, 3H), 2.07 (d, J=2.4 Hz, 1H), 1.95-1.84 (m, 2H), 1.81-1.68 (m, 2H), 1.27 (t, J=7.1 Hz, 3H). ¹³C NMR (176 MHz, CDCl₃) δ 170.45, 87.18, 69.03, 60.59, 59.87, 51.74, 31.40, 26.26, 14.27. HRMS m/z 196.13307 [M+H]⁺ (calcd. for C₁₁H₁₈NO₂, 196.13321).

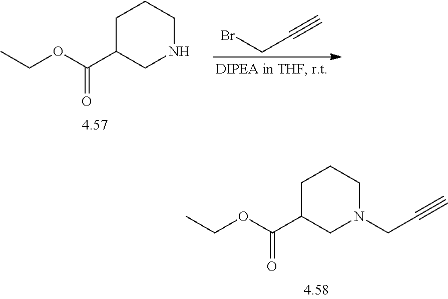

### Ethyl 1-(prop-2-yn-1-yl)piperidine-3-carboxylate (4.58)

Ethyl piperidine-3-carboxylate (2.0 g, 12.7 mmol) was dissolved in THE (0.2 M) with DIPEA (4.93 g, 38.2 mmol) and stirred at room temperature overnight. The crude mixture was concentrated under reduced pressure and purified by column chromatography (SiO₂, 40-60% EtOAc in Hexanes) to provide ethyl 2-(4-ethynylpiperidin-1-yl)acetate 4.56 as a colorless oil (1.66 g, 67% yield). ¹H NMR (700 MHz, CDCl₃) δ 4.18-4.09 (m, 2H), 3.33 (dt, J=2.5, 1.2 Hz, 2H), 3.03-2.98 (m, 1H), 2.80-2.75 (m, 1H), 2.62-2.55 (m, 1H), 2.38 (t, J=10.7 Hz, 1H), 2.25 (td, J=2.5, 0.8 Hz, 1H), 2.23-2.19 (m, 1H), 1.98-1.91 (m, 1H), 1.80-1.73 (m, 1H), 1.65-1.56 (m, 1H), 1.44 (qd, J=11.8, 3.9 Hz, 1H), 1.26 (td, J=7.1, 1.2 Hz, 3H). ¹³C NMR (176 MHz, CDCl₃) δ 173.98, 78.72, 73.22, 60.38, 54.16, 52.23, 47.31, 41.89, 26.47, 24.53, 14.21, HRMS m/z 196.13307 [M+H]⁺ (calcd. for C₁₁H₁₈NO₂, 196.13321).

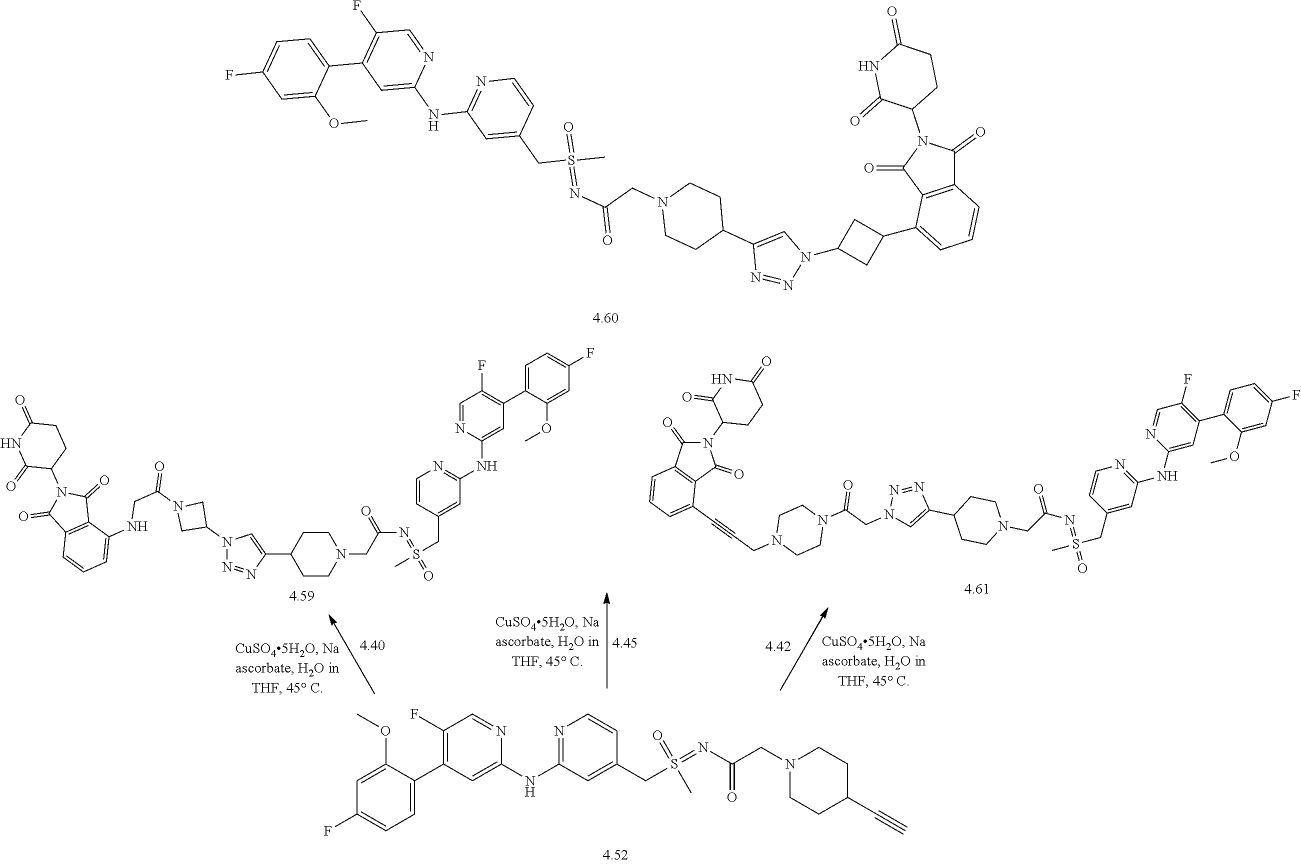 [SMILES: COc1cc(F)ccc1-c1cc(Nc2cc(CS(C)(=O)=NC(=O)CN3CCC(c4cn(C5CC(c6cccc7c6C(=O)N(C6CCC(=O)NC6=O)C7=O)C5)nn4)CC3)ccn2)ncc1F ; COc1cc(F)ccc1-c1cc(Nc2cc(CS(C)(=O)=NC(=O)CN3CCC(c4cn(C5CN(C(=O)CNc6cccc7c6C(=O)N(C6CCC(=O)NC6=O)C7=O)C5)nn4)CC3)ccn2)ncc1F ; COc1cc(F)ccc1-c1cc(Nc2cc(CS(C)(=O)=NC(=O)CN3CCC(c4cn(CC(=O)N5CCN(CC#Cc6cccc7c6C(=O)N(C6CCC(=O)NC6=O)C7=O)CC5)nn4)CC3)ccn2)ncc1F ; C#CC1CCN(CC(=O)N=S(C)(=O)Cc2ccnc(Nc3cc(-c4ccc(F)cc4OC)c(F)cn3)c2)CC1]

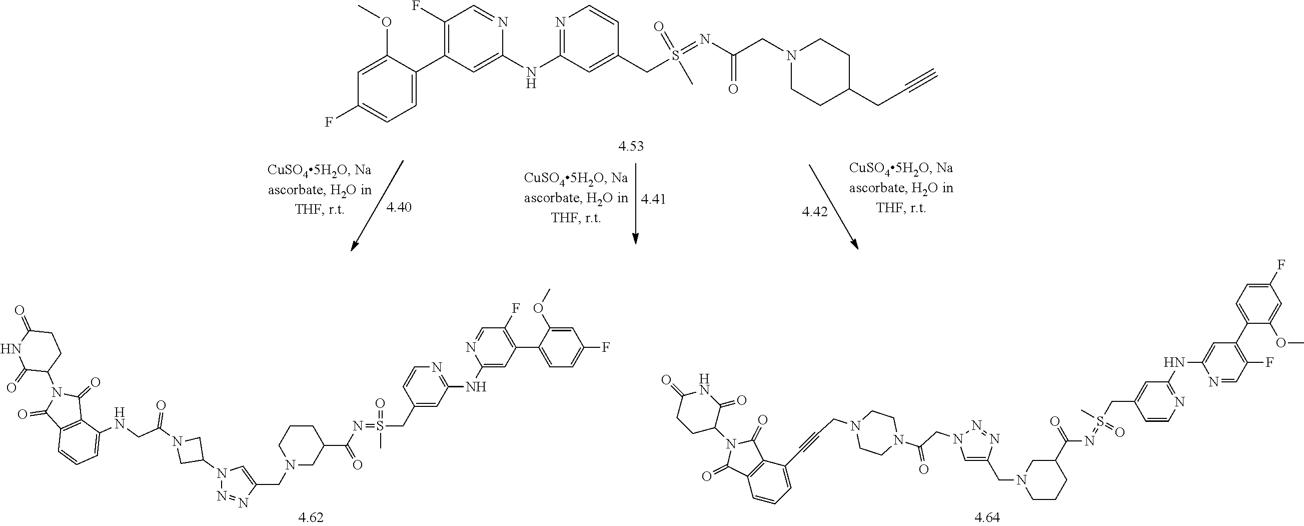 [SMILES: C#CCC1CCN(CC(=O)N=S(C)(=O)Cc2ccnc(Nc3cc(-c4ccc(F)cc4OC)c(F)cn3)c2)CC1 ; COc1cc(F)ccc1-c1cc(Nc2cc(CS(C)(=O)=NC(=O)C3CCCN(Cc4cn(C5CN(C(=O)CNc6cccc7c6C(=O)N(C6CCC(=O)NC6=O)C7=O)C5)nn4)C3)ccn2)ncc1F ; COc1cc(F)ccc1-c1cc(Nc2cc(CS(C)(=O)=NC(=O)C3CCCN(Cc4cn(CC(=O)N5CCN(CC#Cc6cccc7c6C(=O)N(C6CCC(=O)NC6=O)C7=O)CC5)nn4)C3)ccn2)ncc1F]

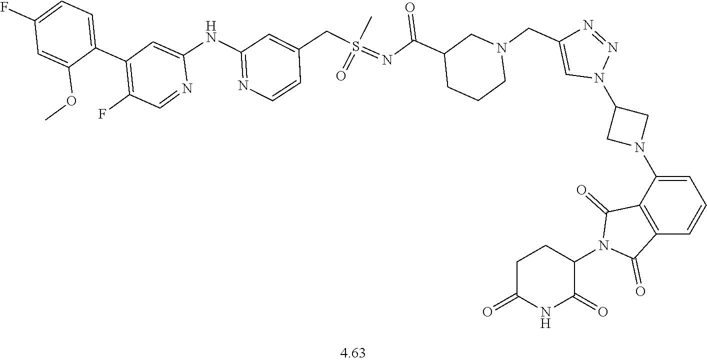 [SMILES: COc1cc(F)ccc1-c1cc(Nc2cc(CS(C)(=O)=NC(=O)C3CCCN(Cc4cn(C5CN(c6cccc7c6C(=O)N(C6CCC(=O)NC6=O)C7=O)C5)nn4)C3)ccn2)ncc1F]

### 2-(4-(1-(1-((2-(2,6-dioxopiperidin-3-yl)-1,3-dioxoisoindolin-4-yl)glycyl)azetidin-3-yl)-1H-1,2,3-triazol-4-yl)piperidin-1-yl)-N-(((2-((5-fluoro-4-(4-fluoro-2-methoxyphenyl)pyridin-2-yl)amino)pyridin-4-yl)methyl)(methyl)(oxo)-16-sulfaneylidene)acetamide (4.59)

Sodium ascorbate (4.3 mg, 0.02 mmol), CuSO₄·5H₂O (54 mg, 0.02 mmol), 2-(4-ethynylpiperidin-1-yl)-N-{[(2-{[5-fluoro-4-(4-fluoro-2-methoxyphenyl)pyridin-2-yl]amino}pyridin-4-yl)methyl](methyl)oxo-λ⁶sulfanylidene}acetamide (4.52) (30 mg, 0.05 mmol) and 4-([2-(3-azidoazetidin-1-yl)-2-oxoethyl]amino}-2-(2,6-dioxopiperidin-3-yl)-2,3-dihydro-1H-isoindole-1,3-dione (4.40) (22.3 mg, 0.05 mmol) were reacted according to general procedure B to provide 4.59 (21 mg, 40% yield) as a green powder. ¹H NMR (700 MHz, DMSO) δ 11.07 (s, 1H), 9.91 (s, 1H), 8.23-8.19 (m, 3H), 8.15 (s, 1H), 7.73-7.69 (m, 2H), 7.63 (dd, J=8.5, 7.0 Hz, 1H), 7.32 (dd, J=8.4, 6.8 Hz, 1H) 7.21 (d, J=7.0 Hz, 1H), 7.09 (dd, 1=11.4, 2.5 Hz, 1H), 6.92-6.89 (m, 3H), 5.57 (tt, 1=7.8, 5.3 Hz, 1H), 5.07 (dd, J=12.9, 55 Hz, 1H), 4.90-4.84 (m, 2H), 4.75 (t, J=8.71 Hz, 2H), 4.49 (td, J=8.6-5.2 Hz, 2H), 3.79 (s, 3H), 3.27 (s, 3H), 3.06 (s, 2H), 2.91-2.83 (m, 4H), 2.61-2.51 (m, 4H), 2.20 (ddd, J=12.0, 8.5, 3.3 Hz, 2H), 2.00 (dtd, J=13.2, 5.5, 2.0 Hz, 1H), 1.87-1.82 (m, 2H), 1.58 (qd, J=12.3, 3.7 Hz, 2H). ¹³C NMR (176 MHz, DMSO) δ 178.74, 178.63, 173.29, 170.54, 169.27, 168.89, 167.78, 164.83, 163.43, 158.39, 158.33, 154.99, 153.12, 152.21, 151.72, 151.16, 148.17, 146.11, 138.50, 136.66, 135.28, 135.19, 134.65, 134.50, 132.51, 132.08, 132.02, 120.83, 119.16, 118.54, 118.44, 114.18, 113.88, 111.51, 110.16, 107.66, 107.54, 100.69, 100.54, 64.06, 57.83, 57.24, 56.69, 55.94, 53.02, 49.07, 48.90, 42.76, 39.18, 33.18, 32.21, 31.90, 31.46, 22.61. HRMS m/z 965.33047 [M+H]⁺ (calcd. for C₄₆H₄₇F₂N₁₂O₈S, 965.33231).

### 2-(4-(1-(1-(2-(2,6-dioxopiperidin-3-yl)-1,3-dioxoisoindolin-4-yl)azetidin-3-yl)-1H-1,2,3-triazol-4-yl)piperidin-1-yl)-N-(((2-((5-fluoro-4-(4-fluoro-2-methoxyphenyl)pyridin-2-yl)amino)pyridin-4-yl)methyl)(methyl)(oxo)-16-sulfaneylidene)acetamide (4.60)

Sodium ascorbate (2.2 mg, 0.01 mmol), CuSO₄·5H₂O (2.7 mg, 0.01 mmol), 2-(4-ethynylpiperidin-1-yl)-N-{[(2-{[5-fluoro-4-(4-fluoro-2-methoxyphenyl)pyridin-2-yl]amino}pyridin-4-yl)methyl](methyl)oxo-λ⁶-sulfanylidene}acetamide (4.52) (15 mg, 0.03 mmol) and 4-(3-azidoazetidin-1-yl)-2-(2,6-dioxopiperidin-3-yl)-2,3-dihydro-1H-isoindole-1,3-dione (4.41) (9.6 mg, 0.03 mmol) were reacted according to general procedure B to provide 4.60 (13 mg, 51% yield) as a green powder. ¹H NMR (400 MHz, DMSO) δ 11.06 (s, 1H), 9.90 (s, 1H), 8.24-8.18 (m, 2H), 8.16 (s, 1H), 7.71 (d, J=6.1 Hz, 2H), 7.64 (dd, J=8.5, 7.1 Hz, 1H), 7.33 (dd, J=8.5, 6.8 Hz, 1H), 7.21 (d, J=7.0 Hz, 1H), 7.09 (dd, J=11.5, 2.5 Hz, 1H), 6.95-6.86 (m, 3H), 5.56 (td, J=7.7, 3.9 Hz, 1H), 5.07 (dd, J=12.9, 5.4 Hz, 1H), 4.88 (s, 2H), 4.76 (t, J=8.7 Hz, 2H), 4.49 (dt, J=9.8, 4.9 Hz, 2H), 3.79 (s, 3H), 3.28 (s, 3H), 3.06 (s, 2H), 2.87 (ddd, J=17.5, 14.0, 5.6 Hz, 3H), 2.61-2.52 (m, 2H), 2.21 (s, 2H), 2.05-1.95 (m, 2H), 1.86 (s, 2H) 1.60 (s, 2H). ¹³C NMR (176 MHz, DMSO) δ 178.75, 173.27, 170.44, 167.63, 166.99, 164.82, 163.43, 158.39, 158.33, 154.99, 153.11, 152.21, 151.71, 151.16, 148.17, 147.82, 138.50, 135.61, 135.28, 135.19, 134.65, 134.50, 133.67, 132.08, 132.02, 120.81, 120.69, 119.17, 119.16, 118.53, 114.18, 113.88, 113.02, 111.59, 107.66, 107.53, 100.68, 100.53, 64.08, 61.11, 57.83, 56.69, 53.02, 49.31, 49.16, 39.18, 33.19, 32.19, 31.91, 31.41, 22.55. HRMS m/z 908.30950 [M+H]⁺ (calcd. for C₄₄H₄₄F₂N₁₁O₇S, 908.31085).

### 2-(4-(1-(2-(4-(3-(2-(2,6-dioxopiperidin-3-yl)-1,3-dioxoisoindolin-4-yl)prop-2-yn-1-yl)piperazin-1-yl)-2-oxoethyl)-1H-1,2,3-triazol-4-yl)piperidin-1-yl)-N-(((2-((5-fluoro-4-(4-fluoro-2-methoxyphenyl)pyridin-2-yl)amino)pyridin-4-yl)methyl)(methyl)(oxo)-16-sulfaneylidene)acetamide (4.61)

Sodium ascorbate (2.2 mg, 0.01 mmol), CuSO₄·5H₂O (2.7 mg, 0.01 mmol), 2-(4-ethynylpiperidin-1-yl)-N-([(2-{[5-fluoro-4-(4-fluoro-2-melhoxyphenyl)pyridin-2-yl]amino}pyridin-4-yl)methyl](methyl)oxo-X⁶-sulfanylidene}acetamide (4.52) (15 mg, 0.03 mmol) and 4-{3-[4-(2-azidoacetyl)piperazin-1-yl]prop-1-yn-1-yl}-2-(2,6-dioxopiperidin-3-yl)-2,3-dihydro-1H-isoindole-1,3-dione (4.42) (13 mg, 0.03 mmol) were reacted according to general procedure B to provide 4.61 (19 mg, 69% yield) as an off-white powder. ¹H NMR (400 MHz, DMSO) δ 11.13 (s, 1H), 9.91 (s, 1H), 8.25-8.19 (m, 2H), 7.95-7.82 (m, 3H), 7.72 (t, J=2.7 Hz, 3H), 7.34 (dd, J=8.4, 6.8 Hz, 1H), 7.10 (dd, J=11.5, 2.5 Hz, 1H), 6.97 6.87 (m, 2H), 5.40 (s, 2H), 5.18 (dd, J=12.9, 5.4 Hz, 1H), 4.95-4.83 (m, 2H), 3.80 (s, 3H), 3.72 (s, 2H), 3.65-3.50 (m, 4H), 3.28 (s, 3H), 3.07 (s, 2H), 2.90 (ddt, J=22.8, 8.9, 5.2 Hz, 3H), 2.76-2.66 (m, 2H), 2.66-2.55 (m, 4H), 2. (t, J=11.3 Hz, 2H), 2.12-2.03 (m, 1H), 1.89-1.81 (m, 2H), 1.58 (q, J=11.7 Hz, 2H). ¹C NMR (176 MHz, DMSO) δ 178.74, 173.25, 170.33, 166.73, 166.18, 164.82, 163.43, 158.40, 158.34, 154.99, 153.12, 151.72, 151.30, 151.16, 148.17, 138.88, 138.50, 135.28, 135.24, 135.19, 134.66, 134.51, 132.55, 132.10, 132.04, 130.79, 123.61, 122.84, 119.47, 119.18, 118.55, 114.19, 113.88, 107.67, 107.55, 100.68, 100.53, 93.51, 80.94, 64.08, 57.83, 56.69, 53.07, 51.61, 51.24, 50.98, 49.48, 47.26, 44.63, 41.99, 39.17, 33.12, 32.25, 31.90, 31.40, 22.38. HRMS m/z 1017.36270 [M+H]⁺ (calcd. for C₅₀H₅₁F₂N₁₂O₈S, 1017.36361).

### 1-(1-(1-((2-(2,6-dioxopiperidin-3-yl)-1,3-dioxoisoindolin-4-yl)glycyl)azetidin-3-yl)-1H-1,2,3-triazol-4-yl)methyl)-N-(((2-((5-fluoro-4-(4-fluoro-2-methoxyphenyl)pyridin-2-yl)amino)pyridin-4-yl)menthyl)(methyl)(oxo)-16-sulfaneylidene)piperidine-3-carboxamide (4.62)

Sodium ascorbate (4.3 mg, 0.02 mmol), CuSO₄·5H₂O (5.4 mg, 0.02 mmol), N-([(-{[5-fluoro-4-(4-fluoro-2-methoxyphenyl)pyridin-2-yl]amino}pyridin-4-yl)methyl](methyl)oxo-λ⁶-sulfanylidene}-1-(prop-2-yn-1-yl)piperidine-3-carboxamide (4.53) (30 mg, 0.05 mmol) and 4-((2-(3-azidoazetidin-1-yl)-2-oxoethyl)amino)-2-(2,6-dioxopiperidin-3-yl)isoindoline-1,3-dione (4.40) (22 mg, 0.05 mmol) were reacted according to general procedure B to provide 4.62 (25 mg, 48% yield) as a green powder. ¹H NMR (700 MHz, DMSO) δ 11.11 (s, 1H), 9.91 (d, J=4.0 Hz, 1H), 8.23-8.19 (m, 3H), 7.72 (d, J=2.9 Hz, 1H), 7.69 (t, J=4.8 Hz, 1H), 7.61 (dd, J=8.5, 7.1 Hz, 1H), 7.34 (ddd, J=8.2, 6.7, 1.4 Hz, 1H), 7.12-7.07 (m, 2H), 7.03 (d, J=8.6 Hz, 1H), 6.92 (tdd, J=8.3, 2.5, 0.9 Hz, 1H), 6.88 (dd, J=5.2, 1.3 Hz, 2H), 5.57 (dddd, J=13.4, 8.2, 5.3, 1.3 Hz, 1H), 5.08 (dd, J=12.9, 5.5 Hz, 1H), 4.89-4.81 (m, 2H), 4.75 (t, J=8.7 Hz, 1H), 4.56 (dd, J=9.5, 5.3 Hz, 1H), 4.49 (t, J=9.2 Hz, 1H), 4.26 (dt, J=8.3, 3.8 Hz, 1H), 4.08 (dd, J=12.2, 5.3 Hz, 2H), 3.80 (s, 3H), 3.56-3.48 (m, 2H), 3.25 (d, J=1.4 Hz, 3H), 3.18 (d, J=5.2 Hz, 1H), 2.89 (ddd, J=16.9, 13.9, 54 Hz, 2H), 2.69 (s, 1H), 2.62-2.53 (m, 2H), 2.28 (dq, J=10.8, 4.8 Hz, 1H), 2.06-1.98 (m, 2H), 1.86 (s, 1H), 1.76 (t, J=12.1 Hz, 1H), 1.58-1.53 (m, 1H), 1.41-1.33 (m, 1H), 1.21 (dd, J=25.0, 13.3 Hz, 2H). ¹³C NMR (176 MHz, DMSO) δ 182.25, 173.28, 170.54, 169.27, 168.91, 167.77, 164.83, 163.43, 158.40, 158.34, 154.93, 153.12, 151.72, 151.17, 148.15, 146.12, 144.66, 138.48, 136.63, 135.31, 135.23, 14.62, 134.47, 132.51, 132.07, 132.01, 123.69, 119.15, 118.63, 118.42, 114.22, 1130.88, 111.49, 110.15, 107.66, 107.54, 100.69, 100.54, 57.64, 5719, 56.69, 56.13, 56.10, 55.95, 53.51, 53.31, 49.06, 48.95, 45.96, 45.90, 42.73, 39.01, 31.46, 27.58, 27.53, 24.80, 22.61. HRMS m/z 965.33129 [M+H]⁺ (calcd. for C₄₆H₄₇F₂N₁₂O₈S, 965.33231).

### 1-((1-(1-((2-(2,6-dioxopiperidin-3-yl)-1,3-dioxoisoindolin-4-yl)glycyl)azetidin-3-yl)-1H-1,2, 3-triazol-4-yl)methyl)-N-(((2-((5-fluoro˜4-(4-fluoro-2-methoxyphenyl)pyridin-2-yl)amino)pyridin-4-yl)methyl)(methyl)(oxo)-16-sulfaneylidene)piperidine-3-carboxamide (4.63)

Sodium ascorbate (4.3 mg, 0.02 mmol), CuSO₄·5H₂O (5.4 mg, 0.02 mmol), N-{[(2-[5-fluoro-4-(4-fluoro-2-methoxyphenyl)pyridin-2-yl]amino}pyridin-4-yl)methyl](methyl)oxo-λ⁶-sulfanylidene}-1-(prop-2-yn-1-yl)piper dine-3-carboxamide (4.53) (30 mg, 0.05 mmol) and 4-(3-[4-(2-azidoacetyl)piperazin-1-yl]prop-1-yn-1-yl}-2-(2,6-dioxopiperidin-3-yl)-2,3-dihydro-1H-isoindole-1,3-dione (4.41) (19 mg, 0.05 mmol) were reacted according to general procedure B to provide 4.63 (23 mg, 47% yield) as a green powder. ¹H NMR (700 MHz, DMSO) δ 11.08 (s, 1H), 9.91 (d, J=4.6 Hz, 1H), 8.23-8.18 (m, 3H), 7.73-7.67 (m, 2H), 7.63 (ddd, J=8.5, 7.0, 1.5 Hz, 1H), 7.33 (dd, J=8.4, 6.7 Hz, 1H), 7.21 (dd, J=7.1, 1.7 Hz, 11H), 7.09 (ddd, J=11.4, 2.5, 1.1 Hz, 1H), 6.94-6.86 (m, 3H), 5.59 (qd, J=7.5, 2.9 Hz, 1H), 5.07 (dd, J=12.9, 5.5 Hz, 1H), 4.88-4.80 (m, 2H), 4.78-4.73 (m, 2H), 4.54-4.48 (m, 2H), 3.79 (d, J=1.0 Hz, 3H), 3.55-3.48 (m, 2H), (d, J=2.3 Hz, 3H), 2.87 (ddd, J=17.1, 13.8, 5.4 Hz, 2H), 2.68 (s, 1H), 2.61-2.54 (m, 1H), 2.28 (tq, J=10.8, 3.7 Hz, 1H), 2.00 (ddd, J=13.0, 6.1, 3.6 Hz, 2H), 1.88-1.84 (m, 1H), 1.74 (d, J=14.1 Hz, 1H), 1.55 (dt, J=12.7, 3.4 Hz, 1H), 1.35 (dd, J=14.0, 10.5 Hz, 1H), 1.21 (dd, J=27.5, 15.0 Hz, 2H). ¹³C NMR (176 MHz, DMSO) δ 182.24, 173.26, 170.44, 167.63, 166.99, 164.82, 163.43, 158.40, 158.33, 154.92, 153.12, 151.72, 151.17, 148.14, 147.80, 144.57, 138.48, 135.59, 135.31, 135.22, 134.63, 134.47, 133.66, 132.06, 132.00, 123.61, 120.79, 119.15, 118.62, 114.20, 113.88, 113.02, 111.61, 107.66, 107.53, 100.68, 100.53, 61.10, 57.64, 56.69, 56.09, 53.46, 53.28, 49.40, 49.16, 45.95, 45.89, 38.99, 31.41, 27.55, 27.48, 24.81, 24.77, 22.54. HRMS m/z 908.31029 [M+H]⁺ (calcd, for C₄₄H₄₄F₂N₁₁O₇S, 908.31085).

### 1-((1-(2-(4-(3-(2-(2,6-dioxopiperidin-3-yl)-1,3-dioxoisoindolin-4-yl)prop-2-yl-1-yl)piperazin-1-yl)-2-oxoethyl)-1H-1,2,3-triazol-4-yl)methyl)-N-(((2-((5-fluoro-4-(4-fluoro-2-methoxyphenyl)pyridin-2-yl)amino)pyridin-4-yl)methyl)(methyl)(oxo)-16-sulfaneylidene)piperidine-3-carboxamide (4.64)

Sodium ascorbate (4.3 mg, 0.02 mmol), CuSO₄·5H₂O (5.4 mg, 0.02 mmol), N-{[(2-{[5-fluoro-4-(4-fluoro-2-methoxyphenyl)pyridin-2-yl]amino}pyridin-4-yl)methyl](methyl)oxo-λ⁶-sulfanylidene}-1-(prop-2-yn-1-yl)piperidine-3-carboxamide (4.53) (30 mg, 0.05 mmol) and 4-{3-[4-(2-azidoacetyl)piperazin-1-yl]prop-1-yn-1-yl}-2-(2,6-dioxopiperidin-3-yl)-2,3-dihydro-1H-isoindole-1,3-dione (4.42) (25 mg, 0.05 mmol) were reacted according to general procedure B to provide 4.64 (42 mg, 75% yield) as a green powder. ¹H NMR (700 MHz, DMSO) δ 11.14 (s, 1H), 9.91 (d, J=2.4 Hz, 1H), 8.21 (dd, J=6.1, 1.9 MHz, 2H), 7.94-7.82 (m, 3H), 7.80 (d, J=3.2 Hz, 1H), 7.70 (dd, J=7.4, 4.6 Hz, 2H), 7.34 (dd, J=8.4, 6.7 Hz, 1H), 7.10 (dd, J=11.4, 2.5 Hz, 1H), 6.92 (td, J=8.5, 2.5 Hz, 1H), 6.89 (dt, J=5.2, 1.6 Hz, 1H), 5.44 (s, 2H), 5.17 (dd, J=12.9, 5.5 MHz, 1H), 4.88-4.81 (m, 2H), 3-80 (s, 3H), 3.71 (s, 2H), 3.56 (d, J=5.4 Hz, 2H), 3.54-3.47 (m, 4H), 3.24 (d, J=1.91 Hz, 3H), 2.91 (ddd, J=16.6, 13.8, 5.4 Hz, 2H), 2.69 (t, J=5.2 MHz, 3H), 2.64-2.52 (m, 5H), 2.28 (tq, J=11.0, 3.8 Hz, 1H), 2.11-2.04 (m, 1H), 1.98 (d, J=9.7 Hz, 1H), 1.84 (d, J=9.2 Hz, 1H), 1.75 (dd, J=14.5, 8.3 Hz, 1H), 1.58-1.53 (m, 1H), 1.40-1.32 (m, 1H). ¹³C NMR (176 MHz, DMSO) δ 182.29, 182.26, 173.25, 170.33, 166.73, 166.18, 164.83, 164.78, 163.43, 158.40, 158.34, 154.93, 153.13, 151.73, 151.17, 148.17, 143.70, 138.89, 138.50, 135.31, 135.23, 134.64, 134.49, 132.55, 132.08, 132.02, 130.79, 125.80, 123.60, 119.47, 119.17, 118.62, 114.20, 113.88, 107.67, 107.55, 100.68, 100.53, 93.51, 80.92, 57.65, 56.69, 56.11, 53.46, 53.22, 51.63, 51.25, 50.99, 49.48, 47.26, 45.99, 45.94, 44.60, 42.00, 38.98, 31.40, 27.57, 24.81, 22.37. HRMS m/z 1017.36313 [M+H]⁺ (calcd. for C₅₀H₅₁F₂N₁₂O₈S, 1017.36361).

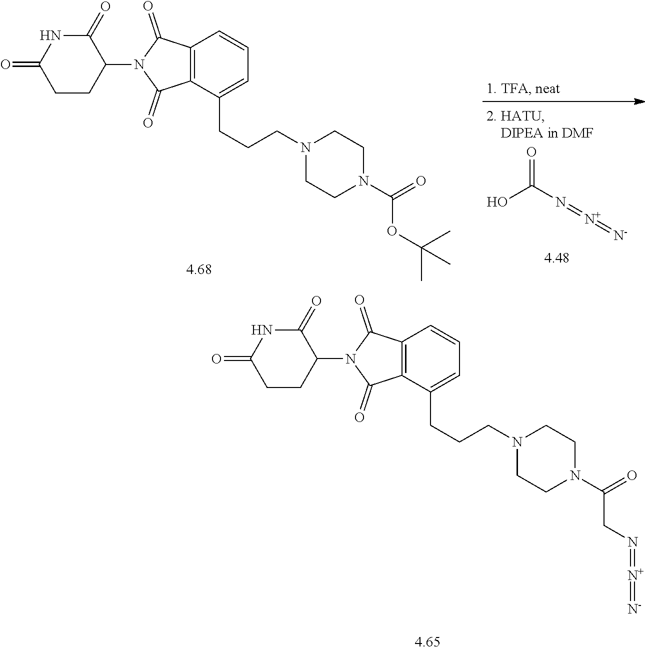

### 4-(3-(4-(2-azidoacetyl)piperazin-1-yl)propyl)-2-(2,6-dioxopiperidin-3-yl)isoindoline-1,3-dione (4.65)

Tert-butyl 4-(3-(2-(2,6-dioxopiperidin-3-yl)-1,3-dioxoisoindolin-4-yl)propyl)piperazine-1-carboxylate 4.68 (132 mg, 0.27 mmol) was dissolved in TFA (1.6 M) and stirred for thirty minutes, after which the mixture was purified by reversed phase chromatography (C18, H₂O:ACN eluent) to provide 2-(2,6-dioxopiperidin-3-yl)-4-(3-(piperazin-1-yl)propyl)isoindoline-1,3-dione, the deprotected version of 4.68 (105 mg, 61% yield) as an off-white powder.

2-(2,6-dioxopiperidin-3-yl)-4-(3-(piperazin-1-yl)propyl)isoindoline-1,3-dione (185 mg, 0.481 mmol), 2-azidoacetic acid (4.48) (77 mg, 0.481 mmol), HATU (291 mg, 0.28 mmol) and DIPEA (197 mg, 1.53 mmol) were dissolved in DMF (0.04 M). The mixture was stirred overnight at room temperature. The reaction mixture was concentrated under reduced pressure and purified by reversed phase chromatography (C18, H₂O:ACN eluent) to provide 4.65 (132 mg, 102% yield) as pale brown solid. ¹H NMR (700 MHz, DMSO) δ 11.14 (s, 1H), 7.86-7.80 (m, 2H), 7.76 (dd, J=7.0, 1.8 Hz, 1H), 5.14 (dd, J=12.9, 5.5 Hz, 1H), 4.42 (s, 1H), 4.19 (d, J=12.9 Hz, 2H), 3.84 (s, 1H), 3.52 (s, 2H), 3.18 (s, 2H), 3.09 (t, J=7.7 Hz, 2H), 3.06-2.86 (m, 4H), 2.66-2.60 (m, 1H), 2.55 (td, J=13.0, 4.0 Hz, 2H), 2.07-1.97 (m, 3H). ¹³C NMR (176 MHz, DMSO) δ 173.27, 170.32, 168.19, 167.42, 166.74, 140.84, 136.54, 135.27, 132.36, 128.30, 12224, 118.54, 55.59, 51.09, 50.04, 4933, 41.64, 39.00, 31.41, 27.92, 24.85, 22.49, 1.63. HRMS m/z 468.19865 [M+H]⁺ (calcd. for C₂₂H₂₆N₇O₅, 468.19899).

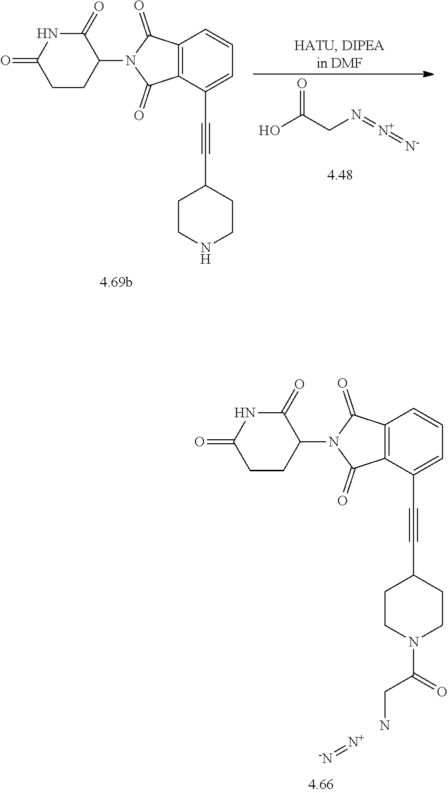

### 4-((1-(2-azidoacetyl)piperidin-4-yl)ethynyl)-2-(2,6-dioxopiperidin-3-yl)isoindoline-1,3-dione (4.66)

Tert-butyl 4-((2-(2,6-dioxopiperidin-3-yl)-1,3-dioxoisoindolin-4-yl)ethynyl)piperidine-1-carboxylate 4.69a (260 mg, 0.89 mmol) was dissolved in TFA (1.6 M) and stirred for thirty minutes, after which the mixture was concentrated under reduced pressure to provide 4.69b which was taken onto the next step without further purification.

2-(2,6-dioxopiperidin-3-yl)-4-(piperidin-4-ylethynyl)isoindoline-1,3-dione 4.69b (185 mg, 0.481 mmol), 2-azidoacetic acid (4.48) (77 mg, 0,481 mmol), HATU (291 mg, 0.28 mmol) and DIPEA (197 mg, 1.53 mmol) were dissolved in DMF (0.04 M). The mixture was stirred overnight at room temperature. The reaction mixture was concentrated tinder reduced pressure and purified by reversed phase chromatography (C18, H₂O:ACN eluent) to provide 4.66 (170 mg, 69% yield) as an off-white powder. ¹H NMR (400 MHz, CDCl₃) δ 8.15 (s, 1H), 7.81 (dd, J=6.1, 23 Hz, 1H), 7.74-7.64 (m, 2H), 4.99 (dd, J=12.3, 5.3 Hz, 1H), 3.96 (s, 2H), 3.86-3.67 (m, 3H), 3.37 (dt, J=13.7, 5.2 Hz, 1H), 3.13-3.04 (m, 1H), 2.82 (dtdd, J=38.0, 17.3, 13.2, 4.2 Hz, 3H), 2.18-2.09 (m, 1H), 1.98-1.83 (m, 4H). ¹³C NMR (176 MHz, CDCl₃) δ 170.74, 167.87, 166.40, 165.91, 165.48, 138.15, 138.12, 134.00, 132.30, 131.03, 123.00, 120.92, 99.46, 78.06, 78.03, 50.81, 49.33, 43.10, 43.05, 40.02, 39.97, 31.41, 31.32, 30.49, 27.35, 27.33, 22.58. HRMS m/z 449.15622 [M+H]⁺ (calcd. for C₂₂H₂₁N₆O₅, 449.15679).

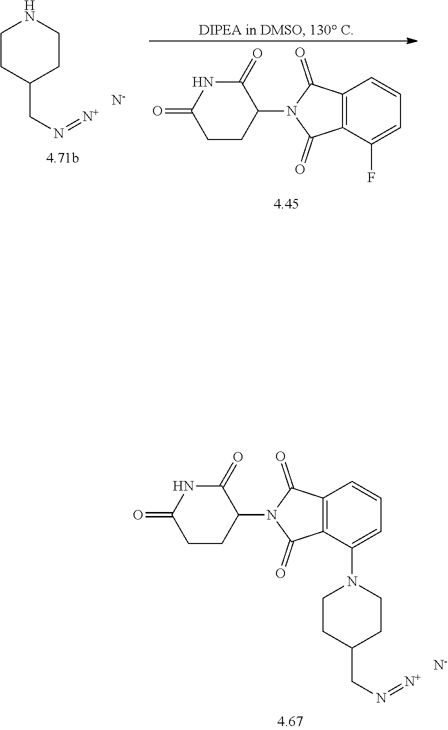

### 4-(4-(azidomethyl)piperidin-1-yl)-2-(2,6-dioxopiperidin-3-yl)isoindoline-1,3-dione (4.67)

4-(azidomethyl)piperidine 4.71b (152 mg 1.1 mmol), 2-(2,6-dioxopiperidin-3-yl)-4-fluoro-2,3-dihydro-1H-isoindole-1,3-dione 4.45 (300 mg, 1.1 mmol) were dissolved in DMSO (0.23 M) with DIPEA (281 mg, 2.2 mmol), heated to 130° (and stirred overnight.

The reaction mixture was then concentrated under reduced pressure and purified by column chromatography (C18, H₂O:ACN eluent) to provide 4.67 (100 mg, 23% yield) as yellow solid. ¹H NMR (400 MHz, CDCl₃) δ 8.00 (s, 1H), 7.58 (dd, J=8.4, 7.2 Hz, 1H), 7.38 (d, J=70 Hz, 1H), 7.17 (d, J=8.4 Hz, 1H), 4.96 (dd, J=12.2, 54 Hz, 1H), 3.82-3.71 (m, 2H), 3.27 (d, J=6.7 Hz, 2H), 2.98-2.66 (m, 5H), 2.19-2.06 (m, 1H), 1.94-1.86 (m, 2H), 1.76 (dtd, J=14.5, 7.5, 3.6 Hz, 11H), 1.59 (dt, J=11.9, 3.4 Hz, 2H). ¹³C NMR (176 MHz, DMSO) δ 173.28, 170.50, 167.56, 166.77, 15049, 136.23, 134.44, 134.13, 124.45, 120.07, 116.92, 114.99, 56.56, 5112, 49.24, 43.36, 35.78, 34.98, 31.43, 30.02, 29.66, 22.54. HRMS m/z 397.16116 [M+H]⁺ (calcd. for C₁₉H₂₁N₆O₄, 397.16188).

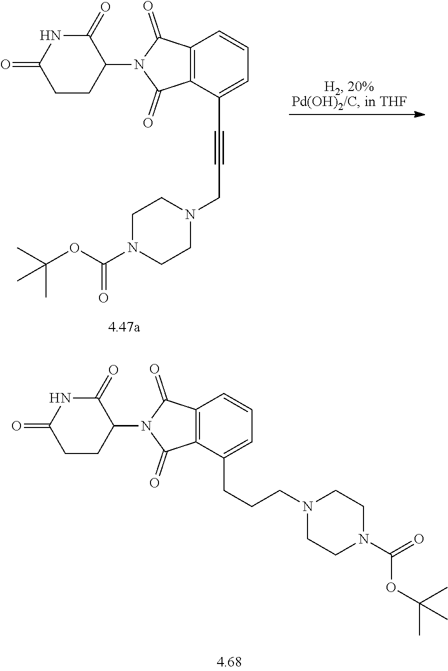 [SMILES: CC(C)(C)OC(=O)N1CCN(CC#Cc2cccc3c2C(=O)N(C2CCC(=O)NC2=O)C3=O)CC1 ; "H2, 20%Pd(OH)2/C, in THF" ; CC(C)(C)OC(=O)N1CCN(CCCc2cccc3c2C(=O)N(C2CCC(=O)NC2=O)C3=O)CC1]

### Tert-butyl 4-(3-(2-(2,6-dioxopiperidin-3-yl)-1,3-dioxoisoindolin-4-yl)propyl)piperazine-1-carboxylate (4.68)

To a solution of tert-butyl 4-(3-(2-(2,6-dioxopiperidin-3-yl)-1,3-dioxoisoindolin-4-yl)prop-2-yn-1-yl)piperazine-1-carboxylate (4.47a) (170 mg, 0.35 mmol) in methanol (0.1 M) was added Palladium hydroxide on carbon (16 mg, 0.11 mmol) under argon protection. The reaction vessel was purged with hydrogen gas and the reaction mixture was stirred at room temperature under hydrogen atmosphere overnight. The reaction was filtered through celite and concentrated under reduced pressure to provide 4.68 as an off-white powder (132 mg, 77% yield). ¹H NMR (700 MHz, CDCl₃) δ 8.23 (s, 1H), 7.77 (d, J=7.3 Hz, 1H), 7.74-7.59 (m, 2H), 4.97 (ddd, J=12.6, 5.4, 3.8 Hz, 1H), 3.87-3.67 (m, 2H), 3.48 (s, 1H), 3.21-3.12 (m, 2H), 3.08-3.03 (m, 1H), 2.99 (s, 1H), 2.94-2.70 (m, 4H), 2.27 (d, J=5.1 Hz, 1H), 2.16 (ddtd, J=17.7, 12.8, 5.2, 2.8 Hz, 1H), 1.81-1.51 (m, 4H), 1.50-1.41 (m, 9H). ¹³C NMR (76 MHz, CDCl₃) δ 170.79, 168.09, 168.04, 166.95, 153.95, 136.12, 136.02, 134.79, 134.06, 132.36, 128.30, 122.51, 121.54, 81.24, 57.01, 52.06, 49.85, 49.30, 49.12, 33.30, 31.42, 28.59, 28.30, 24.01, 22.69, 13.93 HRMS m/z 485.23899 [M+H]⁺ (calcd. for C₂₅H₃₃N₄O₆, 485.23946).

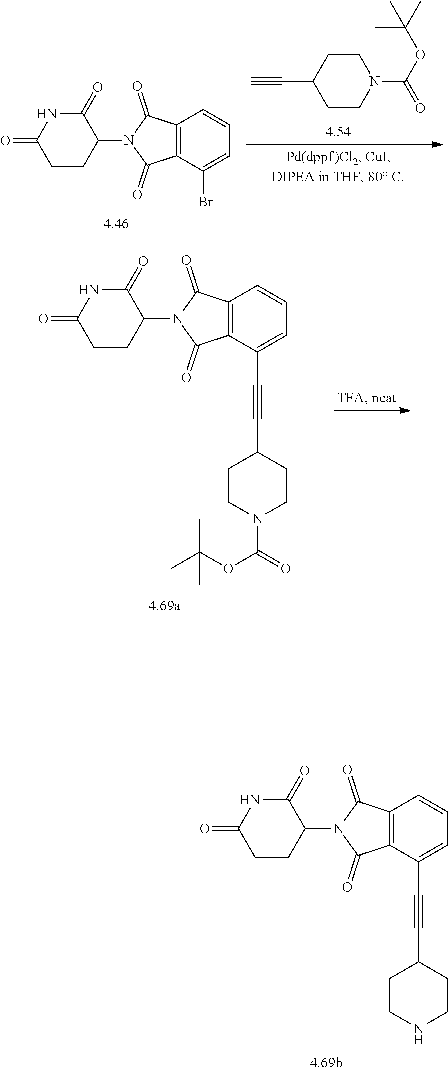 [SMILES: O=C1CCC(N2C(=O)c3cccc(Br)c3C2=O)C(=O)N1 ; C#CC1CCN(C(=O)OC(C)(C)C)CC1 ; CC(C)(C)OC(=O)N1CCC(C#Cc2cccc3c2C(=O)N(C2CCC(=O)NC2=O)C3=O)CC1 ; O=C1CCC(N2C(=O)c3cccc(C#CC4CCNCC4)c3C2=O)C(=O)N1]

### 2-(2,6-dioxopiperidin-3-yl)-4-(piperidin-4-ylethynyl)isoindoline-1,3-dione (4.69b)

4-bromo-2-(2,6-dioxopiperidin-3-yl)isoindoline-1,3-dione (4.46) (300 mg, 0.89 mmol), tert-butyl 4-ethynylpiperidine-1-carboxylate (4.54) (186 mg, 0.89 mmol), [1,1′-Bis(diphenylphosphino)ferrocene]dichloropalladium(II) (65 mg, 0.09 mmol), copper(II)sulfate pentahydrate (88 mg, 0.36) and DIPEA (345 mg, 2.67 mmol) were dissolved in THF (0.2 M) and heated at 80° C. in a sealed tube under argon overnight. The reaction mixture was concentrated under reduced pressure and the crude mixture was purified by reversed phase chromatography (C18, 0-100% ACN in water) to afford 4.69a as a brown crystalline solid (260 mg, 63% yield). ¹H NMR (400 MHz, DMSO) δ 11.14 (s, 1H), 7.96-7.82 (m, 4H), 5.22-5.10 (m, 1H), 3.78 (hept, J=6.1 Hz, 1H), 3.35-3.28 (m, 1H), 3.15 (dd, J=9.0, 5.2 Hz, 2H), 2.90 (ddd, J=16.9, 13.7, 5.4 Hz, 1H), 2.67-2.53 (m, 2-1), 2.08 (ddd, J=14.0, 7.0, 4.7 Hz, 3H), 1.87 (dtd, J-=14.2, 7.6, 3.3 Hz, 1H), 1.33 (d, J=6.2 Hz, 1H), 1.04 (d, J=6.1 Hz, 9H). ¹³C NMR (101 MHz, DMSO) δ 173.23, 170.25, 166.68, 166.38, 159.02, 158.64, 138.50, 135.25, 132.50, 131.05, 123.59, 119.65, 99.18, 78.03, 74.39, 62.48, 49.48, 41.89, 31.74, 31.41, 28.00, 25.93, 25.41, 22.37, 21.38. Tert-butyl 4-((2-(2,6-dioxopiperidin-3-yl)-1,3-dioxoisoindolin-4-yl)ethynyl)piperidine-1-carboxylate 4.69a was dissolved in TFA and stirred at room temperature until completion as determined by LCMS. The solution was concentrated in vacuo to afford 4.69b, which was taken onto the next step without further purification. HRMS m/z 366.14404 [M+H]⁺ (calcd. for C₂₀H₂₀N₃O₄, 366.14483).

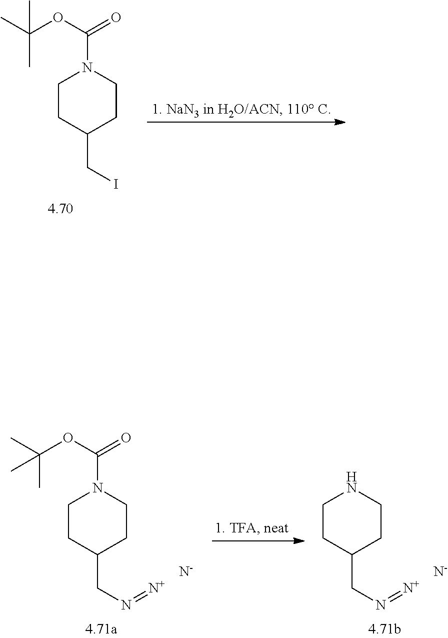

### 4-(azidomethyl)piperidine (4.71b)

Tert-butyl 4-(iodomethyl)piperidine-1-carboxylate (5 g, 15.4 mml) and sodium azide (2.0 g, 30.8 mmol) dissolved in ACN (0.75 M) and 1-120 (2.25 M) and the solution was heated at 110° C. for eight hours. The reaction mixture was concentrated under reduced pressure and dissolved in water, followed by extraction with DCM. The organic layers were dried over Na₂SO₄ and filtered and concentrated under reduced pressure to provide 4.71a in quantitative yield, as a colorless oil. Spectroscopic data are consistent with those previously reported in the literature.^{244 1}H NMR (400 MHz, CDCl₃) δ 4.11 (ddt, J=10.4, 7.1, 3.3 Hz, 2H), 3.17 (d, J=6.2 Hz, 2H), 2.68 (td, J=12.7, 2.5 Hz, 2H), 1.75-1.61 (m, 3H), 1.44 (s, 9H), 1.21-1.07 (m, 2H).

4.71a was dissolved in TFA (1.6 M) and stirred for an hour at room temperature. The solution was concentrated under reduced pressure to obtain 4.71b which was taken onto the next step without further purification.

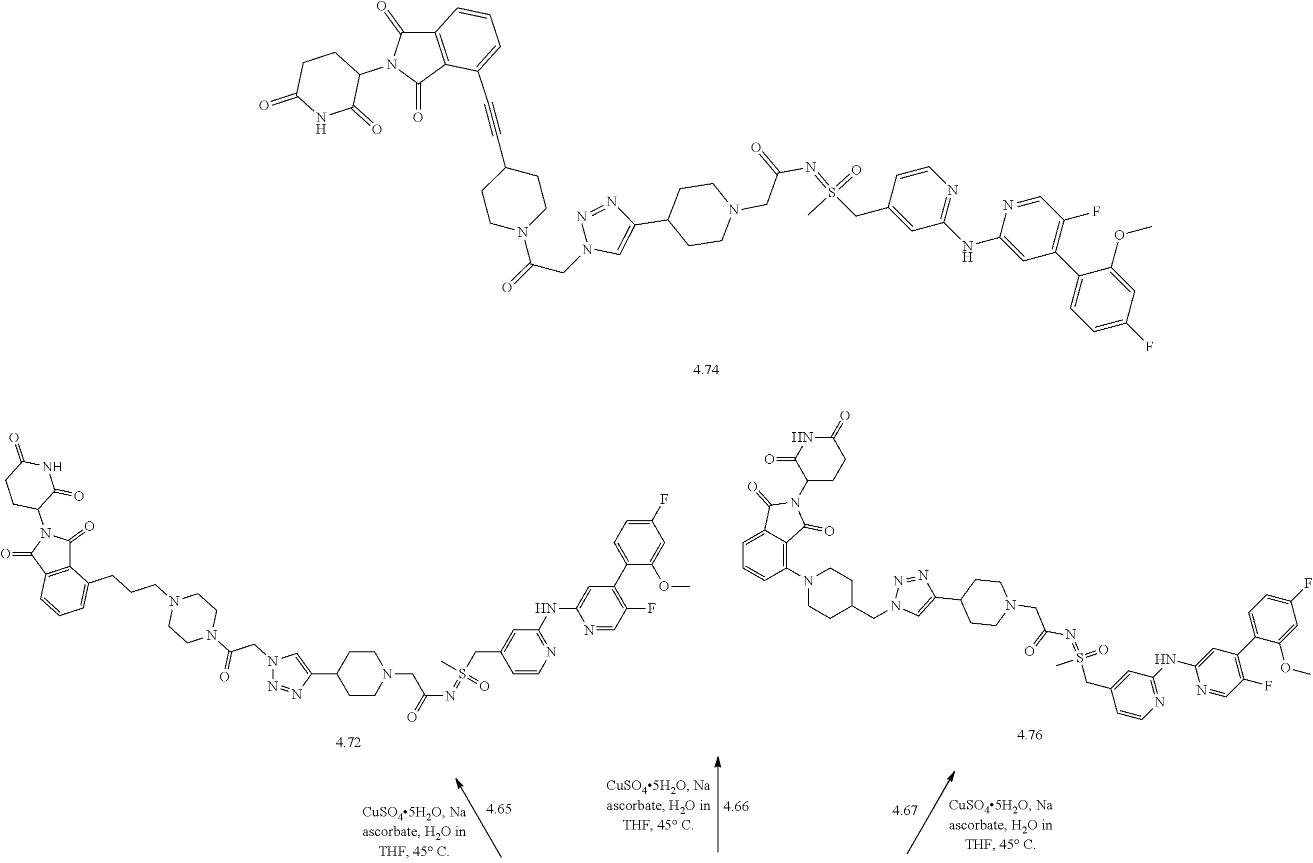 [SMILES: COc1cc(F)ccc1-c1cc(Nc2cc(CS(C)(=O)=NC(=O)CN3CCC(c4cn(CC(=O)N5CCC(C#Cc6cccc7c6C(=O)N(C6CCC(=O)NC6=O)C7=O)CC5)nn4)CC3)ccn2)ncc1F ; COc1cc(F)ccc1-c1cc(Nc2cc(CS(C)(=O)=NC(=O)CN3CCC(c4cn(CC(=O)N5CCN(CCCc6cccc7c6C(=O)N(C6CCC(=O)NC6=O)C7=O)CC5)nn4)CC3)ccn2)ncc1F ; COc1cc(F)ccc1-c1cc(Nc2cc(CS(C)(=O)=NC(=O)CN3CCC(c4cn(CC5CCN(c6cccc7c6C(=O)N(C6CCC(=O)NC6=O)C7=O)CC5)nn4)CC3)ccn2)ncc1F]

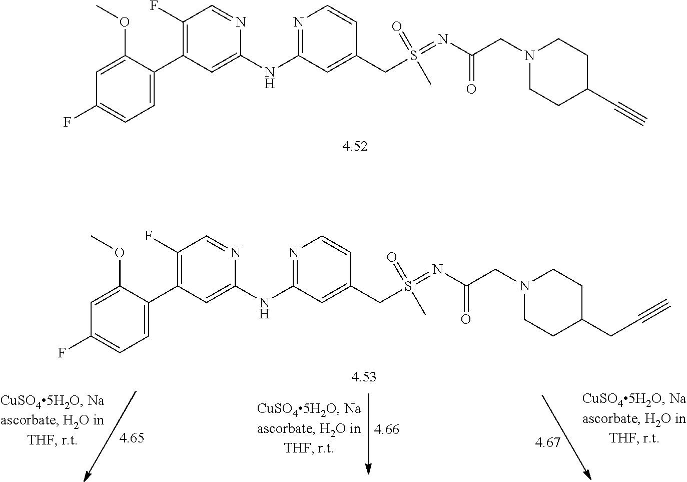 [SMILES: C#CC1CCN(CC(=O)N=S(C)(=O)Cc2ccnc(Nc3cc(-c4ccc(F)cc4OC)c(F)cn3)c2)CC1 ; C#CCC1CCN(CC(=O)N=S(C)(=O)Cc2ccnc(Nc3cc(-c4ccc(F)cc4OC)c(F)cn3)c2)CC1]

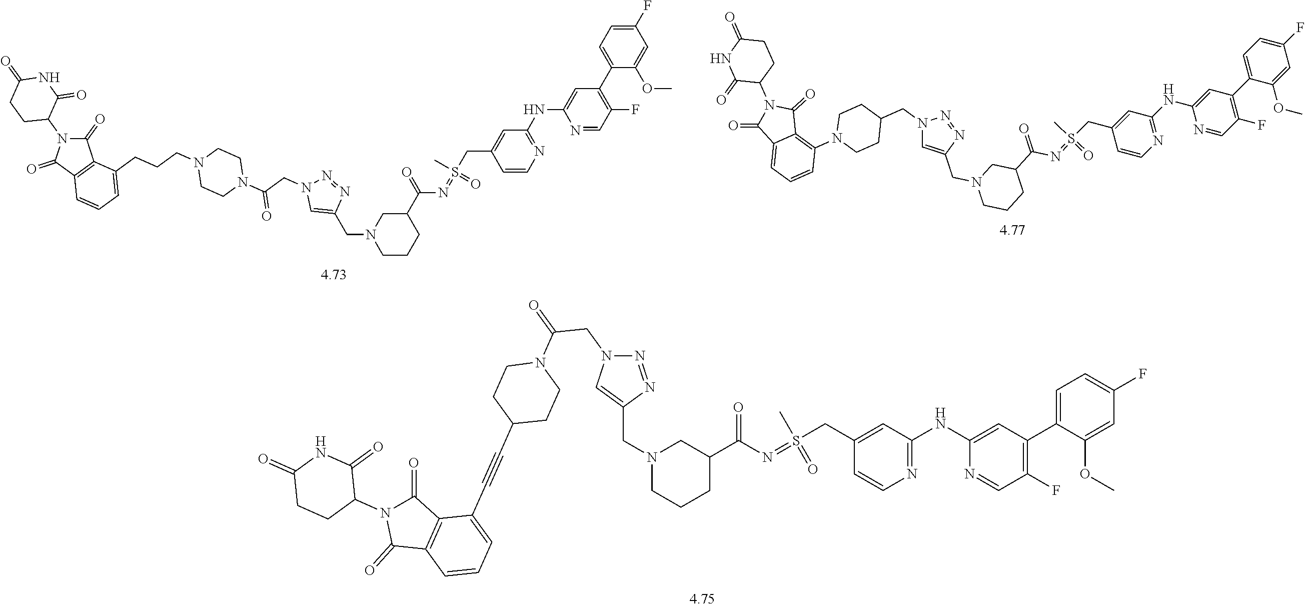 [SMILES: COc1cc(F)ccc1-c1cc(Nc2cc(CS(C)(=O)=NC(=O)C3CCCN(Cc4cn(CC(=O)N5CCN(CCCc6cccc7c6C(=O)N(C6CCC(=O)NC6=O)C7=O)CC5)nn4)C3)ccn2)ncc1F ; COc1cc(F)ccc1-c1cc(Nc2cc(CS(C)(=O)=NC(=O)C3CCCN(Cc4cn(CC5CCN(c6cccc7c6C(=O)N(C6CCC(=O)NC6=O)C7=O)CC5)nn4)C3)ccn2)ncc1F ; COc1cc(F)ccc1-c1cc(Nc2cc(CS(C)(=O)=NC(=O)C3CCCN(Cc4cn(CC(=O)N5CCC(C#Cc6cccc7c6C(=O)N(C6CCC(=O)NC6=O)C7=O)CC5)nn4)C3)ccn2)ncc1F]

### 2-(4-(1-(2-(4-(3-(2-(2,6-dioxopiperidin-3-yl)-1,3-dioxoisoindolin-4-yl) propyl) piperazin-1-yl)-2-oxoethyl)-1H-1,2,3-triazol-4-yl) piperidin-1-yl)-N-(((2-((5-fluoro-4-(4-fluoro-2-methoxyphenyl)pyridin-2-yl)amino)pyridin-4-yl)methyl)(methyl) (oxo)-16-sulfaneylidene) acetamide (4.72)

Sodium ascorbate (4.3 mg, 0.02 mmol), CuSO₄·5H₂O (5.4 mg, 0.02 mmol), 2-(4-ethynylpiperidin-1-yl)-N-{[(2-{[5-fluoro-4-(4-fluoro-2-methoxyphenyl)pyridin-2-ylamino}pyridin-4-yl)methyl](methyl)oxo-λ⁶-sulfanylidene}acetamide (4.52) (25 mg, 0.05 mmol) and 4-{3-[4-(2-azidoacetyl) piperazin-1-yl]propyl}-2-(2,6-dioxopiperidin-3-yl)-2,3-dihydro-1H-isoindole-1,3-dione (4.65) (25 mg, 0.05 mmol) were reacted according to general procedure B to provide 4.72 (13 mg, 28% yield) as an off-white powder. ¹H NMR (700 MHz, DMSO) δ 11.13 (s, 1H), 9.92 (s, 1H), 8.24-8.21 (m, 1H), 7.81-7.71 (m, 5H), 7.37-7.32 (m, 1H), 7.26 (t,]=7.6 Hz, 1H), 7.20-7.13 (m, 2H), 7.10 (dd, J=11.3, 2.4 Hz, 1H), 6.92 (hept, J=2.6 Hz, 1H), 5.37 (s, 1H), 5.14 (dd, J=12.9, 5.5 Hz, 1H), 4.89 (s, 1H), 3.80 (s, 2H), 3.51-3.38 (m, 4H), 3.25 (s, 3H), 3.13-3.02 (m, 3H), 2.90 (ddd, J=17.0, 13.9, 5.5 Hz, 3H), 2.66-2.52 (m, 4H), 2.44-2.32 (m, 5H), 2.31 (s, 2H), 2.21 (s, 2H), 2.07 (dtt, J=12.9, 5.4, 2.3 Hz, 1H), 1.93-1.76 (m, 3H), 1.59 (s, 2H), 1.34-1.09 (m, 3H). ¹³C NMR (176 MHz, DMSO) δ 173.27, 170.40, 168.17, 167.52, 164.82, 164.69, 163.43, 158.40, 158.34, 155.00, 153.12, 151.72, 151.16, 148.17, 142.93, 137.82, 136.67, 135.28, 135.20, 134.93, 134.66, 134.51, 132.26, 132.09, 129.37, 128.68, 128.15, 125.79, 122.86, 121.67, 119.16, 118.54, 114.19, 113.88, 107.68, 107.55, 100.69, 100.53, 57.85, 57.49, 56.70, 53.05, 52.95, 52.60, 50.94, 49.29, 44.72, 42.06, 39.19, 31.42, 28.94, 27.76, 22.48, 21.52. HRMS m/z 1021.39333 [M+H]⁺ (calcd. for C₅₀H₅₅F₂N₁₂O₈S, 1021.39491).

### 1-({1-[2-(4-{3-[2-(2,6-dioxopiperidin-3-yl)-1,3-dioxo-2,3-dihydro-1H-isoindol-4-yl]propyl}piperazin-1-yl)-2-oxoethyl]-1H-1,2,3-triazol-4-yl}methyl)-N-{[1 (2-{[5-fluoro-4-(4-fluoro-2-methoxyphenyl)pyridin-2-yl]amino}pyridin-4-yl)methyl](methyl)oxo-λ⁶-sulfanylidene}piperidine-3-carboxamide (4.73)

Sodium ascorbate (4.3 mg, 0.02 mmol), CuSO₄·5H₂O) (5.4 mg, 0.02 mmol), N-{[(2-{[5-fluoro-4-(4-fluoro-2-methoxyphenyl)pyridin-2-yl]amino}pyridin-4-yl)methyl](methyl)oxo-λ⁶-sulfanylidene}-1-(prop-2-yn-1-yl) piperidine-3-carboxamide (4.53) (30 mg, 0.05 mmol) and 4-{3-[4-(2-azidoacetyl) piperazin-1-yl]propyl}-2-(2,6-dioxopiperidin-3-yl)-2,3-dihydro-1H-isoindole-1,3-dione (4.65) (25 mg, 0.05 mmol) were reacted according to general procedure B to provide 4.73 (7 mg, 13% yield) as an off-white powder. ¹H NMR (700 MHz, DMSO) δ 11.13 (s, 1H), 9.91 (d, J=2.0 Hz, 1H), 8.24-8.20 (m, 1H), 7.81-7.68 (m, 4H), 7.34 (dd, J=8.4, 6.8 Hz, 1H), 7.28-7.23 (m, 1H), 7.20-7.13 (m, 2H), 7.10 (dd, J=11.4, 2.5 Hz, 1H), 6.94-6.86 (m, 2H), 5.40 (s, 1H), 5.14 (dd, J=12.9, 5.5 Hz, 1H), 4.92-4.76 (m, 2H), 3.80 (s, 2H), 3.58-3.38 (m, 5H), 3.32-3.30 (m, 4H), 3.25 (d, J=2.3 Hz, 2H), 3.07 (t, J=7.6 Hz, 2H), 2.94-2.89 (m, 1H), 2.88 (d, J=6.1 Hz, 1H), 2.68 (s, 1H), 2.64-2.52 (m, 2H), 2.41-2.34 (m, 3H), 2.31 (s, 3H), 2.27 (s, 1H), 2.11-2.04 (m, 1H), 1.99 (d, J=15.0 Hz, 1H), 1.93-1.70 (m, 4H), 1.56 (d, J]=12.6 Hz, 1H), 1.36 (d, J=12.1 Hz, 1H), 1.19 (s, 1H). ¹³C NMR (176 MHz, DMSO) δ 182.27, 173.27, 170.40, 168.16, 167.52, 164.83, 164.64, 163.43, 158.40, 158.34, 154.93, 153.12, 151.73, 151.17, 148.18, 143.68, 142.93, 138.50, 137.82, 136.66, 135.31, 135.22, 134.92, 134.64, 134.49, 132.26, 132.08, 132.02, 129.38, 128.68, 128.15, 125.79, 121.67, 119.17, 118.62, 114.20, 113.88, 107.67, 107.55, 100.68, 100.54, 57.65, 57.48, 56.69, 56.10, 53.47, 53.24, 52.95, 52.59, 50.96, 49.29, 45.94, 44.72, 42.08, 38.98, 31.42, 28.93, 27.75, 27.56, 24.81, 22.48, 21.52. HRMS m/z 1021.39389 [M+H]⁺ (calcd. for C₅₀H₅₅F₂N₁₂O₈S, 1021.39491).

### 1-({1-[2-(4-{3-[2-(2,6-dioxopiperidin-3-yl)-1,3-dioxo-2,3-dihydro-1H-isoindol-4-yl]propyl}piperazin-1-yl)-2-oxoethyl]-1H-1,2,3-triazol-4-yl}methyl)-N-{[(2-{[5-fluoro-4-(4-fluoro-2-methoxyphenyl)pyridin-2-yl]amino}pyridin-4-yl)methyl](methyl)oxo-λ⁶-sulfanylidene}piperidine-3-carboxamide (4.74)

Sodium ascorbate (3.6 mg, 0.02 mmol), CuSO₄·5H₂O (4.5 mg, 0.02 mmol), 2-(4-ethynylpiperidin-1-yl)-N-{[(2-{[5-fluoro-4-(4-fluoro-2-methoxyphenyl)pyridin-2-yl]amino}pyridin-4-yl)methyl](methyl)oxo-λ⁶-sulfanylidene}acetamide (4.52) (30 mg, 0.05 mmol) and 4-{2-[1-(2-azidoacetyl) piperidin-4-yl]ethynyl}-2-(2,6-dioxopiperidin-3-yl)-2,3-dihydro-1H-isoindole-1,3-dione (4.66) (24 mg, 0.05 mmol) were reacted according to general procedure B to provide 4.74 (15 mg, 33% yield) as an off-white powder. ¹H NMR (700 MHz, DMSO) δ 11.16 (s, 1H), 9.93 (s, 1H), 8.26-8.22 (m, 2H), 7.91 (dd, J=5.1, 3.4 Hz, 1H), 7.89-7.84 (m, 2H), 7.77-7.72 (m, 3H), 7.36 (dd, J=8.4, 6.7 Hz, 1H), 7.11 (dd, J=11.4, 2.4 Hz, 1H), 6.96-6.91 (m, 2H), 5.43 (s, 2H), 5.19 (dd, J=12.9, 5.5 Hz, 1H), 4.94-4.87 (m, 2H), 3.81 (s, 5H), 3.50 (dd, J=35.7, 8.7 Hz, 2H), 3.30 (s, 3H), 3.15 (tt, J=7.7, 4.1 Hz, 1H), 3.09 (s, 2H), 2.90-2.83 (m, 2H), 2.62 (dp, J=11.5, 6.1 Hz, 2H), 2.56 (dd, J=13.1, 4.4 Hz, 1H), 2.24 (t, J=11.5 Hz, 2H), 2.16-2.05 (m, 1H), 1.99 (ddt, J=14.8, 7.6, 3.8 Hz, 1H), 1.88 (dt, J=12.1, 4.7 Hz, 3H), 1.79 (dtd, J=10.4, 7.4, 3.6 Hz, 1H), 1.70-1.64 (m, 1H), 1.60 (qd, J=12.3, 3.6 Hz, 2H), 1.24-1.13 (m, 1H). ¹³C NMR (176 MHz, DMSO) δ 178.74, 173.25, 170.32, 166.75, 166.32, 164.82, 164.59, 163.43, 158.39, 158.33, 154.99, 153.12, 151.72, 151.27, 151.16, 148.17, 138.56, 138.49, 135.28, 135.20, 134.66, 134.51, 132.53, 132.10, 132.04, 130.92, 123.38, 122.85, 120.02, 119.17, 118.55, 114.19, 113.88, 107.67, 107.55, 100.67, 100.52, 100.47, 77.81, 64.08, 57.83, 56.69, 53.08, 50.98, 49.46, 43.13, 39.18, 33.13, 32.25, 31.42, 30.85, 29.79, 27.28, 22.39. HRMS m/z 1002.35109 [M+H]⁺ (calcd. for C₅₀H₅₀F₂N₁₁O₈S, 1002.35271).

### 1-({1-[2-(4-{2-[2-(2,6-dioxopiperidin-3-yl)-1,3-dioxo-2,3-dihydro-1H-isoindol-4-yl]ethynyl}piperidin-1-yl)-2-oxoethyl]-1H-1,2,3-triazol-4-yl}methyl)-N-{[1 (2-{[5-fluoro-4-(4-fluoro-2-methoxyphenyl)pyridin-2-yl]amino}pyridin-4-yl)methyl](methyl)oxo-λ⁶-sulfanylidene}piperidine-3-carboxamide (4.75)

Sodium ascorbate (3.6 mg, 0.02 mmol), CuSO₄·5H₂O (4.5 mg, 0.02 mmol), N-{[(2-{[5-fluoro-4-(4-fluoro-2-methoxyphenyl)pyridin-2-yl]amino}pyridin-4-yl)methyl](methyl)oxo-A6-sulfanylidene}-1-(prop-2-yn-1-yl) piperidine-3-carboxamide (4.53) (25 mg, 0.05 mmol) and 4-{2-[1-(2-azidoacetyl) piperidin-4-yl]ethynyl}-2-(2,6-dioxopiperidin-3-yl)-2,3-dihydro-1H-isoindole-1,3-dione (4.66) (24 mg, 0.05 mmol) were reacted according to general procedure B to provide 4.75 (33 mg, 73% yield) as an off-white powder. ¹H NMR (400 MHZ, DMSO) δ 11.13 (s, 1H), 9.90 (s, 1H), 8.22 (dd, J=3.4, 2.0 Hz, 2H), 7.94-7.84 (m, 3H), 7.82 (d, J=1.6 Hz, 1H), 7.74-7.67 (m, 2H), 7.34 (dd, J=8.4, 6.8 Hz, 1H), 7.10 (dd, J=11.4, 2.5 Hz, 1H), 6.96-6.86 (m, 2H), 5.45 (s, 2H), 5.17 (dd, J=12.9, 5.4 Hz, 1H), 4.85 (s, 2H), 3.80 (s, 4H), 3.58-3.44 (m, 4H), 3.25 (d, J=1.3 Hz, 3H), 3.12 (dd, J=7.7, 3.9 Hz, 1H), 2.88 (dd, J=13.1, 4.6 Hz, 2H), 2.70 (d, J=10.6 Hz, 1H), 2.66-2.51 (m, 3H), 2.28 (s, 1H), 2.12-2.03 (m, 1H), 2.02-1.94 (m, 2H), 1.85 (s, 1H), 1.77 (d, J=8.9 Hz, 2H), 1.55 (s, 1H), 1.37 (d, J=11.8 Hz, 1H), 1.20 (d, J=11.4 Hz, 3H). ¹³C NMR (101 MHz, DMSO) δ 182.29, 182.26, 173.23, 170.30, 166.74, 166.31, 165.35, 164.54, 162.91, 158.43, 158.32, 154.94, 153.65, 151.18, 148.17, 143.68, 138.55, 138.50, 135.34, 135.18, 134.69, 134.43, 132.53, 132.10, 131.99, 130.92, 125.81, 123.36, 120.03, 119.19, 119.16, 118.61, 114.21, 113.89, 107.71, 107.50, 100.74, 100.45, 77.82, 57.68, 56.69, 56.11, 53.49, 53.24, 51.01, 49.47, 45.97, 43.11, 39.00, 31.42, 30.84, 29.79, 27.58, 27.28, 24.83, 22.40. HRMS m/z 1002.35137 [M+H]⁺ (calcd. for C₅₀H₅₀F₂N₁₁O₈S, 1002.35271).

### 2-{[4-[1-({1-[2-(2,6-dioxopiperidin-3-yl)-1,3-dioxo-2,3-dihydro-1H-isoindol-4-yl]piperidin-4-yl}methyl)-1H-1,2,3-triazol-4-yl]piperidin-1-yl}-N-{[2-{[5-fluoro-4-(4-fluoro-2-methoxyphenyl)pyridin-2-yl]amino}pyridin-4-yl)methyl](methyl)oxoλ⁶-sulfanylidene}acetamide (4.76)

Sodium ascorbate (4.3 mg, 0.02 mmol), CuSO₄·5H₂O (5.4 mg, 0.02 mmol), 2-(4-ethynylpiperidin-1-yl)-N-{[(2˜ {[5-fluoro-4-(4-fluoro-2-methoxyphenyl)pyridin-2-yl]amino}pyridin-4-yl)methyl](methyl)oxo-λ⁶-sulfanylidene}acetamide (4.52) (30 mg, 0.05 mmol) and 4-[4-(azidomethyl) piperidin-1-yl]-2-(2,6-dioxopiperidin-3-yl)-2,3-dihydro-1H-isoindole-1,3-dione (4.67) (24 mg, 0.06 mmol) were reacted according to general procedure B to provide 4.76 (13 mg, 25% yield) as a green powder. ¹H NMR (300 MHz, DMSO) δ 11.10 (s, 1H), 9.94 (s, 1H), 8.23 (d, J=1.6 Hz, 1H), 7.86 (s, 1H), 7.74-7.64 (m, 2H), 7.37-7.29 (m, 2H), 7.29-7.22 (m, 1H), 7.20-7.06 (m, 2H), 6.96-6.87 (m, 1H), 5.09 (dd, J=12.8, 5.4 Hz, 1H), 4.89 (s, 1H), 4.27 (d, J=7.0 Hz, 1H), 3.79 (s, 2H), 3.68 (d, J=11.7 Hz, 1H), 3.28 (s, 3H), 3.06 (s, 1H), 2.85 (d, J=11.7 Hz, 3H), 2.59 (d, J=8.6 Hz, 2H), 2.30 (d, J=0.7 Hz, 2H), 2.20 (t, J=11.3 Hz, 1H), 2.01 (s, 2H), 1.84 (d, J=12.7 Hz, 1H), 1.57 (d, J=9.8 Hz, 3H), 1.43 (d, J=11.8 Hz, 1H), ¹³C NMR (176 MHz, DMSO) δ 178.75, 173.27, 170.49, 167.54, 166.76, 164.82, 163.43, 158.39, 158.33, 154.99, 153.11, 151.71, 151.50, 151.16, 150.45, 148.17, 138.50, 137.82, 136.23, 135.28, 135.19, 134.65, 134.50, 134.11, 132.09, 132.03, 129.37, 128.68, 125.79, 124.43, 121.72, 119.16, 118.54, 116.96, 115.04, 114.17, 113.88, 107.66, 107.54, 100.68, 100.53, 64.11, 57.83, 56.69, 54.74, 53.06, 51.01, 50.93, 49.23, 39.18, 36.53, 33.11, 32.29, 31.42, 29.63, 22.53, 21.52. HRMS m/z 950.35658 [M+H]⁺ (calcd. for C₄₇H₅₀F₂N₁₁O₇S, 950.35780).

### 1˜ {[1-({1-[2-(2,6-dioxopiperidin-3-yl)-1,3-dioxo-2,3-dihydro-1H-isoindol-4-yl]piperidin-4-yl}methyl)-1H-1,2,3-triazol-4-yl]methyl}-N-{[(2-{[5-fluoro-4-(4-fluoro-2-methoxyphenyl)pyridin-2-yl]amino}pyridin-4-yl)methyl](methyl)oxo-λ⁶-sulfanylidene}piperidine-3-carboxamide (4.77)

Sodium ascorbate (4.3 mg, 0.02 mmol), CuSO₄·5H₂O (5.4 mg, 0.02 mmol), 2-(4-ethynylpiperidin-1-yl)-N-{[(2-{[5-fluoro-4-(4-fluoro-2-methoxyphenyl)pyridin-2-ylamino}pyridin-4-yl)methyl](methyl)oxo-λ⁶-sulfanylidene}acetamide (4.52) (30 mg, 0.05 mmol) and 4-[4-(azidomethyl) piperidin-1-yl]-2-(2,6-dioxopiperidin-3-yl)-2,3-dihydro-1H-isoindole-1,3-dione (4.67) (33 mg, 0.08 mmol) were reacted according to general procedure B to provide 4.77 (16 mg, 31% yield) as a green powder. ¹H NMR (700 MHz, DMSO) δ 11.08 (s, 1H), 9.92 (d, J=2.5 Hz, 1H), 8.23-8.19 (m, 2H), 7.92 (d, J=6.7 Hz, 1H), 7.72 (d, J=3.9 Hz, 1H), 7.71-7.67 (m, 1H), 7.67-7.64 (m, 1H), 7.37-7.31 (m, 2H), 7.29 (d, J=8.5 Hz, 1H), 7.10 (dd, J=11.4, 2.5 Hz, 1H), 6.91 (tdd, J=8.4, 2.5, 0.9 Hz, 1H), 6.88 (dt, J=5.2, 1.5 Hz, 1H), 5.08 (dd, J=12.9, 5.5 Hz, 1H), 4.88-4.81 (m, 2H), 4.30 (d, J=7.1 Hz, 2H), 3.79 (s, 3H), 3.67 (dd, J=10.4, 6.7 Hz, 2H), 3.51 (d, J=7.0 Hz, 2H), 3.25 (d, J=6.3 Hz, 3H), 2.91-2.79 (m, 4H), 2.68 (s, 1H), 2.61-2.52 (m, 2H), 2.29 (s, 1H), 2.06-2.00 (m, 2H), 2.00-1.98 (m, 1H), 1.89 (d, J=37.3 Hz, 1H), 1.78-1.72 (m, 1H), 1.56 (d, J=11.1 Hz, 3H), 1.43 (q, J=12.8 Hz, 2H), 1.36 (dd, J=14.0, 10.2 Hz, 1H). ¹³C NMR (176 MHz, DMSO) δ 182.24, 173.27, 170.49, 167.53, 166.75, 164.83, 163.43, 158.40, 158.34, 154.93, 153.12, 151.72, 151.17, 150.42, 148.16, 143.89, 138.48, 138.45, 136.20, 135.31, 135.22, 134.63, 134.48, 134.10, 132.06, 132.01, 127.88, 124.59, 124.40, 119.15, 118.62, 116.95, 115.03, 114.20, 113.87, 107.66, 107.54, 100.68, 100.53, 57.60, 56.69, 56.09, 54.71, 53.49, 53.31, 50.98, 50.91, 49.23, 45.90, 39.01, 38.99, 36.49, 31.42, 29.57, 27.51, 24.76, 22.52. HRMS m/z 950.35631 [M+H]⁺ (calcd. for C₄₇H₅₀F₂N₁₁O₇S, 950.35780).

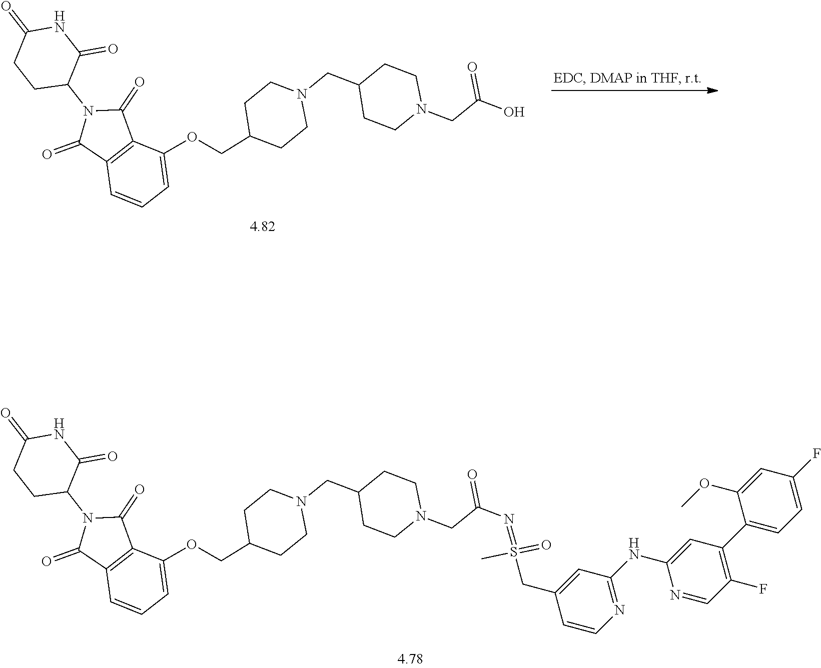 [SMILES: O=C(O)CN1CCC(CN2CCC(COc3cccc4c3C(=O)N(C3CCC(=O)NC3=O)C4=O)CC2)CC1 ; COc1cc(F)ccc1-c1cc(Nc2cc(CS(C)(=O)=NC(=O)CN3CCC(CN4CCC(COc5cccc6c5C(=O)N(C5CCC(=O)NC5=O)C6=O)CC4)CC3)ccn2)ncc1F]

### 2-(4-{[4-({[2-(2,6-dioxopiperidin-3-yl)-1,3-dioxo-2,3-dihydro-1H-isoindol-4-yl]-oxy}methyl) piperidin-1-yl]methyl}piperidin-1-yl)-N-{[(2-{[5-fluoro-4-(4-fluoro-2-methoxyphenyl)pyridin-2-yl]amino}pyridin-4-yl)methyl](methyl)oxo-λ⁶-sulfanylidene}acetamide (4.78)

2-(4-{[4-({[2-(2,6-dioxopiperidin-3-yl)-1,3-dioxo-2,3-dihydro-1H-isoindol-4-yl]oxy}methyl) piperidin-1-yl]methyl}piperidin-1-yl) acetic acid 4.82 (47 mg, 0.09 mmol), VIP152 4.16 (36 mg, 0.09 mmol), HATU (41 mg, 0.11 mmol) and DIPEA (23 mg, 0.18 mmol) were dissolved in THE (0.1 M). The mixture was stirred overnight at room temperature. The reaction mixture was concentrated under reduced pressure and purified by reversed phase chromatography (C18, H₂O:ACN eluent) to provide 4.78 (15 mg, 18% yield) as a white powder. ¹H NMR (700 MHZ, CDCl₃) δ 8.31-8.25 (m, 1H), 8.15 (t, J=1.9 Hz, 1H), 7.98 (d, J=67.8 Hz, 1H), 7.87 (d, J=19.8 Hz, 1H), 7.68 (ddd, J=8.6, 7.2, 1.7 Hz, 1H), 7.46 (d, J=7.4 Hz, 1H), 7.37 (d, J=4.9 Hz, 1H), 7.26-7.22 (m, 1H), 7.22-7.14 (m, 2H), 6.89 (td, J=4.8, 1.5 Hz, 1H), 6.76-6.69 (m, 2H), 4.96 (ddd, J=12.7, 5.4, 1.9 Hz, 1H), 4.91-4.59 (m, 2H), 4.01 (s, 1H), 3.84-3.79 (m, 3H), 3.28-3.18 (m, 2H), 3.15-3.07 (m, 2H), 2.97-2.78 (m, 6H), 2.73 (dddd, J=16.9, 13.5, 5.1, 3.0 Hz, 1H), 2.15-2.10 (m, 2H), 2.06 (q, J=9.3 Hz, 1H), 1.99-1.75 (m, 5H), 1.68 (s, 4H), 1.50 (d, J=51.0 Hz, 3H), 1.31 (d, J=12.3 Hz, 3H). ¹³C NMR (176 MHz, CDCl₃) δ 171.64, 171.45, 168.55, 167.10, 165.69, 164.98, 163.57, 158.17, 158.11, 156.82, 154.63, 154.58, 153.61, 152.20, 149.84, 148.45, 137.72, 137.43, 136.52, 135.78, 134.72, 134.57, 133.83, 131.78, 131.72, 129.05, 128.24, 125.31, 118.90, 118.64, 117.91, 117.81, 117.63, 117.20, 117.18, 115.75, 114.02, 113.01, 112.44, 107.35, 107.23, 99.69, 99.54, 73.78, 73.69, 64.93, 64.50, 64.33, 60.65, 58.96, 58.86, 55.94, 54.08, 53.91, 53.78, 49.14, 38.90, 35.94, 33.12, 31.43, 30.80, 29.72, 28.59, 22.79, 22.77, 1.03. HRMS m/z 913.34897 [M+H]⁺ (calcd. for C₄₆H₅₁F₂N₈O₈S, 913.35131).

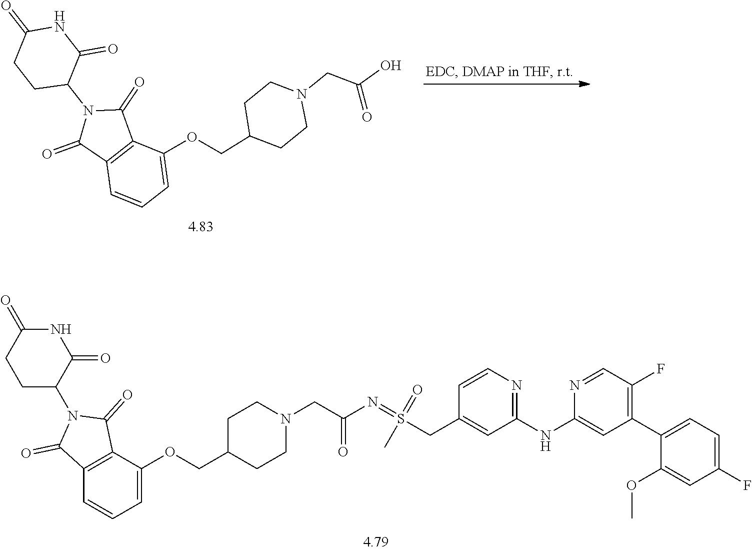

### 2-[4-({[2-(2,6-dioxopiperidin-3-yl)-1,3-dioxo-2,3-dihydro-1H-isoindol-4-yl]oxy}methyl) piperidin-1-yl]-N-{[(2-{[5˜fluoro-4-(4-fluoro-2-methoxyphenyl)pyridin-2-yl]amino}pyridin-4-yl)methyl](methyl)oxo-26-sulfanylidene}acetamide (4.79)

2-[4-({[2-(2,6-dioxopiperidin-3-yl)-1,3-dioxo-2,3-dihydro-1H-isoindol-4-yl]oxy}methyl) piperidin-1-yl]acetic acid 4.83 (50 mg, 0.09 mmol), VIP152 4.16 (47 mg, 0.09 mmol), HATU (53 mg, 0.11 mmol) and DIPEA (30 mg, 0.18 mmol) were dissolved in THF (0.1 M). The mixture was stirred overnight at room temperature. The reaction mixture was concentrated under reduced pressure and purified by reversed phase chromatography (C18, H₂O:ACN eluent) to provide 4.79 (54 mg, 57% yield) as a white powder. ¹H NMR (700 MHz, CDCl₃) δ 9.45 (d, J=210.4 Hz, 1H), 8.27 (t, J=5.0 Hz, 1H), 8.00 (d, J=77.8 Hz, 1H), 7.84 (d, J=52.2 Hz, 1H), 7.65 (ddd, J=8.6, 7.3, 4.9 Hz, 1H), 7.44 (dd, J=7.3, 5.7 Hz, 1H), 7.39 (dd, J=23.4, 5.1 Hz, 1H), 7.24 (dd, J=6.3, 2.1 Hz, 1H), 7.18 (dt, J=10.0, 8.4 Hz, 2H), 6.89 (td, J=5.1, 1.5 Hz, 1H), 6.77-6.70 (m, 2H), 4.96 (dd, J=12.5, 5.5 Hz, 1H), 4.85-4.57 (m, 2H), 4.03-3.93 (m, 2H), 3.81 (d, J=1.8 Hz, 3H), 3.29-3.20 (m, 2H), 3.16 (d, J=22.8 Hz, 3H), 3.11-2.98 (m, 2H), 2.91-2.85 (m, 1H), 2.81 (qdd, J=12.3, 4.0, 2.3 Hz, 1H), 2.73 (dddd, J=17.0, 13.6, 5.0, 1.8 Hz, 1H), 2.35 (s, 1H), 2.20-2.11 (m, 3H), 1.91-1.88 (m, 1H), 1.85 (d, J=4.7 Hz, 1H), 1.53 (q, J=11.1 Hz, 2H). ¹³C NMR (176 MHz, CDCl₃) δ 171.75, 171.47, 168.78, 168.73, 167.10, 165.91, 165.82, 164.99, 163.58, 158.15, 158.09, 156.61, 156.58, 154.63, 154.60, 153.61, 152.20, 149.86, 149.80, 148.50, 148.46, 137.67, 137.24, 136.54, 135.87, 135.82, 135.78, 135.73, 134.72, 134.66, 134.57, 134.50, 133.82, 133.79, 131.76, 131.70, 129.05, 128.24, 125.31, 119.29, 119.07, 118.60, 117.84, 117.80, 117.46, 117.30, 115.98, 115.89, 114.06, 114.03, 113.11, 113.03, 107.40, 107.28, 99.73, 99.58, 73.96, 73.85, 64.12, 63.90, 59.05, 58.85, 55.96, 53.36, 53.28, 49.21, 49.17, 38.86, 38.72, 35.09, 31.38, 31.36, 29.72, 28.42, 22.79, 22.76. HRMS m/z 816.26010 [M+H]⁺ (calcd. for C₄₀H₄₀F₂N₇O₈S, 816.26216).

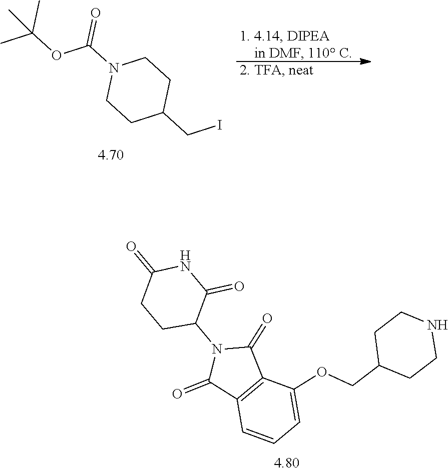 [SMILES: CC(C)(C)OC(=O)N1CCC(CI)CC1 ; O=C1CCC(N2C(=O)c3cccc(OCC4CCNCC4)c3C2=O)C(=O)N1]

### 2-(2,6-dioxopiperidin-3-yl)-4-[(piperidin-4-yl) methoxy]-2,3-dihydro-1H-isoindole-1,3-dione (4.80)

4-hydroxy thalidomide 4.14 (1.5 g, 5.5 mmol), tert-butyl 4-(iodomethyl) piperidine-1-carboxylate 4.70 (1.96 g, 6.0 mmol) and DIPEA (2.12 g, 0.16 mmol) were dissolved in DMF (0.13 M). The mixture was stirred overnight at 110° C. under argon protection. The reaction mixture was concentrated under reduced pressure and purified by reversed phase chromatography (C18, H₂O:ACN eluent) to provide Boc-protected 4.80 (880 mg, 34% yield) as a white powder.

The recovered solid was dissolved in TFA (1.6 M) and stirred at room temperature for one hour. The reaction mixture was then concentrated under reduced pressure to provide 4.80, which was taken onto the next step without further purification. ¹H NMR (400 MHZ, DMSO) δ 11.11 (s, 1H), 7.83 (dd, J=8.5, 7.2 Hz, 1H), 7.54 (d, J=8.5 Hz, 1H), 7.47 (d, J=7.2 Hz, 1H), 5.08 (dd, J=12.8, 5.5 Hz, 1H), 4.12 (d, J=6.3 Hz, 2H), 3.33 (s, 2H), 3.03-2.80 (m, 4H), 2.66-2.53 (m, 2H), 2.20-2.09 (m, 1H), 2.04 (ddd, J=9.8, 5.7, 2.8 Hz, 1H), 1.98 (d, J=13.5 Hz, 2H), 1.53 (dtd, J=15.1, 12.4, 4.0 Hz, 2H). ¹³C NMR (101 MHZ, DMSO) δ 171.17, 168.30, 165.17, 163.70, 154.14, 135.47, 131.56, 118.27, 114.74, 114.08, 113.82, 70.54, 47.12, 46.96, 41.07, 40.91, 31.31, 29.30, 23.65, 23.41, 23.39, 23.00, 20.40. HRMS m/z 372.15418 [M+H]⁺ (calcd. for C₁₉H₂₂N₃O₅, 372.15540).

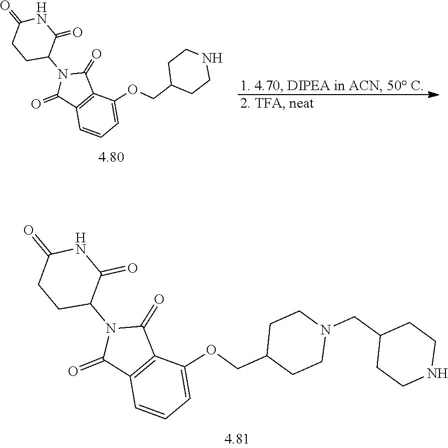 [SMILES: O=C1CCC(N2C(=O)c3cccc(OCC4CCNCC4)c3C2=O)C(=O)N1 ; O=C1CCC(N2C(=O)c3cccc(OCC4CCN(CC5CCNCC5)CC4)c3C2=O)C(=O)N1]

### 2-(2,6-dioxopiperidin-3-yl)-4-((1-(piperidin-4-yl)methyl) piperidin]4-yl) methoxy) isoindoline-1,3-dione (4.81)

2-(2,6-dioxopiperidin-3-yl)-4-[(piperidin-4-yl) methoxy]-2,3-dihydro-1H-isoindole-1,3-dione 4.80 (700 mg, 1.9 mmol), tert-butyl 4-(iodomethyl) piperidine-1-carboxylate 4.70 (797 mg, 2.4 mmol) and DIPEA (585 mg, 4.5 mmol) were dissolved in ACN (0.13 M) and DMF (0.5 M). The mixture was stirred overnight at 50° C. under argon protection. The reaction mixture was concentrated under reduced pressure and purified by reversed phase chromatography (C18, H₂O:ACN eluent) to provide Boc-protected version of 4.81 (407 mg, 38% yield) as a white powder. ¹H NMR (400 MHZ, DMSO) δ 11.16 (d, J=3.5 Hz, 1H), 7.88 (dd, J=8.6, 7.3 Hz, 1H), 7.64-7.50 (m, 2H), 5.14 (dd, J=12.7, 5.4 Hz, 1H), 4.17 (d, J=6.3 Hz, 2H), 3.68 (ddt, J=12.4, 5.7, 2.8 Hz, 2H), 3.20 (qd, J=7.5, 4.4 Hz, 1H), 3.09-3.04 (m, 2H), 3.04-2.88 (m, 5H), 2.79 (d, J=0.6 Hz, 1H), 2.71-2.58 (m, 2H), 2.14-2.04 (m, 3H), 1.97 (d, J=13.7 Hz, 2H), 1.71 (q, J=12.6 Hz, 2H), 1.52-1.45 (m, 1H), 1.42 (d, J=6.1 Hz, 1H).

The recovered solid was dissolved in TFA (1.6 M) and stirred at room temperature for one hour. The reaction mixture was then concentrated under reduced pressure and purified by normal phase chromatography (SiO₂, DCM:MeOH eluent) to provide 4.81. ¹H NMR (400 MHZ, DMSO) δ 11.16 (d, J=3.5 Hz, 1H), 9.62 (s, 1H), 8.95 (d, J=10.9 Hz, 1H), 8.69 (d, J=11.1 Hz, 1H), 7.88 (dd, J=8.6, 7.3 Hz, 1H), 7.64-7.50 (m, 2H), 5.14 (dd, J=12.7, 5.4 Hz, 1H), 4.17 (d, J=6.3 Hz, 2H), 3.68 (ddt, J=12.4, 5.7, 2.8 Hz, 2H), 3.20 (qd, J=7.5, 4.4 Hz, 1H), 3.09-3.04 (m, 2H), 3.04-2.88 (m, 5H), 2.79 (d, J=0.6 Hz, 1H), 2.71-2.58 (m, 2H), 2.14-2.04 (m, 3H), 1.97 (d, J=13.7 Hz, 2H), 1.71 (q, J=12.6 Hz, 2H), 1.52-1.45 (m, 1H), 1.42 (d, J=6.1 Hz, 1H), ¹³C NMR (101 MHZ, DMSO) δ 173.26, 170.39, 169.27, 167.26, 165.81, 162.79, 156.21, 137.56, 133.68, 120.43, 118.53, 116.89, 115.97, 115.58, 72.56, 60.97, 53.97, 52.31, 50.28, 49.23, 42.95, 42.23, 36.24, 33.48, 31.40, 31.23, 28.98, 28.84, 26.79, 25.89, 24.09, 22.50, 18.46. HRMS m/z 469.24409 [M+H]⁺ (calcd. for C₂₅H₃₃N₄O₅, 469.24455).

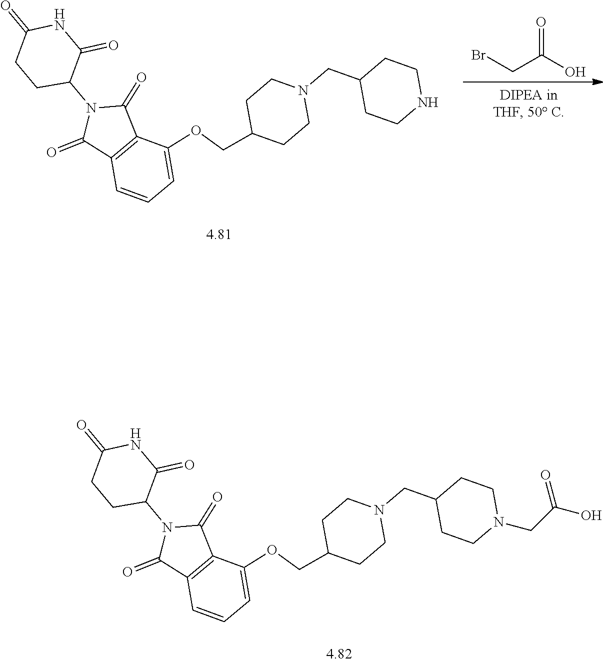 [SMILES: O=C1CCC(N2C(=O)c3cccc(OCC4CCN(CC5CCNCC5)CC4)c3C2=O)C(=O)N1 ; O=C(O)CBr ; O=C(O)CN1CCC(CN2CCC(COc3cccc4c3C(=O)N(C3CCC(=O)NC3=O)C4=O)CC2)CC1]

### 2-(4-{[4-({[2-(2,6-dioxopiperidin-3-yl)-1,3-dioxo-2,3-dihydro-1H-isoindol-4-yl]oxy}methyl) piperidin-1-yl]methyl}piperidin-1-yl) acetic acid (4.82)

2-(2,6-dioxopiperidin-3-yl)-4-({1-[(piperidin-4-yl)methyl]piperidin-4-yl}methoxy)-2,3-dihydro-1H-isoindole-1,3-dione 4.81 (100 mg, 0.21 mmol), 2-bromoacetic acid (34.4 mg, 0.25 mmol) and DIPEA (133 mg, 1.0 mmol) were dissolved in THE (0.1 M). The mixture was stirred overnight at 50° C. under argon protection. The reaction mixture was concentrated under reduced pressure and purified by reversed phase chromatography (C18, H₂O:ACN eluent) to provide 4.82 (50 mg, 46% yield) as a white powder. ¹H NMR (700 MHz, DMSO) δ 11.10 (s, 1H), 8.14 (s, 1H), 7.82 (tt, J=7.6, 3.9 Hz, 1H), 7.53 (dd, J=8.5, 3.2 Hz, 1H), 7.47 (dd, J=7.3, 3.9 Hz, 1H), 5.07 (dd, J=12.9, 5.5 Hz, 1H), 4.20-4.03 (m, 3H), 3.26-3.09 (m, 8H), 2.88 (ddd, J=17.1, 13.9, 5.5 Hz, 2H), 2.62-2.54 (m, 3H), 2.06-2.00 (m, 1H), 1.98-1.86 (m, 3H), 1.86-1.74 (m, 3H), 1.53 (s, 2H), 1.34 (q, J=12.9 Hz, 2H), 1.27-1.23 (m, 1H). ¹³C NMR (176 MHz, DMSO) δ 173.27, 170.42, 169.51, 169.20, 167.29, 165.84, 163.53, 156.34, 137.57, 133.69, 120.31, 116.80, 115.86, 65.39, 61.02, 59.17, 52.21, 50.28, 49.22, 31.42, 29.01, 28.47, 24.13, 22.49. HRMS m/z 527.24925 [M+H]⁺ (calcd. for C₂₇H₃₅N₄O₇, 527.25003).

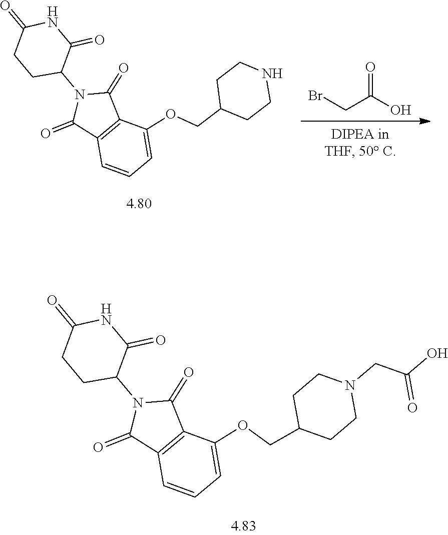 [SMILES: O=C1CCC(N2C(=O)c3cccc(OCC4CCNCC4)c3C2=O)C(=O)N1 ; O=C(O)CBr ; O=C(O)CN1CCC(COc2cccc3c2C(=O)N(C2CCC(=O)NC2=O)C3=O)CC1]

### 2˜ [4-({[2-(2,6-dioxopiperidin-3-yl)-1,3-dioxo-2,3-dihydro-1H-isoindol-4-yl]oxy}methyl) piperidin-1-yl]acetic acid (4.83)

2-(2,6-dioxopiperidin-3-yl)-4-[(piperidin-4-yl) methoxy]-2,3-dihydro-1H-isoindole-1,3-dione 4.80 (150 mg, 0.40 mmol), 2-bromoacetic acid (62 mg, 0.44 mmol) and DIPEA (261 mg, 2.0 mmol) were dissolved in ACN (0.1 M). The mixture was stirred overnight at 50° C. under argon protection. The reaction mixture was concentrated under reduced pressure and purified by reversed phase chromatography (C18, H₂O:ACN eluent) to provide 4.83 (100 mg, 58% yield) as a white powder. ¹H NMR (400 MHZ, DMSO) δ 11.10 (s, 1H), 7.82 (dd, J=8.5, 7.3 Hz, 1H), 7.53 (d, J=8.5 Hz, 1H), 7.46 (d, J=7.2 Hz, 1H), 5.08 (dd, J=12.8, 5.4 Hz, 1H), 4.10 (d, J=6.2 Hz, 2H), 3.22 (d, J=4.2 Hz, 3H), 2.88 (ddd, J=17.0, 13.8, 5.3 Hz, 1H), 2.65-2.53 (m, 4H), 2.09-1.98 (m, 1H), 1.95-1.83 (m, 3H), 1.62-1.47 (m, 2H), 1.12-1.02 (m, 2H). ¹³C NMR (101 MHz, DMSO) § 173.24, 170.41, 168.52, 167.30, 165.80, 156.37, 137.53, 133.69, 120.31, 116.81, 115.78, 73.00, 59.56, 52.62, 52.20, 49.23, 49.07, 40.68, 34.22, 31.42, 26.94, 22.49, 19.74. HRMS m/z 430.15974 [M+H]⁺ (calcd. for C₂₁H₂₄N₃O₇, 430.16088).

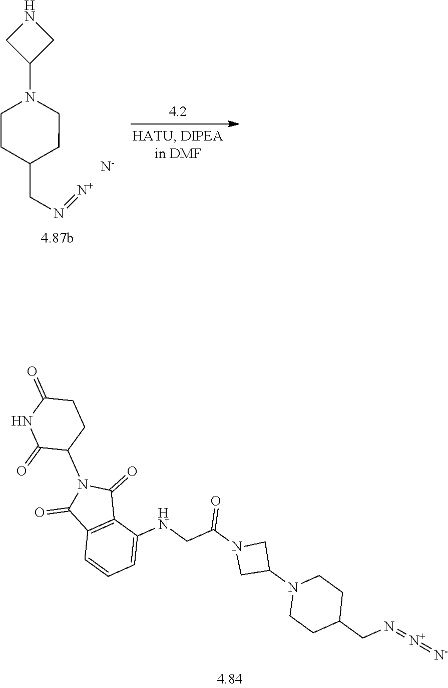

### 4-((2-(3-(4-(azidomethyl) piperidin-1-yl) azetidin-1-yl)-2-oxoethyl)amino)-2-(2,6-dioxopiperidin-3-yl) isoindoline-1,3-dione (4.84)

1-(azetidin-3-yl)-4-(azidomethyl) piperidine 4.87b (32 mg, 0.17 mmol), 4.2 (55 mg, 0.17 mmol), HATU (76 mg, 0.20 mmol) and DIPEA (43 mg, 0.33 mmol) were dissolved in THF (0.2 M). The mixture was stirred overnight at room temperature. The reaction mixture was concentrated under reduced pressure and purified by reversed phase chromatography (C18, H₂O:ACN eluent) to provide 4.84 (20 mg, 24% yield) as a green powder. ¹H NMR (700 MHz, DMSO) δ 11.09 (s, 1H), 7.59 (dd, J=8.5, 7.1 Hz, 1H), 7.07 (d, J=7.0 Hz, 1H), 6.98 (d, J=8.6 Hz, 1H), 6.84 (t, J=5.2 Hz, 1H), 5.06 (dd, J=12.9, 5.5 Hz, 1H), 4.19 (t, J=8.0 Hz, 1H), 4.05-4.02 (m, 1H), 4.02-3.97 (m, 2H), 3.97-3.91 (m, 1H), 3.73 (dd, J=9.9, 5.2 Hz, 1H), 3.18-3.12 (m, 1H), 2.92-2.84 (m, 2H), 2.78 (dd, J=33.8, 11.1 Hz, 2H), 2.62-2.52 (m, 2H), 2.03 (dtt, J=13.9, 5.4, 2.6 Hz, 1H), 1.84-1.75 (m, 2H), 1.66 (q, J=5.1 Hz, 2H), 1.53 (dqd, J=11.0, 6.9, 2.9 Hz, 1H), 1.24-1.12 (m, 3H), ¹³C NMR (176 MHz, DMSO) δ 173.29, 170.52, 169.25, 168.41, 167.79, 146.13, 136.65, 132.49, 118.40, 111.42, 110.08, 56.60, 54.54, 53.71, 52.48, 49.49, 49.26, 49.06, 42.49, 36.08, 31.45, 29.25, 22.61. HRMS m/z 509.22533 [M+H]⁺ (calcd. for C₂₄H₂₉N₈O₅, 509.22554).

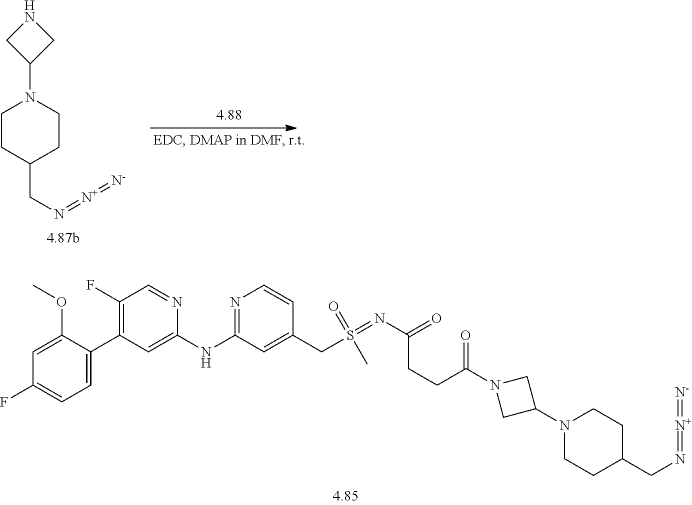 [SMILES: [N-]=[N+]=NCC1CCN(C2CNC2)CC1 ; COc1cc(F)ccc1-c1cc(Nc2cc(CS(C)(=O)=NC(=O)CCC(=O)N3CC(N4CCC(CN=[N+]=[N-])CC4)C3)ccn2)ncc1F]

### 4˜ {3-[4-(azidomethyl) piperidin-1-yl]azetidin-1-yl}-N-{[(2-{[5-fluoro-4-(4-fluoro-2-methoxyphenyl)pyridin-2-yl]amino}pyridin-4-yl)methyl](methyl)oxo-λ⁶-sulfanylidene}-4-oxobutanamide (4.85)

3-({[(2-{[5-fluoro-4-(4-fluoro-2-methoxyphenyl)pyridin-2-yl]amino}pyridin-4-yl)methyl](methyl)oxo-λ⁶-sulfanylidene}carbamoyl) propanoic acid 4.88 (48 mg, 0.10 mmol), DMAP (23 mg, 0.20 mmol), EDC·HCl (36 mg, 0.10 mmol) and 1-(azetidin-3-yl)-4-(azidomethyl) piperidine 4.87b (37 mg, 0.20 mmol) were reacted according to general procedure A to provide 4.85 as a pale yellow powder (40 mg, 62% yield). ¹H NMR (700 MHz, DMSO) δ 9.94-9.88 (m, 1H), 8.23 (dt, J=5.8, 1.3 Hz, 2H), 7.86-7.83 (m, 1H), 7.62 (s, 1H), 7.37 (dd, J=8.4, 6.7 Hz, 1H), 7.12 (dd, J=11.4, 2.5 Hz, 1H), 6.97-6.90 (m, 2H), 4.88 (qd, J=13.2, 3.2 Hz, 2H), 4.13-4.06 (m, 1H), 3.93-3.87 (m, 1H), 3.82 (s, 4H), 3.65-3.59 (m, 1H), 3.24 (td, J=6.4, 3.7 Hz, 5H), 3.06-2.97 (m, 1H), 2.82-2.69 (m, 2H), 2.50-2.40 (m, 2H), 2.31-2.18 (m, 2H), 1.75 (dddt, J=11.2, 8.4, 5.5, 2.8 Hz, 2H), 1.66-1.62 (m, 2H), 1.51 (tq, J=11.1, 3.6 Hz, 1H), 1.21-1.11 (m, 2H). ¹³C NMR (176 MHz, DMSO) δ 180.71, 180.70, 172.05, 164.83, 163.44, 158.42, 158.36, 155.03, 153.12, 151.72, 151.11, 148.17, 138.60, 138.58, 135.21, 135.12, 134.76, 134.61, 132.09, 132.03, 119.25, 119.23, 118.49, 114.14, 113.91, 113.87, 107.67, 107.55, 100.70, 100.55, 57.57, 57.40, 56.70, 56.61, 54.06, 53.77, 51.95, 51.75, 49.49, 49.34, 45.00, 38.97, 38.94, 36.07, 34.01, 33.99, 29.45, 29.26, 29.24, 29.20, 26.55, 26.52, 19.03. HRMS m/z 682.27198 [M+H]⁺ (calcd. for C₃₂H₃₈F₂N₉O₄S, 682.27300).

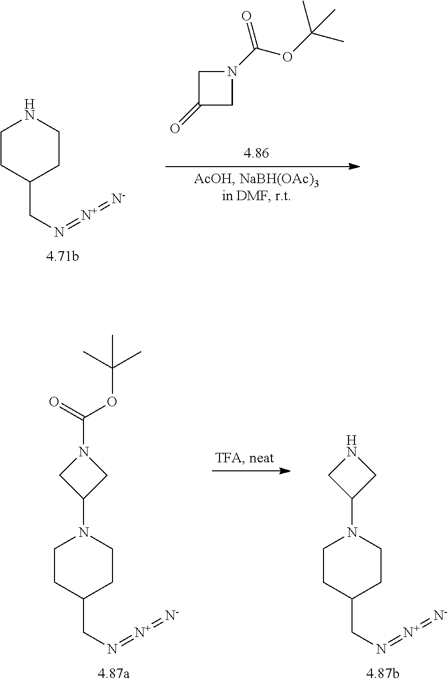 [SMILES: [N-]=[N+]=NCC1CCNCC1 ; CC(C)(C)OC(=O)N1CC(=O)C1 ; CC(C)(C)OC(=O)N1CC(N2CCC(CN=[N+]=[N-])CC2)C1 ; [N-]=[N+]=NCC1CCN(C2CNC2)CC1]

### 1-(azetidin-3-yl)-4-(azidomethyl) piperidine (4.87b)

4-(azidomethyl) piperidine 4.71b (1.25 g, 7.1 mmol) was dissolved in DMF (0.3 M) with tert-butyl 3-oxoazetidine-1-carboxylate 4.86 (1.2 g, 7.0 mmol) and 17 N acetic acid (8.7 M) and stirred at room temperature for 10 minutes. Sodium bis(acetyloxy) boranuidyl acetate (3.0 g, 14 mmol) was then added under inert atmosphere and the reaction mixture was stirred at room temperature overnight. The reaction was quenched with a saturated solution of sodium carbonate and extracted three times with DCM. The combined organic layers were washed with cold brine and dried over Na₂SO₄. The crude mixture was purified by reversed phase chromatography (C18, H₂O:ACN eluent) to provide 4.87a (830 mg, 40% yield) as a clear colorless gel. ¹H NMR (400 MHZ, CDCl₃) δ 4.39 (ddt, J=13.3, 4.3, 2.1 Hz, 1H), 3.85 (dd, J=8.7, 7.1 Hz, 1H), 3.79-3.69 (m, 1H), 3.59 (ddt, J=13.3, 4.4, 2.1 Hz, 1H), 3.19-3.10 (m, 3H), 3.07-2.96 (m, 1H), 2.75 (d, J=5.8 Hz, 1H), 2.55 (td, J=12.9, 3.1 Hz, 1H), 1.85-1.67 (m, 6H), 1.36 (s, 9H). ¹³C NMR (101 MHz, CDCl₃) δ 160.77, 79.49, 77.24, 56.99, 56.67, 54.01, 49.69, 45.59, 39.41, 36.86, 30.34, 28.38. HRMS m/z 296.20713 [M+H]⁺ (calcd. for C₁₄H₂₆N₅O₂, 296.20810).

Tert-butyl 3-[4-(azidomethyl) piperidin-1-yl]azetidine-1-carboxylate 4.87a (830 mg, 2.8 mmol) was dissolved in TFA (1.6 M) and stirred at room temperature overnight. The reaction was then dissolved in saturated sodium carbonate solution which was neutralize with 2 M KOH and extracted with 5% EtOH in chloroform multiple times. The organic layers were dried over Na₂SO₄, filtered and concentrated under reduced pressure to obtain 4.87b as a clear, colorless gel which was taken onto the next step without further purification.

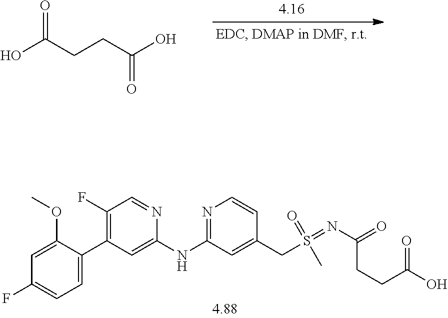 [SMILES: O=C(O)CCC(=O)O ; COc1cc(F)ccc1-c1cc(Nc2cc(CS(C)(=O)=NC(=O)CCC(=O)O)ccn2)ncc1F]

### 3-({[(2-{[5-fluoro-4-(4-fluoro-2-methoxyphenyl)pyridin-2-yl]amino}pyridin-4-yl)methyl](methyl)oxo-λ⁶-sulfanylidene}carbamoyl) propanoic acid (4.88)

VIP-152 4.16 (60 mg, 0.15 mmol), DMAP (36 mg, 0.30 mmol), EDC·HCl (57 mg, 0.30 mmol) and succinic acid (70 mg, 0.60 mmol) were reacted according to general procedure A to provide 4.88 as a pale, yellow powder (50 mg, 67% yield). ¹H NMR (700 MHz, DMSO) δ 9.87 (s, 1H), 8.23-8.18 (m, 2H), 7.78 (d, J=5.4 Hz, 1H), 7.65 (s, 1H), 7.36 (dd, J=8.4, 6.8 Hz, 1H), 7.10 (dd, J=11.4, 2.5 Hz, 1H), 6.95-6.89 (m, 2H), 4.85 (d, J=2.2 Hz, 2H), 3.81 (s, 3H), 3.23 (s, 3H), 2.49-2.40 (m, 4H). ¹³C NMR (176 MHZ, DMSO) δ 180.40, 174.37, 164.83, 163.43, 158.41, 158.35, 154.99, 153.13, 151.73, 151.12, 148.18, 138.53, 135.26, 135.18, 134.69, 134.54, 132.11, 132.05, 119.22, 119.20, 118.57, 114.21, 113.83, 107.68, 107.55, 100.68, 100.53, 57.49, 56.70, 38.94, 34.17, 31.16, 29.74. HRMS m/z 505.13467 [M+H]⁺ (calcd. for C₂₃H₂₃F₂N₄O₅S, 505.13517)

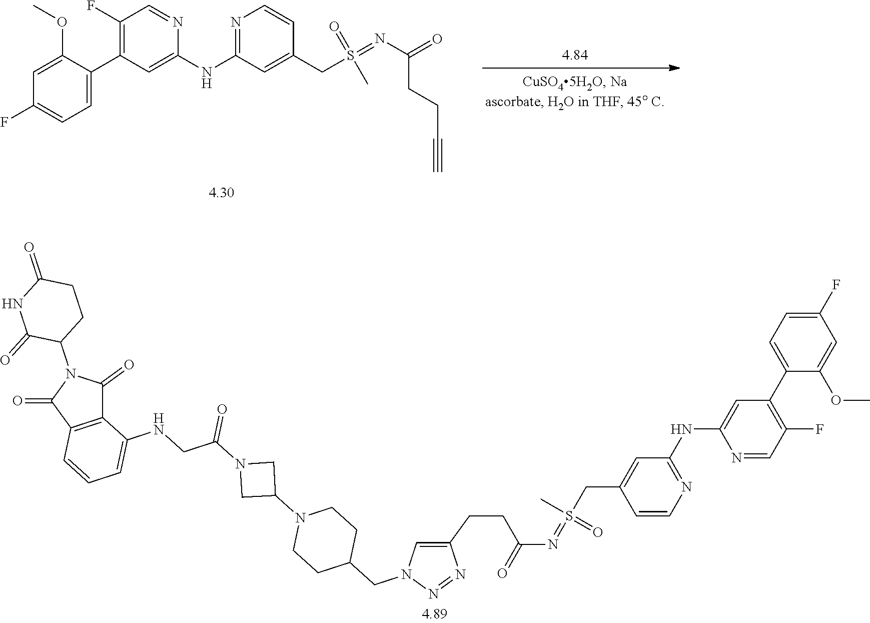 [SMILES: C#CCCC(=O)N=S(C)(=O)Cc1ccnc(Nc2cc(-c3ccc(F)cc3OC)c(F)cn2)c1 ; COc1cc(F)ccc1-c1cc(Nc2cc(CS(C)(=O)=NC(=O)CCc3cn(CC4CCN(C5CN(C(=O)CNc6cccc7c6C(=O)N(C6CCC(=O)NC6=O)C7=O)C5)CC4)nn3)ccn2)ncc1F]

### 3-(1-((1-(1-((2-(2,6-dioxopiperidin-3-yl)-1,3-dioxoisoindolin-4-yl)glycyl) azetidin-3-yl) piperidin-4-yl)methyl)-1H-1,2,3-triazol-4-yl)-N-(((2-((5-fluoro-4-(4-fluoro-2-methoxyphenyl)pyridin-2-yl)amino)pyridin-4-yl)methyl)(methyl) (oxo)-26-sulfaneylidene) propanamide (4.89)

Sodium ascorbate (2.4 mg, 0.01 mmol), CuSO₄·5H₂O (3.0 mg, 0.01 mmol), 2-(4-ethynylpiperidin-1-yl)-N-{[(2-{[5-fluoro-4-(4-fluoro-2-methoxyphenyl)pyridin-2-yl]amino}pyridin-4-yl)methyl](methyl)oxo-λ⁶-sulfanylidene}acetamide (4.30) (21 mg, 0.04 mmol) and 4-((2-(3-(4-(azidomethyl) piperidin-1-yl) azetidin-1-yl)-2-oxoethyl)amino)-2-(2,6-dioxopiperidin-3-yl) isoindoline-1,3-dione (4.84) (18 mg, 0.04 mmol) were reacted according to general procedure B to provide 4.89 (5 mg, 14% yield) as a green powder, 1H NMR (700 MHz, DMSO) δ 11.09 (s, 1H), 9.93 (s, 1H), 8.32 (s, 1H), 8.23-8.19 (m, 2H), 7.74 (d, J=5.4 Hz, 1H), 7.71 (t, J=1.8 Hz, 2H), 7.59 (dd, J=8.5, 7.1 Hz, 1H), 7.35 (dd, J=8.4, 6.8 Hz, 1H), 7.12-7.06 (m, 2H), 6.98 (d, J=8.6 Hz, 1H), 6.92 (td, J=8.4, 2.5 Hz, 1H), 6.89 (dd, J=5.2, 1.5 Hz, 1H), 6.85 (t, J=5.2 Hz, 1H), 5.07 (dd, J=12.9, 5.5 Hz, 1H), 4.86 (s, 2H), 4.17 (dd, J=10.3, 7.4 Hz, 3H), 4.00-3.96 (m, 3H), 3.91 (dd, J=9.6, 7.1 Hz, 1H), 3.80 (s, 3H), 3.70 (dd, J=9.7, 5.2 Hz, 1H), 3.26 (s, 3H), 3.10 (ddd, J=12.5, 7.0, 5.3 Hz, 1H), 2.93-2.86 (m, 1H), 2.84 (t, J=7.5 Hz, 2H), 2.77-2.73 (m, 1H), 2.70 (d, J=11.1 Hz, 1H), 2.58-2.53 (m, 3H), 2.04 (dtd, J=12.9, 5.3, 2.3 Hz, 1H), 1.79-1.68 (m, 3H), 1.45 (d, J=12.3 Hz, 2H), 1.17 (d, J=12.4 Hz, 2H). ¹³C NMR (176 MHz, DMSO) δ 180.56, 173.27, 170.52, 169.25, 168.37, 167.78, 164.83, 163.44, 158.40, 158.35, 154.99, 153.12, 151.72, 151.15, 148.19, 146.36, 146.12, 138.52, 136.63, 135.27, 135.18, 134.65, 134.50, 132.50, 132.09, 132.03, 122.75, 119.19, 118.58, 118.38, 114.23, 113.81, 111.40, 110.09, 107.67, 107.55, 100.69, 100.54, 57.61, 56.70, 54.73, 54.49, 53.68, 52.47, 49.34, 49.12, 49.06, 42.47, 38.98, 38.86, 36.78, 31.46, 29.14, 22.61, 21.76. HRMS m/z 993.36142 [M+H]⁺ (calcd. for C₄₈H₅₁F₂N₁₂O₈S, 993.36361).

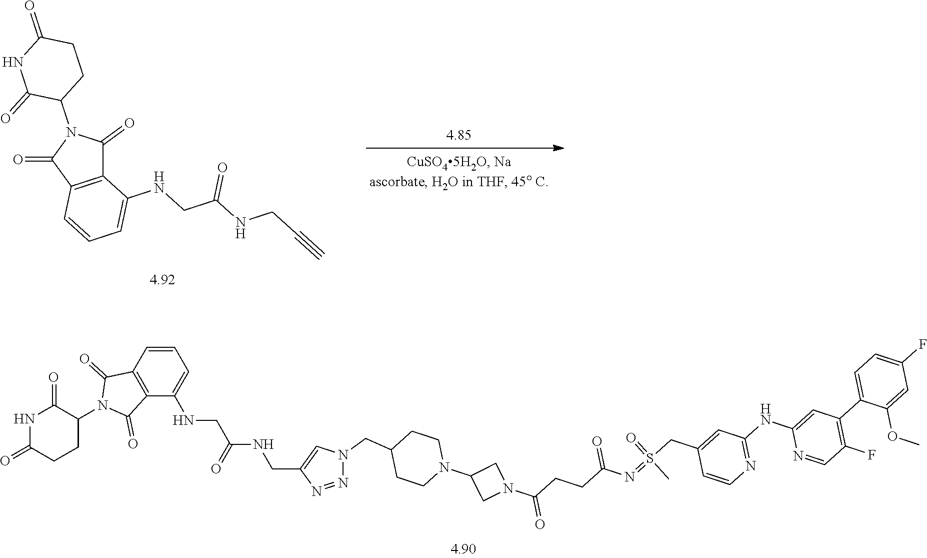 [SMILES: C#CCNC(=O)CNc1cccc2c1C(=O)N(C1CCC(=O)NC1=O)C2=O ; COc1cc(F)ccc1-c1cc(Nc2cc(CS(C)(=O)=NC(=O)CCC(=O)N3CC(N4CCC(Cn5cc(CNC(=O)CNc6cccc7c6C(=O)N(C6CCC(=O)NC6=O)C7=O)nn5)CC4)C3)ccn2)ncc1F]

### 4-{3-[4-({4-[(2˜ {[2˜ (2,6-dioxopiperidin-3-yl)-1,3-dioxo-2,3-dihydro-1H-isoindol-4-yl]amino}acetamido)methyl]-1H-1,2,3-triazol-1-yl}methyl) piperidin-1-yl]azetidin-1-yl}-N-{[(2-{[5-fluoro-4-(4-fluoro-2-methoxyphenyl)pyridin-2-yl]amino}pyridin-4-yl)methyl](methyl)oxo-λ⁶-sulfanylidene}-4-oxobutanamide (4.90)

Sodium ascorbate (2.6 mg, 0.01 mmol), CuSO₄·5H₂O (3.3 mg, 0.01 mmol), 4-{3-[4-(azidomethyl) piperidin-1-yl]azetidin-1-yl}-N-{[(2-{[S-fluoro-4-(4-fluoro-2-methoxyphenyl)pyridin-2-yl]amino}pyridin-4-yl)methyl](methyl)oxo-λ⁶-sulfanylidene}-4-oxobutanamide 4.85 (18 mg, 0.03 mmol) and 2-((2-(2,6-dioxopiperidin-3-yl)-1,3-dioxoisoindolin-4-yl)amino)-N-(prop-2-yn-1-yl) acetamide 4.92 (9.7 mg, 0.03 mmol) were reacted according to general procedure B to provide 4.90 (16 mg, 58% yield) as a green powder. TH NMR (700 MHz, DMSO) & 11.10 (s, 1H), 9.90 (d, J=2.3 Hz, 1H), 8.61 (t, J=5.7 Hz, 1H), 8.23-8.19 (m, 2H), 7.86 (s, 1H), 7.83 (d, J=5.4 Hz, 1H), 7.61-7.56 (m, 2H), 7.35 (dd, J=8.4, 6.8 Hz, 1H), 7.13-7.06 (m, 2H), 6.96 (t, J=5.7 Hz, 1H), 6.95-6.89 (m, 2H), 6.88 (d, J=8.5 Hz, 1H), 5.08 (dd, J=12.9, 5.5 Hz, 1H), 4.86 (qd, J=13.3, 2.5 Hz, 2H), 4.35 (d, J=5.6 Hz, 2H), 4.23-4.19 (m, 2H), 4.10-4.05 (m, 1H), 3.98 (d, J=5.8 Hz, 2H), 3.88 (td, J=9.0, 5.0 Hz, 1H), 3.80 (s, 4H), 3.60 (td, J=9.9, 5.1 Hz, 1H), 3.30 (s, 2H), 3.22 (d, J=5.9 Hz, 3H), 3.00 (h, J=6.5 Hz, 1H), 2.93-2.86 (m, 2H), 2.74 (d, J=0.6 Hz, 1H), 2.71 (s, 1H), 2.63-2.53 (m, 2H), 2.46-2.41 (m, 2H), 2.22 (qq, J=13.8, 6.8 Hz, 2H), 2.03 (dtd, J=12.8, 5.2, 2.3 Hz, 1H), 1.80-1.74 (m, 1H), 1.71-1.68 (m, 2H), 1.45 (d, J=12.5 Hz, 2H), 1.16 (dd, J=14.3, 10.8 Hz, 2H), ¹³C NMR (176 MHz, DMSO) δ 180.70, 173.27, 172.02, 170.50, 169.16, 169.05, 167.78, 164.83, 163.43, 158.41, 158.35, 155.03, 153.11, 151.71, 151.11, 148.17, 146.29, 144.91, 138.60, 138.58, 136.65, 135.21, 135.12, 134.77, 134.62, 132.52, 132.09, 132.03, 123.73, 119.24, 118.49, 117.93, 114.14, 113.90, 113.86, 111.45, 110.38, 107.67, 107.55, 100.70, 100.55, 57.38, 56.70, 54.82, 54.05, 53.73, 51.96, 49.35, 49.20, 49.05, 45.61, 38.96, 38.94, 36.82, 36.25, 34.79, 33.97, 33.94, 31.45, 29.14, 26.52, 26.50, 22.64. HRMS m/z 1050.38274 [M+H]⁺ (calcd. for C₅₀H₅₄F₂N₁₃O₈S, 1050.385071).

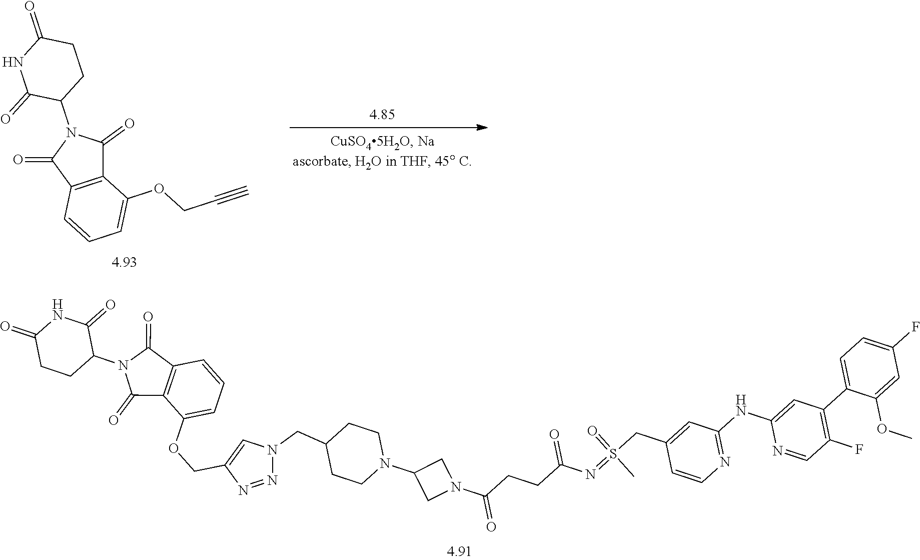 [SMILES: C#CCOc1cccc2c1C(=O)N(C1CCC(=O)NC1=O)C2=O ; COc1cc(F)ccc1-c1cc(Nc2cc(CS(C)(=O)=NC(=O)CCC(=O)N3CC(N4CCC(Cn5cc(COc6cccc7c6C(=O)N(C6CCC(=O)NC6=O)C7=O)nn5)CC4)C3)ccn2)ncc1F]

### 4-[3-(4-{[4-({[2-(2,6-dioxopiperidin-3-yl)-1,3-dioxo-2,3-dihydro-1H-isoindol-4-yloxy}methyl)-1H-1,2,3-triazol-1-yl]methyl}piperidin-1-yl) azetidin-1-yl]-N-{[(2-{[5-fluoro-4-(4-fluoro-2-methoxyphenyl)pyridin-2-yl]amino}pyridin-4-yl)methyl](methyl)oxo-λ⁶-sulfanylidene}-4-oxobutanamide (4.91)

Sodium ascorbate (2.6 mg, 0.01 mmol), CuSO₄·5H₂O (3.3 mg, 0.01 mmol), 4˜ {3-[4-(azidomethyl) piperidin-1-yl]azetidin-1-yl}-N-{[(2-{[5-fluoro-4-(4-fluoro-2-methoxyphenyl)pyridin-2-yl]amino}pyridin-4-yl)methyl](methyl)oxo-λ⁶-sulfanylidene}-4-oxobutanamide 4.85 (18 mg, 0.03 mmol) and 2-(2,6-dioxopiperidin-3-yl)-4-(prop-2-yn-1-yloxy)-2,3-dihydro-1H-isoindole-1,3-dione 4.93 (8.2 mg, 0.03 mmol) were reacted according to general procedure B to provide 4.91 (13 mg, 50% yield) as a white powder, 1H NMR (700 MHZ, DMSO) δ 11.09 (s, 1H), 9.90 (d, J=2.7 Hz, 1H), 8.31 (s, 1H), 8.26-8.20 (m, 3H), 7.86-7.81 (m, 2H), 7.73 (d, J=8.6 Hz, 1H), 7.62-7.58 (m, 1H), 7.48 (d, J=7.3 Hz, 1H), 7.35 (dd, J=8.4, 6.7 Hz, 1H), 7.10 (dd, J=11.4, 2.5 Hz, 1H), 6.95-6.89 (m, 2H), 5.42 (s, 2H), 5.07 (dd, J=12.9, 5.5 Hz, 1H), 4.86 (qd, J=13.4, 2.9 Hz, 2H), 4.28 (dd, J=7.1, 1.9 Hz, 2H), 4.06 (td, J=8.1, 2.7 Hz, 1H), 3.88 (td, J=9.1, 5.0 Hz, 1H), 3.80 (s, 4H), 3.60 (td, J=10.1, 5.1 Hz, 1H), 3.31 (d, J=13.4 Hz, 4H), 3.22 (d, J=6.0 Hz, 3H), 3.00 (h, J=6.8 Hz, 1H), 2.88 (ddd, J=17.2, 14.0, 5.4 Hz, 1H), 2.70 (s, 1H), 2.61-2.56 (m, 1H), 2.46-2.38 (m, 2H), 2.22 (qq, J=13.7, 6.8 Hz, 2H), 2.02 (dtd, J=12.9, 5.3, 2.4 Hz, 1H), 1.85-1.79 (m, 1H), 1.72-1.70 (m, 1H), 1.45 (d, J=12.4 Hz, 2H), 1.18 (q, J=13.7 Hz, 2H). ¹³C NMR (176 MHz, DMSO) δ 180.70, 173.24, 172.04, 170.36, 167.23, 165.71, 164.82, 163.43, 158.41, 158.35, 155.71, 155.02, 153.11, 151.71, 151.10, 148.17, 142.23, 138.60, 138.57, 137.41, 135.21, 135.12, 134.77, 134.62, 133.76, 132.09, 132.03, 125.71, 120.93, 119.24, 118.50, 117.09, 116.17, 114.13, 113.90, 113.86, 107.68, 107.55, 100.69, 100.54, 62.77, 57.39, 56.70, 54.95, 54.04, 53.72, 51.96, 49.32, 49.23, 49.17, 38.97, 38.95, 36.78, 33.98, 33.95, 31.40, 29.11, 26.53, 26.51, 22.45. HRMS m/z 994.34597 [M+H]⁺ (calcd. for C₄₈H₅₀F₂N₁₁O₉S, 994.34763).

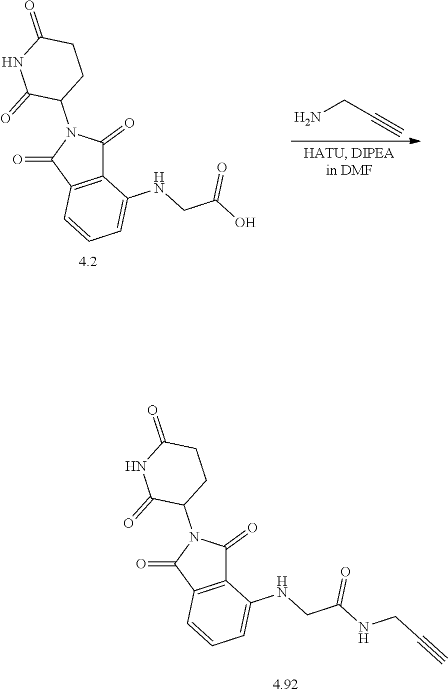

### 2-((2-(2,6-dioxopiperidin-3-yl)-1,3-dioxoisoindolin-4-yl)amino)-N-(prop-2-yn-1-yl) acetamide (4.92)

Propargyl amine (17 mg, 0.17 mmol), (2-(2,6-dioxopiperidin-3-yl)-1,3-dioxoisoindolin-4-yl)glycine 4.2 (100 mg, 0.30 mmol), HATU (138 mg, 0.36 mmol) and DIPEA (78 mg, 0.60 mmol) were dissolved in DMF (0.2 M). The mixture was stirred overnight at room temperature. The reaction mixture was concentrated under reduced pressure and purified by reversed phase chromatography (C18, H₂O:ACN eluent) to provide 4.92 (15 mg, 13% yield) as a green powder. ¹H NMR (700 MHZ, DMSO) δ 11.10 (s, 1H), 8.55 (t, J=5.6 Hz, 1H), 7.60 (dd, J=8.5, 7.1 Hz, 1H), 7.08 (d, J=7.1 Hz, 1H), 6.95 (t, J=5.8 Hz, 1H), 6.87 (d, J=8.5 Hz, 1H), 5.08 (dd, J=12.9, 5.5 Hz, 1H), 3.97 (d, J=5.8 Hz, 2H), 3.90 (dd, J=5.6, 2.5 Hz, 2H), 3.11 (t, J=2.5 Hz, 1H), 2.94-2.86 (m, 1H), 2.63-2.51 (m, 2H), 2.03 (dt, J=8.6, 2.8 Hz, 1H). ¹³C NMR (176 MHz, DMSO) δ 173.27, 170.50, 169.15, 169.03, 167.78, 146.30, 136.66, 132.52, 117.90, 111.49, 110.40, 81.38, 73.56, 49.04, 45.53, 31.45, 31.16, 28.48, 22.63. HRMS m/z 369.11863 [M+H]⁺ (calcd. for C₁₈H₁₇N₄O₅, 369.11935).

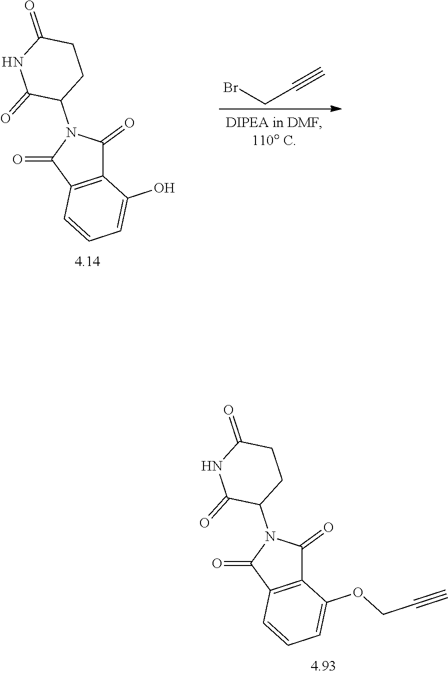

### 2-(2,6-dioxopiperidin-3-yl)-4-(prop-2-yn-1-yloxy)-2,3-dihydro-1H-isoindole-1,3-dione (4.93)

4-hydroxy-thalidomide 4.14 (300 mg, 1.1 mmol) was dissolved in DMF (0.1M) with DIPEA (424 mg, 3.3 mmol) and heated at 110° C., under argon and stirred for 5 minutes before stirring in propargyl bromide (145 mg, 1.2 mmol). The reaction was allowed to proceed overnight. The reaction mixture was concentrated under reduced pressure, then purified by reversed phase chromatography (C18, 10-100% ACN in water) to provide 4.93 as a white powder (200 mg, 59% yield). ¹H NMR (400 MHz, DMSO) & 11.09 (s, 1H), 7.85 (dd, J=8.5, 7.3 Hz, 1H), 7.56 (d, J=8.5 Hz, 1H), 7.50 (d, J=7.2 Hz, 1H), 5.13-5.04 (m, 3H), 3.64 (t, J=2.4 Hz, 1H), 2.89 (ddd, J=17.0, 13.8, 5.2 Hz, 1H), 2.65-2.51 (m, 2H), 2.10-1.98 (m, 1H), ¹³C NMR (101 MHz, DMSO) δ 173.16, 170.29, 167.13, 165.61, 154.76, 137.24, 133.82, 120.67, 117.35, 116.47, 79.78, 79.61, 56.94, 49.27, 40.69, 40.48, 31.42, 22.46. HRMS m/z 313.08079 [M+H]⁺ (calcd. for C₁₆H₁₃N₂O₅, 313.08190),

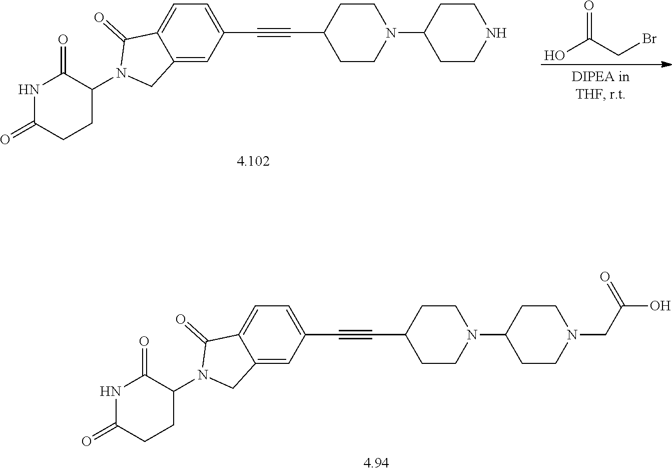 [SMILES: O=C1CCC(N2Cc3cc(C#CC4CCN(C5CCNCC5)CC4)ccc3C2=O)C(=O)N1 ; O=C(O)CBr ; O=C(O)CN1CCC(N2CCC(C#Cc3ccc4c(c3)CN(C3CCC(=O)NC3=O)C4=O)CC2)CC1]

### 2-(4-{2-[2-(2,6-dioxopiperidin-3-yl)-1-oxo-2,3-dihydro-1H-isoindol-5-yl]ethynyl}-[1,4′-bipiperidin]-1′-yl) acetic acid (4,94)

3-[5-(2-{[1,4′-bipiperidin]-4-yl}ethynyl)-1-oxo-2,3-dihydro-1H-isoindol-2-yl]piperidine-2,6-dione 4.102 (100 mg, 0.23 mmol), 2-bromoacetic acid (38 mg, 0.28 mmol) and DIPEA (89 mg, 0.69 mmol) were dissolved in THF (0.1 M). The mixture was stirred overnight at room temperature under argon protection. The reaction mixture was concentrated under reduced pressure and purified by reversed phase chromatography (C18, H₂O:ACN eluent) to provide 4.94 (52 mg, 67% yield) as a white powder. ¹H NMR (700 MHz, DMSO) δ 10.99 (s, 1H), 7.69 (d, J=7.8 Hz, 1H), 7.64-7.62 (m, 1H), 7.51 (ddd, J=7.9, 3.4, 1.3 Hz, 1H), 5.11 (dd, J=13.3, 5.2 Hz, 1H), 4.45 (d, J=17.5 Hz, 1H), 4.33 (d, J=17.4 Hz, 1H), 3.19 (s, 2H), 2.91 (ddt, J=17.3, 9.0, 4.8 Hz, 2H), 2.79 (d, J=8.9 Hz, 1H), 2.70 (s, 1H), 2.63-2.57 (m, 1H), 2.54 (dd, J=12.1, 2.6 Hz, 3H), 2.41-2.33 (m, 4H), 2.17 (td, J=11.5, 2.6 Hz, 1H), 2.02 (ddq, J=10.4, 5.4, 2.7 Hz, 1H), 1.90 (qd, J=9.3, 5.9 Hz, 2H), 1.81-1.74 (m, 2H), 1.63 (qd, J=12.5, 3.9 Hz, 4H), 1.51-1.44 (m, 1H). ¹³C NMR (176 MHz, DMSO) δ 173.32, 171.41, 170.21, 169.53, 168.50, 167.90, 142.87, 131.62, 131.44, 126.87, 126.84, 126.75, 126.72, 123.62, 96.20, 81.36, 61.90, 61.46, 60.29, 59.32, 58.84, 52.22, 52.15, 49.07, 47.95, 47.50, 32.10, 31.67, 27.77, 26.41, 22.90.

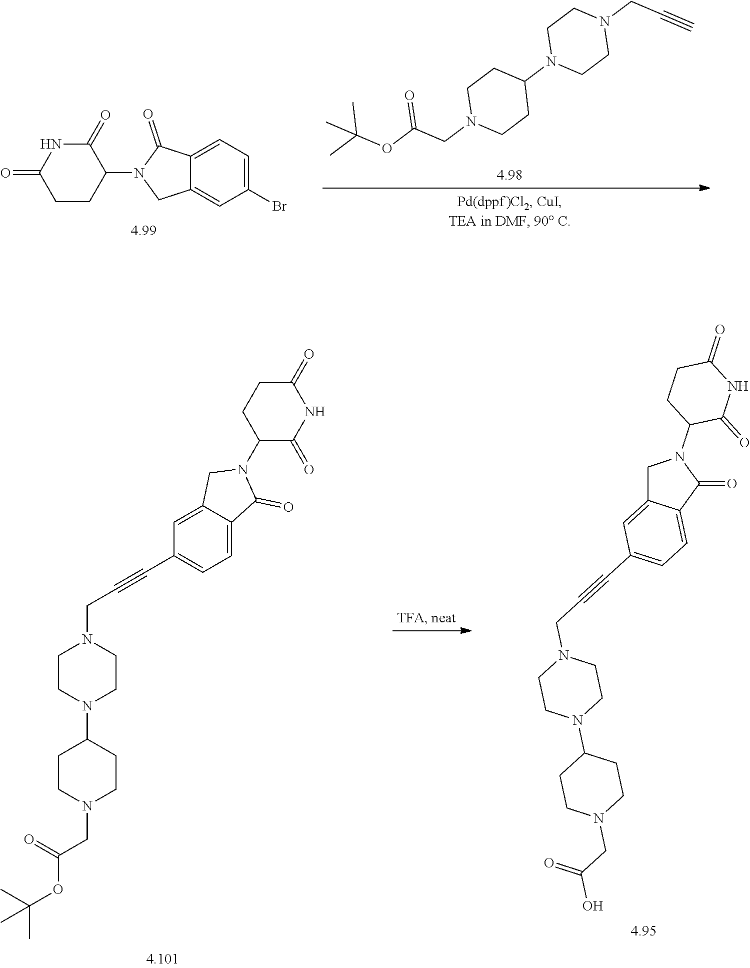 [SMILES: O=C1CCC(N2Cc3cc(Br)ccc3C2=O)C(=O)N1 ; C#CCN1CCN(C2CCN(CC(=O)OC(C)(C)C)CC2)CC1 ; CC(C)(C)OC(=O)CN1CCC(N2CCN(CC#Cc3ccc4c(c3)CN(C3CCC(=O)NC3=O)C4=O)CC2)CC1 ; O=C(O)CN1CCC(N2CCN(CC#Cc3ccc4c(c3)CN(C3CCC(=O)NC3=O)C4=O)CC2)CC1]

### Tert-butyl 2-[4-(4-{3-[2-(2,6-dioxopiperidin-3-yl)-1-oxo-2,3-dihydro-1H-isoindol-5-yl]prop-2-yn-1-yl}piperazin-1-yl) piperidin-1-yl]acetic acid (4.95)

3-(5-bromo-1-oxo-2,3-dihydro-1H-isoindol-2-yl) piperidine-2,6-dione (4.99) (100 mg, 0.31 mmol), tert-butyl 2-{4-[4-(prop-2-yn-1-yl) piperazin-1-yl]piperidin-1-yl}acetate (4.98) (100 mg, 0.31 mmol), [1,1-bis(diphenylphosphino) ferrocene]dichloropalladium (II) (22 mg, 0.03 mmol), diiodocopper (30 mg, 0.09 mmol) and DIPEA (120 mg, 0.93 mmol) were dissolved in DMF (0.2 M) and heated at 80° C. in a sealed tube under argon overnight. The reaction mixture was concentrated under reduced pressure and the crude mixture was purified by reversed phase chromatography (C18, 0-100% ACN in water) to afford 4.101 as a brown crystalline solid (50 mg, 29% yield). ¹H NMR (700 MHZ, DMSO) δ 10.99 (s, 1H), 7.73-7.67 (m, 2H), 7.56 (dd, J=7.9, 1.4 Hz, 1H), 5.12 (dd, J=13.3, 5.2 Hz, 1H), 4.46 (d, J=17.4 Hz, 1H), 4.37-4.30 (m, 1H), 4.09 (q, J=5.3 Hz, 1H), 3.54 (s, 2H), 3.45 (qd, J=7.0, 5.1 Hz, 1H), 3.18 (d, J=5.2 Hz, 2H), 3.05 (s, 2H), 2.95-2.87 (m, 1H), 2.84 (d, J=10.7 Hz, 2H), 2.64-2.58 (m, 2H), 2.54 (s, 1H), 2.38 (dd, J=13.3, 4.5 Hz, 1H), 2.16-2.07 (m, 3H), 2.02 (dtd, J=12.6, 5.3, 2.3 Hz, 1H), 1.70 (d, J=11.7 Hz, 2H), 1.41 (s, 11H), 1.06 (t, J=7.0 Hz, 1H). ¹³C NMR (176 MHz, DMSO) δ 173.31, 171.39, 169.93, 167.84, 142.91, 131.75, 131.71, 127.01, 126.17, 123.69, 88.51, 84.86, 80.47, 61.29, 59.97, 52.38, 52.31, 52.16, 49.07, 49.00, 47.53, 47.22, 31.67, 28.43, 28.29, 22.90. HRMS m/z 564.31750 [M+H]⁺ (calcd. for C₃₁H₄₂N₅O₅, 564.31805).

Tert-butyl 2-[4-(4-{3-[2-(2,6-dioxopiperidin-3-yl)-1-oxo-2,3-dihydro-1H-isoindol-5-yl]prop-2-yn-1-yl}piperazin-1-yl) piperidin-1-yl]acetate 4.101 was dissolved in TFA (1.6 M) and stirred at room temperature until completion as determined by LCMS. The solution was concentrated under reduced pressure and purified by reversed phase chromatography (C18, H₂O:ACN) to afford 4.95 in quantitative yield.

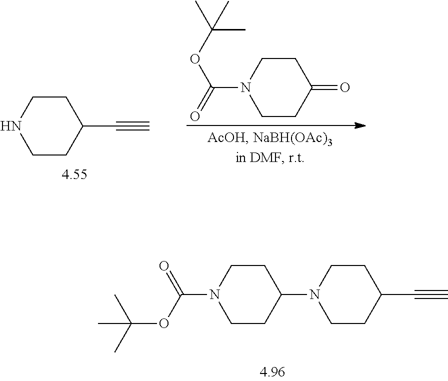

### Tert-butyl 4-ethynyl-[1,4′-bipiperidine]-1′-carboxylate (4.96)

4-(azidomethyl) piperidine 4.55 (750 mg, 6.9 mmol) was dissolved in DMF (0.4 M) with tert-butyl 4-oxopiperidine-1-carboxylate (1.4 g, 6.9 mmol) and 17 N acetic acid (8.7 M) and stirred at room temperature for 10 minutes. Sodium bis(acetyloxy) boranuidyl acetate (2.9 g, 14 mmol) was then added under inert atmosphere and the reaction mixture was stirred at room temperature overnight. The reaction was quenched with a saturated solution of sodium carbonate, concentrated under reduced pressure and purified by reversed phase chromatography (C18, H₂O:ACN eluent) to provide 4.96 (663 mg, 33% yield) as a clear colorless oil. ¹H NMR (400 MHZ, CDCl₃) δ 4.13 (d, J=12.9 Hz, 2H), 2.78 (ddd, J=10.6, 5.9, 3.5 Hz, 2H), 2.68 (t, J=12.8 Hz, 2H), 2.45-2.27 (m, 4H), 2.07 (s, 1H), 1.86 (dp, J=13.4, 3.6 Hz, 2H), 1.81-1.73 (m, 2H), 1.66 (dtt, J=17.4, 11.9, 5.9 Hz, 2H), 1.45 (s, 9H), 1.40 (td, J=12.1, 4.4 Hz, 2H). ¹³C NMR (101 MHz, CDCl₃) δ 154.69, 87.37, 79.35, 77.28, 68.86, 62.34, 47.78, 31.99, 28.42, 28.03, 27.06. HRMS m/z 293.22173 [M+H]+ (calcd. for C₁₇H₂₉N₂O₂, 293.22235).

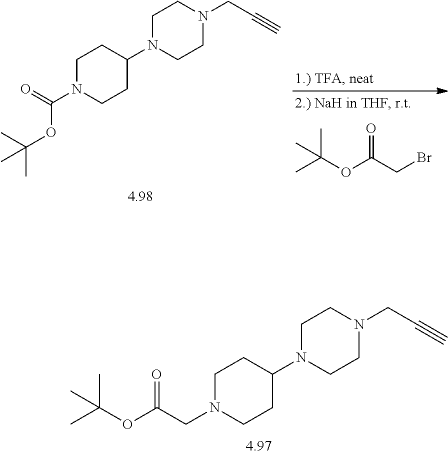

### Tert-butyl 2-(4-(4-(prop-2-yn-1-yl) piperazin-1-yl) piperidin-1-yl) acetate (4.97)

Tert-butyl 2-{4-[4-(prop-2-yn-1-yl) piperazin-1-yl]piperidin-1-yl}acetate 4.98 (2.2 g, 7.1 mmol) was dissolved in TFA (1.6 M) and stirred at room temperature until completion as determined by LCMS. The solution was concentrated under reduced pressure to afford 3-[5-(2˜ {[1,4′-bipiperidin]-4-yl}ethyl)-1-oxo-2,3-dihydro-1H-isoindol-2-yl]piperidine-2,6-dione which was taken forward without further purification.

1-(piperidin-4-yl)-4-(prop-2-yn-1-yl) piperazine (400 mg, 1.9 mmol) was dissolved in THE (0.1 M) and sodium hydride (366 mg, 15.3 mmol) was added under ice cooling. Tert-butyl bromoacetate (376 mg, 1.9 mmol) was then added and the mixture was stirred at room temperature overnight. The reaction was quenched with a saturated solution of ammonium chloride and extracted with 5% EtOH in chloroform three times. The combined organic layers were dried over Na₂SO₄ and concentrated under reduced pressure. The crude mixture was purified by normal phase chromatography (SiO₂, DCM:MeOH) to afford 4.97 in quantitative yield as a clear colorless gel. ¹H NMR (400 MHZ, CDCl₃) δ 3.32 (d, J=2.5 Hz, 2H), 3.12 (s, 2H), 3.09-3.01 (m, 2H), 2.87 (t, J=4.6 Hz, 4H), 2.73 (s, 1H), 2.32-2.20 (m, 3H), 1.99 (s, 2H), 1.78 (qd, J=12.0, 3.9 Hz, 2H), 1.46 (s, 8H), ¹³C NMR (101 MHz, CDCl₃) δ 169.55, 81.25, 78.22, 77.25, 73.71, 62.56, 59.56, 52.25, 50.43, 48.35, 46.55, 28.14, 26.78. HRMS m/z 322.24856 [M+H]⁺ (calcd. for C₁₈H₃₂N₃O₂, 322.24890).

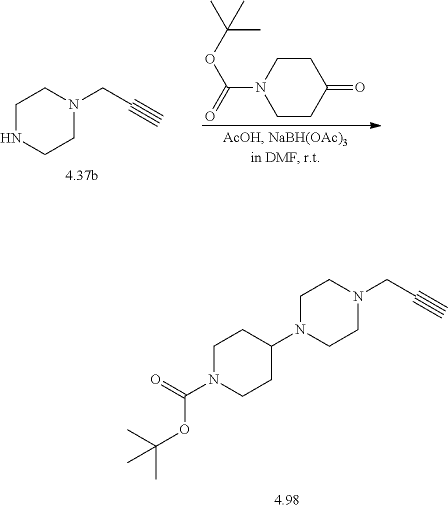

### Tert-butyl 4-(4-(prop-2-yn-1-yl) piperazin-1-yl) piperidine-1-carboxylate (4,98)

1-(prop-2-yn-1-yl) piperazine 4.37b (1.1 g, 8.9 mmol) was dissolved in DMF (0.4 M) with tert-butyl 4-oxopiperidine-1-carboxylate (1.8 g, 8.9 mmol) and 17 N acetic acid (8.7 M) and stirred at room temperature for 10 minutes. Sodium bis(acetyloxy) boranuidyl acetate (3.8 g, 18 mmol) was then added under inert atmosphere and the reaction mixture was stirred at room temperature overnight. The reaction was quenched with a saturated solution of sodium carbonate, concentrated under reduced pressure and purified by reversed phase chromatography (C18, H₂O:ACN eluent) to provide 4.98 (2.3 g, 84% yield) as a clear colorless oil. ¹H NMR (400 MHZ, CDCl₃) δ 4.14 (d, J=12.6 Hz, 2H), 3.46 (d, J=2.2 Hz, 1H), 3.29 (d, J=2.3 Hz, 2H), 2.72 (d, J=12.9 Hz, 2H), 2.63 (s, 7H), 2.41 (dddd, J=14.9, 11.4, 8.1, 5.2 Hz, 1H), 2.25 (t, J=2.5 Hz, 1H), 1.81 (d, J=12.6 Hz, 2H), 1.45 (s, 10H), 1.38 (dd, J=12.3, 4.4 Hz, 1H), ¹³C NMR (101 MHz, CDCl₃) δ 154.67, 79.43, 78.74, 77.26, 73.22, 61.85, 52.13, 48.81, 46.77, 28.43, 28.17. HRMS m/z 308.23877 [M+H]⁺ (calcd. for C₁₇H₃₀N₃O₂, 308.23325).

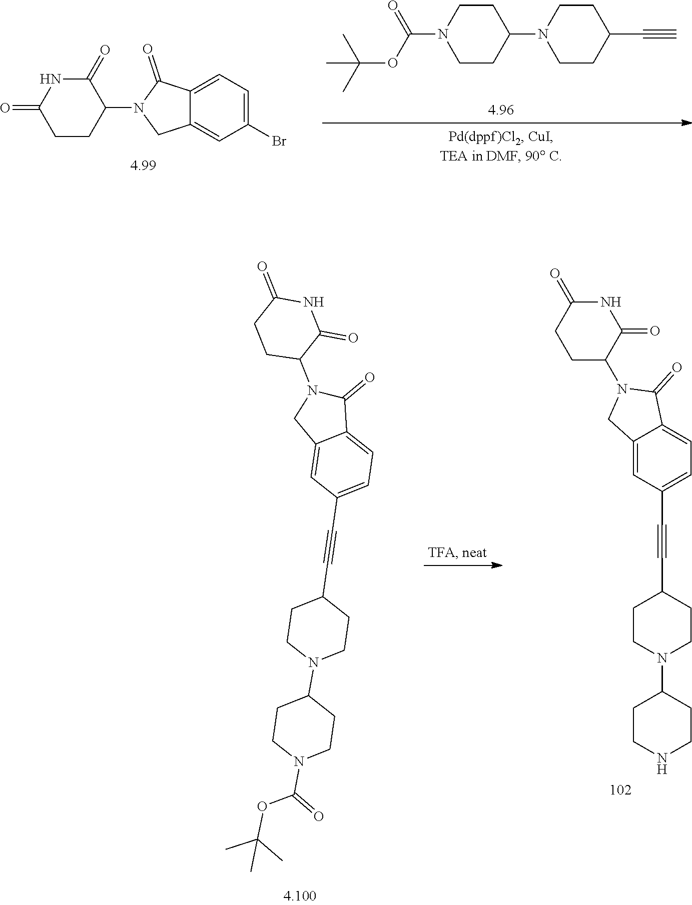 [SMILES: O=C1CCC(N2Cc3cc(Br)ccc3C2=O)C(=O)N1 ; C#CC1CCN(C2CCN(C(=O)OC(C)(C)C)CC2)CC1 ; CC(C)(C)OC(=O)N1CCC(N2CCC(C#Cc3ccc4c(c3)CN(C3CCC(=O)NC3=O)C4=O)CC2)CC1 ; O=C1CCC(N2Cc3cc(C#CC4CCN(C5CCNCC5)CC4)ccc3C2=O)C(=O)N1]

### 3-[5-(2-{[1,4′-bipiperidin]-4-yl}ethynyl)-1-oxo-2,3-dihydro-1H-isoindol-2-yl]piperidine-2,6-dione (4.102)

3-(5-bromo-1-oxo-2,3-dihydro-1H-isoindol-2-yl) piperidine-2,6-dione (4.99) (300 mg, 1.0 mmol), tert-butyl 4-ethynyl-[1,4′-bipiperidine]-1′-carboxylate (4.96) (299 mg, 1.0 mmol), [1,1′-bis(diphenylphosphino) ferrocene]dichloropalladium (II) (72 mg, 0.10 mmol), diiodocopper (97 mg, 0.03 mmol) and DIPEA (396 mg, 3.1 mmol) were dissolved in DMF (0.2 M) and heated at 90° C. under argon overnight. The reaction mixture was quenched with water and extracted with ethyl acetate. The combined organic layers were washed with brine, dried over Na₂SO₄ and filtered through celite. The crude mixture was concentrated under reduced pressure and purified by reversed phase chromatography (C18, 5-32% ACN in water) to afford tert-butyl 4-{2-[2-(2,6-dioxopiperidin-3-yl)-1-oxo-2,3-dihydro-1H-isoindol-5-yl]ethynyl}-[1,4′-bipiperidine]-1′-carboxylate 4.100 as an off-white solid (76 mg, 14% yield). ¹H NMR (400 MHZ, CDCl₃) δ 7.87-7.71 (m, 1H), 7.52-7.40 (m, 2H), 5.21 (dd, J=13.3, 5.1 Hz, 1H), 4.57-4.40 (m, 1H), 4.30 (d, J=16.1 Hz, 1H), 4.16 (s, 2H), 2.94-2.76 (m, 4H), 2.74-2.63 (m, 3H), 2.51-2.42 (m, 3H), 2.42-2.26 (m, 3H), 2.21 (ddd, J=13.0, 6.8, 3.0 Hz, 1H), 1.97 (dt, J=10.4, 3.8 Hz, 2H), 1.78 (tt, J=14.6, 6.4 Hz, 4H), 1.45 (s, 9H), 1.41 (dd, J=11.7, 4.4 Hz, 2H). ¹³C NMR (101 MHz, CDCl₃) δ 171.37, 169.71, 168.84, 154.73, 141.39, 131.79, 130.40, 127.85, 125.96, 123.92, 95.73, 81.06, 79.51, 77.26, 62.38, 51.90, 47.82, 46.83, 32.36, 31.84, 31.58, 28.45, 27.92, 23.39. HRMS m/z 535.29131 [M+H]⁺ (calcd. for C₃₀H₃₉N₄O₃, 535.29150).

Tert-butyl 4-{2-[2-(2,6-dioxopiperidin-3-yl)-1-oxo-2,3-dihydro-1H-isoindol-S-yl]ethynyl}-[1,4′-bipiperidine]-1′-carboxylate 4.100 (200 mg, 0.37 mmol) was dissolved in TFA (1.6 M) and stirred at room temperature until completion as determined by LCMS. The solution was concentrated under reduced pressure and purified by reversed phase chromatography (C18, H₂O:ACN) to afford 4.102 (104 mg, 64% yield) as a white solid.

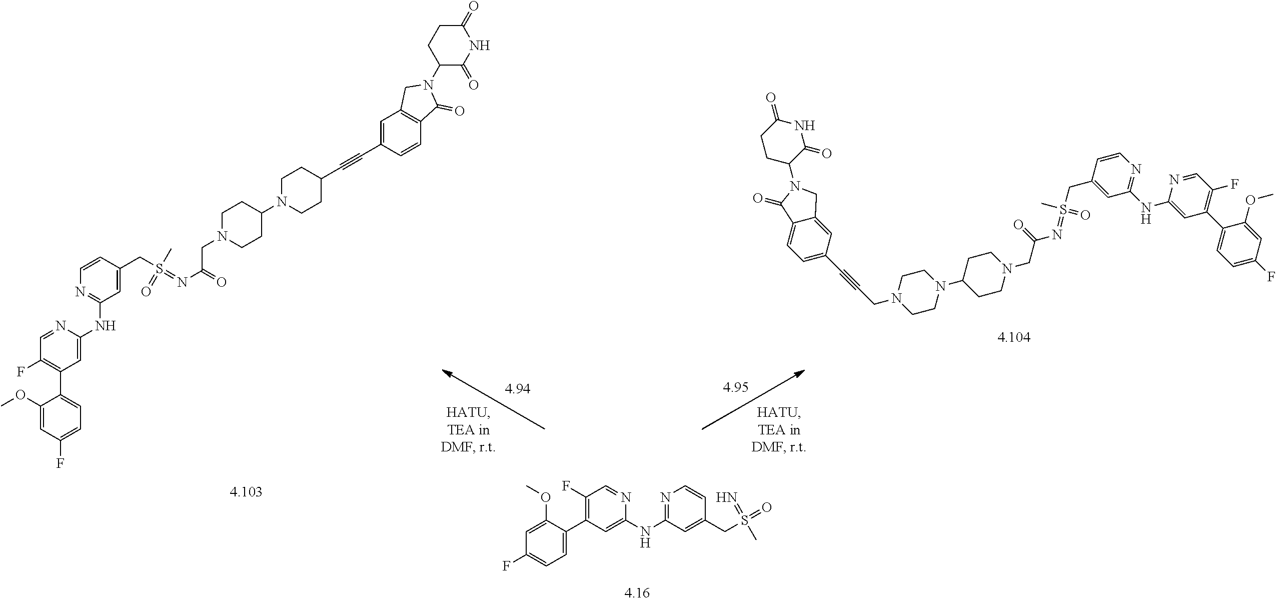 [SMILES: COc1cc(F)ccc1-c1cc(Nc2cc(CS(C)(=O)=NC(=O)CN3CCC(N4CCC(C#Cc5ccc6c(c5)CN(C5CCC(=O)NC5=O)C6=O)CC4)CC3)ccn2)ncc1F ; COc1cc(F)ccc1-c1cc(Nc2cc(CS(C)(=O)=NC(=O)CN3CCC(N4CCN(CC#Cc5ccc6c(c5)CN(C5CCC(=O)NC5=O)C6=O)CC4)CC3)ccn2)ncc1F ; COc1cc(F)ccc1-c1cc(Nc2cc(CS(C)(=N)=O)ccn2)ncc1F]

### 2-(4-{2-[2-(2,6-dioxopiperidin-3-yl)-1-oxo-2,3-dihydro-1H-isoindol-5-yl]ethynyl}-[1,4′-bipiperidin]-1′-yl)-N-{[(2-{[5-fluoro-4-(4-fluoro-2-methoxyphenyl)pyridin-2-yl]amino}pyridin-4-yl)methyl](methyl)oxo-26-sulfanylidene}acetamide (4.103)

2-(4-{2-[2-(2,6-dioxopiperidin-3-yl)-1-oxo-2,3-dihydro-1H-isoindol-5-yl]ethynyl}-[1,4′-bipiperidin]-1′-yl) acetic acid 4.94 (50 mg, 0.10 mmol), VIP152 4.16 (41 mg, 0.10 mmol), HATU (46 mg, 0.12 mmol) and triethylamine (20 mg, 0.19 mmol) were dissolved in THF (0.05 M). The mixture was stirred overnight at room temperature. The reaction mixture was concentrated under reduced pressure and purified by reversed phase chromatography (C18, H₂O:ACN eluent) to provide 4.103 (28 mg, 31% yield) as a white powder. ¹H NMR (700 MHz, CDCl₃) δ 8.30-8.26 (m, 1H), 8.20-8.16 (m, 1H), 7.82 (d, J=7.9 Hz, 1H), 7.76 (dd, J=14.7, 6.7 Hz, 1H), 7.56 (s, 1H), 7.54-7.49 (m, 2H), 7.46 (td, J=9.0, 5.0 Hz, 1H), 7.32-7.29 (m, 2H), 6.92 (ddd, J=4.8, 2.8, 1.5 Hz, 1H), 6.82-6.74 (m, 2H), 5.24 (ddd, J=13.4, 5.1, 3.5 Hz, 1H), 4.76 (d, J=13.6 Hz, 1H), 4.67 (d, J=13.7 Hz, 1H), 4.51 (dd, J=16.0, 4.3 Hz, 1H), 4.34 (d, J=15.9 Hz, 1H), 3.85 (s, 3H), 3.25 (t, J=1.7 Hz, 2H), 3.15 (d, J=1.5 Hz, 3H), 3.12-3.04 (m, 2H), 2.99-2.82 (m, 4H), 2.65 (s, 1H), 2.47-2.32 (m, 4H), 2.25 (dtd, J=12.9, 5.3, 2.4 Hz, 1H), 2.15 (d, J=11.4 Hz, 2H), 1.96 (s, 2H), 1.77 (s, 6H). ¹³C NMR (176 MHz, CDCl₃) δ 179.37, 171.06, 169.57, 169.54, 168.81, 165.03, 163.62, 158.17, 158.11, 154.57, 153.67, 152.26, 149.64, 148.71, 141.36, 137.42, 137.39, 135.88, 135.79, 134.87, 134.71, 131.80, 131.78, 131.72, 130.34, 128.01, 126.00, 123.95, 118.58, 117.88, 113.95, 112.91, 112.88, 107.43, 107.31, 99.76, 99.61, 63.81, 62.30, 58.92, 58.89, 55.98, 53.54, 51.93, 51.91, 47.80, 46.84, 46.82, 38.64, 32.12, 31.59, 27.59, 23.45. HRMS m/z 879.34407 [M+H]⁺ (calcd. for C46H₄₉F₂N₈O₆S 879.34583).

### 2-[4-(4-{3-[2-(2,6-dioxopiperidin-3-yl)-1-oxo-2,3-dihydro-1H-isoindol-5-yl]prop-2-yn-1-yl}piperazin-1-yl) piperidin-1-yl]-N-{[(2-{[5-fluoro-4-(4-fluoro-2-methoxyphenyl)pyridin-2-yl]amino}pyridin-4-yl)methyl](methyl)oxo-16-sulfanylidene}acetamide (4.104)

2-[4-(4-{3-[2-(2,6-dioxopiperidin-3-yl)-1-oxo-2,3-dihydro-1H-isoindol-5-yl]prop-2-yn-1-yl}piperazin-1-yl) piperidin-1-yl]acetic acid 4.95 (30 mg, 0.06 mmol), VIP152 4.16 (24 mg, 0.06 mmol), HATU (29 mg, 0.08 mmol) and triethylamine (17 mg, 0.17 mmol) were dissolved in DMF (0.05 M). The mixture was stirred overnight at room temperature. The reaction mixture was concentrated under reduced pressure and purified by reversed phase chromatography (C18, H₂O:ACN eluent) to provide 4.104 (15 mg, 28% yield) as an off-white solid. ¹H NMR (700 MHz, DMSO) δ 10.99 (s, 1H), 9.90 (s, 1H), 8.25-8.19 (m, 2H), 7.75-7.69 (m, 3H), 7.69-7.63 (m, 1H), 7.55 (dd, J=7.8, 1.4 Hz, 1H), 7.35 (dd, J=8.4, 6.7 Hz, 1H), 7.10 (dd, J=11.4, 2.5 Hz, 1H), 6.95-6.89 (m, 2H), 5.12 (dd, J=13.3, 5.1 Hz, 1H), 4.87 (q, J=13.5 Hz, 2H), 4.45 (d, J=17.4 Hz, 1H), 4.34 (d, J=17.3 Hz, 1H), 3.81 (s, 3H), 3.52 (d, J=3.9 Hz, 2H), 3.28 (s, 3H), 2.99 (s, 2H), 2.96-2.86 (m, 1H), 2.83-2.79 (m, 2H), 2.76 (s, 1H), 2.64-2.56 (m, 2H), 2.50-2.32 (m, 7H), 2.08-1.99 (m, 4H), 1.65-1.60 (m, 2H), 1.34 (q, J=11.9 Hz, 2H). ¹³C NMR (176 MHz, DMSO) δ 178.68, 173.33, 171.41, 167.84, 164.82, 163.43, 158.41, 158.35, 154.99, 153.12, 151.72, 151.16, 148.13, 142.89, 138.49, 135.26, 135.17, 134.66, 134.51, 132.07, 132.02, 131.75, 131.70, 126.99, 12617, 123.67, 119.19, 118.47, 114.17, 113.89, 107.66, 107.54, 100.69, 100.54, 88.50, 84.84, 79.65, 63.86, 61.45, 57.89, 56.70, 52.69, 52.31, 52.16, 48.97, 47.52, 47.23, 41.37, 39.27, 31.67, 28.41, 22.90. HRMS m/z 894.35520 [M+H]⁺ (calcd. for C₄₆H₅₀F₂N₉O₆S 894.35673).

The compounds, compositions, and methods of the appended claims are not limited in scope by the specific compounds, compositions, and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A compound defined by Formula I below 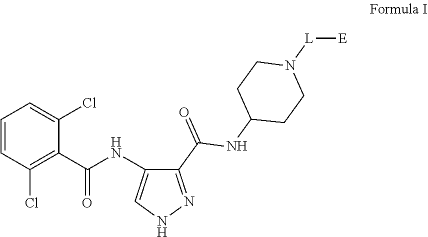
or a pharmaceutically acceptable salt, solvate, hydrate, cocrystal, or prodrug thereof,
wherein
L comprises one or more linking groups selected from optionally substituted —C₁₋₁₀ alkylene-, —O—C1-10 alkylene-, —O—C1-10 alkenylene-, —O—C1-10 alkenylene-, —C1-10 alkynylene-, —O—C₁₋₁₀ alkynylene-, -arylene-, -heteroarylene-, -cycloalkylene-, -heterocycloalkylene-, —O—, —S—, —S—S—, —S(O)_{W}—, —C(O)—, —C(O)O—, —O(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl; and
E comprises an E3 ubiquitin ligase ligand moiety.

2. The compound of claim 1, wherein L comprises one or more linking groups independently selected from optionally substituted —C₁₋₁₀ alkynylene-, —O—C₁₋₁₀ alkynylene-, -arylene-, -heteroarylene-, -cycloalkylene-, and -heterocycloalkylene-.

3. The compound of any of claims 1-2, wherein L comprises at least two linking groups independently selected from optionally substituted —C1-10 alkynylene-, -heteroarylene-, and -heterocycloalkylene-.

4. The compound of any of claims 1-3, wherein L comprises at least two heterocycloalkylene groups.

5. The compound of any of claims 1-4, wherein L comprises at least one piperidine moiety.

6. The compound of any of claims 1-5, wherein L comprises at least one piperazine moiety.

7. The compound of any of claims 1-6, wherein L comprises at least one triazole moiety, such as a 1,2,3-triazole moiety.

8. The compound of any of claims 1-7, wherein L comprises at least one alkynyl moiety.

9. The compound of any of claims 1-8, wherein L comprises at least one azetidine moiety.

10. The compound of any of claims 1-9, wherein L comprises at least two moieties independently selected from a piperidine moiety, a piperazine moiety, a triazole moiety, such as a 1,2,3-triazole moiety, an alkynyl moiety, and an azetidine moiety.

11. The compound of any of claims 1-10, wherein when L comprises a piperidine moiety, L further comprises at least one additional linking group selected from optionally substituted —C1-10 alkynylene-, —O—C₁₋₁₀ alkynylene-, -arylene-, -heteroarylene-, -cycloalkylene-, and -heterocycloalkylene.

12. The compound of any of claims 1-12, wherein L has a length of at least 8 atoms or at least 10 atoms, such as a length of from 8 atoms to 25 atoms, from 10 atoms to 25 atoms, from 8 atoms to 20 atoms, or from 10 atoms to 20 atoms.

13. The compound of any of claims 1-12, wherein L is not defined by the structure below

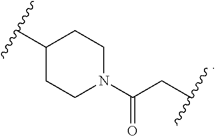 [SMILES: *CC(=O)N1CCC(*)CC1]

14. The compound of any of claims 1-13, wherein the E3 ubiquitin ligase ligand moiety comprises a Cereblon (CRBN) ligand, a mouse double minute 2 (MDM2) ligand, a Von Hippel-Lindau (VHL) ligand, or any substructure thereof.

15. The compound of claim 14, wherein the E3 ubiquitin ligase ligand comprises a CRBN ligand selected from thalidomide, lenalidomide, pomalidomide, and any substructure thereof.

16. The compound of claim 14, wherein the E3 ubiquitin ligase ligand comprises an MDM2 ligand selected from idasanutlin, RG7112, RG7388, MI 773/SAR 405838, AMG 232, DS-3032b, RO6839921, RO5045337, RO5503781, CGM-097, MK-8242, and any substructure thereof.

17. The compound of claim 14, wherein the E3 ubiquitin ligase ligand comprises a VHL ligand selected from VHL ligand 1 (VHL-1), VHL ligand 2 (VHL-2), VH032, and any substructure thereof.

18. The compound of any of claims 1-17, wherein the E3 ubiquitin ligase ligand moiety is selected from thalidomide, lenalidomide, idasanutlin, NIL ligand 1 (VHL-1), and any substructure thereof.

19. The compound of any of claims 1-18, wherein the E3 ubiquitin ligase ligand moiety is selected from 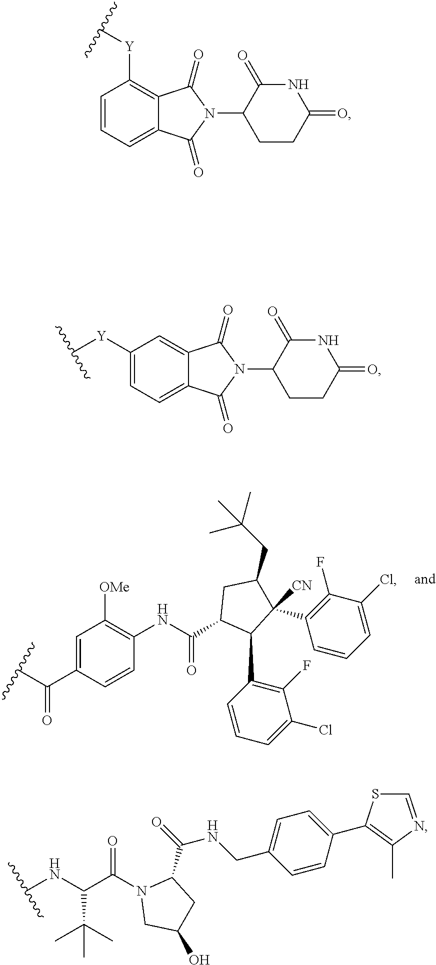
wherein Y is selected from —N(R)—, —N(H)—, and —O— wherein R is optionally substituted C₁₋₆ alkyl.

20. The compound of any of claims 1-19, wherein the E3 ubiquitin ligase ligand moiety is selected from

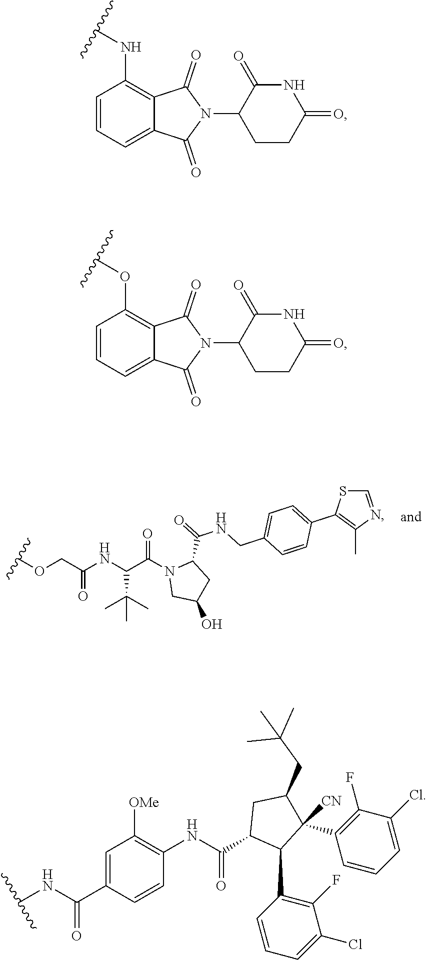

21. The compound of any of claims 1-19, wherein the compound is defined by the formula below 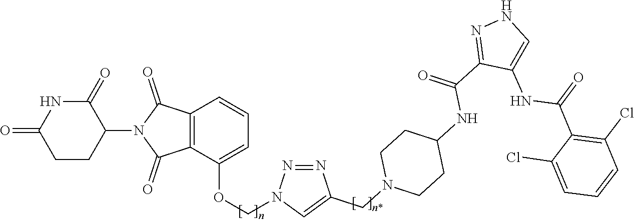
wherein n and n*are each independently an integer from 1 to 20, such as an integer from 1 to 15, an integer from 1 to 12, an integer from 1 to 10, an integer from 2 to 20, an integer from 2 to 15, an integer from 2 to 12, an integer from 2 to 10, an integer from 4 to 20, an integer from 4 to 15, an integer from 4 to 12, or an integer from 4 to 10.

22. The compound of claim 21, wherein the sum of n and n*is from 8 to 14.

23. A compound defined by Formula II below 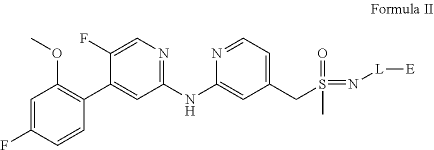
or a pharmaceutically acceptable salt, solvate, hydrate, cocrystal, or prodrug thereof wherein
L comprises one or more linking groups selected from optionally substituted —C₁₋₁₀ alkylene-, —O—C1-10 alkylene-, —C1-10 alkenylene-, —O—C1-10 alkenylene-, —C1-10 alkynylene-, —O—C1-10 alkynylene-, -arylene-, -heteroarylene-, -cycloalkylene-, -heterocycloalkylene-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl; and
E comprises an E3 ubiquitin ligase ligand moiety.

24. The compound of claim 23, wherein L comprises one or more linking groups independently selected from optionally substituted —C1-10 alkynylene-, —O—C₁₋₁₀ alkynylene-, -arylene-, -heteroarylene-, -cycloalkylene-, and -heterocycloalkylene-.

25. The compound of any of claims 23-24, wherein L comprises at least two linking groups independently selected from optionally substituted —C₁₋₁₀ alkynylene-, -heteroarylene-, and -heterocycloalkylene-.

26. The compound of any of claims 23-25, wherein L comprises at least two heterocycloalkylene groups.

27. The compound of any of claims 23-26, wherein L comprises at least one piperidine moiety.

28. The compound of any of claims 23-27, wherein L comprises at least one piperazine moiety.

29. The compound of any of claims 23-28, wherein L comprises at least one triazole moiety, such as a 1,2,3-triazole moiety.

30. The compound of any of claims 23-29, wherein L comprises at least one alkynyl moiety.

31. The compound of any of claims 23-30, wherein L comprises at least one azetidine moiety.

32. The compound of any of claims 23-31, wherein L comprises at least two moieties independently selected from a piperidine moiety, a piperazine moiety, a triazole moiety, such as a 1,2,3-triazole moiety, an alkynyl moiety, and an azetidine moiety.

33. The compound of any of claims 23-32, wherein when L comprises a piperidine moiety, L further comprises at least one additional linking group selected from optionally substituted —C₁₋₁₀ alkynylene-, —O—C₁₋₁₀ alkynylene-, -arylene-, -heteroarylene-, -cycloalkylene-, and heterocycloalkylene.

34. The compound of any of claims 23-33, wherein L has a length of at least 8 atoms or at least 10 atoms, such as a length of from 8 atoms to 25 atoms, from 10 atoms to 25 atoms, from 8 atoms to 20 atoms, or from 10 atoms to 20 atoms.

35. The compound of any of claims 23-34, wherein L is not defined by the structure below

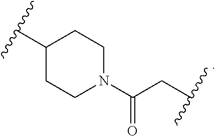 [SMILES: *CC(=O)N1CCC(*)CC1]

36. The compound of any of claims 23-35, wherein the E3 ubiquitin ligase ligand moiety comprises a Cereblon (CRBN) ligand, a mouse double minute 2 (MDM2) ligand, a Von Hippel-Lindau (VHL) ligand, or any substructure thereof.

37. The compound of claim 36, wherein the E3 ubiquitin ligase ligand comprises a CRBN ligand selected from thalidomide, lenalidomide, pomalidomide, and any substructure thereof.

38. The compound of claim 36, wherein the E3 ubiquitin ligase ligand comprises an MDM2 ligand selected from idasanutlin, RG7112, RG7388, MI 773/SAR 405838, AMG 232, DS-3032b, RO6839921, RO5045337, RO5503781, CGM-097, MK-8242, and any substructure thereof.

39. The compound of claim 36, wherein the E3 ubiquitin ligase ligand comprises a VHL ligand selected from VHL ligand 1 (VHL-1), VHL ligand 2 (VHL-2), VH032, and any substructure thereof.

40. The compound of any of claims 23-39, wherein the E3 ubiquitin ligase ligand moiety is selected from thalidomide, lenalidomide, idasanutlin, VHL ligand 1 (VHL-1), and any substructure thereof.

41. The compound of any of claims 23-40, wherein the E3 ubiquitin ligase ligand moiety is selected from 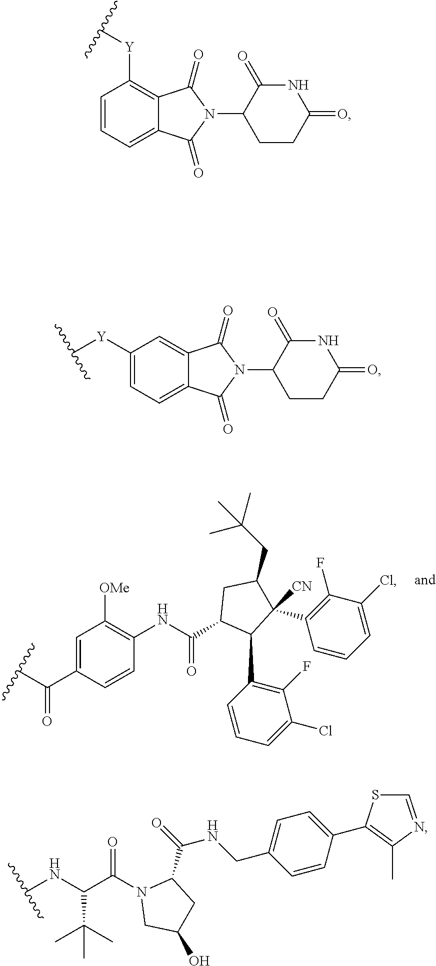
wherein Y is selected from —N(R)—, —N(H)—, and —O—, wherein R is optionally substituted C₁₋₆ alkyl.

42. The compound of any of claims 23-41, wherein the E3 ubiquitin ligase ligand moiety is selected from

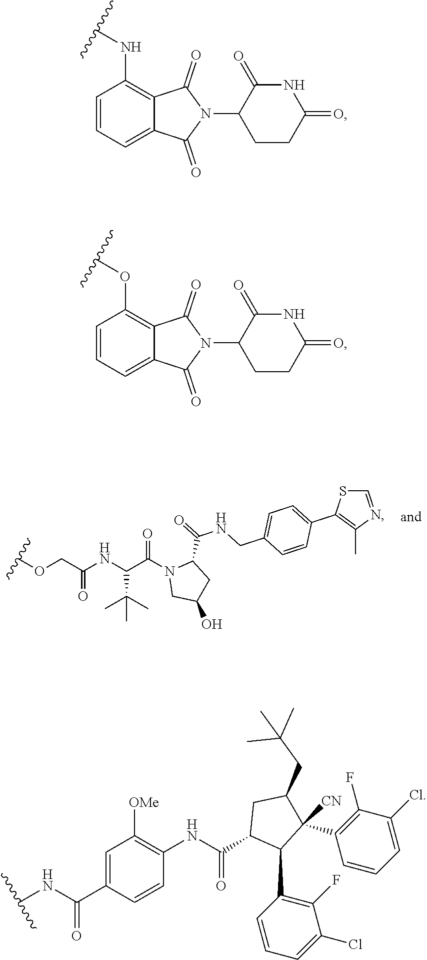

43. The compound of any of claims 23-42, wherein the compound is one of the following: 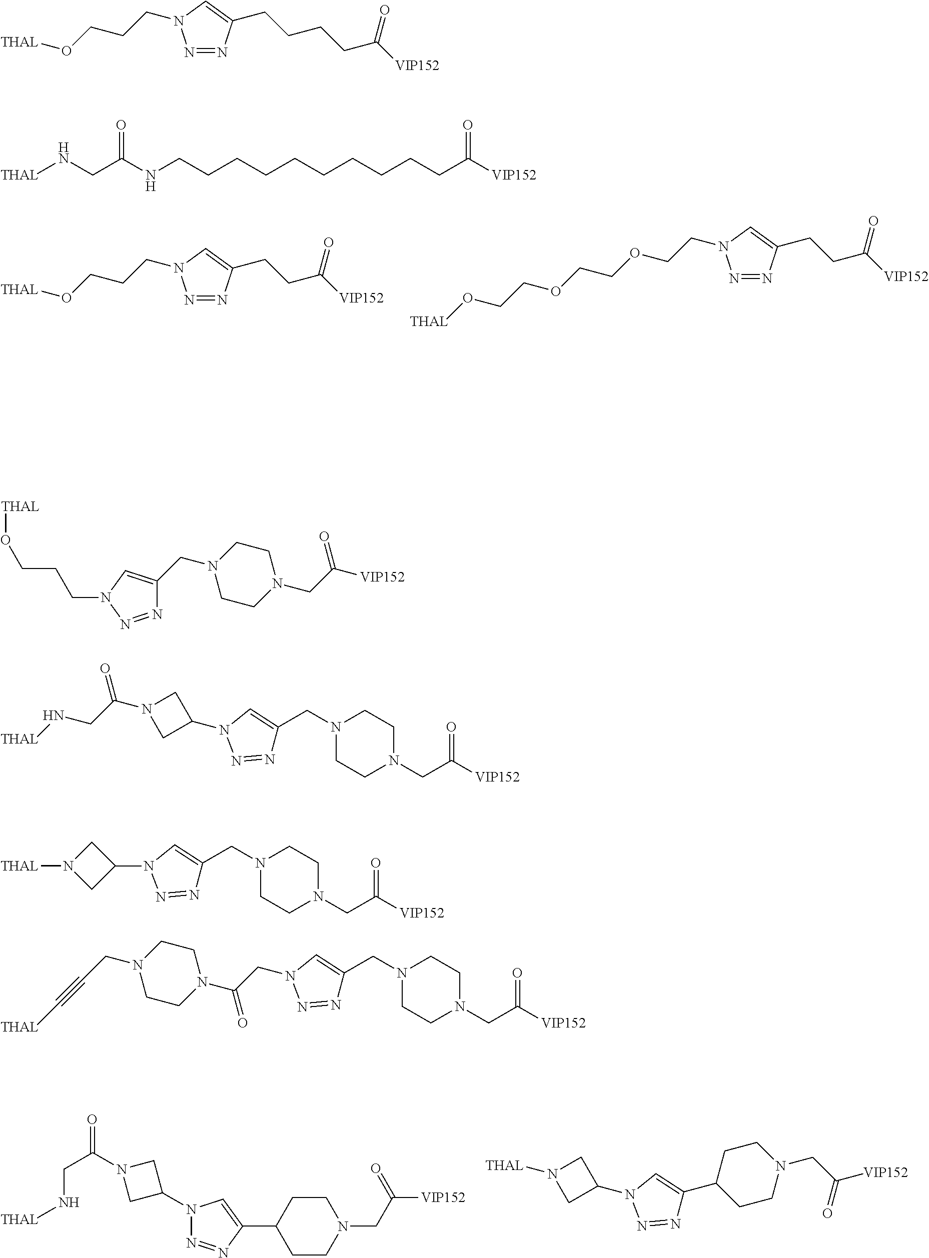 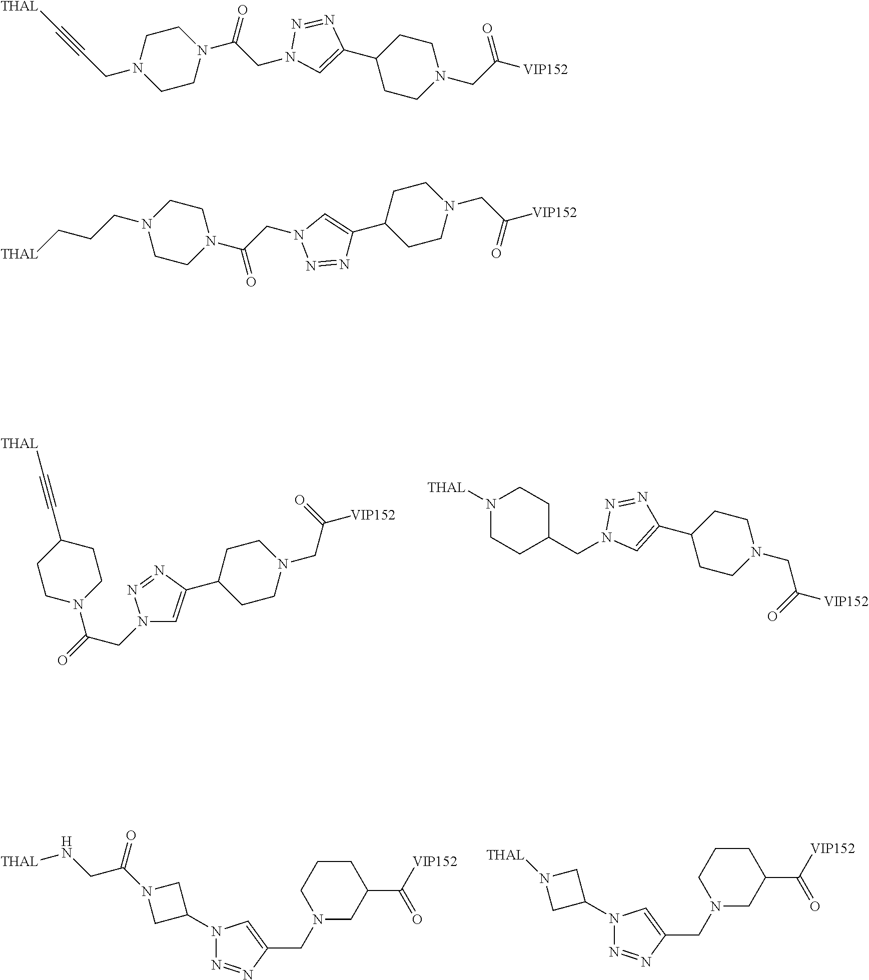 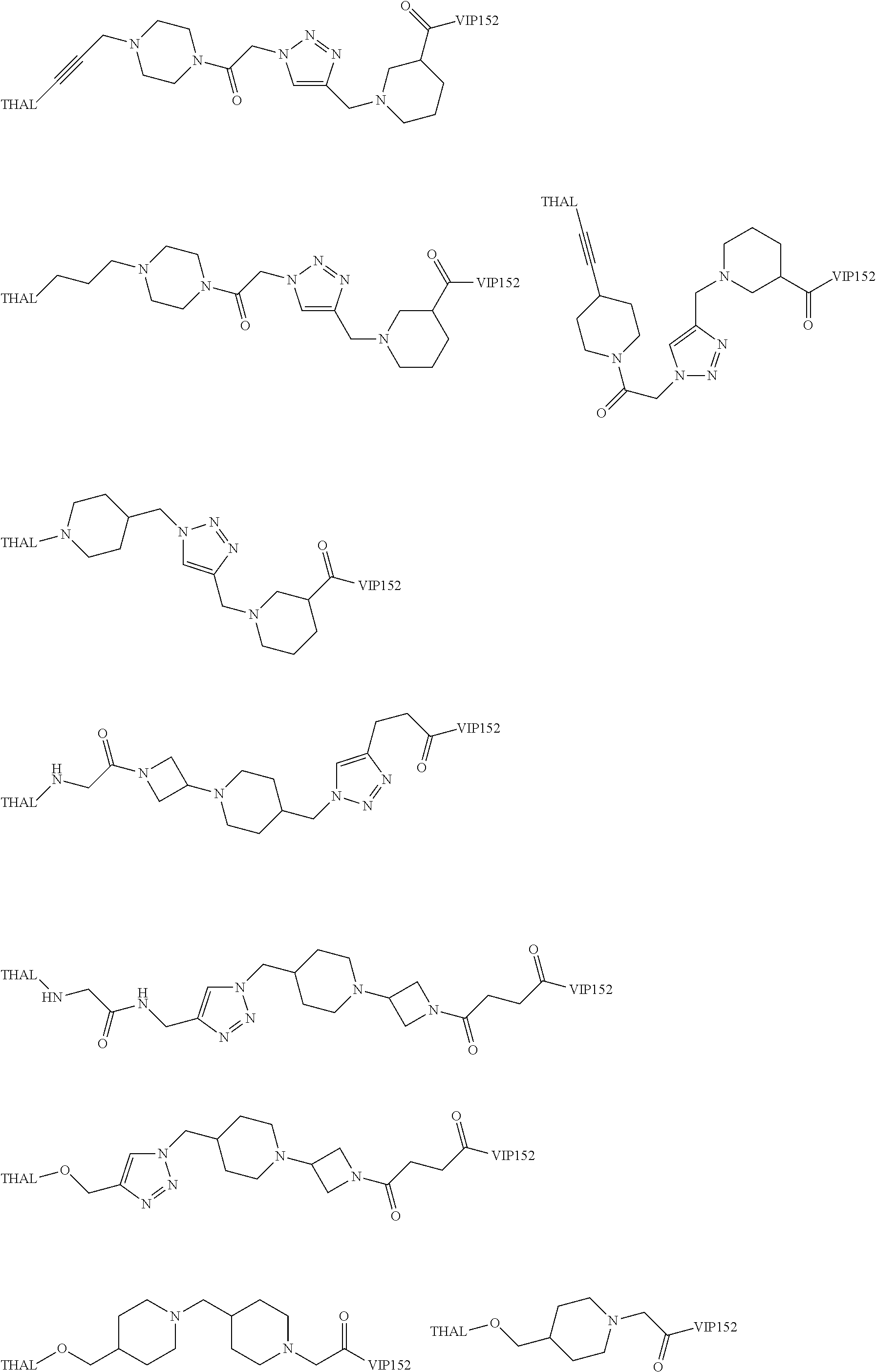 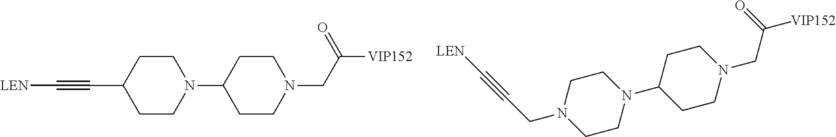
wherein THAL is 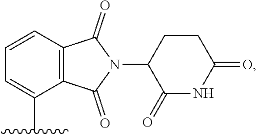 [SMILES: *c1cccc2c1C(=O)N(C1CCC(=O)NC1=O)C2=O]
LEN is 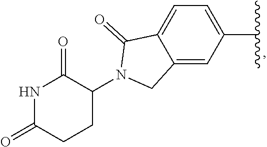 [SMILES: *c1ccc2c(c1)CN(C1CCC(=O)NC1=O)C2=O]
and VIP152 is

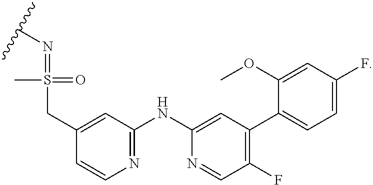 [SMILES: *N=S(C)(=O)Cc1ccnc(Nc2cc(-c3ccc(F)cc3OC)c(F)cn2)c1]

44. A pharmaceutical composition comprising one or more compounds of any one of claims 1-43 and a pharmaceutically acceptable excipient.

45. A pharmaceutical composition for treating or preventing a disease or disorder alleviated by inhibiting and/or indirectly inhibiting CDK9 protein activity, the pharmaceutical composition comprising one or more compounds according to any one of claims 1-43, or a pharmaceutically acceptable salt thereof, and a physiologically compatible carrier medium.

46. The pharmaceutical composition of claim 45, wherein the disease or disorder is cancer.

47. The pharmaceutical composition of claim 46, wherein the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

48. The pharmaceutical composition of any of claims 46-47, wherein the cancer is a blood cancer.

49. The pharmaceutical composition of claim 48, wherein the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

50. The pharmaceutical composition of any one of claims 46-49, wherein the cancer is acute myeloid leukemia (AML).

51. A pharmaceutical composition for treating or preventing acute myeloid leukemia (AML), the pharmaceutical composition comprising one or more compounds according to any one of claims 1-43, or a pharmaceutically acceptable salt thereof, and a physiologically compatible carrier medium.

52. A method of treating or preventing a disease or disorder alleviated by inhibiting and/or indirectly inhibiting CDK9 protein activity in a patient in need of said treatment or prevention, the method comprising administering a therapeutically effective amount of one or more compounds of any one of claims 1-43, or a pharmaceutically acceptable salt thereof.

53. The method of claim 52, wherein the disease or disorder is cancer.

54. The method of claim 53, wherein the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

55. The method of any of claims 53-54, wherein the cancer is a blood cancer.

56. The method of claim 55, wherein the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

57. The method of any one of claims 53-56, wherein the cancer is acute myeloid leukemia (AML).

58. A method of treating or preventing acute myeloid leukemia (AML) in a patient in need of said treatment or prevention, the method comprising administering a therapeutically effective amount of one or more compounds of any one of claims 1-43, or a pharmaceutically acceptable salt thereof.

59. A method of promoting the ubiquitination of cyclin-dependent kinase 9 (CDK9) by an E3 ubiquitin ligase, the method comprising contacting CDK9 with a compound of any one of claims 1-43, or a pharmaceutically acceptable salt thereof.

60. The method of claim 59, wherein the ubiquitination is in a cell.

61. The method of any of claims 59-60, wherein the ubiquitination is in a subject, such as a human subject or a non-human subject.

62. The method of any of claims 59-61, wherein the ubiquitination is in a biological sample.

63. The method of any one of claims 59-62, wherein the E3 ubiquitin ligase is Cereblon or von Hippel-Lindau tumor suppressor (VHL).
